SEQUENCE LISTING (887 sequences)

SEQ ID NO: 1 - protein, 116 aa, Mus musculus
EVQLQQSGPELKKPGETVKISCKASGYTFTDYSMHWVKQAPGKGLKWMGWIKTETGEPTYADDFKGRFAFSLETSATTAYLQINNLKNEDTAKYFCAIYYYGRDSWGQGTTLTVSS

SEQ ID NO: 2 - protein, 27 aa, unidentified
  REGION 1..27: note = Human (Homo sapiens), chimeric or humanized antibody peptide sequence
EVQLQQSGPELKKPGETVKISCKASGY

SEQ ID NO: 3 - protein, 8 aa, unidentified
  REGION 1..8: note = Human (Homo sapiens), chimeric or humanized antibody peptide sequence
TFTDYSMH

SEQ ID NO: 4 - protein, 14 aa, unidentified
  REGION 1..14: note = Human (Homo sapiens), chimeric or humanized antibody peptide sequence
WVKQAPGKGLKWMG

SEQ ID NO: 5 - protein, 17 aa, unidentified
  REGION 1..17: note = Human (Homo sapiens), chimeric or humanized antibody peptide sequence
WIKTETGEPTYADDFKG

SEQ ID NO: 6 - protein, 32 aa, unidentified
  REGION 1..32: note = Human (Homo sapiens), chimeric or humanized antibody peptide sequence
RFAFSLETSATTAYLQINNLKNEDTAKYFCAI

SEQ ID NO: 7 - protein, 7 aa, unidentified
  REGION 1..7: note = Human (Homo sapiens), chimeric or humanized antibody peptide sequence
YYYGRDS

SEQ ID NO: 8 - protein, 11 aa, unidentified
  REGION 1..11: note = Human (Homo sapiens), chimeric or humanized antibody peptide sequence
WGQGTTLTVSS

SEQ ID NO: 9 - protein, 116 aa, Mus musculus
QIQLLQSGPELKKPGETVKISCKASGYTFTDYSMHWVKQAPGKGLKWMGWIKTETGEPTYADDFKGRFAFSLETSATTAYLQINNLKNEDTAKYFCAIYYYGRDSWGQGTTLTVSS

SEQ ID NO: 10 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 11 - protein, 8 aa, unidentified
  REGION 1..8: note = Human (Homo sapiens), chimeric or humanized antibody peptide sequence
TFTDYSMH

SEQ ID NO: 12 - protein, 14 aa, unidentified
  REGION 1..14: note = Human (Homo sapiens), chimeric or humanized antibody peptide sequence
WVKQAPGKGLKWMG

SEQ ID NO: 13 - protein, 17 aa, unidentified
  REGION 1..17: note = Human (Homo sapiens), chimeric or humanized antibody peptide sequence
WIKTETGEPTYADDFKG

SEQ ID NO: 14 - protein, 32 aa, unidentified; identical to SEQ ID NO: 6
  REGION 1..32: note = Human (Homo sapiens), chimeric or humanized antibody peptide sequence

SEQ ID NO: 15 - protein, 7 aa, unidentified
  REGION 1..7: note = Human (Homo sapiens), chimeric or humanized antibody peptide sequence
YYYGRDS

SEQ ID NO: 16 - protein, 11 aa, unidentified
  REGION 1..11: note = Human (Homo sapiens), chimeric or humanized antibody peptide sequence
WGQGTTLTVSS

SEQ ID NO: 17 - protein, 119 aa, unidentified
  REGION 1..119: note = Human (Homo sapiens), chimeric or humanized antibody peptide sequence
QIQLLQSGPELKKPGETVKISCKASGYTFTDYSMHWVKQAPGKGLKWMGWIKTETDEPTYADDFKERFAFSLETSASTANLQIINLKNEDTATYFCAIYYYGRDSWGQGTTLTVSSSEQ

SEQ ID NO: 18 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 19 - protein, 8 aa, unidentified
  REGION 1..8: note = Human (Homo sapiens), chimeric or humanized antibody peptide sequence
TFTDYSMH

SEQ ID NO: 20 - protein, 14 aa, unidentified
  REGION 1..14: note = Human (Homo sapiens), chimeric or humanized antibody peptide sequence
WVKQAPGKGLKWMG

SEQ ID NO: 21 - protein, 17 aa, unidentified
  REGION 1..17: note = Human (Homo sapiens), chimeric or humanized antibody peptide sequence
WIKTETDEPTYADDFKE

SEQ ID NO: 22 - protein, 32 aa, unidentified
  REGION 1..32: note = Human (Homo sapiens), chimeric or humanized antibody peptide sequence
RFAFSLETSASTANLQIINLKNEDTATYFCAI

SEQ ID NO: 23 - protein, 7 aa, unidentified
  REGION 1..7: note = Human (Homo sapiens), chimeric or humanized antibody peptide sequence
YYYGRDS

SEQ ID NO: 24 - protein, 14 aa, unidentified
  REGION 1..14: note = Human (Homo sapiens), chimeric or humanized antibody peptide sequence
WGQGTTLTVSSSEQ

SEQ ID NO: 25 - protein, 116 aa, unidentified
  REGION 1..116: note = Human (Homo sapiens), chimeric or humanized antibody peptide sequence
QIQLLQSGPELKKPGETVKISCKASGYTFTDYSIHWVKQAPGKGLKWMGWIKTETGEPTYADDFNGRFAFSLETSASTAYLQINNLKNEDTATYFCAIYYYGRDSWGQGTTLTVSS

SEQ ID NO: 26 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 27 - protein, 8 aa, unidentified
  REGION 1..8: note = Human (Homo sapiens), chimeric or humanized antibody peptide sequence
TFTDYSIH

SEQ ID NO: 28 - protein, 14 aa, unidentified
  REGION 1..14: note = Human (Homo sapiens), chimeric or humanized antibody peptide sequence
WVKQAPGKGLKWMG

SEQ ID NO: 29 - protein, 17 aa, unidentified
  REGION 1..17: note = Human (Homo sapiens), chimeric or humanized antibody peptide sequence
WIKTETGEPTYADDFNG

SEQ ID NO: 30 - protein, 32 aa, unidentified
  REGION 1..32: note = Human (Homo sapiens), chimeric or humanized antibody peptide sequence
RFAFSLETSASTAYLQINNLKNEDTATYFCAI

SEQ ID NO: 31 - protein, 7 aa, unidentified
  REGION 1..7: note = Human (Homo sapiens), chimeric or humanized antibody peptide sequence
YYYGRDS

SEQ ID NO: 32 - protein, 11 aa, unidentified
  REGION 1..11: note = Human (Homo sapiens), chimeric or humanized antibody peptide sequence
WGQGTTLTVSS

SEQ ID NO: 33 - protein, 116 aa, unidentified
  REGION 1..116: note = Human (Homo sapiens), chimeric or humanized antibody peptide sequence
QIQLLQSGPELKKPGETVKISCKASGYTFTDYSMHWVKQAPGKGLKWVARINTETGEPTFADDFRGRFAVSLETSASTAYLQINNLKNEDTATYFCAIYYYGRDSWGQGTTLTVSS

SEQ ID NO: 34 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 35 - protein, 8 aa, unidentified
  REGION 1..8: note = Human (Homo sapiens), chimeric or humanized antibody peptide sequence
TFTDYSMH

SEQ ID NO: 36 - protein, 14 aa, unidentified
  REGION 1..14: note = Human (Homo sapiens), chimeric or humanized antibody peptide sequence
WVKQAPGKGLKWVA

SEQ ID NO: 37 - protein, 17 aa, unidentified
  REGION 1..17: note = Human (Homo sapiens), chimeric or humanized antibody peptide sequence
RINTETGEPTFADDFRG

SEQ ID NO: 38 - protein, 32 aa, unidentified
  REGION 1..32: note = Human (Homo sapiens), chimeric or humanized antibody peptide sequence
RFAVSLETSASTAYLQINNLKNEDTATYFCAI

SEQ ID NO: 39 - protein, 7 aa, unidentified
  REGION 1..7: note = Human (Homo sapiens), chimeric or humanized antibody peptide sequence
YYYGRDS

SEQ ID NO: 40 - protein, 11 aa, unidentified
  REGION 1..11: note = Human (Homo sapiens), chimeric or humanized antibody peptide sequence
WGQGTTLTVSS

SEQ ID NO: 41 - protein, 116 aa, unidentified
  REGION 1..116: note = Human (Homo sapiens), chimeric or humanized antibody peptide sequence
QIQLLQSGPELKKPGETVKISCLASGYTFTDYSMHWVKQAPGKGLKWVARINTETGEPTFADDFGGRFAVSLETSASTAYLQINNLKNEDTATYFCAIYYYGRDSWGQGTTLTVSS

SEQ ID NO: 42 - protein, 27 aa, unidentified
  REGION 1..27: note = Human (Homo sapiens), chimeric or humanized antibody peptide sequence
QIQLLQSGPELKKPGETVKISCLASGY

SEQ ID NO: 43 - protein, 8 aa, unidentified
  REGION 1..8: note = Human (Homo sapiens), chimeric or humanized antibody peptide sequence
TFTDYSMH

SEQ ID NO: 44 - protein, 14 aa, unidentified
  REGION 1..14: note = Human (Homo sapiens), chimeric or humanized antibody peptide sequence
WVKQAPGKGLKWVA

SEQ ID NO: 45 - protein, 17 aa, unidentified
  REGION 1..17: note = Human (Homo sapiens), chimeric or humanized antibody peptide sequence
RINTETGEPTFADDFGG

SEQ ID NO: 46 - protein, 32 aa, unidentified; identical to SEQ ID NO: 38
  REGION 1..32: note = Human (Homo sapiens), chimeric or humanized antibody peptide sequence

SEQ ID NO: 47 - protein, 7 aa, unidentified
  REGION 1..7: note = Human (Homo sapiens), chimeric or humanized antibody peptide sequence
YYYGRDS

SEQ ID NO: 48 - protein, 11 aa, unidentified
  REGION 1..11: note = Human (Homo sapiens), chimeric or humanized antibody peptide sequence
WGQGTTLTVSS

SEQ ID NO: 49 - protein, 120 aa, unidentified
  REGION 1..120: note = Human (Homo sapiens), chimeric or humanized antibody peptide sequence
EVQLVESGGGLVQPGGSLKLSCAASGFTFSSFGMSWVRQTPDKRLELVATINSNGGSTYYPDNMKGRFTISRDNAKNTLYLQMSSLKSEDTAMYYCASACYRYGAFFDYWGQGTTLTVSS

SEQ ID NO: 50 - protein, 27 aa, unidentified
  REGION 1..27: note = Human (Homo sapiens), chimeric or humanized antibody peptide sequence
EVQLVESGGGLVQPGGSLKLSCAASGF

SEQ ID NO: 51 - protein, 8 aa, unidentified
  REGION 1..8: note = Human (Homo sapiens), chimeric or humanized antibody peptide sequence
TFSSFGMS

SEQ ID NO: 52 - protein, 14 aa, unidentified
  REGION 1..14: note = Human (Homo sapiens), chimeric or humanized antibody peptide sequence
WVRQTPDKRLELVA

SEQ ID NO: 53 - protein, 17 aa, unidentified
  REGION 1..17: note = Human (Homo sapiens), chimeric or humanized antibody peptide sequence
TINSNGGSTYYPDNMKG

SEQ ID NO: 54 - protein, 32 aa, unidentified
  REGION 1..32: note = Human (Homo sapiens), chimeric or humanized antibody peptide sequence
RFTISRDNAKNTLYLQMSSLKSEDTAMYYCAS

SEQ ID NO: 55 - protein, 11 aa, unidentified
  REGION 1..11: note = Human (Homo sapiens), chimeric or humanized antibody peptide sequence
ACYRYGAFFDY

SEQ ID NO: 56 - protein, 11 aa, unidentified
  REGION 1..11: note = Human (Homo sapiens), chimeric or humanized antibody peptide sequence
WGQGTTLTVSS

SEQ ID NO: 57 - protein, 116 aa, unidentified
  REGION 1..116: note = Human (Homo sapiens), chimeric or humanized antibody peptide sequence
EVQLLESGPELKKPGETVKISCKASGYTFTDYSIHWVKQAPGKGLKWMGWIKTETGEPTYADDFKGRFAFSLETSASTAYLQINNLKNEDTATYFCAIYYYGRDSWGQGTTVTVSS

SEQ ID NO: 58 - protein, 27 aa, unidentified
  REGION 1..27: note = Human (Homo sapiens), chimeric or humanized antibody peptide sequence
EVQLLESGPELKKPGETVKISCKASGY

SEQ ID NO: 59 - protein, 8 aa, unidentified
  REGION 1..8: note = Human (Homo sapiens), chimeric or humanized antibody peptide sequence
TFTDYSIH

SEQ ID NO: 60 - protein, 14 aa, unidentified
  REGION 1..14: note = Human (Homo sapiens), chimeric or humanized antibody peptide sequence
WVKQAPGKGLKWMG

SEQ ID NO: 61 - protein, 17 aa, unidentified
  REGION 1..17: note = Human (Homo sapiens), chimeric or humanized antibody peptide sequence
WIKTETGEPTYADDFKG

SEQ ID NO: 62 - protein, 32 aa, unidentified; identical to SEQ ID NO: 30
  REGION 1..32: note = Human (Homo sapiens), chimeric or humanized antibody peptide sequence

SEQ ID NO: 63 - protein, 7 aa, unidentified
  REGION 1..7: note = Human (Homo sapiens), chimeric or humanized antibody peptide sequence
YYYGRDS

SEQ ID NO: 64 - protein, 11 aa, unidentified
  REGION 1..11: note = Human (Homo sapiens), chimeric or humanized antibody peptide sequence
WGQGTTVTVSS

SEQ ID NO: 65 - protein, 116 aa, unidentified; identical to SEQ ID NO: 57
  REGION 1..116: note = Human (Homo sapiens), chimeric or humanized antibody peptide sequence

SEQ ID NO: 66 - protein, 27 aa, unidentified
  REGION 1..27: note = Human (Homo sapiens), chimeric or humanized antibody peptide sequence
EVQLLESGPELKKPGETVKISCKASGY

SEQ ID NO: 67 - protein, 8 aa, unidentified
  REGION 1..8: note = Human (Homo sapiens), chimeric or humanized antibody peptide sequence
TFTDYSIH

SEQ ID NO: 68 - protein, 14 aa, unidentified
  REGION 1..14: note = Human (Homo sapiens), chimeric or humanized antibody peptide sequence
WVKQAPGKGLKWMG

SEQ ID NO: 69 - protein, 17 aa, unidentified
  REGION 1..17: note = Human (Homo sapiens), chimeric or humanized antibody peptide sequence
WIKTETGEPTYADDFKG

SEQ ID NO: 70 - protein, 32 aa, unidentified; identical to SEQ ID NO: 30
  REGION 1..32: note = Human (Homo sapiens), chimeric or humanized antibody peptide sequence

SEQ ID NO: 71 - protein, 7 aa, unidentified
  REGION 1..7: note = Human (Homo sapiens), chimeric or humanized antibody peptide sequence
YYYGRDS

SEQ ID NO: 72 - protein, 11 aa, unidentified
  REGION 1..11: note = Human (Homo sapiens), chimeric or humanized antibody peptide sequence
WGQGTTVTVSS

SEQ ID NO: 73 - protein, 116 aa, unidentified
  REGION 1..116: note = Human (Homo sapiens), chimeric or humanized antibody peptide sequence
QVQLMQSGPELKKPGETVKISCKASGYTFTDYSIHWVKQAPGKGLKWMGWIKTETGEPTYADDFNGRFAFSLETSASTAYLQINNLKNEDTATYFCAIYYYGRDSWGQGTTLTVSS

SEQ ID NO: 74 - protein, 27 aa, unidentified
  REGION 1..27: note = Human (Homo sapiens), chimeric or humanized antibody peptide sequence
QVQLMQSGPELKKPGETVKISCKASGY

SEQ ID NO: 75 - protein, 8 aa, unidentified
  REGION 1..8: note = Human (Homo sapiens), chimeric or humanized antibody peptide sequence
TFTDYSIH

SEQ ID NO: 76 - protein, 14 aa, unidentified
  REGION 1..14: note = Human (Homo sapiens), chimeric or humanized antibody peptide sequence
WVKQAPGKGLKWMG

SEQ ID NO: 77 - protein, 17 aa, unidentified
  REGION 1..17: note = Human (Homo sapiens), chimeric or humanized antibody peptide sequence
WIKTETGEPTYADDFNG

SEQ ID NO: 78 - protein, 32 aa, unidentified; identical to SEQ ID NO: 30
  REGION 1..32: note = Human (Homo sapiens), chimeric or humanized antibody peptide sequence

SEQ ID NO: 79 - protein, 7 aa, unidentified
  REGION 1..7: note = Human (Homo sapiens), chimeric or humanized antibody peptide sequence
YYYGRDS

SEQ ID NO: 80 - protein, 11 aa, unidentified
  REGION 1..11: note = Human (Homo sapiens), chimeric or humanized antibody peptide sequence
WGQGTTLTVSS

SEQ ID NO: 81 - protein, 116 aa, unidentified
  REGION 1..116: note = Human (Homo sapiens), chimeric or humanized antibody peptide sequence
QIQLQQSGPELKKPGETVKISCKASGYTFTDYSMHWVKQAPGKGLKWVARINTETGEPTFADDFRGRFAVSLETSASTAYLQINNLKNEDTATYFCAIYYYGRDSWGQGTTLTVSS

SEQ ID NO: 82 - protein, 27 aa, unidentified
  REGION 1..27: note = Human (Homo sapiens), chimeric or humanized antibody peptide sequence
QIQLQQSGPELKKPGETVKISCKASGY

SEQ ID NO: 83 - protein, 8 aa, unidentified
  REGION 1..8: note = Human (Homo sapiens), chimeric or humanized antibody peptide sequence
TFTDYSMH

SEQ ID NO: 84 - protein, 14 aa, unidentified
  REGION 1..14: note = Human (Homo sapiens), chimeric or humanized antibody peptide sequence
WVKQAPGKGLKWVA

SEQ ID NO: 85 - protein, 17 aa, unidentified
  REGION 1..17: note = Human (Homo sapiens), chimeric or humanized antibody peptide sequence
RINTETGEPTFADDFRG

SEQ ID NO: 86 - protein, 32 aa, unidentified; identical to SEQ ID NO: 38
  REGION 1..32: note = Human (Homo sapiens), chimeric or humanized antibody peptide sequence

SEQ ID NO: 87 - protein, 7 aa, unidentified
  REGION 1..7: note = Human (Homo sapiens), chimeric or humanized antibody peptide sequence
YYYGRDS

SEQ ID NO: 88 - protein, 11 aa, unidentified
  REGION 1..11: note = Human (Homo sapiens), chimeric or humanized antibody peptide sequence
WGQGTTLTVSS

SEQ ID NO: 89 - protein, 116 aa, unidentified; identical to SEQ ID NO: 33
  REGION 1..116: note = Human (Homo sapiens), chimeric or humanized antibody peptide sequence

SEQ ID NO: 90 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 91 - protein, 8 aa, unidentified
  REGION 1..8: note = Human (Homo sapiens), chimeric or humanized antibody peptide sequence
TFTDYSMH

SEQ ID NO: 92 - protein, 14 aa, unidentified
  REGION 1..14: note = Human (Homo sapiens), chimeric or humanized antibody peptide sequence
WVKQAPGKGLKWVA

SEQ ID NO: 93 - protein, 17 aa, unidentified
  REGION 1..17: note = Human (Homo sapiens), chimeric or humanized antibody peptide sequence
RINTETGEPTFADDFRG

SEQ ID NO: 94 - protein, 32 aa, unidentified; identical to SEQ ID NO: 38
  REGION 1..32: note = Human (Homo sapiens), chimeric or humanized antibody peptide sequence

SEQ ID NO: 95 - protein, 7 aa, unidentified
  REGION 1..7: note = Human (Homo sapiens), chimeric or humanized antibody peptide sequence
YYYGRDS

SEQ ID NO: 96 - protein, 11 aa, unidentified
  REGION 1..11: note = Human (Homo sapiens), chimeric or humanized antibody peptide sequence
WGQGTTLTVSS

SEQ ID NO: 97 - protein, 116 aa, unidentified
  REGION 1..116: note = Human (Homo sapiens), chimeric or humanized antibody peptide sequence
QIQLLQSGPELKKPGETVKISCKASGYTFTDYSMHWVKQAPGKGLKWVARINTETGEPTFADDFRGRFAVSLETSASTAYLQINNLKNEDTATYFCAIYYYGRDTWGQGTTLSVSS

SEQ ID NO: 98 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 99 - protein, 8 aa, unidentified
  REGION 1..8: note = Human (Homo sapiens), chimeric or humanized antibody peptide sequence
TFTDYSMH

SEQ ID NO: 100 - protein, 14 aa, unidentified
  REGION 1..14: note = Human (Homo sapiens), chimeric or humanized antibody peptide sequence
WVKQAPGKGLKWVA

SEQ ID NO: 101 - protein, 17 aa, unidentified
  REGION 1..17: note = Human (Homo sapiens), chimeric or humanized antibody peptide sequence
RINTETGEPTFADDFRG

SEQ ID NO: 102 - protein, 32 aa, unidentified; identical to SEQ ID NO: 38
  REGION 1..32: note = Human (Homo sapiens), chimeric or humanized antibody peptide sequence

SEQ ID NO: 103 - protein, 7 aa, unidentified
  REGION 1..7: note = Human (Homo sapiens), chimeric or humanized antibody peptide sequence
YYYGRDT

SEQ ID NO: 104 - protein, 11 aa, unidentified
  REGION 1..11: note = Human (Homo sapiens), chimeric or humanized antibody peptide sequence
WGQGTTLSVSS

SEQ ID NO: 105 - protein, 116 aa, unidentified
  REGION 1..116: note = Human (Homo sapiens), chimeric or humanized antibody peptide sequence
QIQLLQSGPELKKPGETVKISCKASGYTFTDYSMHWVKQAPGKGLKWVARINTETGEPTFADDFRGRFAVSLETSASTAYLQINNLKNEDTATYFCAIFYYGRDSWGQGTALTVSS

SEQ ID NO: 106 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 107 - protein, 8 aa, unidentified
  REGION 1..8: note = Human (Homo sapiens), chimeric or humanized antibody peptide sequence
TFTDYSMH

SEQ ID NO: 108 - protein, 14 aa, unidentified
  REGION 1..14: note = Human (Homo sapiens), chimeric or humanized antibody peptide sequence
WVKQAPGKGLKWVA

SEQ ID NO: 109 - protein, 17 aa, unidentified
  REGION 1..17: note = Human (Homo sapiens), chimeric or humanized antibody peptide sequence
RINTETGEPTFADDFRG

SEQ ID NO: 110 - protein, 32 aa, unidentified; identical to SEQ ID NO: 38
  REGION 1..32: note = Human (Homo sapiens), chimeric or humanized antibody peptide sequence

SEQ ID NO: 111 - protein, 7 aa, unidentified
  REGION 1..7: note = Human (Homo sapiens), chimeric or humanized antibody peptide sequence
FYYGRDS

SEQ ID NO: 112 - protein, 11 aa, unidentified
  REGION 1..11: note = Human (Homo sapiens), chimeric or humanized antibody peptide sequence
WGQGTALTVSS

SEQ ID NO: 113 - protein, 116 aa, unidentified; identical to SEQ ID NO: 33
  REGION 1..116: note = Human (Homo sapiens), chimeric or humanized antibody peptide sequence

SEQ ID NO: 114 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 115 - protein, 8 aa, unidentified
  REGION 1..8: note = Human (Homo sapiens), chimeric or humanized antibody peptide sequence
TFTDYSMH

SEQ ID NO: 116 - protein, 14 aa, unidentified
  REGION 1..14: note = Human (Homo sapiens), chimeric or humanized antibody peptide sequence
WVKQAPGKGLKWVA

SEQ ID NO: 117 - protein, 17 aa, unidentified
  REGION 1..17: note = Human (Homo sapiens), chimeric or humanized antibody peptide sequence
RINTETGEPTFADDFRG

SEQ ID NO: 118 - protein, 32 aa, unidentified; identical to SEQ ID NO: 38
  REGION 1..32: note = Human (Homo sapiens), chimeric or humanized antibody peptide sequence

SEQ ID NO: 119 - protein, 7 aa, unidentified
  REGION 1..7: note = Human (Homo sapiens), chimeric or humanized antibody peptide sequence
YYYGRDS

SEQ ID NO: 120 - protein, 11 aa, unidentified
  REGION 1..11: note = Human (Homo sapiens), chimeric or humanized antibody peptide sequence
WGQGTTLTVSS

SEQ ID NO: 121 - protein, 116 aa, unidentified
  REGION 1..116: note = Human (Homo sapiens), chimeric or humanized antibody peptide sequence
QIQLLQSGPELKKPGETVKISCKASGYTFTDYSMHWVKQAPGKGLKWVARINTETGEPTFADDFRGRFAVSLETSASTAYLQINNLKNEDTATYFCAIFYYGRDSWGQGTTLTVSS

SEQ ID NO: 122 - protein, 27 aa, unidentified
  REGION 1..27: note = Human (Homo sapiens), chimeric or humanized antibody peptide sequence
QIQLLQSGPELKKPGETVKISCKASGY

SEQ ID NO: 123 - protein, 8 aa, unidentified
  REGION 1..8: note = Human (Homo sapiens), chimeric or humanized antibody peptide sequence
TFTDYSMH

SEQ ID NO: 124 - protein, 14 aa, unidentified
  REGION 1..14: note = Human (Homo sapiens), chimeric or humanized antibody peptide sequence
WVKQAPGKGLKWVA

SEQ ID NO: 125 - protein, 17 aa, unidentified
  REGION 1..17: note = Human (Homo sapiens), chimeric or humanized antibody peptide sequence
RINTETGEPTFADDFRG

SEQ ID NO: 126 - protein, 32 aa, unidentified; identical to SEQ ID NO: 38
  REGION 1..32: note = Human (Homo sapiens), chimeric or humanized antibody peptide sequence

SEQ ID NO: 127 - protein, 7 aa, unidentified
  REGION 1..7: note = Human (Homo sapiens), chimeric or humanized antibody peptide sequence
FYYGRDS

SEQ ID NO: 128 - protein, 11 aa, unidentified
  REGION 1..11: note = Human (Homo sapiens), chimeric or humanized antibody peptide sequence
WGQGTTLTVSS

SEQ ID NO: 129 - protein, 109 aa, unidentified
  REGION 1..109: note = Human (Homo sapiens), chimeric or humanized antibody peptide sequence
DIVMSQSPSSMYASLGERVTITCKASQDINSYLSWFQQKPGKSPKTLIYRANRLVDGVPSRFSGSGSGQDYSLTISSLEYEDMGIYYCLQYDEFPPLTFGAGTKLELKA

SEQ ID NO: 130 - protein, 23 aa, unidentified
  REGION 1..23: note = Human (Homo sapiens), chimeric or humanized antibody peptide sequence
DIVMSQSPSSMYASLGERVTITC

SEQ ID NO: 131 - protein, 11 aa, unidentified
  REGION 1..11: note = Human (Homo sapiens), chimeric or humanized antibody peptide sequence
KASQDINSYLS

SEQ ID NO: 132 - protein, 15 aa, unidentified
  REGION 1..15: note = Human (Homo sapiens), chimeric or humanized antibody peptide sequence
WFQQKPGKSPKTLIY

SEQ ID NO: 133 - protein, 7 aa, unidentified
  REGION 1..7: note = Human (Homo sapiens), chimeric or humanized antibody peptide sequence
RANRLVD

SEQ ID NO: 134 - protein, 32 aa, unidentified
  REGION 1..32: note = Human (Homo sapiens), chimeric or humanized antibody peptide sequence
GVPSRFSGSGSGQDYSLTISSLEYEDMGIYYC

SEQ ID NO: 135 - protein, 10 aa, unidentified
  REGION 1..10: note = Human (Homo sapiens), chimeric or humanized antibody peptide sequence
LQYDEFPPLT

SEQ ID NO: 136 - protein, 11 aa, unidentified
  REGION 1..11: note = Human (Homo sapiens), chimeric or humanized antibody peptide sequence
FGAGTKLELKA

SEQ ID NO: 137 - protein, 109 aa, unidentified
  REGION 1..109: note = Human (Homo sapiens), chimeric or humanized antibody peptide sequence
QIVLTQSPSSMYASLGERVTITCKASQDINSYLSWFQQKPGKSPKTLIYRANRLVDGVPSRFSGSGSGQDYSLTISSLEYEDMGIYYCLQYDEFPPLTFGAGTKLELKA

SEQ ID NO: 138 - protein, 23 aa, unidentified
  REGION 1..23: note = Human (Homo sapiens), chimeric or humanized antibody peptide sequence
QIVLTQSPSSMYASLGERVTITC

SEQ ID NO: 139 - protein, 11 aa, unidentified
  REGION 1..11: note = Human (Homo sapiens), chimeric or humanized antibody peptide sequence
KASQDINSYLS

SEQ ID NO: 140 - protein, 15 aa, unidentified
  REGION 1..15: note = Human (Homo sapiens), chimeric or humanized antibody peptide sequence
WFQQKPGKSPKTLIY

SEQ ID NO: 141 - protein, 7 aa, unidentified
  REGION 1..7: note = Human (Homo sapiens), chimeric or humanized antibody peptide sequence
RANRLVD

SEQ ID NO: 142 - protein, 32 aa, unidentified; identical to SEQ ID NO: 134
  REGION 1..32: note = Human (Homo sapiens), chimeric or humanized antibody peptide sequence

SEQ ID NO: 143 - protein, 10 aa, unidentified
  REGION 1..10: note = Human (Homo sapiens), chimeric or humanized antibody peptide sequence
LQYDEFPPLT

SEQ ID NO: 144 - protein, 11 aa, unidentified
  REGION 1..11: note = Human (Homo sapiens), chimeric or humanized antibody peptide sequence
FGAGTKLELKA

SEQ ID NO: 145 - protein, 107 aa, unidentified
  REGION 1..107: note = Human (Homo sapiens), chimeric or humanized antibody peptide sequence
QIVLTQSPAIMSASPGEKVTMTCSASSSVSYMHWYQQKPGTSPKLWIYDTSNLASGVPARFSGSGSGTSYSLTISSMEAEDAATYYCQQWSSNPLTFGSGTKLEIKA

SEQ ID NO: 146 - protein, 23 aa, unidentified
  REGION 1..23: note = Human (Homo sapiens), chimeric or humanized antibody peptide sequence
QIVLTQSPAIMSASPGEKVTMTC

SEQ ID NO: 147 - protein, 10 aa, unidentified
  REGION 1..10: note = Human (Homo sapiens), chimeric or humanized antibody peptide sequence
SASSSVSYMH

SEQ ID NO: 148 - protein, 15 aa, unidentified
  REGION 1..15: note = Human (Homo sapiens), chimeric or humanized antibody peptide sequence
WYQQKPGTSPKLWIY

SEQ ID NO: 149 - protein, 7 aa, unidentified
  REGION 1..7: note = Human (Homo sapiens), chimeric or humanized antibody peptide sequence
DTSNLAS

SEQ ID NO: 150 - protein, 32 aa, unidentified
  REGION 1..32: note = Human (Homo sapiens), chimeric or humanized antibody peptide sequence
GVPARFSGSGSGTSYSLTISSMEAEDAATYYC

SEQ ID NO: 151 - protein, 9 aa, unidentified
  REGION 1..9: note = Human (Homo sapiens), chimeric or humanized antibody peptide sequence
QQWSSNPLT

SEQ ID NO: 152 - protein, 11 aa, unidentified
  REGION 1..11: note = Human (Homo sapiens), chimeric or humanized antibody peptide sequence
FGSGTKLEIKA

SEQ ID NO: 153 - protein, 107 aa, unidentified
  REGION 1..107: note = Human (Homo sapiens), chimeric or humanized antibody peptide sequence
EIVLTQSPAIMSASPGEKVTMTCSASSSVSYMHWYQQKPGSSPKLWIYDTSNLASGVPARFSGSGSGTSYSLTISSMEAEDAATYYCQQWSSNPLTFGDGTRLEIKA

SEQ ID NO: 154 - protein, 23 aa, unidentified
  REGION 1..23: note = Human (Homo sapiens), chimeric or humanized antibody peptide sequence
EIVLTQSPAIMSASPGEKVTMTC

SEQ ID NO: 155 - protein, 10 aa, unidentified
  REGION 1..10: note = Human (Homo sapiens), chimeric or humanized antibody peptide sequence
SASSSVSYMH

SEQ ID NO: 156 - protein, 15 aa, unidentified
  REGION 1..15: note = Human (Homo sapiens), chimeric or humanized antibody peptide sequence
WYQQKPGSSPKLWIY

SEQ ID NO: 157 - protein, 7 aa, unidentified
  REGION 1..7: note = Human (Homo sapiens), chimeric or humanized antibody peptide sequence
DTSNLAS

SEQ ID NO: 158 - protein, 32 aa, unidentified; identical to SEQ ID NO: 150
  REGION 1..32: note = Human (Homo sapiens), chimeric or humanized antibody peptide sequence

SEQ ID NO: 159 - protein, 9 aa, unidentified
  REGION 1..9: note = Human (Homo sapiens), chimeric or humanized antibody peptide sequence
QQWSSNPLT

SEQ ID NO: 160 - protein, 11 aa, unidentified
  REGION 1..11: note = Human (Homo sapiens), chimeric or humanized antibody peptide sequence
FGDGTRLEIKA

SEQ ID NO: 161 - protein, 107 aa, unidentified
  REGION 1..107: note = Human (Homo sapiens), chimeric or humanized antibody peptide sequence
QIVLTQSPAIMSASPGEKVTMTCSASSSVSYMHWYQQKSGTSPKLWIYDTSNLASGVPARFSGSGSGTSYSLTISSMEAEDAATYYCQQWSSNPPTFGAGTKLELKA

SEQ ID NO: 162 - protein, 23 aa, unidentified
  REGION 1..23: note = Human (Homo sapiens), chimeric or humanized antibody peptide sequence
QIVLTQSPAIMSASPGEKVTMTC

SEQ ID NO: 163 - protein, 10 aa, unidentified
  REGION 1..10: note = Human (Homo sapiens), chimeric or humanized antibody peptide sequence
SASSSVSYMH

SEQ ID NO: 164 - protein, 15 aa, unidentified
  REGION 1..15: note = Human (Homo sapiens), chimeric or humanized antibody peptide sequence
WYQQKSGTSPKLWIY

SEQ ID NO: 165 - protein, 7 aa, unidentified
  REGION 1..7: note = Human (Homo sapiens), chimeric or humanized antibody peptide sequence
DTSNLAS

SEQ ID NO: 166 - protein, 32 aa, unidentified; identical to SEQ ID NO: 150
  REGION 1..32: note = Human (Homo sapiens), chimeric or humanized antibody peptide sequence

SEQ ID NO: 167 - protein, 9 aa, unidentified
  REGION 1..9: note = Human (Homo sapiens), chimeric or humanized antibody peptide sequence
QQWSSNPPT

SEQ ID NO: 168 - protein, 11 aa, unidentified
  REGION 1..11: note = Human (Homo sapiens), chimeric or humanized antibody peptide sequence
FGAGTKLELKA

SEQ ID NO: 169 - protein, 107 aa, unidentified
  REGION 1..107: note = Human (Homo sapiens), chimeric or humanized antibody peptide sequence
DIKMTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKA

SEQ ID NO: 170 - protein, 23 aa, unidentified
  REGION 1..23: note = Human (Homo sapiens), chimeric or humanized antibody peptide sequence
DIKMTQSPAIMSASPGEKVTMTC

SEQ ID NO: 171 - protein, 10 aa, unidentified
  REGION 1..10: note = Human (Homo sapiens), chimeric or humanized antibody peptide sequence
SASSSVSYMH

SEQ ID NO: 172 - protein, 15 aa, unidentified
  REGION 1..15: note = Human (Homo sapiens), chimeric or humanized antibody peptide sequence
WYQQKSGTSPKRWIY

SEQ ID NO: 173 - protein, 7 aa, unidentified
  REGION 1..7: note = Human (Homo sapiens), chimeric or humanized antibody peptide sequence
DTSKLAS

SEQ ID NO: 174 - protein, 32 aa, unidentified; identical to SEQ ID NO: 150
  REGION 1..32: note = Human (Homo sapiens), chimeric or humanized antibody peptide sequence

SEQ ID NO: 175 - protein, 9 aa, unidentified
  REGION 1..9: note = Human (Homo sapiens), chimeric or humanized antibody peptide sequence
QQWSSNPFT

SEQ ID NO: 176 - protein, 11 aa, unidentified
  REGION 1..11: note = Human (Homo sapiens), chimeric or humanized antibody peptide sequence
FGSGTKLEIKA

SEQ ID NO: 177 - protein, 107 aa, unidentified
  REGION 1..107: note = Human (Homo sapiens), chimeric or humanized antibody peptide sequence
QMVLTHSPAIMSASPGEKVTMTCSASSSVSYMHWYQQKPGSSPKPWIYGTSNLASGVPARFSGSGSGTSYSLTISRMEAEDAATYYCQQWSSNPPTFGDGTRLEIKA

SEQ ID NO: 178 - protein, 23 aa, unidentified
  REGION 1..23: note = Human (Homo sapiens), chimeric or humanized antibody peptide sequence
QMVLTHSPAIMSASPGEKVTMTC

SEQ ID NO: 179 - protein, 10 aa, unidentified
  REGION 1..10: note = Human (Homo sapiens), chimeric or humanized antibody peptide sequence
SASSSVSYMH

SEQ ID NO: 180 - protein, 15 aa, unidentified
  REGION 1..15: note = Human (Homo sapiens), chimeric or humanized antibody peptide sequence
WYQQKPGSSPKPWIY

SEQ ID NO: 181 - protein, 7 aa, unidentified
  REGION 1..7: note = Human (Homo sapiens), chimeric or humanized antibody peptide sequence
GTSNLAS

SEQ ID NO: 182 - protein, 32 aa, unidentified
  REGION 1..32: note = Human (Homo sapiens), chimeric or humanized antibody peptide sequence
GVPARFSGSGSGTSYSLTISRMEAEDAATYYC

SEQ ID NO: 183 - protein, 9 aa, unidentified
  REGION 1..9: note = Human (Homo sapiens), chimeric or humanized antibody peptide sequence
QQWSSNPPT

SEQ ID NO: 184 - protein, 11 aa, unidentified
  REGION 1..11: note = Human (Homo sapiens), chimeric or humanized antibody peptide sequence
FGDGTRLEIKA

SEQ ID NO: 185 - protein, 113 aa, unidentified
  REGION 1..113: note = Human (Homo sapiens), chimeric or humanized antibody peptide sequence
DIVMSQSPSSLAVSAGEKVTMSCKSSQSLLNSRTRKNYLAWYQQKPGQSPRLLIYWASTRESGVPDRFTGSGSGTDFTLTISSVQAEDLAVYYCKQSYNLLTFGAGTKLELKA

SEQ ID NO: 186 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 187 - protein, 17 aa, unidentified
  REGION 1..17: note = Human (Homo sapiens), chimeric or humanized antibody peptide sequence
KSSQSLLNSRTRKNYLA

SEQ ID NO: 188 - protein, 15 aa, unidentified
  REGION 1..15: note = Human (Homo sapiens), chimeric or humanized antibody peptide sequence
WYQQKPGQSPRLLIY

SEQ ID NO: 189 - protein, 7 aa, unidentified
  REGION 1..7: note = Human (Homo sapiens), chimeric or humanized antibody peptide sequence
WASTRES

SEQ ID NO: 190 - protein, 32 aa, unidentified
  REGION 1..32: note = Human (Homo sapiens), chimeric or humanized antibody peptide sequence
GVPDRFTGSGSGTDFTLTISSVQAEDLAVYYC

SEQ ID NO: 191 - protein, 8 aa, unidentified
  REGION 1..8: note = Human (Homo sapiens), chimeric or humanized antibody peptide sequence
KQSYNLLT

SEQ ID NO: 192 - protein, 11 aa, unidentified
  REGION 1..11: note = Human (Homo sapiens), chimeric or humanized antibody peptide sequence
FGAGTKLELKA

SEQ ID NO: 193 - protein, 113 aa, unidentified
  REGION 1..113: note = Human (Homo sapiens), chimeric or humanized antibody peptide sequence
DIVMSQSPSSLAVSAGENVTVSCKSSQSLLNSRTRKNYLAWYQQKPGQSPKLLIYWASTRESGVPDRFTGSGSGTDFTLTISSVQAEDLAVYFCKQSYNLLTFGAGTKLELKA

SEQ ID NO: 194 - protein, 23 aa, unidentified
  REGION 1..23: note = Human (Homo sapiens), chimeric or humanized antibody peptide sequence
DIVMSQSPSSLAVSAGENVTVSC

SEQ ID NO: 195 - protein, 23 aa, unidentified
  REGION 1..23: note = Human (Homo sapiens), chimeric or humanized antibody peptide sequence
DIVMSQSPSSLAVSAGENVTVSC

SEQ ID NO: 196 - protein, 15 aa, unidentified
  REGION 1..15: note = Human (Homo sapiens), chimeric or humanized antibody peptide sequence
WYQQKPGQSPKLLIY

SEQ ID NO: 197 - protein, 7 aa, unidentified
  REGION 1..7: note = Human (Homo sapiens), chimeric or humanized antibody peptide sequence
WASTRES

SEQ ID NO: 198 - protein, 32 aa, unidentified
  REGION 1..32: note = Human (Homo sapiens), chimeric or humanized antibody peptide sequence
GVPDRFTGSGSGTDFTLTISSVQAEDLAVYFC

SEQ ID NO: 199 - protein, 8 aa, unidentified
  REGION 1..8: note = Human (Homo sapiens), chimeric or humanized antibody peptide sequence
KQSYNLLT

SEQ ID NO: 200 - protein, 11 aa, unidentified
  REGION 1..11: note = Human (Homo sapiens), chimeric or humanized antibody peptide sequence
FGAGTKLELKA

SEQ ID NO: 201 - protein, 113 aa, unidentified
  REGION 1..113: note = Human (Homo sapiens), chimeric or humanized antibody peptide sequence
DIKMTQSPSSLAVSPGEKVTMSCKSSQSLLHSRTRKNYLAWYQQKPGQSPKLLIYWASTRESGVPDRFTGSGSGTDFTLTISSVQAEDLAVYYCKQSYNLLTFGAGTKLELKA

SEQ ID NO: 202 - protein, 23 aa, unidentified
  REGION 1..23: note = Human (Homo sapiens), chimeric or humanized antibody peptide sequence
DIKMTQSPSSLAVSPGEKVTMSC

SEQ ID NO: 203 - protein, 17 aa, unidentified
  REGION 1..17: note = Human (Homo sapiens), chimeric or humanized antibody peptide sequence
KSSQSLLHSRTRKNYLA

SEQ ID NO: 204 - protein, 15 aa, unidentified
  REGION 1..15: note = Human (Homo sapiens), chimeric or humanized antibody peptide sequence
WYQQKPGQSPKLLIY

SEQ ID NO: 205 - protein, 7 aa, unidentified
  REGION 1..7: note = Human (Homo sapiens), chimeric or humanized antibody peptide sequence
WASTRES

SEQ ID NO: 206 - protein, 32 aa, unidentified; identical to SEQ ID NO: 190
  REGION 1..32: note = Human (Homo sapiens), chimeric or humanized antibody peptide sequence

SEQ ID NO: 207 - protein, 8 aa, unidentified
  REGION 1..8: note = Human (Homo sapiens), chimeric or humanized antibody peptide sequence
KQSYNLLT

SEQ ID NO: 208 - protein, 11 aa, unidentified
  REGION 1..11: note = Human (Homo sapiens), chimeric or humanized antibody peptide sequence
FGAGTKLELKA

SEQ ID NO: 209 - protein, 113 aa, unidentified
  REGION 1..113: note = Human (Homo sapiens), chimeric or humanized antibody peptide sequence
DIVMSQSPSSLAVSPGEKVTMSCKSSQSLLHSRTRKNYLAWYQQKPGQSPKLLIYWASTRESGVPDRFTGSGSGTDFTLTISSVQAEDLAVYYCKQSYNLLTFGAGTKLELKA

SEQ ID NO: 210 - protein, 23 aa, unidentified
  REGION 1..23: note = Human (Homo sapiens), chimeric or humanized antibody peptide sequence
DIVMSQSPSSLAVSPGEKVTMSC

SEQ ID NO: 211 - protein, 17 aa, unidentified
  REGION 1..17: note = Human (Homo sapiens), chimeric or humanized antibody peptide sequence
KSSQSLLHSRTRKNYLA

SEQ ID NO: 212 - protein, 15 aa, unidentified
  REGION 1..15: note = Human (Homo sapiens), chimeric or humanized antibody peptide sequence
WYQQKPGQSPKLLIY

SEQ ID NO: 213 - protein, 7 aa, unidentified
  REGION 1..7: note = Human (Homo sapiens), chimeric or humanized antibody peptide sequence
WASTRES

SEQ ID NO: 214 - protein, 32 aa, unidentified; identical to SEQ ID NO: 190
  REGION 1..32: note = Human (Homo sapiens), chimeric or humanized antibody peptide sequence

SEQ ID NO: 215 - protein, 8 aa, unidentified
  REGION 1..8: note = Human (Homo sapiens), chimeric or humanized antibody peptide sequence
KQSYNLLT

SEQ ID NO: 216 - protein, 11 aa, unidentified
  REGION 1..11: note = Human (Homo sapiens), chimeric or humanized antibody peptide sequence
FGAGTKLELKA

SEQ ID NO: 217 - protein, 113 aa, unidentified
  REGION 1..113: note = Human (Homo sapiens), chimeric or humanized antibody peptide sequence
DIVMTQSPSSLAVSAGEKVTMSCKSSQSLLNSRTRKNYLAWYQQKPGQSPRLLIYWASTRESGVPDRFTGSGSGTDFTLTISSVQAEDLAVYYCKQSYNLLTFGAGTKLELKR

SEQ ID NO: 218 - protein, 23 aa, unidentified
  REGION 1..23: note = Human (Homo sapiens), chimeric or humanized antibody peptide sequence
DIVMTQSPSSLAVSAGEKVTMSC

SEQ ID NO: 219 - protein, 17 aa, unidentified
  REGION 1..17: note = Human (Homo sapiens), chimeric or humanized antibody peptide sequence
KSSQSLLNSRTRKNYLA

SEQ ID NO: 220 - protein, 15 aa, unidentified
  REGION 1..15: note = Human (Homo sapiens), chimeric or humanized antibody peptide sequence
WYQQKPGQSPRLLIY

SEQ ID NO: 221 - protein, 7 aa, unidentified
  REGION 1..7: note = Human (Homo sapiens), chimeric or humanized antibody peptide sequence
WASTRES

SEQ ID NO: 222 - protein, 32 aa, unidentified; identical to SEQ ID NO: 190
  REGION 1..32: note = Human (Homo sapiens), chimeric or humanized antibody peptide sequence

SEQ ID NO: 223 - protein, 8 aa, unidentified
  REGION 1..8: note = Human (Homo sapiens), chimeric or humanized antibody peptide sequence
KQSYNLLT

SEQ ID NO: 224 - protein, 11 aa, unidentified
  REGION 1..11: note = Human (Homo sapiens), chimeric or humanized antibody peptide sequence
FGAGTKLELKR

SEQ ID NO: 225 - protein, 113 aa, unidentified
  REGION 1..113: note = Human (Homo sapiens), chimeric or humanized antibody peptide sequence
DIVMTQSPSSLAVSAGEKVTMSCKSSQSLLNSRTRKNYLAWYQQKPGQSPRLLIYWASTRESGVPDRFTGSGSGTDFTLTISSVQAEDLAVYYCKQSYNLLTFGAGTRLEIKR

SEQ ID NO: 226 - protein, 23 aa, unidentified
  REGION 1..23: note = Human (Homo sapiens), chimeric or humanized antibody peptide sequence
DIVMTQSPSSLAVSAGEKVTMSC

SEQ ID NO: 227 - protein, 17 aa, unidentified
  REGION 1..17: note = Human (Homo sapiens), chimeric or humanized antibody peptide sequence
KSSQSLLNSRTRKNYLA

SEQ ID NO: 228 - protein, 15 aa, unidentified
  REGION 1..15: note = Human (Homo sapiens), chimeric or humanized antibody peptide sequence
WYQQKPGQSPRLLIY

SEQ ID NO: 229 - protein, 7 aa, unidentified
  REGION 1..7: note = Human (Homo sapiens), chimeric or humanized antibody peptide sequence
WASTRES

SEQ ID NO: 230 - protein, 32 aa, unidentified; identical to SEQ ID NO: 190
  REGION 1..32: note = Human (Homo sapiens), chimeric or humanized antibody peptide sequence

SEQ ID NO: 231 - protein, 8 aa, unidentified
  REGION 1..8: note = Human (Homo sapiens), chimeric or humanized antibody peptide sequence
KQSYNLLT

SEQ ID NO: 232 - protein, 11 aa, unidentified
  REGION 1..11: note = Human (Homo sapiens), chimeric or humanized antibody peptide sequence
FGAGTRLEIKR

SEQ ID NO: 233 - protein, 113 aa, unidentified
  REGION 1..113: note = Human (Homo sapiens), chimeric or humanized antibody peptide sequence
DIVMTQSPSSLAVSAGENVTVSCKSSQSLLNSRTRKNYLAWYQQKPGQSPKLLIYWASTRESGVPDRFTGSGSGTGFTLTISSVQAEDLAVYFCKQSYNLLTFGAGTRLEIKR

SEQ ID NO: 234 - protein, 23 aa, unidentified
  REGION 1..23: note = Human (Homo sapiens), chimeric or humanized antibody peptide sequence
DIVMTQSPSSLAVSAGENVTVSC

SEQ ID NO: 235 - protein, 17 aa, unidentified
  REGION 1..17: note = Human (Homo sapiens), chimeric or humanized antibody peptide sequence
KSSQSLLNSRTRKNYLA

SEQ ID NO: 236 - protein, 15 aa, unidentified
  REGION 1..15: note = Human (Homo sapiens), chimeric or humanized antibody peptide sequence
WYQQKPGQSPKLLIY

SEQ ID NO: 237 - protein, 7 aa, unidentified
  REGION 1..7: note = Human (Homo sapiens), chimeric or humanized antibody peptide sequence
WASTRES

SEQ ID NO: 238 - protein, 32 aa, unidentified
  REGION 1..32: note = Human (Homo sapiens), chimeric or humanized antibody peptide sequence
GVPDRFTGSGSGTGFTLTISSVQAEDLAVYFC

SEQ ID NO: 239 - protein, 8 aa, unidentified
  REGION 1..8: note = Human (Homo sapiens), chimeric or humanized antibody peptide sequence
KQSYNLLT

SEQ ID NO: 240 - protein, 11 aa, unidentified
  REGION 1..11: note = Human (Homo sapiens), chimeric or humanized antibody peptide sequence
FGAGTRLEIKR

SEQ ID NO: 241 - protein, 113 aa, unidentified
  REGION 1..113: note = Human (Homo sapiens), chimeric or humanized antibody peptide sequence
DIVMSQSPSSLAVSPGEKVTMSCKSSQSLLHSRTRKNYLAWYQQKPGQSPKLLIYWASTRESGVPDRFTGSGSGTDFTLTISSVQAEDLAVYYCKQSYNLLTFGAGTKLELKR

SEQ ID NO: 242 - protein, 23 aa, unidentified
  REGION 1..23: note = Human (Homo sapiens), chimeric or humanized antibody peptide sequence
DIVMSQSPSSLAVSPGEKVTMSC

SEQ ID NO: 243 - protein, 17 aa, unidentified
  REGION 1..17: note = Human (Homo sapiens), chimeric or humanized antibody peptide sequence
KSSQSLLHSRTRKNYLA

SEQ ID NO: 244 - protein, 15 aa, unidentified
  REGION 1..15: note = Human (Homo sapiens), chimeric or humanized antibody peptide sequence
WYQQKPGQSPKLLIY

SEQ ID NO: 245 - protein, 7 aa, unidentified
  REGION 1..7: note = Human (Homo sapiens), chimeric or humanized antibody peptide sequence
WASTRES

SEQ ID NO: 246 - protein, 32 aa, unidentified; identical to SEQ ID NO: 190
  REGION 1..32: note = Human (Homo sapiens), chimeric or humanized antibody peptide sequence

SEQ ID NO: 247 - protein, 8 aa, unidentified
  REGION 1..8: note = Human (Homo sapiens), chimeric or humanized antibody peptide sequence
KQSYNLLT

SEQ ID NO: 248 - protein, 11 aa, unidentified
  REGION 1..11: note = Human (Homo sapiens), chimeric or humanized antibody peptide sequence
FGAGTKLELKR

SEQ ID NO: 249 - protein, 113 aa, unidentified; identical to SEQ ID NO: 185
  REGION 1..113: note = Human (Homo sapiens), chimeric or humanized antibody peptide sequence

SEQ ID NO: 250 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 251 - protein, 17 aa, unidentified
  REGION 1..17: note = Human (Homo sapiens), chimeric or humanized antibody peptide sequence
KSSQSLLNSRTRKNYLA

SEQ ID NO: 252 - protein, 15 aa, unidentified
  REGION 1..15: note = Human (Homo sapiens), chimeric or humanized antibody peptide sequence
WYQQKPGQSPRLLIY

SEQ ID NO: 253 - protein, 7 aa, unidentified
  REGION 1..7: note = Human (Homo sapiens), chimeric or humanized antibody peptide sequence
WASTRES

SEQ ID NO: 254 - protein, 32 aa, unidentified; identical to SEQ ID NO: 190
  REGION 1..32: note = Human (Homo sapiens), chimeric or humanized antibody peptide sequence

SEQ ID NO: 255 - protein, 8 aa, unidentified
  REGION 1..8: note = Human (Homo sapiens), chimeric or humanized antibody peptide sequence
KQSYNLLT

SEQ ID NO: 256 - protein, 11 aa, unidentified
  REGION 1..11: note = Human (Homo sapiens), chimeric or humanized antibody peptide sequence
FGAGTKLELKA

SEQ ID NO: 257 - protein, 113 aa, unidentified
  REGION 1..113: note = Human (Homo sapiens), chimeric or humanized antibody peptide sequence
DIVMSQSPSSLAVSAGEKVTMSCKSSQSLLNSRTRKNYLAWYQQKPGQSPRLLIYWASTRESGVPDRFTGSGSGTDFTLTISSVQDEDLAVYYCKQSYNLLTFGAGTKLELKA

SEQ ID NO: 258 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 259 - protein, 17 aa, unidentified
  REGION 1..17: note = Human (Homo sapiens), chimeric or humanized antibody peptide sequence
KSSQSLLNSRTRKNYLA

SEQ ID NO: 260 - protein, 15 aa, unidentified
  REGION 1..15: note = Human (Homo sapiens), chimeric or humanized antibody peptide sequence
WYQQKPGQSPRLLIY

SEQ ID NO: 261 - protein, 7 aa, unidentified
  REGION 1..7: note = Human (Homo sapiens), chimeric or humanized antibody peptide sequence
WASTRES

SEQ ID NO: 262 - protein, 32 aa, unidentified
  REGION 1..32: note = Human (Homo sapiens), chimeric or humanized antibody peptide sequence
GVPDRFTGSGSGTDFTLTISSVQDEDLAVYYC

SEQ ID NO: 263 - protein, 8 aa, unidentified
  REGION 1..8: note = Human (Homo sapiens), chimeric or humanized antibody peptide sequence
KQSYNLLT

SEQ ID NO: 264 - protein, 11 aa, unidentified
  REGION 1..11: note = Human (Homo sapiens), chimeric or humanized antibody peptide sequence
FGAGTKLELKA

SEQ ID NO: 265 - protein, 113 aa, unidentified; identical to SEQ ID NO: 185
  REGION 1..113: note = Human (Homo sapiens), chimeric or humanized antibody peptide sequence

SEQ ID NO: 266 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 267 - protein, 17 aa, unidentified
  REGION 1..17: note = Human (Homo sapiens), chimeric or humanized antibody peptide sequence
KSSQSLLNSRTRKNYLA

SEQ ID NO: 268 - protein, 15 aa, unidentified
  REGION 1..15: note = Human (Homo sapiens), chimeric or humanized antibody peptide sequence
WYQQKPGQSPRLLIY

SEQ ID NO: 269 - protein, 7 aa, unidentified
  REGION 1..7: note = Human (Homo sapiens), chimeric or humanized antibody peptide sequence
WASTRES

SEQ ID NO: 270 - protein, 32 aa, unidentified; identical to SEQ ID NO: 190
  REGION 1..32: note = Human (Homo sapiens), chimeric or humanized antibody peptide sequence

SEQ ID NO: 271 - protein, 8 aa, unidentified
  REGION 1..8: note = Human (Homo sapiens), chimeric or humanized antibody peptide sequence
KQSYNLLT

SEQ ID NO: 272 - protein, 11 aa, unidentified
  REGION 1..11: note = Human (Homo sapiens), chimeric or humanized antibody peptide sequence
FGAGTKLELKA

SEQ ID NO: 273 - protein, 113 aa, unidentified; identical to SEQ ID NO: 185
  REGION 1..113: note = Human (Homo sapiens), chimeric or humanized antibody peptide sequence

SEQ ID NO: 274 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 275 - protein, 17 aa, unidentified
  REGION 1..17: note = Human (Homo sapiens), chimeric or humanized antibody peptide sequence
KSSQSLLNSRTRKNYLA

SEQ ID NO: 276 - protein, 15 aa, unidentified
  REGION 1..15: note = Human (Homo sapiens), chimeric or humanized antibody peptide sequence
WYQQKPGQSPRLLIY

SEQ ID NO: 277 - protein, 7 aa, unidentified
  REGION 1..7: note = Human (Homo sapiens), chimeric or humanized antibody peptide sequence
WASTRES

SEQ ID NO: 278 - protein, 32 aa, unidentified; identical to SEQ ID NO: 190
  REGION 1..32: note = Human (Homo sapiens), chimeric or humanized antibody peptide sequence

SEQ ID NO: 279 - protein, 8 aa, unidentified
  REGION 1..8: note = Human (Homo sapiens), chimeric or humanized antibody peptide sequence
KQSYNLLT

SEQ ID NO: 280 - protein, 11 aa, unidentified
  REGION 1..11: note = Human (Homo sapiens), chimeric or humanized antibody peptide sequence
FGAGTKLELKA

SEQ ID NO: 281 - protein, 113 aa, unidentified
  REGION 1..113: note = Human (Homo sapiens), chimeric or humanized antibody peptide sequence
  VARIANT 49: note = misc_feature - Xaa can be any naturally occurring amino acid
DIVMSQSPSSLAVSAGEKVTMSCKSSQSLLNSRTRKNYLAWYQQKPGQXPRLLIYWASTRESGVPDRFTGSGSGTDFTLTISSVQDEDLAVYYCKQSYNLLSFGAGTKLELKA

SEQ ID NO: 282 - protein, 23 aa, unidentified
  REGION 1..23: note = Human (Homo sapiens), chimeric or humanized antibody peptide sequence
DIVMSQSPSSLAVSAGEKVTMSC

SEQ ID NO: 283 - protein, 17 aa, unidentified
  REGION 1..17: note = Human (Homo sapiens), chimeric or humanized antibody peptide sequence
KSSQSLLNSRTRKNYLA

SEQ ID NO: 284 - protein, 15 aa, unidentified
  REGION 1..15: note = Human (Homo sapiens), chimeric or humanized antibody peptide sequence
  VARIANT 9: note = misc_feature - Xaa can be any naturally occurring amino acid
WYQQKPGQXPRLLIY

SEQ ID NO: 285 - protein, 7 aa, unidentified
  REGION 1..7: note = Human (Homo sapiens), chimeric or humanized antibody peptide sequence
WASTRES

SEQ ID NO: 286 - protein, 32 aa, unidentified; identical to SEQ ID NO: 262
  REGION 1..32: note = Human (Homo sapiens), chimeric or humanized antibody peptide sequence

SEQ ID NO: 287 - protein, 8 aa, unidentified
  REGION 1..8: note = Human (Homo sapiens), chimeric or humanized antibody peptide sequence
KQSYNLLS

SEQ ID NO: 288 - protein, 11 aa, unidentified
  REGION 1..11: note = Human (Homo sapiens), chimeric or humanized antibody peptide sequence
FGAGTKLELKA

SEQ ID NO: 289 - protein, 113 aa, unidentified
  REGION 1..113: note = Human (Homo sapiens), chimeric or humanized antibody peptide sequence
DIVMTQSPSSLAVSAGEKVTMSCKSSQSLLNSRTRKNYLAWYQQKPGQSPRLLIYWASTRESGVPDRFTGSGSGTDFTLTISSVQAEDLAVYYCKQSYNLLSFGAGTKLELKR

SEQ ID NO: 290 - protein, 23 aa, unidentified
  REGION 1..23: note = Human (Homo sapiens), chimeric or humanized antibody peptide sequence
DIVMTQSPSSLAVSAGEKVTMSC

SEQ ID NO: 291 - protein, 17 aa, unidentified
  REGION 1..17: note = Human (Homo sapiens), chimeric or humanized antibody peptide sequence
KSSQSLLNSRTRKNYLA

SEQ ID NO: 292 - protein, 15 aa, unidentified
  REGION 1..15: note = Human (Homo sapiens), chimeric or humanized antibody peptide sequence
WYQQKPGQSPRLLIY

SEQ ID NO: 293 - protein, 7 aa, unidentified
  REGION 1..7: note = Human (Homo sapiens), chimeric or humanized antibody peptide sequence
WASTRES

SEQ ID NO: 294 - protein, 32 aa, unidentified; identical to SEQ ID NO: 190
  REGION 1..32: note = Human (Homo sapiens), chimeric or humanized antibody peptide sequence

SEQ ID NO: 295 - protein, 8 aa, unidentified
  REGION 1..8: note = Human (Homo sapiens), chimeric or humanized antibody peptide sequence
KQSYNLLS

SEQ ID NO: 296 - protein, 11 aa, unidentified
  REGION 1..11: note = Human (Homo sapiens), chimeric or humanized antibody peptide sequence
FGAGTKLELKR

SEQ ID NO: 297 - protein, 120 aa, unidentified
  REGION 1..120: note = Human (Homo sapiens), chimeric or humanized antibody peptide sequence
QVQLQQSGAELVRPGTSVKVSCKASGYAFTDYLIQWVKQRPGQGLEWIGVINPETGGTNYNAKFRGKATLTADKSSSSAYMQLSSLTSGDSAVYFCAREAGNYIYAMDYWGQGTSVTVSS

SEQ ID NO: 298 - protein, 27 aa, unidentified
  REGION 1..27: note = Human (Homo sapiens), chimeric or humanized antibody peptide sequence
QVQLQQSGAELVRPGTSVKVSCKASGY

SEQ ID NO: 299 - protein, 8 aa, unidentified
  REGION 1..8: note = Human (Homo sapiens), chimeric or humanized antibody peptide sequence
AFTDYLIQ

SEQ ID NO: 300 - protein, 14 aa, unidentified
  REGION 1..14: note = Human (Homo sapiens), chimeric or humanized antibody peptide sequence
WVKQRPGQGLEWIG

SEQ ID NO: 301 - protein, 17 aa, unidentified
  REGION 1..17: note = Human (Homo sapiens), chimeric or humanized antibody peptide sequence
VINPETGGTNYNAKFRG

SEQ ID NO: 302 - protein, 32 aa, unidentified
  REGION 1..32: note = Human (Homo sapiens), chimeric or humanized antibody peptide sequence
KATLTADKSSSSAYMQLSSLTSGDSAVYFCAR

SEQ ID NO: 303 - protein, 11 aa, unidentified
  REGION 1..11: note = Human (Homo sapiens), chimeric or humanized antibody peptide sequence
EAGNYIYAMDY

SEQ ID NO: 304 - protein, 11 aa, unidentified
  REGION 1..11: note = Human (Homo sapiens), chimeric or humanized antibody peptide sequence
WGQGTSVTVSS

SEQ ID NO: 305 - protein, 108 aa, unidentified
  REGION 1..108: note = Human (Homo sapiens), chimeric or humanized antibody peptide sequence
DIVMTQSPAFLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKR

SEQ ID NO: 306 - protein, 23 aa, unidentified
  REGION 1..23: note = Human (Homo sapiens), chimeric or humanized antibody peptide sequence
DIVMTQSPAFLSASVGETVTITC

SEQ ID NO: 307 - protein, 11 aa, unidentified
  REGION 1..11: note = Human (Homo sapiens), chimeric or humanized antibody peptide sequence
RASVNIYSYLV

SEQ ID NO: 308 - protein, 15 aa, unidentified
  REGION 1..15: note = Human (Homo sapiens), chimeric or humanized antibody peptide sequence
WYQQKQGKSPQLLVH

SEQ ID NO: 309 - protein, 7 aa, unidentified
  REGION 1..7: note = Human (Homo sapiens), chimeric or humanized antibody peptide sequence
NAKTLAE

SEQ ID NO: 310 - protein, 32 aa, unidentified
  REGION 1..32: note = Human (Homo sapiens), chimeric or humanized antibody peptide sequence
GVPSRFSGSGSGTQFSLKINSLQPEDFGSYYC

SEQ ID NO: 311 - protein, 9 aa, unidentified
  REGION 1..9: note = Human (Homo sapiens), chimeric or humanized antibody peptide sequence
QHHHGTPYT

SEQ ID NO: 312 - protein, 11 aa, unidentified
  REGION 1..11: note = Human (Homo sapiens), chimeric or humanized antibody peptide sequence
FGGGTKLEIKR

SEQ ID NO: 313 - protein, 8 aa, unidentified
  REGION 1..8: note = Human (Homo sapiens), chimeric or humanized antibody peptide sequence
TFTDYSMH

SEQ ID NO: 314 - protein, 17 aa, unidentified
  REGION 1..17: note = Human (Homo sapiens), chimeric or humanized antibody peptide sequence
RINTETGEPTFADDFGG

SEQ ID NO: 315 - protein, 7 aa, unidentified
  REGION 1..7: note = Human (Homo sapiens), chimeric or humanized antibody peptide sequence
YYYGRDS

SEQ ID NO: 316 - protein, 8 aa, unidentified
  REGION 1..8: note = Human (Homo sapiens), chimeric or humanized antibody peptide sequence
TFTDYSMH

SEQ ID NO: 317 - protein, 17 aa, unidentified
  REGION 1..17: note = Human (Homo sapiens), chimeric or humanized antibody peptide sequence
WIKTETGEPTYADDFKG

SEQ ID NO: 318 - protein, 7 aa, unidentified
  REGION 1..7: note = Human (Homo sapiens), chimeric or humanized antibody peptide sequence
YYYGRDS

SEQ ID NO: 319 - protein, 8 aa, unidentified
  REGION 1..8: note = Human (Homo sapiens), chimeric or humanized antibody peptide sequence
TFTDYSMH

SEQ ID NO: 320 - protein, 17 aa, unidentified
  REGION 1..17: note = Human (Homo sapiens), chimeric or humanized antibody peptide sequence
WIKTETDEPTYADDFKE

SEQ ID NO: 321 - protein, 7 aa, unidentified
  REGION 1..7: note = Human (Homo sapiens), chimeric or humanized antibody peptide sequence
YYYGRDS

SEQ ID NO: 322 - protein, 8 aa, unidentified
  REGION 1..8: note = Human (Homo sapiens), chimeric or humanized antibody peptide sequence
TFTDYSMH

SEQ ID NO: 323 - protein, 17 aa, unidentified
  REGION 1..17: note = Human (Homo sapiens), chimeric or humanized antibody peptide sequence
RINTETGEPTFADDFRG

SEQ ID NO: 324 - protein, 7 aa, unidentified
  REGION 1..7: note = Human (Homo sapiens), chimeric or humanized antibody peptide sequence
YYYGRDS

SEQ ID NO: 325 - protein, 8 aa, unidentified
  REGION 1..8: note = Human (Homo sapiens), chimeric or humanized antibody peptide sequence
TFTDYSIH

SEQ ID NO: 326 - protein, 17 aa, unidentified
  REGION 1..17: note = Human (Homo sapiens), chimeric or humanized antibody peptide sequence
WIKTETGEPTYADDFNG

SEQ ID NO: 327 - protein, 7 aa, unidentified
  REGION 1..7: note = Human (Homo sapiens), chimeric or humanized antibody peptide sequence
YYYGRDS

SEQ ID NO: 328 - protein, 8 aa, unidentified
  REGION 1..8: note = Human (Homo sapiens), chimeric or humanized antibody peptide sequence
TFTDYSMH

SEQ ID NO: 329 - protein, 17 aa, unidentified
  REGION 1..17: note = Human (Homo sapiens), chimeric or humanized antibody peptide sequence
RINTETGEPTFADDFRG

SEQ ID NO: 330 - protein, 7 aa, unidentified
  REGION 1..7: note = Human (Homo sapiens), chimeric or humanized antibody peptide sequence
YYYGRDT

SEQ ID NO: 331 - protein, 8 aa, unidentified
  REGION 1..8: note = Human (Homo sapiens), chimeric or humanized antibody peptide sequence
TFTDYSMH

SEQ ID NO: 332 - protein, 17 aa, unidentified
  REGION 1..17: note = Human (Homo sapiens), chimeric or humanized antibody peptide sequence
RINTETGEPTFADDFRG

SEQ ID NO: 333 - protein, 7 aa, unidentified
  REGION 1..7: note = Human (Homo sapiens), chimeric or humanized antibody peptide sequence
FYYGRDS

SEQ ID NO: 334 - protein, 11 aa, unidentified
  REGION 1..11: note = Human (Homo sapiens), chimeric or humanized antibody peptide sequence
KASQDINSYLS

SEQ ID NO: 335 - protein, 7 aa, unidentified
  REGION 1..7: note = Human (Homo sapiens), chimeric or humanized antibody peptide sequence
RANRLVD

SEQ ID NO: 336 - protein, 10 aa, unidentified
  REGION 1..10: note = Human (Homo sapiens), chimeric or humanized antibody peptide sequence
LQYDEFPPLT

SEQ ID NO: 337 - protein, 17 aa, unidentified
  REGION 1..17: note = Human (Homo sapiens), chimeric or humanized antibody peptide sequence
KSSQSLLNSRTRKNYLA

SEQ ID NO: 338 - protein, 7 aa, unidentified
  REGION 1..7: note = Human (Homo sapiens), chimeric or humanized antibody peptide sequence
WASTRES

SEQ ID NO: 339 - protein, 8 aa, unidentified
  REGION 1..8: note = Human (Homo sapiens), chimeric or humanized antibody peptide sequence
KQSYNLLT

SEQ ID NO: 340 - protein, 17 aa, unidentified
  REGION 1..17: note = Human (Homo sapiens), chimeric or humanized antibody peptide sequence
KSSQSLLNSRIRKNYLA

SEQ ID NO: 341 - protein, 7 aa, unidentified
  REGION 1..7: note = Human (Homo sapiens), chimeric or humanized antibody peptide sequence
WASTRES

SEQ ID NO: 342 - protein, 8 aa, unidentified
  REGION 1..8: note = Human (Homo sapiens), chimeric or humanized antibody peptide sequence
KQSYNLLT

SEQ ID NO: 343 - protein, 10 aa, unidentified
  REGION 1..10: note = Human (Homo sapiens), chimeric or humanized antibody peptide sequence
SASSSVSYMH

SEQ ID NO: 344 - protein, 7 aa, unidentified
  REGION 1..7: note = Human (Homo sapiens), chimeric or humanized antibody peptide sequence
DTSNLAS

SEQ ID NO: 345 - protein, 9 aa, unidentified
  REGION 1..9: note = Human (Homo sapiens), chimeric or humanized antibody peptide sequence
QQWSSNPLT

SEQ ID NO: 346 - protein, 10 aa, unidentified
  REGION 1..10: note = Human (Homo sapiens), chimeric or humanized antibody peptide sequence
SASSSVSYMH

SEQ ID NO: 347 - protein, 7 aa, unidentified
  REGION 1..7: note = Human (Homo sapiens), chimeric or humanized antibody peptide sequence
DTSNLAS

SEQ ID NO: 348 - protein, 9 aa, unidentified
  REGION 1..9: note = Human (Homo sapiens), chimeric or humanized antibody peptide sequence
QQWSSNPPT

SEQ ID NO: 349 - protein, 17 aa, unidentified
  REGION 1..17: note = Human (Homo sapiens), chimeric or humanized antibody peptide sequence
KSSQSLLNSRTRKNYLA

SEQ ID NO: 350 - protein, 7 aa, unidentified
  REGION 1..7: note = Human (Homo sapiens), chimeric or humanized antibody peptide sequence
WASTRES

SEQ ID NO: 351 - protein, 8 aa, unidentified
  REGION 1..8: note = Human (Homo sapiens), chimeric or humanized antibody peptide sequence
KQSYNLLT

SEQ ID NO: 352 - protein, 10 aa, unidentified
  REGION 1..10: note = Human (Homo sapiens), chimeric or humanized antibody peptide sequence
SASSSVSYMH

SEQ ID NO: 353 - protein, 7 aa, unidentified
  REGION 1..7: note = Human (Homo sapiens), chimeric or humanized antibody peptide sequence
DTSKLAS

SEQ ID NO: 354 - protein, 9 aa, unidentified
  REGION 1..9: note = Human (Homo sapiens), chimeric or humanized antibody peptide sequence
QQWSSNPFT

SEQ ID NO: 355 - protein, 10 aa, unidentified
  REGION 1..10: note = Human (Homo sapiens), chimeric or humanized antibody peptide sequence
SASSSVSYMH

SEQ ID NO: 356 - protein, 7 aa, unidentified
  REGION 1..7: note = Human (Homo sapiens), chimeric or humanized antibody peptide sequence
GTSNLAS

SEQ ID NO: 357 - protein, 9 aa, unidentified
  REGION 1..9: note = Human (Homo sapiens), chimeric or humanized antibody peptide sequence
QQWSSNPPT

SEQ ID NO: 358 - protein, 17 aa, unidentified
  REGION 1..17: note = Human (Homo sapiens), chimeric or humanized antibody peptide sequence
KSSQSLLHSRTRKNYLA

SEQ ID NO: 359 - protein, 7 aa, unidentified
  REGION 1..7: note = Human (Homo sapiens), chimeric or humanized antibody peptide sequence
WASTRES

SEQ ID NO: 360 - protein, 8 aa, unidentified
  REGION 1..8: note = Human (Homo sapiens), chimeric or humanized antibody peptide sequence
KQSYNLLT

SEQ ID NO: 361 - protein, 18 aa, unidentified
  REGION 1..18: note = Human (Homo sapiens), chimeric or humanized antibody peptide sequence
DKSSQSLLNSRTRKNYLA

SEQ ID NO: 362 - protein, 8 aa, unidentified
  REGION 1..8: note = Human (Homo sapiens), chimeric or humanized antibody peptide sequence
DWASTRES

SEQ ID NO: 363 - protein, 9 aa, unidentified
  REGION 1..9: note = Human (Homo sapiens), chimeric or humanized antibody peptide sequence
DKQSYNLLS

SEQ ID NO: 364 - protein, 8 aa, unidentified
  REGION 1..8: note = Human (Homo sapiens), chimeric or humanized antibody peptide sequence
TFTDYSIH

SEQ ID NO: 365 - protein, 17 aa, unidentified
  REGION 1..17: note = Human (Homo sapiens), chimeric or humanized antibody peptide sequence
WIKTETGEPTYADDFKG

SEQ ID NO: 366 - protein, 7 aa, unidentified
  REGION 1..7: note = Human (Homo sapiens), chimeric or humanized antibody peptide sequence
YYYGRDS

SEQ ID NO: 367 - protein, 8 aa, unidentified
  REGION 1..8: note = Human (Homo sapiens), chimeric or humanized antibody peptide sequence
TFTDYSIH

SEQ ID NO: 368 - protein, 17 aa, unidentified
  REGION 1..17: note = Human (Homo sapiens), chimeric or humanized antibody peptide sequence
WIKTETGEPTYADDFNG

SEQ ID NO: 369 - protein, 7 aa, unidentified
  REGION 1..7: note = Human (Homo sapiens), chimeric or humanized antibody peptide sequence
YYYGRDS

SEQ ID NO: 370 - protein, 8 aa, unidentified
  REGION 1..8: note = Human (Homo sapiens), chimeric or humanized antibody peptide sequence
TFTDYSMH

SEQ ID NO: 371 - protein, 17 aa, unidentified
  REGION 1..17: note = Human (Homo sapiens), chimeric or humanized antibody peptide sequence
RINTETGEPTFADDFRG

SEQ ID NO: 372 - protein, 7 aa, unidentified
  REGION 1..7: note = Human (Homo sapiens), chimeric or humanized antibody peptide sequence
YYYGRDS

SEQ ID NO: 373 - protein, 17 aa, unidentified
  REGION 1..17: note = Human (Homo sapiens), chimeric or humanized antibody peptide sequence
KSSQSLLNSRTRKNYLA

SEQ ID NO: 374 - protein, 7 aa, unidentified
  REGION 1..7: note = Human (Homo sapiens), chimeric or humanized antibody peptide sequence
WASTRES

SEQ ID NO: 375 - protein, 8 aa, unidentified
  REGION 1..8: note = Human (Homo sapiens), chimeric or humanized antibody peptide sequence
KQSYNLLT

SEQ ID NO: 376 - protein, 17 aa, unidentified
  REGION 1..17: note = Human (Homo sapiens), chimeric or humanized antibody peptide sequence
KSSQSLLHSRTRKNYLA

SEQ ID NO: 377 - protein, 7 aa, unidentified
  REGION 1..7: note = Human (Homo sapiens), chimeric or humanized antibody peptide sequence
WASTRES

SEQ ID NO: 378 - protein, 8 aa, unidentified
  REGION 1..8: note = Human (Homo sapiens), chimeric or humanized antibody peptide sequence
KQSYNLLT

SEQ ID NO: 379 - protein, 8 aa, unidentified
  REGION 1..8: note = Human (Homo sapiens), chimeric or humanized antibody peptide sequence
TFTDYSMH

SEQ ID NO: 380 - protein, 17 aa, unidentified
  REGION 1..17: note = Human (Homo sapiens), chimeric or humanized antibody peptide sequence
RINTETGEPTFADDFRG

SEQ ID NO: 381 - protein, 7 aa, unidentified
  REGION 1..7: note = Human (Homo sapiens), chimeric or humanized antibody peptide sequence
YYYGRDS

SEQ ID NO: 382 - protein, 17 aa, unidentified
  REGION 1..17: note = Human (Homo sapiens), chimeric or humanized antibody peptide sequence
KSSQSLLNSRTRKNYLA

SEQ ID NO: 383 - protein, 7 aa, unidentified
  REGION 1..7: note = Human (Homo sapiens), chimeric or humanized antibody peptide sequence
WASTRES

SEQ ID NO: 384 - protein, 8 aa, unidentified
  REGION 1..8: note = Human (Homo sapiens), chimeric or humanized antibody peptide sequence
KQSYNLLT

SEQ ID NO: 385 - protein, 27 aa, unidentified
  REGION 1..27: note = Human (Homo sapiens), chimeric or humanized antibody peptide sequence
  VARIANT 5: note = MISC_FEATURE - Xaa is Leu or Met
ZIQLXZSGPELKKPGETVKISCKASGY

SEQ ID NO: 386 - protein, 14 aa, unidentified
  REGION 1..14: note = Human (Homo sapiens), chimeric or humanized antibody peptide sequence
  VARIANT 13: note = MISC_FEATURE - Xaa is Val or Met
WVKQAPGKGLKWXA

SEQ ID NO: 387 - protein, 32 aa, unidentified
  REGION 1..32: note = Human (Homo sapiens), chimeric or humanized antibody peptide sequence
  VARIANT 4: note = MISC_FEATURE - Xaa is Val or Phe
RFAXSLETSASTAYLQINNLKNEDTATYFCAI

SEQ ID NO: 388 - protein, 15 aa, unidentified
  REGION 1..15: note = Human (Homo sapiens), chimeric or humanized antibody peptide sequence
  VARIANT 11: note = MISC_FEATURE - Xaa is Lys or Arg
WYQQKPGQSPXLLIY

SEQ ID NO: 389 - protein, 23 aa, unidentified
  REGION 1..23: note = Human (Homo sapiens), chimeric or humanized antibody peptide sequence
  VARIANT 1: note = MISC_FEATURE - Xaa is Asp or Glu
  VARIANT 5: note = MISC_FEATURE - Xaa is Thr or Ser
  VARIANT 14: note = MISC_FEATURE - Xaa is Pro or Ser
  VARIANT 18: note = MISC_FEATURE - Xaa is Lys or Asn
  VARIANT 21: note = MISC_FEATURE - Xaa is Met or Val
XIVMXQSPSSLAVXAGEXVTXSC

SEQ ID NO: 390 - protein, 15 aa, unidentified
  REGION 1..15: note = Human (Homo sapiens), chimeric or humanized antibody peptide sequence
  VARIANT 11: note = MISC_FEATURE - Xaa is Lys or Arg
WYQQKPGQSPXLLIY

SEQ ID NO: 391 - protein, 32 aa, unidentified
  REGION 1..32: note = Human (Homo sapiens), chimeric or humanized antibody peptide sequence
  VARIANT 31: note = MISC_FEATURE - Xaa is Tyr or Phe
GVPDRFTGSGSGTDFTLTISSVQAEDLAVYXC

SEQ ID NO: 392 - protein, 10 aa, unidentified
  REGION 1..10: note = Human (Homo sapiens), chimeric or humanized antibody peptide sequence
  VARIANT 6: note = MISC_FEATURE - Xaa is Lys or Arg
  VARIANT 9: note = MISC_FEATURE - Xaa is Leu or Ile
FGAGTXLEXK

SEQ ID NO: 393 - protein, 1018 aa, Homo sapiens
FNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 394 - protein, 727 aa, Homo sapiens
EDNRCASSNAASCARCLALGPECGWCVQEDFISGGSRSERCDIVSNLISKGCSVDSIEYPSVHVIIPTENEINTQVTPGEVSIQLRPGAEANFMLKVHPLKKYPVDLYYLVDVSASMHNNIEKLNSVGNDLSRKMAFFSRDFRLGFGSYVDKTVSPYISIHPERIHNQCSDYNLDCMPPHGYIHVLSLTENITEFEKAVHRQKISGNIDTPEGGFDAMLQAAVCESHIGWRKEAKRLLLVMTDQTSHLALDSKLAGIVVPNDGNCHLKNNVYVKSTTMEHPSLGQLSEKLIDNNINVIFAVQGKQFHWYKDLLPLLPGTIAGEIESKAANLNNLVVEAYQKLISEVKVQVENQVQGIYFNITAICPDGSRKPGMEGCRNVTSNDEVLFNVTVTMKKCDVTGGKNYAIIKPIGFNETAKIHIHRNCSCQCEDNRGPKGKCVDETFLDSKCFQCDENKCHFDEDQFSSESCKSHKDQPVCSGRGVCVCGKCSCHKIKLGKVYGKYCEKDDFSCPYHHGNLCAGHGECEAGRCQCFSGWEGDRCQCPSAAAQHCVNSKGQVCSGRGTCVCGRCECTDPRSIGRFCEHCPTCYTACKENWNCMQCLHPHNLSQAILDQCKTSCALMEQQHYVDQTSECFSSPSYLRIFFIIFIVTFLIGLLKVLIIRQVILQWNSNKIKSSSDYRVSASKKDKLILQSVCTRAVTYRREKPEEIKMDISKLNAHETFRCNF

SEQ ID NO: 395 - protein, 116 aa, unidentified
  REGION 1..116: note = Human (Homo sapiens), chimeric or humanized antibody peptide sequence
QIQLVQSGAEVKKPGASVKISCKASGYTFTDYSMHWVRQAPGQGLEWVARINTETGEPTFADDFRGRFTVTLDTSTSTAYLEIRSLRSDDTAVYFCAIFYYGRDSWGQGTTLTVSS

SEQ ID NO: 396 - protein, 30 aa, unidentified
  REGION 1..30: note = Human (Homo sapiens), chimeric or humanized antibody peptide sequence
QIQLVQSGAEVKKPGASVKISCKASGYTFT

SEQ ID NO: 397 - protein, 5 aa, unidentified
  REGION 1..5: note = Human (Homo sapiens), chimeric or humanized antibody peptide sequence
DYSMH

SEQ ID NO: 398 - protein, 14 aa, unidentified
  REGION 1..14: note = Human (Homo sapiens), chimeric or humanized antibody peptide sequence
WVRQAPGQGLEWVA

SEQ ID NO: 399 - protein, 17 aa, unidentified
  REGION 1..17: note = Human (Homo sapiens), chimeric or humanized antibody peptide sequence
RINTETGEPTFADDFRG

SEQ ID NO: 400 - protein, 32 aa, unidentified
  REGION 1..32: note = Human (Homo sapiens), chimeric or humanized antibody peptide sequence
RFTVTLDTSTSTAYLEIRSLRSDDTAVYFCAI

SEQ ID NO: 401 - protein, 7 aa, unidentified
  REGION 1..7: note = Human (Homo sapiens), chimeric or humanized antibody peptide sequence
FYYGRDS

SEQ ID NO: 402 - protein, 11 aa, unidentified
  REGION 1..11: note = Human (Homo sapiens), chimeric or humanized antibody peptide sequence
WGQGTTLTVSS

SEQ ID NO: 403 - protein, 116 aa, unidentified; identical to SEQ ID NO: 395
  REGION 1..116: note = Human (Homo sapiens), chimeric or humanized antibody peptide sequence

SEQ ID NO: 404 - protein, 30 aa, unidentified; identical to SEQ ID NO: 396
  REGION 1..30: note = Human (Homo sapiens), chimeric or humanized antibody peptide sequence

SEQ ID NO: 405 - protein, 5 aa, unidentified
  REGION 1..5: note = Human (Homo sapiens), chimeric or humanized antibody peptide sequence
DYSMH

SEQ ID NO: 406 - protein, 14 aa, unidentified
  REGION 1..14: note = Human (Homo sapiens), chimeric or humanized antibody peptide sequence
WVRQAPGQGLEWVA

SEQ ID NO: 407 - protein, 17 aa, unidentified
  REGION 1..17: note = Human (Homo sapiens), chimeric or humanized antibody peptide sequence
RINTETGEPTFADDFRG

SEQ ID NO: 408 - protein, 32 aa, unidentified; identical to SEQ ID NO: 400
  REGION 1..32: note = Human (Homo sapiens), chimeric or humanized antibody peptide sequence

SEQ ID NO: 409 - protein, 7 aa, unidentified
  REGION 1..7: note = Human (Homo sapiens), chimeric or humanized antibody peptide sequence
FYYGRDS

SEQ ID NO: 410 - protein, 11 aa, unidentified
  REGION 1..11: note = Human (Homo sapiens), chimeric or humanized antibody peptide sequence
WGQGTTLTVSS

SEQ ID NO: 411 - protein, 116 aa, unidentified
  REGION 1..116: note = Human (Homo sapiens), chimeric or humanized antibody peptide sequence
QIQLVQSGAKVKKPGASVKISCKASGYTFTDYSMHWVRQAPGQGLEWVARINTETGEPTFADDFRGRFSVTLDTSTSTAYLEITSLRSDDTAVYFCAIFYYGRDTWGQGTALTVSS

SEQ ID NO: 412 - protein, 30 aa, unidentified
  REGION 1..30: note = Human (Homo sapiens), chimeric or humanized antibody peptide sequence
QIQLVQSGAKVKKPGASVKISCKASGYTFT

SEQ ID NO: 413 - protein, 5 aa, unidentified
  REGION 1..5: note = Human (Homo sapiens), chimeric or humanized antibody peptide sequence
DYSMH

SEQ ID NO: 414 - protein, 14 aa, unidentified
  REGION 1..14: note = Human (Homo sapiens), chimeric or humanized antibody peptide sequence
WVRQAPGQGLEWVA

SEQ ID NO: 415 - protein, 17 aa, unidentified
  REGION 1..17: note = Human (Homo sapiens), chimeric or humanized antibody peptide sequence
RINTETGEPTFADDFRG

SEQ ID NO: 416 - protein, 32 aa, unidentified
  REGION 1..32: note = Human (Homo sapiens), chimeric or humanized antibody peptide sequence
RFSVTLDTSTSTAYLEITSLRSDDTAVYFCAI

SEQ ID NO: 417 - protein, 7 aa, unidentified
  REGION 1..7: note = Human (Homo sapiens), chimeric or humanized antibody peptide sequence
FYYGRDT

SEQ ID NO: 418 - protein, 11 aa, unidentified
  REGION 1..11: note = Human (Homo sapiens), chimeric or humanized antibody peptide sequence
WGQGTALTVSS

SEQ ID NO: 419 - protein, 116 aa, unidentified
  REGION 1..116: note = Human (Homo sapiens), chimeric or humanized antibody peptide sequence
QIQLVQSGAEVKKPGASVKISCKASGYTFTDYSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 420 - protein, 30 aa, unidentified; identical to SEQ ID NO: 396
  REGION 1..30: note = Human (Homo sapiens), chimeric or humanized antibody peptide sequence

SEQ ID NO: 421 - protein, 5 aa, unidentified
  REGION 1..5: note = Human (Homo sapiens), chimeric or humanized antibody peptide sequence
DYSMH

SEQ ID NO: 422 - protein, 14 aa, unidentified
  REGION 1..14: note = Human (Homo sapiens), chimeric or humanized antibody peptide sequence
WVRQAPGQGLEWVA

SEQ ID NO: 423 - protein, 17 aa, unidentified
  REGION 1..17: note = Human (Homo sapiens), chimeric or humanized antibody peptide sequence
RINTETGEPTFADDFRG

SEQ ID NO: 424 - protein, 32 aa, unidentified; identical to SEQ ID NO: 400
  REGION 1..32: note = Human (Homo sapiens), chimeric or humanized antibody peptide sequence

SEQ ID NO: 425 - protein, 7 aa, unidentified
  REGION 1..7: note = Human (Homo sapiens), chimeric or humanized antibody peptide sequence
FYYGRDT

SEQ ID NO: 426 - protein, 11 aa, unidentified
  REGION 1..11: note = Human (Homo sapiens), chimeric or humanized antibody peptide sequence
WGQGTTLTVSS

SEQ ID NO: 427 - protein, 116 aa, unidentified; identical to SEQ ID NO: 419
  REGION 1..116: note = Human (Homo sapiens), chimeric or humanized antibody peptide sequence

SEQ ID NO: 428 - protein, 30 aa, unidentified; identical to SEQ ID NO: 396
  REGION 1..30: note = Human (Homo sapiens), chimeric or humanized antibody peptide sequence

SEQ ID NO: 429 - protein, 5 aa, unidentified
  REGION 1..5: note = Human (Homo sapiens), chimeric or humanized antibody peptide sequence
DYSMH

SEQ ID NO: 430 - protein, 14 aa, unidentified
  REGION 1..14: note = Human (Homo sapiens), chimeric or humanized antibody peptide sequence
WVRQAPGQGLEWVA

SEQ ID NO: 431 - protein, 17 aa, unidentified
  REGION 1..17: note = Human (Homo sapiens), chimeric or humanized antibody peptide sequence
RINTETGEPTFADDFRG

SEQ ID NO: 432 - protein, 32 aa, unidentified; identical to SEQ ID NO: 400
  REGION 1..32: note = Human (Homo sapiens), chimeric or humanized antibody peptide sequence

SEQ ID NO: 433 - protein, 7 aa, unidentified
  REGION 1..7: note = Human (Homo sapiens), chimeric or humanized antibody peptide sequence
FYYGRDT

SEQ ID NO: 434 - protein, 11 aa, unidentified
  REGION 1..11: note = Human (Homo sapiens), chimeric or humanized antibody peptide sequence
WGQGTTLTVSS

SEQ ID NO: 435 - protein, 116 aa, unidentified; identical to SEQ ID NO: 121
  REGION 1..116: note = Human (Homo sapiens), chimeric or humanized antibody peptide sequence

SEQ ID NO: 436 - protein, 30 aa, unidentified
  REGION 1..30: note = Human (Homo sapiens), chimeric or humanized antibody peptide sequence
QIQLLQSGPELKKPGETVKISCKASGYTFT

SEQ ID NO: 437 - protein, 5 aa, unidentified
  REGION 1..5: note = Human (Homo sapiens), chimeric or humanized antibody peptide sequence
DYSMH

SEQ ID NO: 438 - protein, 14 aa, unidentified
  REGION 1..14: note = Human (Homo sapiens), chimeric or humanized antibody peptide sequence
WVKQAPGKGLKWVA

SEQ ID NO: 439 - protein, 17 aa, unidentified
  REGION 1..17: note = Human (Homo sapiens), chimeric or humanized antibody peptide sequence
RINTETGEPTFADDFRG

SEQ ID NO: 440 - protein, 32 aa, unidentified; identical to SEQ ID NO: 38
  REGION 1..32: note = Human (Homo sapiens), chimeric or humanized antibody peptide sequence

SEQ ID NO: 441 - protein, 7 aa, unidentified
  REGION 1..7: note = Human (Homo sapiens), chimeric or humanized antibody peptide sequence
FYYGRDS

SEQ ID NO: 442 - protein, 11 aa, unidentified
  REGION 1..11: note = Human (Homo sapiens), chimeric or humanized antibody peptide sequence
WGQGTTLTVSS

SEQ ID NO: 443 - protein, 116 aa, unidentified; identical to SEQ ID NO: 419
  REGION 1..116: note = Human (Homo sapiens), chimeric or humanized antibody peptide sequence

SEQ ID NO: 444 - protein, 30 aa, unidentified; identical to SEQ ID NO: 396
  REGION 1..30: note = Human (Homo sapiens), chimeric or humanized antibody peptide sequence

SEQ ID NO: 445 - protein, 5 aa, unidentified
  REGION 1..5: note = Human (Homo sapiens), chimeric or humanized antibody peptide sequence
DYSMH

SEQ ID NO: 446 - protein, 14 aa, unidentified
  REGION 1..14: note = Human (Homo sapiens), chimeric or humanized antibody peptide sequence
WVRQAPGQGLEWVA

SEQ ID NO: 447 - protein, 17 aa, unidentified
  REGION 1..17: note = Human (Homo sapiens), chimeric or humanized antibody peptide sequence
RINTETGEPTFADDFRG

SEQ ID NO: 448 - protein, 32 aa, unidentified; identical to SEQ ID NO: 400
  REGION 1..32: note = Human (Homo sapiens), chimeric or humanized antibody peptide sequence

SEQ ID NO: 449 - protein, 7 aa, unidentified
  REGION 1..7: note = Human (Homo sapiens), chimeric or humanized antibody peptide sequence
FYYGRDT

SEQ ID NO: 450 - protein, 11 aa, unidentified
  REGION 1..11: note = Human (Homo sapiens), chimeric or humanized antibody peptide sequence
WGQGTTLTVSS

SEQ ID NO: 451 - protein, 113 aa, unidentified
  REGION 1..113: note = Human (Homo sapiens), chimeric or humanized antibody peptide sequence
EIVMTQSPATLSVSPGERVTMSCKSSQSLLNSRTRKNYLAWYQQKPGQAPRLLIYWASTRESGVPARFSGSGSGTEFTLTISSVQSEDFAVYYCKQSYNLLSFGQGTVLEIKR

SEQ ID NO: 452 - protein, 23 aa, unidentified
  REGION 1..23: note = Human (Homo sapiens), chimeric or humanized antibody peptide sequence
EIVMTQSPATLSVSPGERVTMSC

SEQ ID NO: 453 - protein, 17 aa, unidentified
  REGION 1..17: note = Human (Homo sapiens), chimeric or humanized antibody peptide sequence
KSSQSLLNSRTRKNYLA

SEQ ID NO: 454 - protein, 15 aa, unidentified
  REGION 1..15: note = Human (Homo sapiens), chimeric or humanized antibody peptide sequence
WYQQKPGQAPRLLIY

SEQ ID NO: 455 - protein, 7 aa, unidentified
  REGION 1..7: note = Human (Homo sapiens), chimeric or humanized antibody peptide sequence
WASTRES

SEQ ID NO: 456 - protein, 32 aa, unidentified
  REGION 1..32: note = Human (Homo sapiens), chimeric or humanized antibody peptide sequence
GVPARFSGSGSGTEFTLTISSVQSEDFAVYYC

SEQ ID NO: 457 - protein, 8 aa, unidentified
  REGION 1..8: note = Human (Homo sapiens), chimeric or humanized antibody peptide sequence
KQSYNLLS

SEQ ID NO: 458 - protein, 11 aa, unidentified
  REGION 1..11: note = Human (Homo sapiens), chimeric or humanized antibody peptide sequence
FGQGTVLEIKR

SEQ ID NO: 459 - protein, 113 aa, unidentified
  REGION 1..113: note = Human (Homo sapiens), chimeric or humanized antibody peptide sequence
EIVMTQSPATLSVSPGEIVTMSCKSSQSLLNSRSRKNYLAWYQQKPGQAPRLLIYWASTRESGVPARFSGSGSGTEFTLTISSVQSEDFAVYYCKQSYNLISFGQGTVLEIKR

SEQ ID NO: 460 - protein, 23 aa, unidentified
  REGION 1..23: note = Human (Homo sapiens), chimeric or humanized antibody peptide sequence
EIVMTQSPATLSVSPGEIVTMSC

SEQ ID NO: 461 - protein, 17 aa, unidentified
  REGION 1..17: note = Human (Homo sapiens), chimeric or humanized antibody peptide sequence
KSSQSLLNSRSRKNYLA

SEQ ID NO: 462 - protein, 15 aa, unidentified
  REGION 1..15: note = Human (Homo sapiens), chimeric or humanized antibody peptide sequence
WYQQKPGQAPRLLIY

SEQ ID NO: 463 - protein, 7 aa, unidentified
  REGION 1..7: note = Human (Homo sapiens), chimeric or humanized antibody peptide sequence
WASTRES

SEQ ID NO: 464 - protein, 32 aa, unidentified; identical to SEQ ID NO: 456
  REGION 1..32: note = Human (Homo sapiens), chimeric or humanized antibody peptide sequence

SEQ ID NO: 465 - protein, 8 aa, unidentified
  REGION 1..8: note = Human (Homo sapiens), chimeric or humanized antibody peptide sequence
KQSYNLIS

SEQ ID NO: 466 - protein, 11 aa, unidentified
  REGION 1..11: note = Human (Homo sapiens), chimeric or humanized antibody peptide sequence
FGQGTVLEIKR

SEQ ID NO: 467 - protein, 113 aa, unidentified
  REGION 1..113: note = Human (Homo sapiens), chimeric or humanized antibody peptide sequence
EIVMTQTPVTLSVSPGERVTMSCKSSQSLLNSRTRKNYLAWYQQKPGQAPRLLIYWASTRESDVPARFSGSGSGTEFTLTISSVQSEDFAVYYCKQSSNLISFGQGTVLEIKR

SEQ ID NO: 468 - protein, 23 aa, unidentified
  REGION 1..23: note = Human (Homo sapiens), chimeric or humanized antibody peptide sequence
EIVMTQTPVTLSVSPGERVTMSC

SEQ ID NO: 469 - protein, 17 aa, unidentified
  REGION 1..17: note = Human (Homo sapiens), chimeric or humanized antibody peptide sequence
KSSQSLLNSRTRKNYLA

SEQ ID NO: 470 - protein, 15 aa, unidentified
  REGION 1..15: note = Human (Homo sapiens), chimeric or humanized antibody peptide sequence
WYQQKPGQAPRLLIY

SEQ ID NO: 471 - protein, 7 aa, unidentified
  REGION 1..7: note = Human (Homo sapiens), chimeric or humanized antibody peptide sequence
WASTRES

SEQ ID NO: 472 - protein, 32 aa, unidentified
  REGION 1..32: note = Human (Homo sapiens), chimeric or humanized antibody peptide sequence
DVPARFSGSGSGTEFTLTISSVQSEDFAVYYC

SEQ ID NO: 473 - protein, 8 aa, unidentified
  REGION 1..8: note = Human (Homo sapiens), chimeric or humanized antibody peptide sequence
KQSSNLIS

SEQ ID NO: 474 - protein, 11 aa, unidentified
  REGION 1..11: note = Human (Homo sapiens), chimeric or humanized antibody peptide sequence
FGQGTVLEIKR

SEQ ID NO: 475 - protein, 113 aa, unidentified
  REGION 1..113: note = Human (Homo sapiens), chimeric or humanized antibody peptide sequence
EIVMTQSPATLSVSPGERVTMSCKSSQSLLNSRSRKNYLAWYQQKPGQAPRLLIYWASTRESGVPARFSGSGSGTEFTLTISSVQSEDFAVYYCKQSYNLLSFGQGTVLEIKR

SEQ ID NO: 476 - protein, 23 aa, unidentified
  REGION 1..23: note = Human (Homo sapiens), chimeric or humanized antibody peptide sequence
EIVMTQSPATLSVSPGERVTMSC

SEQ ID NO: 477 - protein, 5 aa, unidentified
  REGION 1..5: note = Human (Homo sapiens), chimeric or humanized antibody peptide sequence
AVHRQ

SEQ ID NO: 478 - protein, 17 aa, unidentified
  REGION 1..17: note = Human (Homo sapiens), chimeric or humanized antibody peptide sequence
KSSQSLLNSRSRKNYLA

SEQ ID NO: 479 - protein, 15 aa, unidentified
  REGION 1..15: note = Human (Homo sapiens), chimeric or humanized antibody peptide sequence
WYQQKPGQAPRLLIY

SEQ ID NO: 480 - protein, 7 aa, unidentified
  REGION 1..7: note = Human (Homo sapiens), chimeric or humanized antibody peptide sequence
WASTRES

SEQ ID NO: 481 - protein, 32 aa, unidentified; identical to SEQ ID NO: 456
  REGION 1..32: note = Human (Homo sapiens), chimeric or humanized antibody peptide sequence

SEQ ID NO: 482 - protein, 8 aa, unidentified
  REGION 1..8: note = Human (Homo sapiens), chimeric or humanized antibody peptide sequence
KQSYNLLS

SEQ ID NO: 483 - protein, 11 aa, unidentified
  REGION 1..11: note = Human (Homo sapiens), chimeric or humanized antibody peptide sequence
FGQGTVLEIKR

SEQ ID NO: 484 - protein, 113 aa, unidentified; identical to SEQ ID NO: 475
  REGION 1..113: note = Human (Homo sapiens), chimeric or humanized antibody peptide sequence

SEQ ID NO: 485 - protein, 23 aa, unidentified
  REGION 1..23: note = Human (Homo sapiens), chimeric or humanized antibody peptide sequence
EIVMTQSPATLSVSPGERVTMSC

SEQ ID NO: 486 - protein, 17 aa, unidentified
  REGION 1..17: note = Human (Homo sapiens), chimeric or humanized antibody peptide sequence
KSSQSLLNSRSRKNYLA

SEQ ID NO: 487 - protein, 15 aa, unidentified
  REGION 1..15: note = Human (Homo sapiens), chimeric or humanized antibody peptide sequence
WYQQKPGQAPRLLIY

SEQ ID NO: 488 - protein, 7 aa, unidentified
  REGION 1..7: note = Human (Homo sapiens), chimeric or humanized antibody peptide sequence
WASTRES

SEQ ID NO: 489 - protein, 32 aa, unidentified; identical to SEQ ID NO: 456
  REGION 1..32: note = Human (Homo sapiens), chimeric or humanized antibody peptide sequence

SEQ ID NO: 490 - protein, 8 aa, unidentified
  REGION 1..8: note = Human (Homo sapiens), chimeric or humanized antibody peptide sequence
KQSYNLLS

SEQ ID NO: 491 - protein, 11 aa, unidentified
  REGION 1..11: note = Human (Homo sapiens), chimeric or humanized antibody peptide sequence
FGQGTVLEIKR

SEQ ID NO: 492 - protein, 117 aa, unidentified
  REGION 1..117: note = Human (Homo sapiens), chimeric or humanized antibody peptide sequence
DIVMTQSPSSLAVSAGEKVTMSCKSSQSLLGRGDLGRLKKNALAWYQQKPGQSPRLLIYWASTRESGVPDRFTGSGSGTDFTLTISSVQAEDLAVYYCKQSYNLLSFGAGTKLELKR

SEQ ID NO: 493 - protein, 23 aa, unidentified
  REGION 1..23: note = Human (Homo sapiens), chimeric or humanized antibody peptide sequence
DIVMTQSPSSLAVSAGEKVTMSC

SEQ ID NO: 494 - protein, 21 aa, unidentified
  REGION 1..21: note = Human (Homo sapiens), chimeric or humanized antibody peptide sequence
KSSQSLLGRGDLGRLKKNALA

SEQ ID NO: 495 - protein, 15 aa, unidentified
  REGION 1..15: note = Human (Homo sapiens), chimeric or humanized antibody peptide sequence
WYQQKPGQSPRLLIY

SEQ ID NO: 496 - protein, 7 aa, unidentified
  REGION 1..7: note = Human (Homo sapiens), chimeric or humanized antibody peptide sequence
WASTRES

SEQ ID NO: 497 - protein, 32 aa, unidentified; identical to SEQ ID NO: 190
  REGION 1..32: note = Human (Homo sapiens), chimeric or humanized antibody peptide sequence

SEQ ID NO: 498 - protein, 8 aa, unidentified
  REGION 1..8: note = Human (Homo sapiens), chimeric or humanized antibody peptide sequence
KQSYNLLS

SEQ ID NO: 499 - protein, 11 aa, unidentified
  REGION 1..11: note = Human (Homo sapiens), chimeric or humanized antibody peptide sequence
FGAGTKLELKR

SEQ ID NO: 500 - protein, 117 aa, unidentified
  REGION 1..117: note = Human (Homo sapiens), chimeric or humanized antibody peptide sequence
EIVMTQSPATLSVSPGERVTMSCKSSQSLLGRGDLGRLKKNALAWYQQKPGQAPRLLIYWASTRESGVPARFSGSGSGTEFTLTISSVQSEDFAVYYCKQSYNLLSFGQGTVLEIKR

SEQ ID NO: 501 - protein, 23 aa, unidentified
  REGION 1..23: note = Human (Homo sapiens), chimeric or humanized antibody peptide sequence
EIVMTQSPATLSVSPGERVTMSC

SEQ ID NO: 502 - protein, 21 aa, unidentified
  REGION 1..21: note = Human (Homo sapiens), chimeric or humanized antibody peptide sequence
KSSQSLLGRGDLGRLKKNALA

SEQ ID NO: 503 - protein, 15 aa, unidentified
  REGION 1..15: note = Human (Homo sapiens), chimeric or humanized antibody peptide sequence
WYQQKPGQAPRLLIY

SEQ ID NO: 504 - protein, 7 aa, unidentified
  REGION 1..7: note = Human (Homo sapiens), chimeric or humanized antibody peptide sequence
WASTRES

SEQ ID NO: 505 - protein, 32 aa, unidentified; identical to SEQ ID NO: 456
  REGION 1..32: note = Human (Homo sapiens), chimeric or humanized antibody peptide sequence

SEQ ID NO: 506 - protein, 8 aa, unidentified
  REGION 1..8: note = Human (Homo sapiens), chimeric or humanized antibody peptide sequence
KQSYNLLS

SEQ ID NO: 507 - protein, 11 aa, unidentified
  REGION 1..11: note = Human (Homo sapiens), chimeric or humanized antibody peptide sequence
FGQGTVLEIKR

SEQ ID NO: 508 - protein, 5 aa, unidentified
  REGION 1..5: note = Human (Homo sapiens), chimeric or humanized antibody peptide sequence
DYSMH

SEQ ID NO: 509 - protein, 17 aa, unidentified
  REGION 1..17: note = Human (Homo sapiens), chimeric or humanized antibody peptide sequence
RINTETGEPTFADDFRG

SEQ ID NO: 510 - protein, 7 aa, unidentified
  REGION 1..7: note = Human (Homo sapiens), chimeric or humanized antibody peptide sequence
FYYGRDS

SEQ ID NO: 511 - protein, 5 aa, unidentified
  REGION 1..5: note = Human (Homo sapiens), chimeric or humanized antibody peptide sequence
DYSMH

SEQ ID NO: 512 - protein, 17 aa, unidentified
  REGION 1..17: note = Human (Homo sapiens), chimeric or humanized antibody peptide sequence
RINTETGEPTFADDFRG

SEQ ID NO: 513 - protein, 7 aa, unidentified
  REGION 1..7: note = Human (Homo sapiens), chimeric or humanized antibody peptide sequence
FYYGRDS

SEQ ID NO: 514 - protein, 5 aa, unidentified
  REGION 1..5: note = Human (Homo sapiens), chimeric or humanized antibody peptide sequence
DYSMH

SEQ ID NO: 515 - protein, 17 aa, unidentified
  REGION 1..17: note = Human (Homo sapiens), chimeric or humanized antibody peptide sequence
RINTETGEPTFADDFRG

SEQ ID NO: 516 - protein, 7 aa, unidentified
  REGION 1..7: note = Human (Homo sapiens), chimeric or humanized antibody peptide sequence
FYYGRDT

SEQ ID NO: 517 - protein, 5 aa, unidentified
  REGION 1..5: note = Human (Homo sapiens), chimeric or humanized antibody peptide sequence
DYSMH

SEQ ID NO: 518 - protein, 17 aa, unidentified
  REGION 1..17: note = Human (Homo sapiens), chimeric or humanized antibody peptide sequence
RINTETGEPTFADDFRG

SEQ ID NO: 519 - protein, 7 aa, unidentified
  REGION 1..7: note = Human (Homo sapiens), chimeric or humanized antibody peptide sequence
FYYGRDT

SEQ ID NO: 520 - protein, 5 aa, unidentified
  REGION 1..5: note = Human (Homo sapiens), chimeric or humanized antibody peptide sequence
DYSMH

SEQ ID NO: 521 - protein, 17 aa, unidentified
  REGION 1..17: note = Human (Homo sapiens), chimeric or humanized antibody peptide sequence
RINTETGEPTFADDFRG

SEQ ID NO: 522 - protein, 7 aa, unidentified
  REGION 1..7: note = Human (Homo sapiens), chimeric or humanized antibody peptide sequence
FYYGRDT

SEQ ID NO: 523 - protein, 5 aa, unidentified
  REGION 1..5: note = Human (Homo sapiens), chimeric or humanized antibody peptide sequence
DYSMH

SEQ ID NO: 524 - protein, 17 aa, unidentified
  REGION 1..17: note = Human (Homo sapiens), chimeric or humanized antibody peptide sequence
RINTETGEPTFADDFRG

SEQ ID NO: 525 - protein, 7 aa, unidentified
  REGION 1..7: note = Human (Homo sapiens), chimeric or humanized antibody peptide sequence
FYYGRDS

SEQ ID NO: 526 - protein, 5 aa, unidentified
  REGION 1..5: note = Human (Homo sapiens), chimeric or humanized antibody peptide sequence
DYSMH

SEQ ID NO: 527 - protein, 17 aa, unidentified
  REGION 1..17: note = Human (Homo sapiens), chimeric or humanized antibody peptide sequence
RINTETGEPTFADDFRG

SEQ ID NO: 528 - protein, 7 aa, unidentified
  REGION 1..7: note = Human (Homo sapiens), chimeric or humanized antibody peptide sequence
FYYGRDT

SEQ ID NO: 529 - protein, 17 aa, unidentified
  REGION 1..17: note = Human (Homo sapiens), chimeric or humanized antibody peptide sequence
KSSQSLLNSRTRKNYLA

SEQ ID NO: 530 - protein, 7 aa, unidentified
  REGION 1..7: note = Human (Homo sapiens), chimeric or humanized antibody peptide sequence
WASTRES

SEQ ID NO: 531 - protein, 8 aa, unidentified
  REGION 1..8: note = Human (Homo sapiens), chimeric or humanized antibody peptide sequence
KQSYNLLS

SEQ ID NO: 532 - protein, 17 aa, unidentified
  REGION 1..17: note = Human (Homo sapiens), chimeric or humanized antibody peptide sequence
KSSQSLLNSRSRKNYLA

SEQ ID NO: 533 - protein, 7 aa, unidentified
  REGION 1..7: note = Human (Homo sapiens), chimeric or humanized antibody peptide sequence
WASTRES

SEQ ID NO: 534 - protein, 8 aa, unidentified
  REGION 1..8: note = Human (Homo sapiens), chimeric or humanized antibody peptide sequence
KQSYNLIS

SEQ ID NO: 535 - protein, 17 aa, unidentified
  REGION 1..17: note = Human (Homo sapiens), chimeric or humanized antibody peptide sequence
KSSQSLLNSRTRKNYLA

SEQ ID NO: 536 - protein, 7 aa, unidentified
  REGION 1..7: note = Human (Homo sapiens), chimeric or humanized antibody peptide sequence
WASTRES

SEQ ID NO: 537 - protein, 8 aa, unidentified
  REGION 1..8: note = Human (Homo sapiens), chimeric or humanized antibody peptide sequence
KQSSNLIS

SEQ ID NO: 538 - protein, 17 aa, unidentified
  REGION 1..17: note = Human (Homo sapiens), chimeric or humanized antibody peptide sequence
KSSQSLLNSRSRKNYLA

SEQ ID NO: 539 - protein, 7 aa, unidentified
  REGION 1..7: note = Human (Homo sapiens), chimeric or humanized antibody peptide sequence
WASTRES

SEQ ID NO: 540 - protein, 8 aa, unidentified
  REGION 1..8: note = Human (Homo sapiens), chimeric or humanized antibody peptide sequence
KQSYNLLS

SEQ ID NO: 541 - protein, 17 aa, unidentified
  REGION 1..17: note = Human (Homo sapiens), chimeric or humanized antibody peptide sequence
KSSQSLLNSRSRKNYLA

SEQ ID NO: 542 - protein, 7 aa, unidentified
  REGION 1..7: note = Human (Homo sapiens), chimeric or humanized antibody peptide sequence
WASTRES

SEQ ID NO: 543 - protein, 8 aa, unidentified
  REGION 1..8: note = Human (Homo sapiens), chimeric or humanized antibody peptide sequence
KQSYNLLS

SEQ ID NO: 544 - protein, 21 aa, unidentified
  REGION 1..21: note = Human (Homo sapiens), chimeric or humanized antibody peptide sequence
KSSQSLLGRGDLGRLKKNALA

SEQ ID NO: 545 - protein, 7 aa, unidentified
  REGION 1..7: note = Human (Homo sapiens), chimeric or humanized antibody peptide sequence
WASTRES

SEQ ID NO: 546 - protein, 8 aa, unidentified
  REGION 1..8: note = Human (Homo sapiens), chimeric or humanized antibody peptide sequence
KQSYNLLS

SEQ ID NO: 547 - protein, 21 aa, unidentified
  REGION 1..21: note = Human (Homo sapiens), chimeric or humanized antibody peptide sequence
KSSQSLLGRGDLGRLKKNALA

SEQ ID NO: 548 - protein, 7 aa, unidentified
  REGION 1..7: note = Human (Homo sapiens), chimeric or humanized antibody peptide sequence
WASTRES

SEQ ID NO: 549 - protein, 8 aa, unidentified
  REGION 1..8: note = Human (Homo sapiens), chimeric or humanized antibody peptide sequence
KQSYNLLS

SEQ ID NO: 550 - protein, 30 aa, unidentified
  REGION 1..30: note = Human (Homo sapiens), chimeric or humanized antibody peptide sequence
  VARIANT 9: note = MISC_FEATURE - Xaa is Pro or Ala
  VARIANT 10: note = MISC_FEATURE - Xaa is Glu or Lys
  VARIANT 11: note = MISC_FEATURE - Xaa is Lys or Val
  VARIANT 16: note = MISC_FEATURE - Xaa is Glu or Ala
  VARIANT 17: note = MISC_FEATURE - Xaa is Thr or Ser
QIQLVQSGXXXKKPGXXVKISCKASGYTFT

SEQ ID NO: 551 - protein, 14 aa, unidentified
  REGION 1..14: note = Human (Homo sapiens), chimeric or humanized antibody peptide sequence
  VARIANT 3: note = MISC_FEATURE - Xaa is Lys or Arg
  VARIANT 8: note = MISC_FEATURE - Xaa is Lys or Gln
  VARIANT 11: note = MISC_FEATURE - Xaa is Lys or Glu
WVXQAPGXGLXWVA

SEQ ID NO: 552 - protein, 32 aa, unidentified
  REGION 1..32: note = Human (Homo sapiens), chimeric or humanized antibody peptide sequence
  VARIANT 3: note = MISC_FEATURE - Xaa is Ala, Thr or Ser
  VARIANT 5: note = MISC_FEATURE - Xaa is Ser or Thr
  VARIANT 7: note = MISC_FEATURE - Xaa is Glu or Asp
  VARIANT 10: note = MISC_FEATURE - Xaa is Ala or Thr
  VARIANT 18: note = MISC_FEATURE - Xaa is Asn , Arg or Thr
  VARIANT 19: note = MISC_FEATURE - Xaa is Asn or Ser
  VARIANT 21: note = MISC_FEATURE - Xaa is Lys or Arg
  VARIANT 22: note = MISC_FEATURE - Xaa is Asn or Ser
  VARIANT 23: note = MISC_FEATURE - Xaa is Glu or Asp
  VARIANT 27: note = MISC_FEATURE - Xaa is Thr or Val
RFXVXLXTSXSTAYLZIXXLXXXDTAXYFCAI

SEQ ID NO: 553 - protein, 11 aa, unidentified
  REGION 1..11: note = Human (Homo sapiens), chimeric or humanized antibody peptide sequence
  VARIANT 6: note = MISC_FEATURE - Xaa is Ala or Thr
WGQGTXLTVSS

SEQ ID NO: 554 - protein, 23 aa, unidentified
  REGION 1..23: note = Human (Homo sapiens), chimeric or humanized antibody peptide sequence
  VARIANT 1: note = MISC_FEATURE - Xaa is Asp or Glu
  VARIANT 7: note = MISC_FEATURE - Xaa is Ser or Thr
  VARIANT 9: note = MISC_FEATURE - Xaa is Ser , Ala or Val
  VARIANT 10: note = MISC_FEATURE - Xaa is Ser or Thr
  VARIANT 12: note = MISC_FEATURE - Xaa is Ala or Ser
  VARIANT 15: note = MISC_FEATURE - Xaa is Ala or Pro
  VARIANT 18: note = MISC_FEATURE - Xaa is Lys , Arg or Ile
XIVMTQXPXXLXVSXGEXVTMSC

SEQ ID NO: 555 - protein, 15 aa, unidentified
  REGION 1..15: note = Human (Homo sapiens), chimeric or humanized antibody peptide sequence
  VARIANT 9: note = MISC_FEATURE - Xaa is Ser or Ala
WYQQKPGQXPRLLIY

SEQ ID NO: 556 - protein, 32 aa, unidentified
  REGION 1..32: note = Human (Homo sapiens), chimeric or humanized antibody peptide sequence
  VARIANT 1: note = MISC_FEATURE - Xaa is Glu or Asp
  VARIANT 4: note = MISC_FEATURE - Xaa is Asp or Ala
  VARIANT 7: note = MISC_FEATURE - Xaa is Thr or Ser
  VARIANT 14: note = MISC_FEATURE - Xaa is Asp or Glu
  VARIANT 24: note = MISC_FEATURE - Xaa is Ala or Ser
  VARIANT 27: note = MISC_FEATURE - Xaa is Leu or Phe
XVPXRFXGSGSGTXFTLTISSVQXEDXAVYYC

SEQ ID NO: 557 - protein, 11 aa, unidentified
  REGION 1..11: note = Human (Homo sapiens), chimeric or humanized antibody peptide sequence
  VARIANT 3: note = MISC_FEATURE - Xaa is Ala or Gln
  VARIANT 6: note = MISC_FEATURE - Xaa is Lys or Val
  VARIANT 9: note = MISC_FEATURE - Xaa is Leu or Ile
FGXGTXLEXKR

SEQ ID NO: 558 - protein, 30 aa, unidentified
  REGION 1..30: note = Human (Homo sapiens), chimeric or humanized antibody peptide sequence
  VARIANT 5: note = misc_feature - Xaa can be any naturally occurring amino acid
  VARIANT 9..11: note = misc_feature - Xaa can be any naturally occurring amino acid
  VARIANT 16..17: note = misc_feature - Xaa can be any naturally occurring amino acid
QIQLXQSGXXXKKPGXXVKISCKASGYTFT

SEQ ID NO: 559 - protein, 14 aa, unidentified
  REGION 1..14: note = Human (Homo sapiens), chimeric or humanized antibody peptide sequence
  VARIANT 3: note = MISC_FEATURE - Xaa is Arg or Lys
  VARIANT 8: note = MISC_FEATURE - Xaa is Gln or Lys
  VARIANT 11: note = MISC_FEATURE - Xaa is Gly or Lys
  VARIANT 13: note = MISC_FEATURE - Xaa is Val or Met
  VARIANT 14: note = MISC_FEATURE - Xaa is Ala or Gly
WVXQAPGXGLXWXX

SEQ ID NO: 560 - protein, 32 aa, unidentified
  REGION 1..32: note = Human (Homo sapiens), chimeric or humanized antibody peptide sequence
  VARIANT 3: note = MISC_FEATURE - Xaa is Thr , Ala or Ser
  VARIANT 4: note = MISC_FEATURE - Xaa is Val or Phe
  VARIANT 5: note = MISC_FEATURE - Xaa is Thr or Ser
  VARIANT 7: note = MISC_FEATURE - Xaa is Asp or Glu
  VARIANT 10: note = MISC_FEATURE - Xaa is Ala or Thr
  VARIANT 11: note = MISC_FEATURE - Xaa is Ser or Thr
  VARIANT 14: note = MISC_FEATURE - Xaa is Tyr or Asn
  VARIANT 16: note = misc_feature - Xaa can be any naturally occurring amino acid
  VARIANT 18: note = MISC_FEATURE - Xaa is Arg, Asn, Ile or Thr
  VARIANT 19: note = misc_feature - Xaa can be any naturally occurring amino acid
  VARIANT 20: note = MISC_FEATURE - Xaa is Arg or Lys
  VARIANT 21: note = MISC_FEATURE - Xaa is Ser or Asn
  VARIANT 22: note = MISC_FEATURE - Xaa is Asp or Glu
  VARIANT 22..23: note = misc_feature - Xaa can be any naturally occurring amino acid
  VARIANT 26: note = MISC_FEATURE - Xaa is Val, Thr or Lys
  VARIANT 27: note = misc_feature - Xaa can be any naturally occurring amino acid
RFXXXLXTSXXTAXLXIXXLXXXDTAXYFCAI

SEQ ID NO: 561 - protein, 12 aa, unidentified
  REGION 1..12: note = Human (Homo sapiens), chimeric or humanized antibody peptide sequence
  VARIANT 6: note = misc_feature - Xaa can be any naturally occurring amino acid
  VARIANT 9: note = MISC_FEATURE - Xaa is Thr or Ala
WGQGTXLVTVSS

SEQ ID NO: 562 - protein, 5 aa, unidentified
  REGION 1..5: note = Human (Homo sapiens), chimeric or humanized antibody peptide sequence
DYSMH

SEQ ID NO: 563 - protein, 17 aa, unidentified
  REGION 1..17: note = Human (Homo sapiens), chimeric or humanized antibody peptide sequence
  VARIANT 1: note = MISC_FEATURE - Xaa is Arg or Trp
  VARIANT 3: note = MISC_FEATURE - Xaa is Asn or Lys
  VARIANT 7: note = MISC_FEATURE - Xaa is Gly or Asp
  VARIANT 11: note = MISC_FEATURE - Xaa is Phe or Tyr
  VARIANT 16: note = MISC_FEATURE - Xaa is Arg, Asn, Lys or Gly
  VARIANT 17: note = MISC_FEATURE - Xaa is Gly or Glu
XIXTETXEPTXADDFXX

SEQ ID NO: 564 - protein, 7 aa, unidentified
  REGION 1..7: note = Human (Homo sapiens), chimeric or humanized antibody peptide sequence
  VARIANT 1: note = MISC_FEATURE - Xaa is Phe or Tyr
  VARIANT 7: note = MISC_FEATURE - Xaa is Ser or Thr
XYYGRDX

SEQ ID NO: 565 - protein, 23 aa, unidentified
  REGION 1..23: note = Human (Homo sapiens), chimeric or humanized antibody peptide sequence
  VARIANT 1: note = MISC_FEATURE - Xaa is Glu or Asp
  VARIANT 5: note = MISC_FEATURE - Xaa is Thr or Ser
  VARIANT 7: note = MISC_FEATURE - Xaa is Ser or Thr
  VARIANT 9: note = MISC_FEATURE - Xaa is Ala, Ser or Val
  VARIANT 10: note = MISC_FEATURE - Xaa is Thr or Ser
  VARIANT 12: note = MISC_FEATURE - Xaa is Ser or Ala
  VARIANT 15: note = MISC_FEATURE - Xaa is Pro or Ala
  VARIANT 18: note = MISC_FEATURE - Xaa is Arg, Lys or Ile
XIVMXQXPXXLXVSXGEXVTMSC

SEQ ID NO: 566 - protein, 15 aa, unidentified
  REGION 1..15: note = Human (Homo sapiens), chimeric or humanized antibody peptide sequence
  VARIANT 9: note = MISC_FEATURE - Xaa is Ala or Ser
WYQQKPGQXPRLLIY

SEQ ID NO: 567 - protein, 32 aa, unidentified; identical to SEQ ID NO: 556
  REGION 1..32: note = Human (Homo sapiens), chimeric or humanized antibody peptide sequence
  VARIANT 1: note = MISC_FEATURE - Xaa is Glu or Asp
  VARIANT 4: note = MISC_FEATURE - Xaa is Ala or Asp
  VARIANT 7: note = MISC_FEATURE - Xaa is Ser or Thr
  VARIANT 14: note = MISC_FEATURE - Xaa is Glu or Asp
  VARIANT 24: note = MISC_FEATURE - Xaa is Ser, Asp or Ala
  VARIANT 27: note = MISC_FEATURE - Xaa is Phe or Leu

SEQ ID NO: 568 - protein, 11 aa, unidentified
  REGION 1..11: note = Human (Homo sapiens), chimeric or humanized antibody peptide sequence
  VARIANT 3: note = MISC_FEATURE - Xaa is Gln or Ala
  VARIANT 6: note = MISC_FEATURE - Xaa is Val or Lys
  VARIANT 9: note = MISC_FEATURE - Xaa is Ile or Leu
FGXGTXLEXKR

SEQ ID NO: 569 - protein, 17 aa, unidentified
  REGION 1..17: note = Human (Homo sapiens), chimeric or humanized antibody peptide sequence
  VARIANT 11: note = MISC_FEATURE - Xaa is Ser or Thr
KSSQSLLNSRXRKNYLA

SEQ ID NO: 570 - protein, 7 aa, unidentified
  REGION 1..7: note = Human (Homo sapiens), chimeric or humanized antibody peptide sequence
WASTRES

SEQ ID NO: 571 - protein, 8 aa, unidentified
  REGION 1..8: note = Human (Homo sapiens), chimeric or humanized antibody peptide sequence
KQSYNLLS

SEQ ID NO: 572 - protein, 17 aa, unidentified
  REGION 1..17: note = Human (Homo sapiens), chimeric or humanized antibody peptide sequence
KSSQSLLNSRSRKNYLA

SEQ ID NO: 573 - protein, 17 aa, unidentified
  REGION 1..17: note = Human (Homo sapiens), chimeric or humanized antibody peptide sequence
KSSQSLLGRGDLGNALA

SEQ ID NO: 574 - protein, 19 aa, unidentified
  REGION 1..19: note = Human (Homo sapiens), chimeric or humanized antibody peptide sequence
KSSQSLLNSGRGDLGNALA

SEQ ID NO: 575 - protein, 21 aa, unidentified
  REGION 1..21: note = Human (Homo sapiens), chimeric or humanized antibody peptide sequence
KSSQSLLGRGDLGRLKKNALA

SEQ ID NO: 576 - protein, 25 aa, unidentified
  REGION 1..25: note = Human (Homo sapiens), chimeric or humanized antibody peptide sequence
KSSQSLLGRGDLGRLKKQKDHNALA

SEQ ID NO: 577 - protein, 24 aa, unidentified
  REGION 1..24: note = Human (Homo sapiens), chimeric or humanized antibody peptide sequence
KSSQSLLGRGDLGRLKKQKDNALA

SEQ ID NO: 578 - protein, 23 aa, unidentified
  REGION 1..23: note = Human (Homo sapiens), chimeric or humanized antibody peptide sequence
KSSQSLLGRGDLGRLKKQKNALA

SEQ ID NO: 579 - protein, 22 aa, unidentified
  REGION 1..22: note = Human (Homo sapiens), chimeric or humanized antibody peptide sequence
KSSQSLLGRGDLGRLKKQNALA

SEQ ID NO: 580 - protein, 20 aa, unidentified
  REGION 1..20: note = Human (Homo sapiens), chimeric or humanized antibody peptide sequence
KSSQSLLGRGDLGRLKNALA

SEQ ID NO: 581 - protein, 19 aa, unidentified
  REGION 1..19: note = Human (Homo sapiens), chimeric or humanized antibody peptide sequence
KSSQSLLGRGDLGRLNALA

SEQ ID NO: 582 - protein, 18 aa, unidentified
  REGION 1..18: note = Human (Homo sapiens), chimeric or humanized antibody peptide sequence
KSSQSLLGRGDLGRNALA

SEQ ID NO: 583 - protein, 22 aa, unidentified
  REGION 1..22: note = Human (Homo sapiens), chimeric or humanized antibody peptide sequence
KSSQSLLGRGDLGRLKKQKDHH

SEQ ID NO: 584 - protein, 21 aa, unidentified
  REGION 1..21: note = Human (Homo sapiens), chimeric or humanized antibody peptide sequence
KSSQSLLGRGDLGRLKKQKDH

SEQ ID NO: 585 - protein, 20 aa, unidentified
  REGION 1..20: note = Human (Homo sapiens), chimeric or humanized antibody peptide sequence
KSSQSLLGRGDLGRLKKQKD

SEQ ID NO: 586 - protein, 19 aa, unidentified
  REGION 1..19: note = Human (Homo sapiens), chimeric or humanized antibody peptide sequence
KSSQSLLGRGDLGRLKKQK

SEQ ID NO: 587 - protein, 18 aa, unidentified
  REGION 1..18: note = Human (Homo sapiens), chimeric or humanized antibody peptide sequence
KSSQSLLGRGDLGRLKKQ

SEQ ID NO: 588 - protein, 17 aa, unidentified
  REGION 1..17: note = Human (Homo sapiens), chimeric or humanized antibody peptide sequence
KSSQSLLGRGDLGRLKK

SEQ ID NO: 589 - protein, 16 aa, unidentified
  REGION 1..16: note = Human (Homo sapiens), chimeric or humanized antibody peptide sequence
KSSQSLLGRGDLGRLK

SEQ ID NO: 590 - protein, 15 aa, unidentified
  REGION 1..15: note = Human (Homo sapiens), chimeric or humanized antibody peptide sequence
KSSQSLLGRGDLGRL

SEQ ID NO: 591 - protein, 54 aa, Homo sapiens
FLQDGTKTVEYAPCRSQDIDADGQGFCQGGFSIDFTKADRVLLGGPGSFYWQGQ

SEQ ID NO: 592 - protein, 54 aa, Pan troglodytes; identical to SEQ ID NO: 591

SEQ ID NO: 593 - protein, 54 aa, Macaca mulatta; identical to SEQ ID NO: 591

SEQ ID NO: 594 - protein, 54 aa, Macaca fascicularis; identical to SEQ ID NO: 591

SEQ ID NO: 595 - protein, 54 aa, Bos taurus
FLQDGTKTVEYAPCRSKNIDADGQGFCQGGFSIDFTKADRVLLGGPGSFYWQGQ

SEQ ID NO: 596 - protein, 54 aa, unidentified; identical to SEQ ID NO: 595
  REGION 1..54: note = Sus sp.

SEQ ID NO: 597 - protein, 54 aa, Equus ferus
FLQDGAKTVEYAPCRSKNIDADGQGFCQGGFSIDFTKADRVLLGGPGSFYWQGQ

SEQ ID NO: 598 - protein, 54 aa, Mus musculus; identical to SEQ ID NO: 595

SEQ ID NO: 599 - protein, 54 aa, unidentified; identical to SEQ ID NO: 595
  REGION 1..54: note = Rattus sp.

SEQ ID NO: 600 - protein, 54 aa, unidentified; identical to SEQ ID NO: 595
  REGION 1..54: note = Dasypus sp.

SEQ ID NO: 601 - protein, 54 aa, Ornithorhynchus anatinus
FLQDGTKTVEYAPCRSRSIDADGQGFCQGGFSIDFTKADRVLLGGPGSFYWQGQ

SEQ ID NO: 602 - protein, 92 aa, Homo sapiens
SASMHNNIEKLNSVGNDLSRKMAFFSRDFRLGFGSYVDKTVSPYISIHPERIHNQCSDYNLDCMPPHGYIHVLSLTENITEFEKAVHRQKIS

SEQ ID NO: 603 - protein, 92 aa, Pan troglodytes
SASMHNNIEKLNSVGNDLSRKMAFFSRDFRLGFGSYVDKTVSPYISIHPERIHNQCSDYNLDCMPPHGYIHVLSLTENITEFERAVHRQKIS

SEQ ID NO: 604 - protein, 92 aa, Macaca mulatta; identical to SEQ ID NO: 602

SEQ ID NO: 605 - protein, 92 aa, Macaca fascicularis; identical to SEQ ID NO: 602

SEQ ID NO: 606 - protein, 92 aa, Bos taurus; identical to SEQ ID NO: 602

SEQ ID NO: 607 - protein, 92 aa, unidentified
  REGION 1..92: note = Sus sp.
SASMHNNIEKLNTVGNDLSRKMAFFSRDFRLGFGSYVDKTVSPYISIHPERIHNQCSDYNLDCMPPHGYIHVLSLTENITEFEKAVHRQKIS

SEQ ID NO: 608 - protein, 92 aa, Equus ferus; identical to SEQ ID NO: 602

SEQ ID NO: 609 - protein, 92 aa, Mus musculus
SASMHNNIEKLNSVGNDLSKKMALYSRDFRLGFGSYVDKTVSPYISIHPERIHNQCSDYNLDCMPPHGYIHVLSLTENITEFEKAVHRQKIS

SEQ ID NO: 610 - protein, 92 aa, unidentified
  REGION 1..92: note = Rattus sp.
SASMHNNIEKLNSVGNDLSKKMALFSHDFRLGFGSYVDKTVSPYISIHPERIHNQCSDYNLDCMPPHGYIHVLSLTENITEFEKAVHRQKIS

SEQ ID NO: 611 - protein, 92 aa, unidentified
  REGION 1..92: note = Dasypus sp.
SASMHNNIEKLNSVGNDLSRKMAFFSLDFRLGFGSYVDKTVSPYISIHPERIHNQCSDYNLDCMPPHGYIHVLSLTENITEFAKAVHRQKIS

SEQ ID NO: 612 - protein, 92 aa, Ornithorhynchus anatinus
SASMHNNIEKLNSVGNDLSQKMADFTRDFRLGFGSYVDKTVSPYISIHPGRIRNQCSQYDLDCMPPHGYIHVLPLTENVTEFEKAVNKQKIS

SEQ ID NO: 613 - protein, 9 aa, unidentified
  REGION 1..9: note = Human (Homo sapiens), chimeric or humanized antibody peptide sequence
YTFTDYSMH

SEQ ID NO: 614 - protein, 17 aa, unidentified
  REGION 1..17: note = Human (Homo sapiens), chimeric or humanized antibody peptide sequence
RINTETGEPTFADDFRG

SEQ ID NO: 615 - protein, 7 aa, unidentified
  REGION 1..7: note = Human (Homo sapiens), chimeric or humanized antibody peptide sequence
FYYGRDS

SEQ ID NO: 616 - protein, 17 aa, unidentified
  REGION 1..17: note = Human (Homo sapiens), chimeric or humanized antibody peptide sequence
KSSQSLLNSRTRKNYLA

SEQ ID NO: 617 - protein, 8 aa, unidentified
  REGION 1..8: note = Human (Homo sapiens), chimeric or humanized antibody peptide sequence
YWASTRES

SEQ ID NO: 618 - protein, 8 aa, unidentified
  REGION 1..8: note = Human (Homo sapiens), chimeric or humanized antibody peptide sequence
KQSYNLLS

SEQ ID NO: 619 - protein, 26 aa, unidentified
  REGION 1..26: note = Human (Homo sapiens), chimeric or humanized antibody peptide sequence
SASMHNNIEKLNSVGNDLSRKMAFFS

SEQ ID NO: 620 - protein, 28 aa, unidentified
  REGION 1..28: note = Human (Homo sapiens), chimeric or humanized antibody peptide sequence
TVSPYISIHPERIHNQCSDYNLDCMPPH

SEQ ID NO: 621 - protein, 11 aa, unidentified
  REGION 1..11: note = Human (Homo sapiens), chimeric or humanized antibody peptide sequence
NITEFEKAVHR

SEQ ID NO: 622 - protein, 118 aa, unidentified
  REGION 1..118: note = Human (Homo sapiens), chimeric or humanized antibody peptide sequence
KQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFD

SEQ ID NO: 623 - protein, 439 aa, Homo sapiens
FNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARP

SEQ ID NO: 624 - protein, 122 aa, unidentified
  REGION 1..122: note = Human (Homo sapiens), chimeric or humanized antibody peptide sequence
QVQLQQSGAELVRPGTSVKVSCKASGYAFTNYLIEWVKQRPGQGLEWIGVINPGTGGTNYNKKFKVKATLTADKSSSTAYMQLGGLTFDDSAVYFCAREGNARTYYYAMDYWGQGTSVTVSS

SEQ ID NO: 625 - protein, 27 aa, unidentified
  REGION 1..27: note = Human (Homo sapiens), chimeric or humanized antibody peptide sequence
QVQLQQSGAELVRPGTSVKVSCKASGY

SEQ ID NO: 626 - protein, 27 aa, unidentified
  REGION 1..27: note = Human (Homo sapiens), chimeric or humanized antibody peptide sequence
QVQLQQSGAELVRPGASVKVSCKASGY

SEQ ID NO: 627 - protein, 27 aa, unidentified
  REGION 1..27: note = Human (Homo sapiens), chimeric or humanized antibody peptide sequence
QVQLQQSGAELVRPGTSVKVPCKASGY

SEQ ID NO: 628 - protein, 8 aa, unidentified
  REGION 1..8: note = Human (Homo sapiens), chimeric or humanized antibody peptide sequence
AFTNYLIE

SEQ ID NO: 629 - protein, 8 aa, unidentified
  REGION 1..8: note = Human (Homo sapiens), chimeric or humanized antibody peptide sequence
AFTDYLIE

SEQ ID NO: 630 - protein, 8 aa, unidentified
  REGION 1..8: note = Human (Homo sapiens), chimeric or humanized antibody peptide sequence
AFTDNLIE

SEQ ID NO: 631 - protein, 8 aa, unidentified
  REGION 1..8: note = Human (Homo sapiens), chimeric or humanized antibody peptide sequence
AFTDYLIQ

SEQ ID NO: 632 - protein, 14 aa, unidentified
  REGION 1..14: note = Human (Homo sapiens), chimeric or humanized antibody peptide sequence
WVKQRPGQGLEWIG

SEQ ID NO: 633 - protein, 14 aa, unidentified
  REGION 1..14: note = Human (Homo sapiens), chimeric or humanized antibody peptide sequence
WVRQRPGQGLEWIG

SEQ ID NO: 634 - protein, 17 aa, unidentified
  REGION 1..17: note = Human (Homo sapiens), chimeric or humanized antibody peptide sequence
VINPGTGGTNYNKKFKV

SEQ ID NO: 635 - protein, 17 aa, unidentified
  REGION 1..17: note = Human (Homo sapiens), chimeric or humanized antibody peptide sequence
VINPETGGTNYNAKFKG

SEQ ID NO: 636 - protein, 17 aa, unidentified
  REGION 1..17: note = Human (Homo sapiens), chimeric or humanized antibody peptide sequence
VINPETGGTNYNAKFRG

SEQ ID NO: 637 - protein, 32 aa, unidentified
  REGION 1..32: note = Human (Homo sapiens), chimeric or humanized antibody peptide sequence
KATLTADKSSSTAYMQLGGLTFDDSAVYFCAR

SEQ ID NO: 638 - protein, 32 aa, unidentified; identical to SEQ ID NO: 302
  REGION 1..32: note = Human (Homo sapiens), chimeric or humanized antibody peptide sequence

SEQ ID NO: 639 - protein, 32 aa, unidentified
  REGION 1..32: note = Human (Homo sapiens), chimeric or humanized antibody peptide sequence
KATLTADKSSSSVYMQLSSLTSGDSAVYFCAR

SEQ ID NO: 640 - protein, 32 aa, unidentified
  REGION 1..32: note = Human (Homo sapiens), chimeric or humanized antibody peptide sequence
KATLTADKSSSTAYMQLSSLTSGDSAVYFCAR

SEQ ID NO: 641 - protein, 32 aa, unidentified
  REGION 1..32: note = Human (Homo sapiens), chimeric or humanized antibody peptide sequence
KATLTADKSSSSAYMQLSGLTSGDSAVYFCAR

SEQ ID NO: 642 - protein, 32 aa, unidentified
  REGION 1..32: note = Human (Homo sapiens), chimeric or humanized antibody peptide sequence
KATLTADKSSSSAYMQLSSLSSGDSAVYFCAR

SEQ ID NO: 643 - protein, 32 aa, unidentified; identical to SEQ ID NO: 641
  REGION 1..32: note = Human (Homo sapiens), chimeric or humanized antibody peptide sequence

SEQ ID NO: 644 - protein, 32 aa, unidentified
  REGION 1..32: note = Human (Homo sapiens), chimeric or humanized antibody peptide sequence
RATLTADKSSSSAYMQLSSLTSGDSAVYFCAR

SEQ ID NO: 645 - protein, 32 aa, unidentified
  REGION 1..32: note = Human (Homo sapiens), chimeric or humanized antibody peptide sequence
KATLTADKSSSSAYMQLSSLTSDDSAVYFCAR

SEQ ID NO: 646 - protein, 32 aa, unidentified
  REGION 1..32: note = Human (Homo sapiens), chimeric or humanized antibody peptide sequence
KVTLTADKTSSSAYMQLSSLTSGDSAVYFCAR

SEQ ID NO: 647 - protein, 32 aa, unidentified
  REGION 1..32: note = Human (Homo sapiens), chimeric or humanized antibody peptide sequence
KVTLTADKSSSSAYMQLNSLTSGDSAVYFCAR

SEQ ID NO: 648 - protein, 32 aa, unidentified
  REGION 1..32: note = Human (Homo sapiens), chimeric or humanized antibody peptide sequence
KATLTANKSSSSAYMQLSSLTSGDSAVYFCAR

SEQ ID NO: 649 - protein, 32 aa, unidentified
  REGION 1..32: note = Human (Homo sapiens), chimeric or humanized antibody peptide sequence
KATLTADKSSSSAYLQLSSLTSGDSAVYFCAR

SEQ ID NO: 650 - protein, 32 aa, unidentified; identical to SEQ ID NO: 644
  REGION 1..32: note = Human (Homo sapiens), chimeric or humanized antibody peptide sequence

SEQ ID NO: 651 - protein, 13 aa, unidentified
  REGION 1..13: note = Human (Homo sapiens), chimeric or humanized antibody peptide sequence
EGNARTYYYAMDY

SEQ ID NO: 652 - protein, 11 aa, unidentified
  REGION 1..11: note = Human (Homo sapiens), chimeric or humanized antibody peptide sequence
EAGNYIYAMDY

SEQ ID NO: 653 - protein, 10 aa, unidentified
  REGION 1..10: note = Human (Homo sapiens), chimeric or humanized antibody peptide sequence
AGNYIYAMDY

SEQ ID NO: 654 - protein, 11 aa, unidentified
  REGION 1..11: note = Human (Homo sapiens), chimeric or humanized antibody peptide sequence
EAGNYIYAMDY

SEQ ID NO: 655 - protein, 11 aa, unidentified
  REGION 1..11: note = Human (Homo sapiens), chimeric or humanized antibody peptide sequence
WGQGTSVTVSS

SEQ ID NO: 656 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVKQRPGQGLEWIGVINPETGGTNYNAKFKGKATLTADKSSSSAYMQLSSLTSGDSAVYFCAREAGNYIYAMDYWGQGTSVTVSS

SEQ ID NO: 657 - protein, 119 aa, unidentified
  REGION 1..119: note = Human (Homo sapiens), chimeric or humanized antibody peptide sequence
QVQLQQSGAELVRPGTSVKVSCKASGYAFTDYLIEWVKQRPGQGLEWIGVINPETGGTNYNAKFRGKATLTADKSSSSAYMQLSSLTSGDSAVYFCARAGNYIYAMDYWGQGTSVTVSS

SEQ ID NO: 658 - protein, 120 aa, unidentified
  REGION 1..120: note = Human (Homo sapiens), chimeric or humanized antibody peptide sequence
QVQLQQSGAELVRPGASVKVSCKASGYAFTDYLIEWVRQRPGQGLEWIGVINPETGGTNYNAKFRGKATLTADKSSSSVYMQLSSLTSGDSAVYFCAREAGNYIYAMDYWGQGTSVTVSS

SEQ ID NO: 659 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKATLTADKSSSTAYMQLSSLTSGDSAVYFCAREAGNYIYAMDYWGQGTSVTVSS

SEQ ID NO: 660 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKATLTADKSSSSAYMQLSGLTSGDSAVYFCAREAGNYIYAMDYWGQGTSVTVSS

SEQ ID NO: 661 - protein, 120 aa, unidentified
  REGION 1..120: note = Human (Homo sapiens), chimeric or humanized antibody peptide sequence
QVQLQQSGAELVRPGTSVKVSCKASGYAFTDNLIEWVRQRPGQGLEWIGVINPETGGTNYNAKFRGKATLTADKSSSSAYMQLSSLTSGDSAVYFCAREAGNYIYAMDYWGQGTSVTVSS

SEQ ID NO: 662 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVKQRPGQGLEWIGVINPETGGTNYNAKFRGKATLTADKSSSSAYMQLSSLSSGDSAVYFCAREAGNYIYAMDYWGQGTSVTVSS

SEQ ID NO: 663 - protein, 120 aa, unidentified; identical to SEQ ID NO: 660
  REGION 1..120: note = Human (Homo sapiens), chimeric or humanized antibody peptide sequence

SEQ ID NO: 664 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGRATLTADKSSSSAYMQLSSLTSGDSAVYFCAREAGNYIYAMDYWGQGTSVTVSS

SEQ ID NO: 665 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKATLTADKSSGSAYMQLSSLTSGDSAVYFCAREAGNYIYAMDYWGQGTSVTVSS

SEQ ID NO: 666 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKATLTADKSSSSAYMQLSSLTSDDSAVYFCAREAGNYIYAMDYWGQGTSVTVSS

SEQ ID NO: 667 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKVTLTADKTSSSAYMQLSSLTSGDSAVYFCAREAGNYIYAMDYWGQGTSVTVSS

SEQ ID NO: 668 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVRQRPGQGLEWIGVINPETGGTNYNAKFRGKVTLTADKSSSSAYMQLNSLTSGDSAVYFCAREAGNYIYAMDYWGQGTSVTVSS

SEQ ID NO: 669 - protein, 120 aa, unidentified; identical to SEQ ID NO: 664
  REGION 1..120: note = Human (Homo sapiens), chimeric or humanized antibody peptide sequence

SEQ ID NO: 670 - protein, 120 aa, unidentified; identical to SEQ ID NO: 667
  REGION 1..120: note = Human (Homo sapiens), chimeric or humanized antibody peptide sequence

SEQ ID NO: 671 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVKQRPGQGLEWIGVINPETGGTNYNAKFRGRATLTADKSSSSAYMQLSSLTSGDSAVYFCAREAGNYIYAMDYWGQGTSVTVSS

SEQ ID NO: 672 - protein, 120 aa, unidentified; identical to SEQ ID NO: 297
  REGION 1..120: note = Human (Homo sapiens), chimeric or humanized antibody peptide sequence

SEQ ID NO: 673 - protein, 120 aa, unidentified
  REGION 1..120: note = Human (Homo sapiens), chimeric or humanized antibody peptide sequence
QVQLQQSGAELVRPGTSVRVSCKASGYAFTDYLIQWVKQRPGQGLEWIGVINPETGGTNYNAKFRGKATLTANKSSSSAYMQLSSLTSGDSAVYFCAREAGNYIYAMDYWGQGTSVTVSS

SEQ ID NO: 674 - protein, 120 aa, unidentified
  REGION 1..120: note = Human (Homo sapiens), chimeric or humanized antibody peptide sequence
QVQLQQSGAELVRPGTSVKVPCKASGYAFTDYLIQWVKQRPGQGLEWIGVINPETGGTNYNAKFRGKATLTADKSSSSAYMQLSSLTSGDSAVYFCAREAGNYIYAMDYWGQGTSVTVSS

SEQ ID NO: 675 - protein, 120 aa, unidentified
  REGION 1..120: note = Human (Homo sapiens), chimeric or humanized antibody peptide sequence
QVQLQQSGAELVRPGTSVKVSCKASGYAFTDYLIEWVKQRPGQGLEWIGVINPETGGTNYNAKFRGKATLTADKSSSSAYMQLSSLTSGDSAVYFCAREAGNYIYAMDYWGQGTSVTVSS

SEQ ID NO: 676 - protein, 120 aa, unidentified
  REGION 1..120: note = Human (Homo sapiens), chimeric or humanized antibody peptide sequence
QVQLQQSGAELVRPGTSVKVSCKASGYAFTDYLIQWVKQRPGQGLEWIGVINPETGGTNYNAKFRGKATLTADKSSSSAYLQLSSLTSGDSAVYFCAREAGNYIYAMDYWGQGTSVTVSS

SEQ ID NO: 677 - protein, 120 aa, unidentified; identical to SEQ ID NO: 297
  REGION 1..120: note = Human (Homo sapiens), chimeric or humanized antibody peptide sequence

SEQ ID NO: 678 - protein, 108 aa, unidentified
  REGION 1..108: note = Human (Homo sapiens), chimeric or humanized antibody peptide sequence
DIQMTQSPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKA

SEQ ID NO: 679 - protein, 108 aa, unidentified
  REGION 1..108: note = Human (Homo sapiens), chimeric or humanized antibody peptide sequence
DIEMTQTPASLSASVGETVTITCRASENIYSYLVWYQQKQGKSPQVLVYNAKTLAEGVPSRFSGSGSGTQFSLKINSLQPEDFGSYYCQHHNGTPYTFGGGTKLEIKA

SEQ ID NO: 680 - protein, 108 aa, unidentified
  REGION 1..108: note = Human (Homo sapiens), chimeric or humanized antibody peptide sequence
DIVMTQSPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKA

SEQ ID NO: 681 - protein, 108 aa, unidentified; identical to SEQ ID NO: 680
  REGION 1..108: note = Human (Homo sapiens), chimeric or humanized antibody peptide sequence

SEQ ID NO: 682 - protein, 108 aa, unidentified
  REGION 1..108: note = Human (Homo sapiens), chimeric or humanized antibody peptide sequence
DIVMTQSPASLSASVGETVTITCRASVNIYSYLVWYQQKQGKSPQLLVHNAKTLAEGVPSRFSGSGSGTQFSLKINSVQPEDFGSYYCQHHHGTPYTFGGGTKLEIKA

SEQ ID NO: 683 - protein, 108 aa, unidentified
  REGION 1..108: note = Human (Homo sapiens), chimeric or humanized antibody peptide sequence
HIVMTQSPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKA

SEQ ID NO: 684 - protein, 108 aa, unidentified
  REGION 1..108: note = Human (Homo sapiens), chimeric or humanized antibody peptide sequence
DIVMTQSPASLSASVGETVTITCRASVNIYSYLVWYQQKQGKSPQLLVHNAKTLAEGVPSRFSGSGSGTQFSLKINSLQPEDVGSYYCQHHHGTPYTFGGGTKLEIKA

SEQ ID NO: 685 - protein, 108 aa, unidentified; identical to SEQ ID NO: 680
  REGION 1..108: note = Human (Homo sapiens), chimeric or humanized antibody peptide sequence

SEQ ID NO: 686 - protein, 108 aa, unidentified; identical to SEQ ID NO: 680
  REGION 1..108: note = Human (Homo sapiens), chimeric or humanized antibody peptide sequence

SEQ ID NO: 687 - protein, 108 aa, unidentified
  REGION 1..108: note = Human (Homo sapiens), chimeric or humanized antibody peptide sequence
DIVMTQSPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKR

SEQ ID NO: 688 - protein, 108 aa, unidentified
  REGION 1..108: note = Human (Homo sapiens), chimeric or humanized antibody peptide sequence
DIEMTQTPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKR

SEQ ID NO: 689 - protein, 108 aa, unidentified; identical to SEQ ID NO: 680
  REGION 1..108: note = Human (Homo sapiens), chimeric or humanized antibody peptide sequence

SEQ ID NO: 690 - protein, 108 aa, unidentified
  REGION 1..108: note = Human (Homo sapiens), chimeric or humanized antibody peptide sequence
DIVVTQSPASLSASVGETVTITCRASVNIYSYLVWYQQKQGKSPQLLVHNAKTLAEGVPSRFSGSGSGTQFSLKINSLQPEDFGSYYCQHHHGTPYTFGGGTKLEIKA

SEQ ID NO: 691 - protein, 108 aa, unidentified; identical to SEQ ID NO: 305
  REGION 1..108: note = Human (Homo sapiens), chimeric or humanized antibody peptide sequence

SEQ ID NO: 692 - protein, 23 aa, unidentified
  REGION 1..23: note = Human (Homo sapiens), chimeric or humanized antibody peptide sequence
DIQMTQSPASLSASVGETVTITC

SEQ ID NO: 693 - protein, 11 aa, unidentified
  REGION 1..11: note = Human (Homo sapiens), chimeric or humanized antibody peptide sequence
RASVNIYSYLV

SEQ ID NO: 694 - protein, 15 aa, unidentified
  REGION 1..15: note = Human (Homo sapiens), chimeric or humanized antibody peptide sequence
WYQQKQGKSPQLLVH

SEQ ID NO: 695 - protein, 7 aa, unidentified
  REGION 1..7: note = Human (Homo sapiens), chimeric or humanized antibody peptide sequence
NAKTLAE

SEQ ID NO: 696 - protein, 32 aa, unidentified; identical to SEQ ID NO: 310
  REGION 1..32: note = Human (Homo sapiens), chimeric or humanized antibody peptide sequence

SEQ ID NO: 697 - protein, 9 aa, unidentified
  REGION 1..9: note = Human (Homo sapiens), chimeric or humanized antibody peptide sequence
QHHHGTPYT

SEQ ID NO: 698 - protein, 11 aa, unidentified
  REGION 1..11: note = Human (Homo sapiens), chimeric or humanized antibody peptide sequence
FGGGTKLEIKA

SEQ ID NO: 699 - protein, 23 aa, unidentified
  REGION 1..23: note = Human (Homo sapiens), chimeric or humanized antibody peptide sequence
DIEMTQTPASLSASVGETVTITC

SEQ ID NO: 700 - protein, 11 aa, unidentified
  REGION 1..11: note = Human (Homo sapiens), chimeric or humanized antibody peptide sequence
RASENIYSYLV

SEQ ID NO: 701 - protein, 15 aa, unidentified
  REGION 1..15: note = Human (Homo sapiens), chimeric or humanized antibody peptide sequence
WYQQKQGKSPQVLVY

SEQ ID NO: 702 - protein, 9 aa, unidentified
  REGION 1..9: note = Human (Homo sapiens), chimeric or humanized antibody peptide sequence
QHHNGTPYT

SEQ ID NO: 703 - protein, 23 aa, unidentified
  REGION 1..23: note = Human (Homo sapiens), chimeric or humanized antibody peptide sequence
DIVMTQSPASLSASVGETVTITC

SEQ ID NO: 704 - protein, 23 aa, unidentified
  REGION 1..23: note = Human (Homo sapiens), chimeric or humanized antibody peptide sequence
HIVMTQSPASLSASVGETVTITC

SEQ ID NO: 705 - protein, 23 aa, unidentified
  REGION 1..23: note = Human (Homo sapiens), chimeric or humanized antibody peptide sequence
DIVVTQSPASLSASVGETVTITC

SEQ ID NO: 706 - protein, 11 aa, unidentified
  REGION 1..11: note = Human (Homo sapiens), chimeric or humanized antibody peptide sequence
FGGGTKLEIKR

SEQ ID NO: 707 - protein, 5 aa, unidentified
  REGION 1..5: note = Human (Homo sapiens), chimeric or humanized antibody peptide sequence
DYSMH

SEQ ID NO: 708 - protein, 6 aa, unidentified
  REGION 1..6: note = Human (Homo sapiens), chimeric or humanized antibody peptide sequence
ASTRES

SEQ ID NO: 709 - protein, 10 aa, unidentified
  REGION 1..10: note = Human (Homo sapiens), chimeric or humanized antibody peptide sequence
GRGDLGRLKK

SEQ ID NO: 710 - protein, 6 aa, unidentified
  REGION 1..6: note = Human (Homo sapiens), chimeric or humanized antibody peptide sequence
GRGDSP

SEQ ID NO: 711 - protein, 7 aa, unidentified
  REGION 1..7: note = Human (Homo sapiens), chimeric or humanized antibody peptide sequence
AKQRGDV

SEQ ID NO: 712 - protein, 9 aa, unidentified
  REGION 1..9: note = Human (Homo sapiens), chimeric or humanized antibody peptide sequence
RGDLGRLKK

SEQ ID NO: 713 - protein, 12 aa, unidentified
  REGION 1..12: note = Human (Homo sapiens), chimeric or humanized antibody peptide sequence
DDHGRGDLGRLK

SEQ ID NO: 714 - protein, 390 aa, unidentified
  REGION 1..390: note = Human (Homo sapiens), chimeric or humanized antibody peptide sequence
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 715 - protein, 338 aa, unidentified
  REGION 1..338: note = Human (Homo sapiens), chimeric or humanized antibody peptide sequence
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLPSYRLESQQTNRRKKRALDAAYCFRVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 716 - protein, 411 aa, unidentified
  REGION 1..411: note = Human (Homo sapiens), chimeric or humanized antibody peptide sequence
MKMHLQRALVVLALLNFATVSLSLSTCTTLDFGHIKKKRVEAIRGQILSKLRLTSPPEPTVTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKQHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 717 - protein, 219 aa, unidentified
  REGION 1..219: note = Human (Homo sapiens), chimeric or humanized antibody peptide sequence
DIVMTQSPSSLAVSAGEKVTMSCKSSQSLLNSRTRKNYLAWYQQKPGQSPRLLIYWASTRESGVPDRFTGSGSGTDFTLTISSVQAEDLAVYYCKQSYNLLSFGAGTKLELKAADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 718 - protein, 17 aa, unidentified
  REGION 1..17: note = Human (Homo sapiens), chimeric or humanized antibody peptide sequence
KSSQSLLGRGDLGNALA

SEQ ID NO: 719 - protein, 19 aa, unidentified
  REGION 1..19: note = Human (Homo sapiens), chimeric or humanized antibody peptide sequence
KSSQSLLNSGRGDLGNALA

SEQ ID NO: 720 - protein, 21 aa, unidentified
  REGION 1..21: note = Human (Homo sapiens), chimeric or humanized antibody peptide sequence
KSSQSLLGRGDLGRLKKNALA

SEQ ID NO: 721 - protein, 9 aa, unidentified
  REGION 1..9: note = Human (Homo sapiens), chimeric or humanized antibody peptide sequence
GRGDLGRLK

SEQ ID NO: 722 - protein, 116 aa, unidentified
  REGION 1..116: note = Human (Homo sapiens), chimeric or humanized antibody peptide sequence
QIQLVQSGPELKKPGETVKISCKASGYTFTDYSMHWVKQAPGKGLKWVARINTETGEPTFADDFRGRFAVSLETSASTAYLQINNLKNEDTATYFCAIFYYGRDSWGQGTTLTVSS

SEQ ID NO: 723 - protein, 116 aa, unidentified; identical to SEQ ID NO: 395
  REGION 1..116: note = Human (Homo sapiens), chimeric or humanized antibody peptide sequence

SEQ ID NO: 724 - protein, 116 aa, unidentified; identical to SEQ ID NO: 395
  REGION 1..116: note = Human (Homo sapiens), chimeric or humanized antibody peptide sequence

SEQ ID NO: 725 - protein, 116 aa, unidentified
  REGION 1..116: note = Human (Homo sapiens), chimeric or humanized antibody peptide sequence
QIQLVQSGAKVKKPGASVKISCKASGYTFTDYSMHWVRQAPGQGLEWVARINTETGEPTFADDFRGRFSVTLDTSTSTAYLEITSLRSDDTAVYFCAIFYYGRDTWGQGTTLTVSS

SEQ ID NO: 726 - protein, 116 aa, unidentified; identical to SEQ ID NO: 419
  REGION 1..116: note = Human (Homo sapiens), chimeric or humanized antibody peptide sequence

SEQ ID NO: 727 - protein, 113 aa, unidentified; identical to SEQ ID NO: 289
  REGION 1..113: note = Human (Homo sapiens), chimeric or humanized antibody peptide sequence

SEQ ID NO: 728 - protein, 113 aa, unidentified; identical to SEQ ID NO: 451
  REGION 1..113: note = Human (Homo sapiens), chimeric or humanized antibody peptide sequence

SEQ ID NO: 729 - protein, 113 aa, unidentified
  REGION 1..113: note = Human (Homo sapiens), chimeric or humanized antibody peptide sequence
EIVMTQSPATLSVSPGEIVTMSCKSSQSLLNSRSRKNYLAWYQQKPGQAPRLLIYWASTRESGVPARFSGSGSGTEFTLTISSVQSEDFAVYYCKQSYNLLSFGQGTVLEIKR

SEQ ID NO: 730 - protein, 113 aa, unidentified
  REGION 1..113: note = Human (Homo sapiens), chimeric or humanized antibody peptide sequence
EIVMTQTPVTLSVSPGERVTMSCKSSQSLLNSRTRKNYLAWYQQKPGQAPRLLIYWASTRESDVPARFSGSGSGTEFTLTISSVQSEDFAVYYCKQSSNLLSFGQGTVLEIKR

SEQ ID NO: 731 - protein, 113 aa, unidentified; identical to SEQ ID NO: 475
  REGION 1..113: note = Human (Homo sapiens), chimeric or humanized antibody peptide sequence

SEQ ID NO: 732 - protein, 30 aa, unidentified
  REGION 1..30: note = Human (Homo sapiens), chimeric or humanized antibody peptide sequence
QIQLVQSGPELKKPGETVKISCKASGYTFT

SEQ ID NO: 733 - protein, 5 aa, unidentified
  REGION 1..5: note = Human (Homo sapiens), chimeric or humanized antibody peptide sequence
DYSMH

SEQ ID NO: 734 - protein, 14 aa, unidentified
  REGION 1..14: note = Human (Homo sapiens), chimeric or humanized antibody peptide sequence
WVKQAPGKGLKWVA

SEQ ID NO: 735 - protein, 17 aa, unidentified
  REGION 1..17: note = Human (Homo sapiens), chimeric or humanized antibody peptide sequence
RINTETGEPTFADDFRG

SEQ ID NO: 736 - protein, 32 aa, unidentified; identical to SEQ ID NO: 38
  REGION 1..32: note = Human (Homo sapiens), chimeric or humanized antibody peptide sequence

SEQ ID NO: 737 - protein, 7 aa, unidentified
  REGION 1..7: note = Human (Homo sapiens), chimeric or humanized antibody peptide sequence
FYYGRDS

SEQ ID NO: 738 - protein, 11 aa, unidentified
  REGION 1..11: note = Human (Homo sapiens), chimeric or humanized antibody peptide sequence
WGQGTTLTVSS

SEQ ID NO: 739 - protein, 30 aa, unidentified; identical to SEQ ID NO: 396
  REGION 1..30: note = Human (Homo sapiens), chimeric or humanized antibody peptide sequence

SEQ ID NO: 740 - protein, 14 aa, unidentified
  REGION 1..14: note = Human (Homo sapiens), chimeric or humanized antibody peptide sequence
WVRQAPGQGLEWVA

SEQ ID NO: 741 - protein, 32 aa, unidentified; identical to SEQ ID NO: 400
  REGION 1..32: note = Human (Homo sapiens), chimeric or humanized antibody peptide sequence

SEQ ID NO: 742 - protein, 30 aa, unidentified; identical to SEQ ID NO: 412
  REGION 1..30: note = Human (Homo sapiens), chimeric or humanized antibody peptide sequence

SEQ ID NO: 743 - protein, 32 aa, unidentified; identical to SEQ ID NO: 416
  REGION 1..32: note = Human (Homo sapiens), chimeric or humanized antibody peptide sequence

SEQ ID NO: 744 - protein, 7 aa, unidentified
  REGION 1..7: note = Human (Homo sapiens), chimeric or humanized antibody peptide sequence
FYYGRDT

SEQ ID NO: 745 - protein, 23 aa, unidentified
  REGION 1..23: note = Human (Homo sapiens), chimeric or humanized antibody peptide sequence
DIVMTQSPSSLAVSAGEKVTMSC

SEQ ID NO: 746 - protein, 17 aa, unidentified
  REGION 1..17: note = Human (Homo sapiens), chimeric or humanized antibody peptide sequence
KSSQSLLNSRTRKNYLA

SEQ ID NO: 747 - protein, 15 aa, unidentified
  REGION 1..15: note = Human (Homo sapiens), chimeric or humanized antibody peptide sequence
WYQQKPGQSPRLLIY

SEQ ID NO: 748 - protein, 7 aa, unidentified
  REGION 1..7: note = Human (Homo sapiens), chimeric or humanized antibody peptide sequence
WASTRES

SEQ ID NO: 749 - protein, 32 aa, unidentified; identical to SEQ ID NO: 190
  REGION 1..32: note = Human (Homo sapiens), chimeric or humanized antibody peptide sequence

SEQ ID NO: 750 - protein, 8 aa, unidentified
  REGION 1..8: note = Human (Homo sapiens), chimeric or humanized antibody peptide sequence
KQSYNLLS

SEQ ID NO: 751 - protein, 11 aa, unidentified
  REGION 1..11: note = Human (Homo sapiens), chimeric or humanized antibody peptide sequence
FGAGTKLELKR

SEQ ID NO: 752 - protein, 23 aa, unidentified
  REGION 1..23: note = Human (Homo sapiens), chimeric or humanized antibody peptide sequence
EIVMTQSPATLSVSPGERVTMSC

SEQ ID NO: 753 - protein, 32 aa, unidentified; identical to SEQ ID NO: 456
  REGION 1..32: note = Human (Homo sapiens), chimeric or humanized antibody peptide sequence

SEQ ID NO: 754 - protein, 11 aa, unidentified
  REGION 1..11: note = Human (Homo sapiens), chimeric or humanized antibody peptide sequence
FGQGTVLEIKR

SEQ ID NO: 755 - protein, 23 aa, unidentified
  REGION 1..23: note = Human (Homo sapiens), chimeric or humanized antibody peptide sequence
EIVMTQSPATLSVSPGEIVTMSC

SEQ ID NO: 756 - protein, 4 aa, unidentified
  REGION 1..4: note = Human (Homo sapiens), chimeric or humanized antibody peptide sequence
RGDL

SEQ ID NO: 757 - protein, 23 aa, unidentified
  REGION 1..23: note = Human (Homo sapiens), chimeric or humanized antibody peptide sequence
EIVMTQTPVTLSVSPGERVTMSC

SEQ ID NO: 758 - protein, 32 aa, unidentified; identical to SEQ ID NO: 472
  REGION 1..32: note = Human (Homo sapiens), chimeric or humanized antibody peptide sequence

SEQ ID NO: 759 - protein, 5 aa, unidentified
  REGION 1..5: note = Human (Homo sapiens), chimeric or humanized antibody peptide sequence
FDDSY

SEQ ID NO: 760 - protein, 118 aa, unidentified; identical to SEQ ID NO: 622
  REGION 1..118: note = Human (Homo sapiens), chimeric or humanized antibody peptide sequence

SEQ ID NO: 761 - protein, 5 aa, unidentified
  REGION 1..5: note = Human (Homo sapiens), chimeric or humanized antibody peptide sequence
NLDCM

SEQ ID NO: 762 - protein, 5 aa, unidentified
  REGION 1..5: note = Human (Homo sapiens), chimeric or humanized antibody peptide sequence
YNLDC

SEQ ID NO: 763 - protein, 7 aa, unidentified
  REGION 1..7: note = Human (Homo sapiens), chimeric or humanized antibody peptide sequence
QCSDYNL

SEQ ID NO: 764 - protein, 5 aa, unidentified
  REGION 1..5: note = Human (Homo sapiens), chimeric or humanized antibody peptide sequence
SMHNN

SEQ ID NO: 765 - protein, 17 aa, unidentified
  REGION 1..17: note = Human (Homo sapiens), chimeric or humanized antibody peptide sequence
KSSQSLLGRGDLGRLKK

SEQ ID NO: 766 - protein, 8 aa, unidentified
  REGION 1..8: note = Human (Homo sapiens), chimeric or humanized antibody peptide sequence
TFTDYSMH

SEQ ID NO: 767 - protein, 17 aa, unidentified
  REGION 1..17: note = Human (Homo sapiens), chimeric or humanized antibody peptide sequence
RINTETGEPTFADDFRG

SEQ ID NO: 768 - protein, 7 aa, unidentified
  REGION 1..7: note = Human (Homo sapiens), chimeric or humanized antibody peptide sequence
FYYGRDS

SEQ ID NO: 769 - protein, 5 aa, unidentified
  REGION 1..5: note = Human (Homo sapiens), chimeric or humanized antibody peptide sequence
DYSMH

SEQ ID NO: 770 - protein, 17 aa, unidentified
  REGION 1..17: note = Human (Homo sapiens), chimeric or humanized antibody peptide sequence
RINTETGEPTFADDFRG

SEQ ID NO: 771 - protein, 7 aa, unidentified
  REGION 1..7: note = Human (Homo sapiens), chimeric or humanized antibody peptide sequence
FYYGRDS

SEQ ID NO: 772 - protein, 19 aa, unidentified
  REGION 1..19: note = Human (Homo sapiens), chimeric or humanized antibody peptide sequence
KSSQSLLNSGRGDLGNALA

SEQ ID NO: 773 - protein, 7 aa, unidentified
  REGION 1..7: note = Human (Homo sapiens), chimeric or humanized antibody peptide sequence
WASTRES

SEQ ID NO: 774 - protein, 8 aa, unidentified
  REGION 1..8: note = Human (Homo sapiens), chimeric or humanized antibody peptide sequence
KQSYNLIS

SEQ ID NO: 775 - protein, 5 aa, unidentified
  REGION 1..5: note = Human (Homo sapiens), chimeric or humanized antibody peptide sequence
DYSMH

SEQ ID NO: 776 - protein, 17 aa, unidentified
  REGION 1..17: note = Human (Homo sapiens), chimeric or humanized antibody peptide sequence
RINTETGEPTFADDFRG

SEQ ID NO: 777 - protein, 7 aa, unidentified
  REGION 1..7: note = Human (Homo sapiens), chimeric or humanized antibody peptide sequence
FYYGRDT

SEQ ID NO: 778 - protein, 25 aa, unidentified
  REGION 1..25: note = Human (Homo sapiens), chimeric or humanized antibody peptide sequence
KSSQSLLGRGDLGRLKKQKDHNALA

SEQ ID NO: 779 - protein, 7 aa, unidentified
  REGION 1..7: note = Human (Homo sapiens), chimeric or humanized antibody peptide sequence
WASTRES

SEQ ID NO: 780 - protein, 8 aa, unidentified
  REGION 1..8: note = Human (Homo sapiens), chimeric or humanized antibody peptide sequence
KQSSNLIS

SEQ ID NO: 781 - protein, 5 aa, unidentified
  REGION 1..5: note = Human (Homo sapiens), chimeric or humanized antibody peptide sequence
DYSMH

SEQ ID NO: 782 - protein, 17 aa, unidentified
  REGION 1..17: note = Human (Homo sapiens), chimeric or humanized antibody peptide sequence
RINTETGEPTFADDFRG

SEQ ID NO: 783 - protein, 7 aa, unidentified
  REGION 1..7: note = Human (Homo sapiens), chimeric or humanized antibody peptide sequence
FYYGRDY

SEQ ID NO: 784 - protein, 24 aa, unidentified
  REGION 1..24: note = Human (Homo sapiens), chimeric or humanized antibody peptide sequence
KSSQSLLGRGDLGRLKKQKDNALA

SEQ ID NO: 785 - protein, 7 aa, unidentified
  REGION 1..7: note = Human (Homo sapiens), chimeric or humanized antibody peptide sequence
WASTRES

SEQ ID NO: 786 - protein, 8 aa, unidentified
  REGION 1..8: note = Human (Homo sapiens), chimeric or humanized antibody peptide sequence
KQSYNLLS

SEQ ID NO: 787 - protein, 5 aa, unidentified
  REGION 1..5: note = Human (Homo sapiens), chimeric or humanized antibody peptide sequence
DYSMH

SEQ ID NO: 788 - protein, 17 aa, unidentified
  REGION 1..17: note = Human (Homo sapiens), chimeric or humanized antibody peptide sequence
RINTETGEPTFADDFRG

SEQ ID NO: 789 - protein, 7 aa, unidentified
  REGION 1..7: note = Human (Homo sapiens), chimeric or humanized antibody peptide sequence
FYYGRDT

SEQ ID NO: 790 - protein, 23 aa, unidentified
  REGION 1..23: note = Human (Homo sapiens), chimeric or humanized antibody peptide sequence
KSSQSLLGRGDLGRLKKQKNALA

SEQ ID NO: 791 - protein, 7 aa, unidentified
  REGION 1..7: note = Human (Homo sapiens), chimeric or humanized antibody peptide sequence
WASTRES

SEQ ID NO: 792 - protein, 8 aa, unidentified
  REGION 1..8: note = Human (Homo sapiens), chimeric or humanized antibody peptide sequence
KQSYNLLS

SEQ ID NO: 793 - protein, 5 aa, unidentified
  REGION 1..5: note = Human (Homo sapiens), chimeric or humanized antibody peptide sequence
DYSMH

SEQ ID NO: 794 - protein, 17 aa, unidentified
  REGION 1..17: note = Human (Homo sapiens), chimeric or humanized antibody peptide sequence
RINTETGEPTFADDFRG

SEQ ID NO: 795 - protein, 7 aa, unidentified
  REGION 1..7: note = Human (Homo sapiens), chimeric or humanized antibody peptide sequence
FYYGRDS

SEQ ID NO: 796 - protein, 22 aa, unidentified
  REGION 1..22: note = Human (Homo sapiens), chimeric or humanized antibody peptide sequence
KSSQSLLGRGDLGRLKKQNALA

SEQ ID NO: 797 - protein, 7 aa, unidentified
  REGION 1..7: note = Human (Homo sapiens), chimeric or humanized antibody peptide sequence
WASTRES

SEQ ID NO: 798 - protein, 8 aa, unidentified
  REGION 1..8: note = Human (Homo sapiens), chimeric or humanized antibody peptide sequence
KQSYNLLS

SEQ ID NO: 799 - protein, 5 aa, unidentified
  REGION 1..5: note = Human (Homo sapiens), chimeric or humanized antibody peptide sequence
DYSMH

SEQ ID NO: 800 - protein, 17 aa, unidentified
  REGION 1..17: note = Human (Homo sapiens), chimeric or humanized antibody peptide sequence
RINTETGEPTFADDFRG

SEQ ID NO: 801 - protein, 7 aa, unidentified
  REGION 1..7: note = Human (Homo sapiens), chimeric or humanized antibody peptide sequence
FYYGRDT

SEQ ID NO: 802 - protein, 21 aa, unidentified
  REGION 1..21: note = Human (Homo sapiens), chimeric or humanized antibody peptide sequence
KSSQSLLGRGDLGRLKKNALA

SEQ ID NO: 803 - protein, 7 aa, unidentified
  REGION 1..7: note = Human (Homo sapiens), chimeric or humanized antibody peptide sequence
WASTRES

SEQ ID NO: 804 - protein, 8 aa, unidentified
  REGION 1..8: note = Human (Homo sapiens), chimeric or humanized antibody peptide sequence
KQSYNLLS

SEQ ID NO: 805 - protein, 5 aa, unidentified
  REGION 1..5: note = Human (Homo sapiens), chimeric or humanized antibody peptide sequence
DYSMH

SEQ ID NO: 806 - protein, 17 aa, unidentified
  REGION 1..17: note = Human (Homo sapiens), chimeric or humanized antibody peptide sequence
RINTETGEPTFADDFRG

SEQ ID NO: 807 - protein, 7 aa, unidentified
  REGION 1..7: note = Human (Homo sapiens), chimeric or humanized antibody peptide sequence
FYYGRDS

SEQ ID NO: 808 - protein, 20 aa, unidentified
  REGION 1..20: note = Human (Homo sapiens), chimeric or humanized antibody peptide sequence
KSSQSLLGRGDLGRLKNALA

SEQ ID NO: 809 - protein, 7 aa, unidentified
  REGION 1..7: note = Human (Homo sapiens), chimeric or humanized antibody peptide sequence
WASTRES

SEQ ID NO: 810 - protein, 8 aa, unidentified
  REGION 1..8: note = Human (Homo sapiens), chimeric or humanized antibody peptide sequence
KQSYNLLS

SEQ ID NO: 811 - protein, 5 aa, unidentified
  REGION 1..5: note = Human (Homo sapiens), chimeric or humanized antibody peptide sequence
DYSMH

SEQ ID NO: 812 - protein, 17 aa, unidentified
  REGION 1..17: note = Human (Homo sapiens), chimeric or humanized antibody peptide sequence
RINTETGEPTFADDFRG

SEQ ID NO: 813 - protein, 7 aa, unidentified
  REGION 1..7: note = Human (Homo sapiens), chimeric or humanized antibody peptide sequence
FYYGRDS

SEQ ID NO: 814 - protein, 19 aa, unidentified
  REGION 1..19: note = Human (Homo sapiens), chimeric or humanized antibody peptide sequence
KSSQSLLGRGDLGRLNALA

SEQ ID NO: 815 - protein, 7 aa, unidentified
  REGION 1..7: note = Human (Homo sapiens), chimeric or humanized antibody peptide sequence
WASTRES

SEQ ID NO: 816 - protein, 8 aa, unidentified
  REGION 1..8: note = Human (Homo sapiens), chimeric or humanized antibody peptide sequence
KQSYNLIS

SEQ ID NO: 817 - protein, 5 aa, unidentified
  REGION 1..5: note = Human (Homo sapiens), chimeric or humanized antibody peptide sequence
DYSMH

SEQ ID NO: 818 - protein, 17 aa, unidentified
  REGION 1..17: note = Human (Homo sapiens), chimeric or humanized antibody peptide sequence
RINTETGEPTFADDFRG

SEQ ID NO: 819 - protein, 7 aa, unidentified
  REGION 1..7: note = Human (Homo sapiens), chimeric or humanized antibody peptide sequence
FYYGRDT

SEQ ID NO: 820 - protein, 18 aa, unidentified
  REGION 1..18: note = Human (Homo sapiens), chimeric or humanized antibody peptide sequence
KSSQSLLGRGDLGRNALA

SEQ ID NO: 821 - protein, 7 aa, unidentified
  REGION 1..7: note = Human (Homo sapiens), chimeric or humanized antibody peptide sequence
WASTRES

SEQ ID NO: 822 - protein, 8 aa, unidentified
  REGION 1..8: note = Human (Homo sapiens), chimeric or humanized antibody peptide sequence
KQSSNLIS

SEQ ID NO: 823 - protein, 5 aa, unidentified
  REGION 1..5: note = Human (Homo sapiens), chimeric or humanized antibody peptide sequence
DYSMH

SEQ ID NO: 824 - protein, 17 aa, unidentified
  REGION 1..17: note = Human (Homo sapiens), chimeric or humanized antibody peptide sequence
RINTETGEPTFADDFRG

SEQ ID NO: 825 - protein, 7 aa, unidentified
  REGION 1..7: note = Human (Homo sapiens), chimeric or humanized antibody peptide sequence
FYYGRDY

SEQ ID NO: 826 - protein, 17 aa, unidentified
  REGION 1..17: note = Human (Homo sapiens), chimeric or humanized antibody peptide sequence
KSSQSLLGRGDLGNALA

SEQ ID NO: 827 - protein, 7 aa, unidentified
  REGION 1..7: note = Human (Homo sapiens), chimeric or humanized antibody peptide sequence
WASTRES

SEQ ID NO: 828 - protein, 8 aa, unidentified
  REGION 1..8: note = Human (Homo sapiens), chimeric or humanized antibody peptide sequence
KQSYNLLS

SEQ ID NO: 829 - protein, 5 aa, unidentified
  REGION 1..5: note = Human (Homo sapiens), chimeric or humanized antibody peptide sequence
DYSMH

SEQ ID NO: 830 - protein, 17 aa, unidentified
  REGION 1..17: note = Human (Homo sapiens), chimeric or humanized antibody peptide sequence
RINTETGEPTFADDFRG

SEQ ID NO: 831 - protein, 7 aa, unidentified
  REGION 1..7: note = Human (Homo sapiens), chimeric or humanized antibody peptide sequence
FYYGRDT

SEQ ID NO: 832 - protein, 22 aa, unidentified
  REGION 1..22: note = Human (Homo sapiens), chimeric or humanized antibody peptide sequence
KSSQSLLGRGDLGRLKKQKDHH

SEQ ID NO: 833 - protein, 7 aa, unidentified
  REGION 1..7: note = Human (Homo sapiens), chimeric or humanized antibody peptide sequence
WASTRES

SEQ ID NO: 834 - protein, 8 aa, unidentified
  REGION 1..8: note = Human (Homo sapiens), chimeric or humanized antibody peptide sequence
KQSYNLLS

SEQ ID NO: 835 - protein, 5 aa, unidentified
  REGION 1..5: note = Human (Homo sapiens), chimeric or humanized antibody peptide sequence
DYSMH

SEQ ID NO: 836 - protein, 17 aa, unidentified
  REGION 1..17: note = Human (Homo sapiens), chimeric or humanized antibody peptide sequence
RINTETGEPTFADDFRG

SEQ ID NO: 837 - protein, 7 aa, unidentified
  REGION 1..7: note = Human (Homo sapiens), chimeric or humanized antibody peptide sequence
FYYGRDS

SEQ ID NO: 838 - protein, 21 aa, unidentified
  REGION 1..21: note = Human (Homo sapiens), chimeric or humanized antibody peptide sequence
KSSQSLLGRGDLGRLKKQKDH

SEQ ID NO: 839 - protein, 7 aa, unidentified
  REGION 1..7: note = Human (Homo sapiens), chimeric or humanized antibody peptide sequence
WASTRES

SEQ ID NO: 840 - protein, 8 aa, unidentified
  REGION 1..8: note = Human (Homo sapiens), chimeric or humanized antibody peptide sequence
KQSYNLLS

SEQ ID NO: 841 - protein, 5 aa, unidentified
  REGION 1..5: note = Human (Homo sapiens), chimeric or humanized antibody peptide sequence
DYSMH

SEQ ID NO: 842 - protein, 17 aa, unidentified
  REGION 1..17: note = Human (Homo sapiens), chimeric or humanized antibody peptide sequence
RINTETGEPTFADDFRG

SEQ ID NO: 843 - protein, 7 aa, unidentified
  REGION 1..7: note = Human (Homo sapiens), chimeric or humanized antibody peptide sequence
FYYGRDT

SEQ ID NO: 844 - protein, 20 aa, unidentified
  REGION 1..20: note = Human (Homo sapiens), chimeric or humanized antibody peptide sequence
KSSQSLLGRGDLGRLKKQKD

SEQ ID NO: 845 - protein, 7 aa, unidentified
  REGION 1..7: note = Human (Homo sapiens), chimeric or humanized antibody peptide sequence
WASTRES

SEQ ID NO: 846 - protein, 8 aa, unidentified
  REGION 1..8: note = Human (Homo sapiens), chimeric or humanized antibody peptide sequence
KQSYNLLS

SEQ ID NO: 847 - protein, 5 aa, unidentified
  REGION 1..5: note = Human (Homo sapiens), chimeric or humanized antibody peptide sequence
DYSMH

SEQ ID NO: 848 - protein, 17 aa, unidentified
  REGION 1..17: note = Human (Homo sapiens), chimeric or humanized antibody peptide sequence
RINTETGEPTFADDFRG

SEQ ID NO: 849 - protein, 7 aa, unidentified
  REGION 1..7: note = Human (Homo sapiens), chimeric or humanized antibody peptide sequence
FYYGRDT

SEQ ID NO: 850 - protein, 19 aa, unidentified
  REGION 1..19: note = Human (Homo sapiens), chimeric or humanized antibody peptide sequence
KSSQSLLGRGDLGRLKKQK

SEQ ID NO: 851 - protein, 7 aa, unidentified
  REGION 1..7: note = Human (Homo sapiens), chimeric or humanized antibody peptide sequence
WASTRES

SEQ ID NO: 852 - protein, 8 aa, unidentified
  REGION 1..8: note = Human (Homo sapiens), chimeric or humanized antibody peptide sequence
KQSSNLIS

SEQ ID NO: 853 - protein, 5 aa, unidentified
  REGION 1..5: note = Human (Homo sapiens), chimeric or humanized antibody peptide sequence
DYSMH

SEQ ID NO: 854 - protein, 17 aa, unidentified
  REGION 1..17: note = Human (Homo sapiens), chimeric or humanized antibody peptide sequence
RINTETGEPTFADDFRG

SEQ ID NO: 855 - protein, 7 aa, unidentified
  REGION 1..7: note = Human (Homo sapiens), chimeric or humanized antibody peptide sequence
FYYGRDY

SEQ ID NO: 856 - protein, 18 aa, unidentified
  REGION 1..18: note = Human (Homo sapiens), chimeric or humanized antibody peptide sequence
KSSQSLLGRGDLGRLKKQ

SEQ ID NO: 857 - protein, 7 aa, unidentified
  REGION 1..7: note = Human (Homo sapiens), chimeric or humanized antibody peptide sequence
WASTRES

SEQ ID NO: 858 - protein, 8 aa, unidentified
  REGION 1..8: note = Human (Homo sapiens), chimeric or humanized antibody peptide sequence
KQSYNLLS

SEQ ID NO: 859 - protein, 5 aa, unidentified
  REGION 1..5: note = Human (Homo sapiens), chimeric or humanized antibody peptide sequence
DYSMH

SEQ ID NO: 860 - protein, 17 aa, unidentified
  REGION 1..17: note = Human (Homo sapiens), chimeric or humanized antibody peptide sequence
RINTETGEPTFADDFRG

SEQ ID NO: 861 - protein, 7 aa, unidentified
  REGION 1..7: note = Human (Homo sapiens), chimeric or humanized antibody peptide sequence
FYYGRDT

SEQ ID NO: 862 - protein, 17 aa, unidentified
  REGION 1..17: note = Human (Homo sapiens), chimeric or humanized antibody peptide sequence
KSSQSLLGRGDLGRLKK

SEQ ID NO: 863 - protein, 7 aa, unidentified
  REGION 1..7: note = Human (Homo sapiens), chimeric or humanized antibody peptide sequence
WASTRES

SEQ ID NO: 864 - protein, 8 aa, unidentified
  REGION 1..8: note = Human (Homo sapiens), chimeric or humanized antibody peptide sequence
KQSYNLLS

SEQ ID NO: 865 - protein, 5 aa, unidentified
  REGION 1..5: note = Human (Homo sapiens), chimeric or humanized antibody peptide sequence
DYSMH

SEQ ID NO: 866 - protein, 17 aa, unidentified
  REGION 1..17: note = Human (Homo sapiens), chimeric or humanized antibody peptide sequence
RINTETGEPTFADDFRG

SEQ ID NO: 867 - protein, 7 aa, unidentified
  REGION 1..7: note = Human (Homo sapiens), chimeric or humanized antibody peptide sequence
FYYGRDS

SEQ ID NO: 868 - protein, 16 aa, unidentified
  REGION 1..16: note = Human (Homo sapiens), chimeric or humanized antibody peptide sequence
KSSQSLLGRGDLGRLK

SEQ ID NO: 869 - protein, 7 aa, unidentified
  REGION 1..7: note = Human (Homo sapiens), chimeric or humanized antibody peptide sequence
WASTRES

SEQ ID NO: 870 - protein, 8 aa, unidentified
  REGION 1..8: note = Human (Homo sapiens), chimeric or humanized antibody peptide sequence
KQSYNLLS

SEQ ID NO: 871 - protein, 5 aa, unidentified
  REGION 1..5: note = Human (Homo sapiens), chimeric or humanized antibody peptide sequence
DYSMH

SEQ ID NO: 872 - protein, 17 aa, unidentified
  REGION 1..17: note = Human (Homo sapiens), chimeric or humanized antibody peptide sequence
RINTETGEPTFADDFRG

SEQ ID NO: 873 - protein, 7 aa, unidentified
  REGION 1..7: note = Human (Homo sapiens), chimeric or humanized antibody peptide sequence
FYYGRDT

SEQ ID NO: 874 - protein, 15 aa, unidentified
  REGION 1..15: note = Human (Homo sapiens), chimeric or humanized antibody peptide sequence
KSSQSLLGRGDLGRL

SEQ ID NO: 875 - protein, 7 aa, unidentified
  REGION 1..7: note = Human (Homo sapiens), chimeric or humanized antibody peptide sequence
WASTRES

SEQ ID NO: 876 - protein, 8 aa, unidentified
  REGION 1..8: note = Human (Homo sapiens), chimeric or humanized antibody peptide sequence
KQSYNLLS

SEQ ID NO: 877 - protein, 14 aa, unidentified
  REGION 1..14: note = Human (Homo sapiens), chimeric or humanized antibody peptide sequence
  VARIANT 3: note = MISC_FEATURE - Xaa is Lys or Arg
  VARIANT 8: note = MISC_FEATURE - Xaa is Lys or Gln
  VARIANT 11: note = MISC_FEATURE - Xaa is Lys or Glu
  VARIANT 13: note = MISC_FEATURE - Xaa is Val or Met
  VARIANT 14: note = MISC_FEATURE - Xaa is Ala or Gly
WVXQAPGXGLXWXX

SEQ ID NO: 878 - protein, 32 aa, unidentified
  REGION 1..32: note = Human (Homo sapiens), chimeric or humanized antibody peptide sequence
  VARIANT 3: note = MISC_FEATURE - Xaa is Ala, Thr or Ser
  VARIANT 4: note = MISC_FEATURE - Xaa is Val or Phe
  VARIANT 5: note = MISC_FEATURE - Xaa is Ser or Thr
  VARIANT 7: note = MISC_FEATURE - Xaa is Glu or Asp
  VARIANT 10: note = MISC_FEATURE - Xaa is Ala or Thr
  VARIANT 11: note = MISC_FEATURE - Xaa is Ser or Thr
  VARIANT 14: note = misc_feature - Xaa can be any naturally occurring amino acid
  VARIANT 15: note = MISC_FEATURE - Xaa is Tyr or Asn
  VARIANT 18: note = misc_feature - Xaa can be any naturally occurring amino acid
  VARIANT 19: note = MISC_FEATURE - Xaa is Asn, Arg, Ile or Thr
  VARIANT 20: note = MISC_FEATURE - Xaa is Asn or Ser
  VARIANT 21: note = misc_feature - Xaa can be any naturally occurring amino acid
  VARIANT 22: note = MISC_FEATURE - Xaa is Lys or Arg
  VARIANT 23: note = MISC_FEATURE - Xaa is Asn or Ser
  VARIANT 24: note = MISC_FEATURE - Xaa is Glu or Asp
  VARIANT 27: note = MISC_FEATURE - Xaa is Thr, Val or Lys
RFXXXLXTSXXTAXLZIXXLXXXDTAXYFCAI

SEQ ID NO: 879 - protein, 11 aa, unidentified
  REGION 1..11: note = Human (Homo sapiens), chimeric or humanized antibody peptide sequence
  VARIANT 6: note = MISC_FEATURE - Xaa is Thr or Ala
WGQGTXLTVSS

SEQ ID NO: 880 - protein, 23 aa, unidentified
  REGION 1..23: note = Human (Homo sapiens), chimeric or humanized antibody peptide sequence
  VARIANT 1: note = MISC_FEATURE - Xaa is Asp or Glu
  VARIANT 5: note = MISC_FEATURE - Xaa is Thr or Ser
  VARIANT 7: note = MISC_FEATURE - Xaa is Ser or Thr
  VARIANT 9: note = MISC_FEATURE - Xaa is Ser, Ala or Val
  VARIANT 10: note = MISC_FEATURE - Xaa is Ser or Thr
  VARIANT 12: note = MISC_FEATURE - Xaa is Ala or Ser
  VARIANT 15: note = MISC_FEATURE - Xaa is Ala or Pro
  VARIANT 18: note = MISC_FEATURE - Xaa is Lys, Arg or Ile
XIVMXQXPXXLXVSXGEXVTMSC

SEQ ID NO: 881 - protein, 15 aa, unidentified
  REGION 1..15: note = Human (Homo sapiens), chimeric or humanized antibody peptide sequence
  VARIANT 9: note = MISC_FEATURE - Xaa is Ser or Ala
WYQQKPGQXPRLLIY

SEQ ID NO: 882 - protein, 32 aa, unidentified; identical to SEQ ID NO: 556
  REGION 1..32: note = Human (Homo sapiens), chimeric or humanized antibody peptide sequence
  VARIANT 1: note = MISC_FEATURE - Xaa is Gly or Asp
  VARIANT 4: note = MISC_FEATURE - Xaa is Asp or Ala
  VARIANT 7: note = MISC_FEATURE - Xaa is Thr or Ser
  VARIANT 14: note = MISC_FEATURE - Xaa is Asp or Glu
  VARIANT 24: note = MISC_FEATURE - Xaa is Ala, Ser or Asp
  VARIANT 27: note = MISC_FEATURE - Xaa is Leu or Phe

SEQ ID NO: 883 - protein, 11 aa, unidentified
  REGION 1..11: note = Human (Homo sapiens), chimeric or humanized antibody peptide sequence
  VARIANT 3: note = MISC_FEATURE - Xaa is Ala or Gln
  VARIANT 6: note = MISC_FEATURE - Xaa is Lys or Val
  VARIANT 9: note = MISC_FEATURE - Xaa is Leu or Ile
FGXGTXLEXKR

SEQ ID NO: 884 - protein, 32 aa, unidentified
  REGION 1..32: note = Human (Homo sapiens), chimeric or humanized antibody peptide sequence
GVPSRFSGSGSGTRFSLKINSLQPEDFGSYYC

SEQ ID NO: 885 - protein, 17 aa, unidentified
  REGION 1..17: note = Human (Homo sapiens), chimeric or humanized antibody peptide sequence
KSSQSLLNSRSRKNYLA

SEQ ID NO: 886 - protein, 5 aa, unidentified
  REGION 1..5: note = Human (Homo sapiens), chimeric or humanized antibody peptide sequence
DADGQ

SEQ ID NO: 887 - protein, 5 aa, unidentified
  REGION 1..5: note = Human (Homo sapiens), chimeric or humanized antibody peptide sequence
SFYWQ

(19) United States
(12) Patent Application Publication
(10) Pub. No.: US 2025/0011435 A1
(43) Pub. Date: Jan. 9, 2025

(54) NEUTRALIZING ANTIBODIES TO THE ALPHA V BETA 8 INTEGRIN COMPLEX FOR IMMUNOTHERAPY

(71) Applicant: The Regents of the University of California, Oakland, CA (US)

(72) Inventors: Stephen L. Nishimura, Mill Valley, CA (US); Jianlong Lou, San Francisco, CA (US); James D. Marks, San Francisco, CA (US); Jody L. Baron, San Francisco, CA (US); Yifan Cheng, San Francisco, CA (US); Shenping Wu, San Francisco, CA (US); Anthony Cormier, San Francisco, CA (US); Naoki Takasaka, San Francisco, CA (US)

(73) Assignee: The Regents of the University of California, Oakland, CA (US)

(21) Appl. No.: 18/784,586

(22) Filed: Jul. 25, 2024

Related U.S. Application Data
(63) Continuation of application No. 18/153,277, filed on Jan. 11, 2023, now Pat. No. 12,110,334.
Application No. 18/153,277 is a division of application No. 17/155,784, filed on Jan. 22, 2021, now Pat. No. 11,591,402.
Application No. 17/155,784 is a division of application No. 16/331,902, filed on Mar. 8, 2019, now Pat. No. 10,954,304.
(60) Provisional application No. 62/529,381, filed on Jul. 6, 2017.
(60) Provisional application No. 62/401,570, filed on Sep. 29, 2016.

(51) Int. Cl. C07K 16/28; A61K 39/00; A61P 1/04; A61P 31/20; A61P 35/04; A61P 37/04; C07K 14/705; C07K 16/22; G01N 33/563

(52) U.S. Cl. CPC C07K 16/2839; A61P 1/04; A61P 31/20; A61P 35/04; A61P 37/04; C07K 14/705; C07K 16/22; G01N 33/563; A61K 2039/505; C07K 2317/24; C07K 2317/34; C07K 2317/56; C07K 2317/622; C07K 2317/76; C07K 2317/92

(57) ABSTRACT

New antibodies and methods of use are described.

### CROSS-REFERENCE TO RELATED PATENT APPLICATIONS

This application is a continuation of Ser. No. 18/153,277 filed Jan. 11, 2023 which is a divisional of Ser. No. 17/155,784 filed Jan. 22, 2021, which is a divisional of Ser. No. 16/331,902, filed Mar. 8, 2019, which is a US National Stage entry of International Application No. PCT/US2017/054306, filed Sep. 29, 2017, which claims benefit of priority to U.S. Provisional Patent Application No. 62/401,570, filed Sep. 29, 2016 and U.S. Provisional Patent Application No. 62/529,381, filed Jul. 6, 2017, all of which are incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under grant no. U54 HL119893, awarded by the National Institutes of Health. The government has certain rights in the invention.

### REFERENCE TO A “SEQUENCE LISTING” SUBMITTED AS ASCII TEXT FILES VIA EFS-WEB

The Sequence Listing written in file 081906-1369766-224040US created on May 18, 2023, 1,212,017 bytes, is hereby incorporated by reference in its entirety for all purposes.

### BACKGROUND OF THE INVENTION

Transforming growth factor β (TGFβ) was originally characterized as an oncogene capable of inducing a transformed phenotype in non-neoplastic cells. A number of TGFβ family members have since been characterized, based on the presence of similar amino acid domains.

Some TGF-β isoforms are expressed ubiquitously in mammals (TGF-β 1-3), but are maintained in an inactive form by non-covalent interaction with a propeptide, the latency associated domain of TGF-β (LAP). For TGFβ to signal, it must be released from its inactive complex by a process called TGFβ activation. The latent TGF complex includes 3 components: the active (mature) TGFβ dimmer, LAP (latency associated peptide) and LTBP (latent TGFβ binding protein). LAP is a dimer, linked by a disulfide bond, that represents the N-terminal end of the TGFβ precursor protein. The mature TGFβ protein represents the C terminal end (about 25 kD) of the precursor. The bond between the TGFβs and LAP is proteolytically cleaved within the Golgi, but the TGF-β propeptide remains bound to TGFβ by non-covalent interactions. The complex of TGFβ and LAP is called the small latent complex (SLC). It is the association of LAP and TGFβ that confers latency. LAP-TGFβ binding is reversible and the isolated purified components can recombine to form an inactive SLC. Both the SLC and the larger complex are referred to herein as latent TGFβ, as both are inactive.

In general, integrins are adhesion molecules and mediate the attachment of cells to extracellular matrix proteins. Integrin αvβ8 binds to the LAP of TGF-β and mediates the activation of TGF-β1 and 3 (Mu et al. (2002) J. Cell Biol. 159:493). Integrin αvβ8-mediated activation of TGF-β is required for in vivo activation of TGF-β (i.e., release of the mature TGF-β polypeptide), thus αvβ8 is a gatekeeper of TGF-β function. Integrin αvβ8 is expressed in normal epithelia (e.g., airway epithelia), mesenchymal cells, and neuronal tissues.

The integrin β8 (Itgb8) has been associated with forkhead box P3 (Foxp3)-positive T cells and T-regulatory-specific epigenetic remodeling. See, e.g., Vandenbon, et al., Proc. Natl. Acad. Sci. USA vol. 113 no. 17 pp. E2393-E2402 (2016). FoxP3 is a transcription factor involved in the development of T-regulatory (Treg) cells. Human and mouse effector Treg cells express functional TGF-β-activating integrin αvβ8. See, Worthington, Immunity Volume 42, Issue 5, pp. 903-915 (May 2015). Treg cell integrin αvβ8-mediated TGF-β activation is not needed for T cell homeostasis and integrin αvβ8 expression by Treg cells suppresses active inflammation.

### Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The terms “anti-αvβ8 antibody,” “αvβ8 specific antibody,” “αvβ8 antibody,” and “anti-αvβ8” are used synonymously herein to refer to an antibody that specifically binds to αvβ8. Similarly, an anti-β8 antibody (and like terms) refer to an antibody that specifically binds to β8. The anti-αvβ8 antibodies and anti-β8 antibodies described herein bind to the protein expressed on αvβ8 expressing cells.

An αvβ8-associated disorder is a condition characterized by the presence of αvβ8-expressing cells, either cells expressing an increased level of αvβ8, or increased number of αvβ8-expressing cells relative to a normal, non-diseased control. TGFβ-associated disorders (disorders characterized by higher than normal TGFβ activity) include αvβ8-associated disorders, as αvβ8 is involved in activating TGFβ in certain circumstances, as described herein.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or

BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “antibody” refers to a polypeptide comprising a framework region encoded by an immunoglobulin gene, or fragments thereof, that specifically bind and recognize an antigen, e.g., human αvβ8, a particular cell surface marker, or any desired target. Typically, the “variable region” contains the antigen-binding region of the antibody (or its functional equivalent) and is most critical in specificity and affinity of binding. See Paul, Fundamental Immunology (2003).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

An “isotype” is a class of antibodies defined by the heavy chain constant region. Antibodies described herein can be of any isotype of isotype class. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, (1992)).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementary determining region (“CDR”) residues and possibly some framework (“FR”) residues are substituted by residues from analogous sites in rodent antibodies.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

The various antibodies or antigen-binding fragments described herein can be produced by enzymatic or chemical modification of the intact antibodies, or synthesized de novo using recombinant DNA methodologies (e.g., single chain Fv), or identified using phage display libraries (see, e.g., McCafferty et al., Nature 348:552-554, 1990). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries or ribosome display libraries, gene shuffled libraries. Such libraries can be constructed from synthetic, semi-synthetic or native and immunocompetent sources.

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “V-region” refers to an antibody variable region domain comprising the segments of Framework 1, CDR1, Framework 2, CDR2, Framework 3, CDR3, and Framework 4. These segments are included in the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson and Wu, Nucleic Acids Res. 2000 Jan. 1; 28 (1): 214-218 and Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; A1-Lazikani et al., J. Mol. Biol 1997, 273 (4)). Unless otherwise indicated, CDRs are determined according to Kabat. Definitions of antigen combining sites are also described in the following: Ruiz et al. Nucleic Acids Res., 28, 219-221 (2000); and Lefranc Nucleic Acids Res. January 1; 29 (1): 207-9 (2001); MacCallum et al., J. Mol. Biol., 262:732-745 (1996); and Martin et al, Proc. Natl Acad. Sci. USA, 86, 9268-9272 (1989); Martin, et al, Methods Enzymol., 203:121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” is an antibody molecule in which (a) the constant region, or a portion thereof, is altered, replaced or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31 (3): 169-217 (1994).

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds β8 will typically bind to β8 with at least a 2-fold greater affinity than a non-β8 target (e.g., a different integrin subunit, e.g., β6).

The term “binds” with respect to a cell type (e.g., an antibody that binds fibrotic cells, hepatocytes, chondrocytes, etc.), typically indicates that an agent binds a majority of the cells in a pure population of those cells. For example, an antibody that binds a given cell type typically binds to at least ⅔ of the cells in a population of the indicated cells (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

The term “differentially expressed” or “differentially regulated” refers generally to a protein or nucleic acid biomarker that is overexpressed (upregulated) or underexpressed (downregulated) in one sample compared to at least one other sample. In the context of the present invention, the term generally refers to overexpression of a biomarker (e.g., αvβ8) on a diseased cell compared to a normal cell.

For example, the terms “overexpressed” or “upregulated” interchangeably refer to a protein or nucleic acid, generally a biomarker, that is transcribed or translated at a detectably greater than control level. The term includes overexpression due to transcription, post transcriptional processing, translation, post-translational processing, cellular localization (e.g., organelle, cytoplasm, nucleus, cell surface), and RNA and protein stability. Overexpression can be detected using conventional techniques for detecting biomarkers, whether mRNA (i.e., RT-PCR, hybridization) or protein (i.e., flow cytometry, imaging, ELISA, immunohistochemical techniques). Overexpression can be 10%, 20%, 30%, 40%, 50%, 60%, 70%, 80%, 90% or more in comparison to a normal cell.

The terms “agonist,” “activator,” “inducer” and like terms refer to molecules that increase activity or expression as compared to a control. Agonists are agents that, e.g., bind to, stimulate, increase, activate, enhance activation, sensitize or upregulate the activity of the target. The expression or activity can be increased 10%, 20%, 30%, 40%, 50%, 60%, 70%, 80%, 90% 100% or more than that in a control. In certain instances, the activation is 1.5-fold, 2-fold, 3-fold, 4-fold, 5-fold, 10-fold, or more in comparison to a control.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

The term “diagnosis” refers to a relative probability that a disorder such as cancer or an inflammatory condition is present in the subject. Similarly, the term “prognosis” refers to a relative probability that a certain future outcome may occur in the subject. For example, prognosis can refer to the likelihood that an individual will develop a TGFβ or αvβ8 associated disorder, have recurrence, or the likely severity of the disease (e.g., severity of symptoms, rate of functional decline, survival, etc.). The terms are not intended to be absolute, as will be appreciated by any one of skill in the field of medical diagnostics.

“Biopsy” or “biological sample from a patient” as used herein refers to a sample obtained from a patient having, or suspected of having, a TGFβ or αvβ8 associated disorder. In some embodiments, the sample may be a tissue biopsy, such as needle biopsy, fine needle biopsy, surgical biopsy, etc. The sample can also be a blood sample or blood fraction, e.g., white blood cell fraction, serum, or plasma. The sample can comprise a tissue sample harboring a lesion or suspected lesion, although the biological sample may be also be derived from another site, e.g., a site of suspected metastasis, a lymph node, or from the blood. In some cases, the biological sample may also be from a region adjacent to the lesion or suspected lesion.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, urine, feces, etc.

The terms “therapy,” “treatment,” and “amelioration” refer to any reduction in the severity of symptoms. In the case of treating an inflammatory condition, the treatment can refer to reducing, e.g., blood levels of inflammatory cytokines, blood levels of active mature TGFβ, pain, swelling, recruitment of immune cells, etc. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

An “inflammatory condition” refers to any inflammation in an individual, and can be transient (e.g., in response to exposure to a pathogen or allergen) or chronic. Inflammation is characterized by inflammatory cytokines such as IFN-gamma, IL-6, and TNF-alpha that recruit and activate macrophages and other leukocytes. In some cases, inflammation can develop into a chronic, harmful condition or autoimmune condition (e.g., MS, lupus, rheumatoid arthritis, Crohn's disease). Inflammation can be evident locally (e.g., at a localized site of infection or exposure) or systemically (e.g., atherosclerosis, high blood pressure). In some embodiments, the antibody compositions and methods described herein can be used to treat inflammatory conditions.

“Cancer”, “tumor,” “transformed” and like terms include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to carcinomas, sarcomas, adenocarcinomas, lymphomas, leukemias, solid and lymphoid cancers, etc. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia (AML), chronic myeloid leukemia (CML), and multiple myeloma. In some embodiments, the antibody compositions and methods described herein can be used for treating cancer.

The term “co-administer” refers to the simultaneous presence of two active agents in the blood of an individual. Active agents that are co-administered can be concurrently or sequentially delivered.

### BRIEF SUMMARY OF THE INVENTION

In some aspects, an antibody is provided that specifically binds human αvβ8 and blocks binding of TGFβ peptide to αvβ8, wherein the antibody binds to an epitope on human αvβ8 comprising amino acids D148, A149, D150, G151, and Y178 of human αv as occurs in SEQ ID NO:393 and amino acids H118, S170, D171, Y172, N173 L174, D175, H200, and R201 of human β8 as occurs in SEQ ID NO:394.

In some embodiments, an antibody (optionally a chimeric or humanized antibody) is provided that comprises heavy chain CDRs SEQ ID NO:562, SEQ ID NO: 563, and SEQ ID NO; 564 and light chain CDRs SEQ ID NO:569, SEQ ID NO: 570, and SEQ ID NO: 571.

In some embodiments, an antibody (optionally a chimeric or humanized antibody) is provided that comprises:
heavy chain CDRs SEQ ID NO:313, SEQ ID NO:314, and SEQ ID NO:315; and light chain CDRs SEQ ID NO:334, SEQ ID NO:335, and SEQ ID NO:336; or
heavy chain CDRs SEQ ID NO:319, SEQ ID NO:320, and SEQ ID NO:321; and light chain CDRs SEQ ID NO:340, SEQ ID NO:341, and SEQ ID NO:342; or
heavy chain CDRs SEQ ID NO:316, SEQ ID NO:317, and SEQ ID NO:318; and light chain CDRs SEQ ID NO:337, SEQ ID NO:338, and SEQ ID NO:339; or
heavy chain CDRs SEQ ID NO:322, SEQ ID NO:323, and SEQ ID NO:324; and light chain CDRs SEQ ID NO:343, SEQ ID NO:344, and SEQ ID NO:345; or
heavy chain CDRs SEQ ID NO:322, SEQ ID NO:323, and SEQ ID NO:324; and light chain CDRs SEQ ID NO:346, SEQ ID NO:347, and SEQ ID NO:348; or
heavy chain CDRs SEQ ID NO:322, SEQ ID NO:323, and SEQ ID NO:324; and light chain CDRs SEQ ID NO:349, SEQ ID NO:350, and SEQ ID NO:351; or
heavy chain CDRs SEQ ID NO:325, SEQ ID NO:326, and SEQ ID NO:327; and light chain CDRs SEQ ID NO:352, SEQ ID NO:353, and SEQ ID NO:354; or
heavy chain CDRs SEQ ID NO:325, SEQ ID NO:326, and SEQ ID NO:327; and light chain CDRs SEQ ID NO:355, SEQ ID NO:356, and SEQ ID NO:357; or
heavy chain CDRs SEQ ID NO:325, SEQ ID NO:326, and SEQ ID NO:327; and light chain CDRs SEQ ID NO:358, SEQ ID NO:359, and SEQ ID NO:360; or
heavy chain CDRs SEQ ID NO:367, SEQ ID NO:368, and SEQ ID NO:369; and light chain CDRs SEQ ID NO:373, SEQ ID NO:374, and SEQ ID NO:375; or
heavy chain CDRs SEQ ID NO:364, SEQ ID NO:365, and SEQ ID NO:366; and light chain CDRs SEQ ID NO:373, SEQ ID NO:374, and SEQ ID NO:375; or
heavy chain CDRs SEQ ID NO:367, SEQ ID NO:368, and SEQ ID NO:369; and light chain CDRs SEQ ID NO:376, SEQ ID NO:377, and SEQ ID NO:378; or
heavy chain CDRs SEQ ID NO:370, SEQ ID NO:371, and SEQ ID NO:372; and light chain CDRs SEQ ID NO:373, SEQ ID NO:374, and SEQ ID NO:375; or
heavy chain CDRs SEQ ID NO:331, SEQ ID NO:332, and SEQ ID NO:333; and light chain CDRs SEQ ID NO:382, SEQ ID NO:383, and SEQ ID NO:384; or
heavy chain CDRs SEQ ID NO:379, SEQ ID NO:380, and SEQ ID NO:381; and light chain CDRs SEQ ID NO:361, SEQ ID NO:362, and SEQ ID NO:363; or
heavy chain CDRs SEQ ID NO:331, SEQ ID NO:332, and SEQ ID NO:333; and light chain CDRs SEQ ID NO:361, SEQ ID NO:362, and SEQ ID NO:363; or
heavy chain CDRs SEQ ID NO:508, SEQ ID NO:509, and SEQ ID NO:510; and light chain CDRs SEQ ID NO:529, SEQ ID NO:530, and SEQ ID NO:531; or
heavy chain CDRs SEQ ID NO:511, SEQ ID NO:512, and SEQ ID NO:513; and light chain CDRs SEQ ID NO:532, SEQ ID NO:533, and SEQ ID NO:534; or
heavy chain CDRs SEQ ID NO:514, SEQ ID NO:515, and SEQ ID NO:516; and light chain CDRs SEQ ID NO:535, SEQ ID NO:536, and SEQ ID NO:537; or
heavy chain CDRs SEQ ID NO:517, SEQ ID NO:518, and SEQ ID NO:519; and light chain CDRs SEQ ID NO:538, SEQ ID NO:539, and SEQ ID NO:540; or
heavy chain CDRs SEQ ID NO:520, SEQ ID NO:521, and SEQ ID NO:522; and light chain CDRs SEQ ID NO:541, SEQ ID NO:542, and SEQ ID NO:543; or
heavy chain CDRs SEQ ID NO:523, SEQ ID NO:524, and SEQ ID NO:525; and light chain CDRs SEQ ID NO:544, SEQ ID NO: 545, and SEQ ID NO:546; or
heavy chain CDRs SEQ ID NO:526, SEQ ID NO:527, and SEQ ID NO:528; and light chain CDRs SEQ ID NO:547, SEQ ID NO: 548, and SEQ ID NO:549; or
other antibodies described herein.

In some embodiments, the antibody is linked to a detectable label.

In some embodiments, the antibody further comprises heavy chain framework sequences FR1, FR2, FR3, and FR4 as SEQ ID NO: 558, SEQ ID NO: 559, SEQ ID NO: 560, and SEQ ID NO: 561, respectively, and light chain framework sequences FR1, FR2, FR3, and FR4 as SEQ ID NO: 565, SEQ ID NO: 566, SEQ ID NO: 567, and SEQ ID NO: 568, respectively.

In some embodiments, the antibody further comprises heavy chain framework sequences FR1, FR2, FR3, and FR4 as SEQ ID NO: 550, SEQ ID NO: 551, SEQ ID NO: 552, and SEQ ID NO: 553, respectively, and light chain framework sequences FR1, FR2, FR3, and FR4 as SEQ ID NO: 554, SEQ ID NO: 555, SEQ ID NO: 556, and SEQ ID NO: 557, respectively.

In some embodiments, the antibody is humanized. In some embodiments, the humanized antibody comprises SEQ ID NO:395, SEQ ID NO:403, SEQ ID NO:411; SEQ ID NO: 419, SEQ ID NO:427, SEQ ID NO:443, SEQ ID NO:451, SEQ ID NO:459, SEQ ID NO: 467; SEQ ID NO:475, SEQ ID NO:484, or SEQ ID NO:500.

Also provided is an antibody that binds to αvβ8 and αvβ6 and comprising a light chain CDR1 comprising the sequence RGDL. In some embodiments, the antibody comprises variable regions comprising heavy chain CDRs SEQ ID NO:523, SEQ ID NO:524, and SEQ ID NO:525; and light chain CDRs SEQ ID NO:544, SEQ ID NO:545, and SEQ ID NO:546; or heavy chain CDRs SEQ ID NO:526, SEQ ID NO:527, and SEQ ID NO:528; and light chain CDRs SEQ ID NO: 547, SEQ ID NO:548, and SEQ ID NO:549.

In some embodiments, the antibody further comprises heavy chain framework sequences FR1, FR2, FR3, and FR4 as SEQ ID NO: 558, SEQ ID NO: 559, SEQ ID NO: 560, and SEQ ID NO: 561, respectively, and light chain framework sequences FR1, FR2, FR3, and FR4 as SEQ ID NO: 565, SEQ ID NO: 566, SEQ ID NO: 567, and SEQ ID NO: 568, respectively.

In some embodiments, the antibody further comprises heavy chain framework sequences FR1, FR2, FR3, and FR4 as SEQ ID NO: 550, SEQ ID NO: 551, SEQ ID NO: 552, and SEQ ID NO: 553, respectively, and light chain framework sequences FR1, FR2, FR3, and FR4 as SEQ ID NO: 554, SEQ ID NO: 555, SEQ ID NO: 556, and SEQ ID NO: 557, respectively.

In some embodiments, the antibody is humanized.

In some embodiments, the antibody is linked to a detectable label.

Also provided is an antibody that specifically binds human αvβ8 and blocks binding of TGFβ peptide to αvβ8, wherein the antibody binds to the specificity determining loop (SDL) of human β8. In some embodiments, the antibody further binds to one, two, or all three of the human αv-head domain, the al helix of human β8, or the α2 helix of human β8. In some embodiments, the antibody is humanized or chimeric. In some embodiments, the antibody is linked to a detectable label.

Also provided is a pharmaceutical composition comprising an antibody as described above or elsewhere herein in a pharmaceutically acceptable excipient.

Also provided is a method of enhancing an immune response to a viral infection in a human individual. In some embodiments, the method comprises administering a sufficient amount of an antibody as described above or elsewhere herein to the individual, thereby enhancing an immune response to the viral infection.

In some embodiments, the viral infection is a hepatitis infection. In some embodiments, the viral infection is a hepatitis B infection.

Also provided is a method of enhancing an immune response to a viral infection in a human individual, the method comprising administering a sufficient amount of the antibody to the individual, wherein the antibody specifically binds to human αvβ8 and blocks binding of TGFβ peptide to αvβ8 or blocks activation of αvβ8 by binding of TGFβ human αvβ8, thereby enhancing an immune response to the viral infection.

Also provided is a method of enhancing an immune response to cancer in a human individual, the method comprising administering a sufficient amount of an antibody as described above or elsewhere herein to the individual, thereby enhancing an immune response to the cancer.

In some embodiments, the cancer is lung cancer. In some embodiments, the cancer is a metastatic cancer. In some embodiments, the cancer is a primary cancer.

Also provided is a method of enhancing an immune response to H. pyroli in a human individual, the method comprising administering a sufficient amount of an antibody as described above or elsewhere herein to the individual, thereby enhancing an immune response to H. pyroli.

In some embodiments, the human individual has a peptide ulcer, gastric carcinoma or MALT lymphoma.

Also provided is an antibody that specifically binds to human αvβ8 and that comprises human heavy chain CDRs SEQ ID NO:299, SEQ ID NO:301, and SEQ ID NO:303; and light chain CDRs SEQ ID NO:307, SEQ ID NO:309, and SEQ ID NO:311. Alternatively, any antibodies having heavy chain CDRs or a heavy chain variable region as set forth in FIG. 53 and light chain CDRs or a light chain variable region from a corresponding sequence as set forth in FIG. 54 can be used

In some embodiments, the antibody is linked to a detectable label.

Also provided is a method of detecting the presence, absence, or quantity of human in a sample, the method comprising, contacting to the sample an antibody that specifically binds to human αvβ8 and that comprises human heavy chain CDRs SEQ ID NO:299, SEQ ID NO:301, and SEQ ID NO:303; and light chain CDRs SEQ ID NO:307, SEQ ID NO:309, and SEQ ID NO: 311, and detecting or quantifying binding of the antibody to the sample.

In some embodiments, the sample is a formalin-fixed sample.

### BRIEF DESCRIPTION OF THE DRAWINGS

FIG. 1 illustrates heavy chain amino acid sequences for clones used in the construction of the composite antibody C6D4. B13C4 15-8: all sequences (SEQ ID NO:1), Framework 1 (SEQ ID NO:2), CDR1 (SEQ ID NO:3), Framework 2 (SEQ ID NO:4), CDR2 (SEQ ID NO:5), Framework 3 (SEQ ID NO:6), CRD3 (SEQ ID NO:7), and Framework 4 (SEQ ID NO:8); B13C4 15-10: all sequences (SEQ ID NO:9), Framework 1 (SEQ ID NO:10), CDR1 (SEQ ID NO: 11), Framework 2 (SEQ ID NO:12), CDR2 (SEQ ID NO:13), Framework 3 (SEQ ID NO: 14), CRD3 (SEQ ID NO:15), and Framework 4 (SEQ ID NO:16); B13H3.2: all sequences (SEQ ID NO:17), Framework 1 (SEQ ID NO:18), CDR1 (SEQ ID NO:19), Framework 2 (SEQ ID NO: 20), CDR2 (SEQ ID NO:21), Framework 3 (SEQ ID NO:22), CRD3 (SEQ ID NO:23), and Framework 4 (SEQ ID NO:24); B13C1231015: all sequences (SEQ ID NO:25), Framework 1 (SEQ ID NO:26), CDR1 (SEQ ID NO:27), Framework 2 (SEQ ID NO:28), CDR2 (SEQ ID NO: 29), Framework 3 (SEQ ID NO:30), CRD3 (SEQ ID NO:31), and Framework 4 (SEQ ID NO: 32); B15B11VH: all sequences (SEQ ID NO:33), Framework 1 (SEQ ID NO:34), CDR1 (SEQ ID NO:35), Framework 2 (SEQ ID NO:36), CDR2 (SEQ ID NO:37), Framework 3 (SEQ ID NO: 38), CRD3 (SEQ ID NO:39), and Framework 4 (SEQ ID NO:40); B2B2 15-9: all sequences (SEQ ID NO:41), Framework 1 (SEQ ID NO:42), CDR1 (SEQ ID NO:43), Framework 2 (SEQ ID NO:44), CDR2 (SEQ ID NO:45), Framework 3 (SEQ ID NO:46), CRD3 (SEQ ID NO:47), and Framework 4 (SEQ ID NO:48); R11D12715.3: all sequences (SEQ ID NO: 49), Framework 1 (SEQ ID NO:50), CDR1 (SEQ ID NO:51), Framework 2 (SEQ ID NO: 52), CDR2 (SEQ ID NO:53), Framework 3 (SEQ ID NO:54), CRD3 (SEQ ID NO:55), and Framework 4 (SEQ ID NO:56); RSDLVH-1: all sequences (SEQ ID NO:57 and SEQ ID NO: 65), Framework 1 (SEQ ID NO:58 and SEQ ID NO:66), CDR1 (SEQ ID NO:59 and SEQ ID NO: 67), Framework 2 (SEQ ID NO:60 and SEQ ID NO:68), CDR2 (SEQ ID NO:61 and SEQ ID NO:69), Framework 3 (SEQ ID NO:62 and SEQ ID NO:70), CRD3 (SEQ ID NO:63 and SEQ ID NO:71), and Framework 4 (SEQ ID NO:64 and SEQ ID NO:72); RSDLVH-3: all sequences (SEQ ID NO:73), Framework 1 (SEQ ID NO:74), CDR1 (SEQ ID NO:75), Framework 2 (SEQ ID NO:76), CDR2 (SEQ ID NO:77), Framework 3 (SEQ ID NO:78), CRD3 (SEQ ID NO:79), and Framework 4 (SEQ ID NO:80); RSDLVH-16: all sequences (SEQ ID NO: 81), Framework 1 (SEQ ID NO:82), CDR1 (SEQ ID NO:83), Framework 2 (SEQ ID NO: 84), CDR2 (SEQ ID NO:85), Framework 3 (SEQ ID NO:86), CRD3 (SEQ ID NO:87), and Framework 4 (SEQ ID NO:88); both 29 and 44: all sequences (SEQ ID NO:89), Framework 1 (SEQ ID NO:90), CDR1 (SEQ ID NO:91), Framework 2 (SEQ ID NO:92), CDR2 (SEQ ID NO: 93), Framework 3 (SEQ ID NO:94), CRD3 (SEQ ID NO:95), and Framework 4 (SEQ ID NO: 96); A1=B4=F9: all sequences (SEQ ID NO:97), Framework 1 (SEQ ID NO:98), CDR1 (SEQ ID NO:99), Framework 2 (SEQ ID NO:100), CDR2 (SEQ ID NO:101), Framework 3 (SEQ ID NO:102), CRD3 (SEQ ID NO:103), and Framework 4 (SEQ ID NO:104); A5=C6: all sequences (SEQ ID NO:105), Framework 1 (SEQ ID NO:106), CDR1 (SEQ ID NO:107), Framework 2 (SEQ ID NO:108), CDR2 (SEQ ID NO:109), Framework 3 (SEQ ID NO:110), CRD3 (SEQ ID NO:111), and Framework 4 (SEQ ID NO:112); D4=E6: all sequences (SEQ ID NO: 113), Framework 1 (SEQ ID NO:114), CDR1 (SEQ ID NO:115), Framework 2 (SEQ ID NO: 116), CDR2 (SEQ ID NO:117), Framework 3 (SEQ ID NO:118), CRD3 (SEQ ID NO:119), and Framework 4 (SEQ ID NO:120); and C6D4: all sequences (SEQ ID NO:121), Framework 1 (SEQ ID NO:122), CDR1 (SEQ ID NO:123), Framework 2 (SEQ ID NO:124), CDR2 (SEQ ID NO: 125), Framework 3 (SEQ ID NO: 126), CRD3 (SEQ ID NO:127), and Framework 4 (SEQ ID NO: 128).

FIG. 2 illustrates light chain amino acid sequences for clones used in the construction of the composite antibody C6D4. B2B2 35-20: all sequences (SEQ ID NO: 129), Framework 1 (SEQ ID NO:130), CDR1 (SEQ ID NO:131), Framework 2 (SEQ ID NO:132), CDR2 (SEQ ID NO: 133), Framework 3 (SEQ ID NO:134), CRD3 (SEQ ID NO:135), and Framework 4 (SEQ ID NO: 136); B2B2 35-26: all sequences (SEQ ID NO:137), Framework 1 (SEQ ID NO:138), CDR1 (SEQ ID NO:139), Framework 2 (SEQ ID NO:140), CDR2 (SEQ ID NO:141), Framework 3 (SEQ ID NO:142), CRD3 (SEQ ID NO:143), and Framework 4 (SEQ ID NO:144); B15B11vk34-26: all sequences (SEQ ID NO: 145), Framework 1 (SEQ ID NO:146), CDR1 (SEQ ID NO:147), Framework 2 (SEQ ID NO:148), CDR2 (SEQ ID NO:149), Framework 3 (SEQ ID NO:150), CRD3 (SEQ ID NO:151), and Framework 4 (SEQ ID NO:152); B15B11vk33-24: all sequences (SEQ ID NO:153), Framework 1 (SEQ ID NO:154), CDR1 (SEQ ID NO:155), Framework 2 (SEQ ID NO:156), CDR2 (SEQ ID NO:157), Framework 3 (SEQ ID NO:158), CRD3 (SEQ ID NO:159), and Framework 4 (SEQ ID NO:160); B15B11vk35-26: all sequences (SEQ ID NO: 161), Framework 1 (SEQ ID NO:162), CDR1 (SEQ ID NO:163), Framework 2 (SEQ ID NO:164), CDR2 (SEQ ID NO:165), Framework 3 (SEQ ID NO:166), CRD3 (SEQ ID NO:167), and Framework 4 (SEQ ID NO:168); B13C12134-25: all sequences (SEQ ID NO:169), Framework 1 (SEQ ID NO:170), CDR1 (SEQ ID NO:171), Framework 2 (SEQ ID NO:172), CDR2 (SEQ ID NO:173), Framework 3 (SEQ ID NO:174), CRD3 (SEQ ID NO:175), and Framework 4 (SEQ ID NO:176); B13C12133-26: all sequences (SEQ ID NO:177), Framework 1 (SEQ ID NO:178), CDR1 (SEQ ID NO:179), Framework 2 (SEQ ID NO:180), CDR2 (SEQ ID NO:181), Framework 3 (SEQ ID NO:182), CRD3 (SEQ ID NO: 183), and Framework 4 (SEQ ID NO:184); B13C4 35-20: all sequences (SEQ ID NO:185), Framework 1 (SEQ ID NO:186), CDR1 (SEQ ID NO:187), Framework 2 (SEQ ID NO:188), CDR2 (SEQ ID NO:189), Framework 3 (SEQ ID NO:190), CRD3 (SEQ ID NO:191), and Framework 4 (SEQ ID NO:192); B15B11vk35-20: all sequences (SEQ ID NO:193), Framework 1 (SEQ ID NO:194), CDR1 (SEQ ID NO:195), Framework 2 (SEQ ID NO:196), CDR2 (SEQ ID NO: 197), Framework 3 (SEQ ID NO: 198), CRD3 (SEQ ID NO: 199), and Framework 4 (SEQ ID NO: 200); B13C12335-25: all sequences (SEQ ID NO:201), Framework 1 (SEQ ID NO:202), CDR1 (SEQ ID NO:203), Framework 2 (SEQ ID NO:204), CDR2 (SEQ ID NO:205), Framework 3 (SEQ ID NO:206), CRD3 (SEQ ID NO:207), and Framework 4 (SEQ ID NO:208); B13C1233520: all sequences (SEQ ID NO:209), Framework 1 (SEQ ID NO:210), CDR1 (SEQ ID NO: 211), Framework 2 (SEQ ID NO:212), CDR2 (SEQ ID NO:213), Framework 3 (SEQ ID NO: 214), CRD3 (SEQ ID NO:215), and Framework 4 (SEQ ID NO:216); RSDLVK-1: all sequences (SEQ ID NO:217), Framework 1 (SEQ ID NO:218), CDR1 (SEQ ID NO:219), Framework 2 (SEQ ID NO:220), CDR2 (SEQ ID NO:221), Framework 3 (SEQ ID NO:222), CRD3 (SEQ ID NO:223), and Framework 4 (SEQ ID NO:224); RSDLVK-6: all sequences (SEQ ID NO: 225), Framework 1 (SEQ ID NO:226), CDR1 (SEQ ID NO:227), Framework 2 (SEQ ID NO: 228), CDR2 (SEQ ID NO:229), Framework 3 (SEQ ID NO:230), CRD3 (SEQ ID NO:231), and Framework 4 (SEQ ID NO:232); RSDLVK-10: all sequences (SEQ ID NO:233), Framework 1 (SEQ ID NO:234), CDR1 (SEQ ID NO:235), Framework 2 (SEQ ID NO:236), CDR2 (SEQ ID NO:237), Framework 3 (SEQ ID NO:238), CRD3 (SEQ ID NO:239), and Framework 4 (SEQ ID NO:240); RSDLVK-13: all sequences (SEQ ID NO:241), Framework 1 (SEQ ID NO:242), CDR1 (SEQ ID NO:243), Framework 2 (SEQ ID NO:244), CDR2 (SEQ ID NO: 245), Framework 3 (SEQ ID NO:246), CRD3 (SEQ ID NO:247), and Framework 4 (SEQ ID NO: 248); 29: all sequences (SEQ ID NO:249), Framework 1 (SEQ ID NO:250), CDR1 (SEQ ID NO: 251), Framework 2 (SEQ ID NO:252), CDR2 (SEQ ID NO:253), Framework 3 (SEQ ID NO: 254), CRD3 (SEQ ID NO:255), and Framework 4 (SEQ ID NO:256); 44: all sequences (SEQ ID NO:257), Framework 1 (SEQ ID NO:258), CDR1 (SEQ ID NO:259), Framework 2 (SEQ ID NO:260), CDR2 (SEQ ID NO:261), Framework 3 (SEQ ID NO:262), CRD3 (SEQ ID NO: 263), and Framework 4 (SEQ ID NO:264); A1=B4-F9: all sequences (SEQ ID NO:265), Framework 1 (SEQ ID NO:266), CDR1 (SEQ ID NO:267), Framework 2 (SEQ ID NO:268), CDR2 (SEQ ID NO:269), Framework 3 (SEQ ID NO:270), CRD3 (SEQ ID NO:271), and Framework 4 (SEQ ID NO:272); A5=C6: all sequences (SEQ ID NO:273), Framework 1 (SEQ ID NO: 274), CDR1 (SEQ ID NO:275), Framework 2 (SEQ ID NO:276), CDR2 (SEQ ID NO: 277), Framework 3 (SEQ ID NO:278), CRD3 (SEQ ID NO:279), and Framework 4 (SEQ ID NO: 280); D4=E6: all sequences (SEQ ID NO:281), Framework 1 (SEQ ID NO:282), CDR1 (SEQ ID NO:283), Framework 2 (SEQ ID NO:284), CDR2 (SEQ ID NO:285), Framework 3 (SEQ ID NO:286), CRD3 (SEQ ID NO:287), and Framework 4 (SEQ ID NO:288); and C6D4: all sequences (SEQ ID NO:289), Framework 1 (SEQ ID NO:290), CDR1 (SEQ ID NO:291), Framework 2 (SEQ ID NO:292), CDR2 (SEQ ID NO:293), Framework 3 (SEQ ID NO:294), CRD3 (SEQ ID NO:295), and Framework 4 (SEQ ID NO:296).

FIG. 3 is a plot of transforming growth factor-beta (TGF-β) binding inhibition percentages for different concentrations of the allosteric inhibitor B5 and the composite antibody C6D4.

FIG. 4 illustrates conservation of epitope among mammals, indicating the antibodies can be useful in multiple preclinical animal models and have broad utility, including in veterinary applications. Human αv (SEQ ID NO:591); Chimp αv (SEQ ID NO:592); Rhesus αv (SEQ ID NO:593); Cyno αv (SEQ ID NO:594); Cow αv (SEQ ID NO:595); Pig αv (SEQ ID NO: 596); Horse αv (SEQ ID NO:597); Mouse αv (SEQ ID NO:598); Rat αv (SEQ ID NO: 599); Armadillo αv (SEQ ID NO:600); Platypus αv (SEQ ID NO:601); Human β8 (SEQ ID NO: 602); Chimp β8 (SEQ ID NO:603); Rhesus β8 (SEQ ID NO:604); Cyno β8 (SEQ ID NO: 605); Cow β8 (SEQ ID NO:606); Pig β8 (SEQ ID NO:607); Horse β8 (SEQ ID NO:608); Mouse β8 (SEQ ID NO:609); Rat β8 (SEQ ID NO:610); Armadillo β8 (SEQ ID NO:611); and Platypus β8 (SEQ ID NO:612).

FIG. 5 illustrates integrin alphaV (SEQ ID NO:394) and beta8 (SEQ ID NO:395) sequences. The epitope for C6D4 is in bold underlined italics.

FIG. 6 illustrates cryoEM results, highlighting the interactions between C6D4 and the (SDL) loop of β8, the α1 and α2 helices of β8, and the head of αv.

FIG. 7 illustrates the residues of the SDL and β8 α1 and α2 helices and αv head of integrin αvβ8 that directly interact with C6D4 upon binding. The head sequence of integrin αv is FNLDVDSPAEYSGPEGSYFGFAVDFFVPSASSRMFLLVGAPKANTTQPGIVEGGQVLKC DWSSTRRCQPIEFDATGNRDYAKDDPLEFKSHQWFGASVRSKQDKILACAPLYHWRTE MKQEREPVGTCFLQDGTKTVEYAPCRSQDIDADGQGFCQGGFSIDFTKADRVLLGGPGS FYWQGQLISDQVAEIVSKYDPNVYSIKYNNQLATRTAQAIFDDSYLGYSVAVGDENGD GIDDFVSGVPRAARTLGMVYIYDGKNMSSLYNFTGEQMAAYFGFSVAATDINGDDYAD VFIGAPLFMDRGSDGKLQEVGQVSVSLQRASGDFQTTKLNGFEVFARFGSAIAPLGDLD QDGFNDIAIAAPYGGEDKKGIVYIFNGRSTGLNAVPSQILEGQWAARSMPPSFGYSMKG ATDIDKNGYPDLIVGAFGVDRAILYRARP (SEQ ID NO:623). Sequences C6D4 VH CDR1 (SEQ ID NO:613); C6D4 VH CDR2 (SEQ ID NO:614); C6D4 VH CDR3 (SEQ ID NO:615); C6D4 Vk CDR1 (SEQ ID NO:616); C6D4 Vk CDR2 (SEQ ID NO:617); C6D4 Vk CDR3 (SEQ ID NO: 618); β8, α1 helix (SEQ ID NO:619); β8, SDL (SEQ ID NO:620); β8, α2 helix (SEQ ID NO: 621); and αV, β-propeller domain blade W3 (SEQ ID NO:622).

FIG. 8 illustrates the overlapping of the C6D4 epitope with the ligand binding pocket of integrin αvβ8, in relation to the association of the integrin with latent TGF-β.

FIG. 9 is a plot of percent survival of mice injected with Lewis lung carcinoma (LLC) cells. The primary tumors were removed and the animals treated with C6D4 murine IgG2a or SV5 isotype control at a dosage of 7 mg/kg once per week. In this model, mice are euthanized after losing 20% body weight due to recurrence of the primary tumor or due to metastasis.

FIG. 10 is a table of HepB surface antigen (HBSag) clearance from a chronic infection mouse model as a result of treatment with C6D4.

FIG. 11A-B illustrate amino acid sequences for clones used in the construction of the engineered antibody 4F1F9, an antibody used for detection of αvβ8 in human tissues. FIG. 11A Sequences-4F1: all sequences (SEQ ID NO:624), Framework 1 (SEQ ID NO:625), CDR1 (SEQ ID NO:628), Framework 2 (SEQ ID NO:632), CDR2 (SEQ ID NO:634), Framework 3 (SEQ ID NO:637), CDR3 (SEQ ID NO:651), Framework 4 (SEQ ID NO:655), 6B9: all sequences (SEQ ID NO:656), Framework 1 (SEQ ID NO:625), CDR1 (SEQ ID NO:629), Framework 2 (SEQ ID NO:632), CDR2 (SEQ ID NO:635), Framework 3 (SEQ ID NO:638), CDR3 (SEQ ID NO:652), Framework 4 (SEQ ID NO:655), 6B9.1: all sequences (SEQ ID NO: 657), Framework 1 (SEQ ID NO:625), CDR1 (SEQ ID NO:629), Framework 2 (SEQ ID NO: 632), CDR2 (SEQ ID NO:636), Framework 3 (SEQ ID NO:638), CDR3 (SEQ ID NO:653), Framework 4 (SEQ ID NO:655), A1: all sequences (SEQ ID NO:658), Framework 1 (SEQ ID NO: 626), CDR1 (SEQ ID NO:629), Framework 2 (SEQ ID NO:633), CDR2 (SEQ ID NO:636), Framework 3 (SEQ ID NO:639), CDR3 (SEQ ID NO:654), Framework 4 (SEQ ID NO:655), A2: all sequences (SEQ ID NO:659), Framework 1 (SEQ ID NO:625), CDR1 (SEQ ID NO:629), Framework 2 (SEQ ID NO:633), CDR2 (SEQ ID NO:636), Framework 3 (SEQ ID NO:640), CDR3 (SEQ ID NO:654), Framework 4 (SEQ ID NO:655), A8: all sequences (SEQ ID NO: 660), Framework 1 (SEQ ID NO:625), CDR1 (SEQ ID NO:629), Framework 2 (SEQ ID NO: 633), CDR2 (SEQ ID NO:636), Framework 3 (SEQ ID NO:641), CDR3 (SEQ ID NO:654), Framework 4 (SEQ ID NO:655), A11: all sequences (SEQ ID NO:661), Framework 1 (SEQ ID NO: 625), CDR1 (SEQ ID NO:630), Framework 2 (SEQ ID NO:633), CDR2 (SEQ ID NO:636), Framework 3 (SEQ ID NO:638), CDR3 (SEQ ID NO:654), Framework 4 (SEQ ID NO:655), B1: all sequences (SEQ ID NO:662), Framework 1 (SEQ ID NO:625), CDR1 (SEQ ID NO:629), Framework 2 (SEQ ID NO:632), CDR2 (SEQ ID NO:636), Framework 3 (SEQ ID NO:642), CDR3 (SEQ ID NO:654), Framework 4 (SEQ ID NO:655), B3: all sequences (SEQ ID NO: 663), Framework 1 (SEQ ID NO:625), CDR1 (SEQ ID NO:629), Framework 2 (SEQ ID NO: 633), CDR2 (SEQ ID NO:636), Framework 3 (SEQ ID NO:643), CDR3 (SEQ ID NO:654), Framework 4 (SEQ ID NO:655), C4=F10: all sequences (SEQ ID NO:664), Framework 1 (SEQ ID NO: 625), CDR1 (SEQ ID NO:629), Framework 2 (SEQ ID NO:633), CDR2 (SEQ ID NO: 636), Framework 3 (SEQ ID NO:644), CDR3 (SEQ ID NO:654), Framework 4 (SEQ ID NO: 655), C7=D1: all sequences (SEQ ID NO:665), Framework 1 (SEQ ID NO:625), CDR1 (SEQ ID NO:629), Framework 2 (SEQ ID NO:633), CDR2 (SEQ ID NO:636), Framework 3 (SEQ ID NO:644), CDR3 (SEQ ID NO:654), Framework 4 (SEQ ID NO:655), D3=F1: all sequences (SEQ ID NO:666), Framework 1 (SEQ ID NO:625), CDR1 (SEQ ID NO:629), Framework 2 (SEQ ID NO:633), CDR2 (SEQ ID NO:636), Framework 3 (SEQ ID NO:645), CDR3 (SEQ ID NO:654), Framework 4 (SEQ ID NO:655), D10=E5: all sequences (SEQ ID NO: 667), Framework 1 (SEQ ID NO:625), CDR1 (SEQ ID NO:629), Framework 2 (SEQ ID NO: 633), CDR2 (SEQ ID NO:636), Framework 3 (SEQ ID NO:646), CDR3 (SEQ ID NO:654), Framework 4 (SEQ ID NO:655), G4: all sequences (SEQ ID NO:668), Framework 1 (SEQ ID NO: 625), CDR1 (SEQ ID NO:629), Framework 2 (SEQ ID NO:633), CDR2 (SEQ ID NO:636), Framework 3 (SEQ ID NO:647), CDR3 (SEQ ID NO:654), Framework 4 (SEQ ID NO:655), C4: all sequences (SEQ ID NO:669), Framework 1 (SEQ ID NO:625), CDR1 (SEQ ID NO:629), Framework 2 (SEQ ID NO:633), CDR2 (SEQ ID NO:636), Framework 3 (SEQ ID NO:650), CDR3 (SEQ ID NO:654), Framework 4 (SEQ ID NO:655), D10: all sequences (SEQ ID NO: 670), Framework 1 (SEQ ID NO:625), CDR1 (SEQ ID NO:629), Framework 2 (SEQ ID NO: 633), CDR2 (SEQ ID NO:636), Framework 3 (SEQ ID NO:646), CDR3 (SEQ ID NO:654), Framework 4 (SEQ ID NO:655), 4F1A11: all sequences (SEQ ID NO:671), Framework 1 (SEQ ID NO: 625), CDR1 (SEQ ID NO:629), Framework 2 (SEQ ID NO:632), CDR2 (SEQ ID NO: 636), Framework 3 (SEQ ID NO:650), CDR3 (SEQ ID NO:654), Framework 4 (SEQ ID NO: 655), 4F1E1: all sequences (SEQ ID NO:672), Framework 1 (SEQ ID NO:625), CDR1 (SEQ ID NO:631), Framework 2 (SEQ ID NO:632), CDR2 (SEQ ID NO:636), Framework 3 (SEQ ID NO:638), CDR3 (SEQ ID NO:654), Framework 4 (SEQ ID NO:655), 4F1G3: all sequences (SEQ ID NO:673), Framework 1 (SEQ ID NO:625), CDR1 (SEQ ID NO:631), Framework 2 (SEQ ID NO:632), CDR2 (SEQ ID NO:636), Framework 3 (SEQ ID NO:648), CDR3 (SEQ ID NO:654), Framework 4 (SEQ ID NO:655), 4F1E10: all sequences (SEQ ID NO: 674), Framework 1 (SEQ ID NO:627), CDR1 (SEQ ID NO:631), Framework 2 (SEQ ID NO: 632), CDR2 (SEQ ID NO:636), Framework 3 (SEQ ID NO:638), CDR3 (SEQ ID NO:654), Framework 4 (SEQ ID NO:655), 4F1E9: all sequences (SEQ ID NO:675), Framework 1 (SEQ ID NO: 625), CDR1 (SEQ ID NO:629), Framework 2 (SEQ ID NO:632), CDR2 (SEQ ID NO: 636), Framework 3 (SEQ ID NO:638), CDR3 (SEQ ID NO:654), Framework 4 (SEQ ID NO: 655), 4F1H12: all sequences (SEQ ID NO:676), Framework 1 (SEQ ID NO:625), CDR1 (SEQ ID NO:631), Framework 2 (SEQ ID NO:632), CDR2 (SEQ ID NO:636), Framework 3 (SEQ ID NO:649), CDR3 (SEQ ID NO:654), Framework 4 (SEQ ID NO:655), F9: all sequences (SEQ ID NO:677), Framework 1 (SEQ ID NO:625), CDR1 (SEQ ID NO:631), Framework 2 (SEQ ID NO:632), CDR2 (SEQ ID NO:636), Framework 3 (SEQ ID NO:638), CDR3 (SEQ ID NO:654), and Framework 4 (SEQ ID NO:655). FIG. 11B Sequences-4F1: all sequences (SEQ ID NO:678), Framework 1 (SEQ ID NO:692), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), 6B9: all sequences (SEQ ID NO: 679), Framework 1 (SEQ ID NO:699), CDR1 (SEQ ID NO:700), Framework 2 (SEQ ID NO: 701), CDR2 (SEQ ID NO:695), Framework 3 (SEQ ID NO:696), CDR3 (SEQ ID NO: 702), Framework 4 (SEQ ID NO:698), 6B9.1: all sequences (SEQ ID NO:680), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), A1=A2=C4=C7=D1=D10=E5=F1=F10=G4: all sequences (SEQ ID NO:681), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), A8: all sequences (SEQ ID NO: 682), Framework 1 (SEQ ID NO:703), CDR1 (SEQ ID NO:693), Framework 2 (SEQ ID NO: 694), CDR2 (SEQ ID NO:695), Framework 3 (SEQ ID NO:696), CDR3 (SEQ ID NO: 697), Framework 4 (SEQ ID NO:698), A11: all sequences (SEQ ID NO:683), Framework 1 (SEQ ID NO:704), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 698), B1: all sequences (SEQ ID NO:684), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), B3: all sequences (SEQ ID NO:685), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), D10=E5: all sequences (SEQ ID NO: 686), Framework 1 (SEQ ID NO:703), CDR1 (SEQ ID NO:693), Framework 2 (SEQ ID NO: 694), CDR2 (SEQ ID NO:695), Framework 3 (SEQ ID NO:696), CDR3 (SEQ ID NO: 697), Framework 4 (SEQ ID NO:698), C4: all sequences (SEQ ID NO:687), Framework 1 (SEQ ID NO:703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 706), D10: all sequences (SEQ ID NO:688), Framework 1 (SEQ ID NO:699), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:706), 4F1E1=1F1G3=4F1B5=4F1G11=4F1A9=4F1B9=4F1H9=4F1D10=4F1E9=4F1F10=4F1H11=4F1H12: all sequences (SEQ ID NO:689), Framework 1 (SEQ ID NO:703), CDR1 (SEQ ID NO: 693), Framework 2 (SEQ ID NO:694), CDR2 (SEQ ID NO:695), Framework 3 (SEQ ID NO: 696), CDR3 (SEQ ID NO:697), Framework 4 (SEQ ID NO:698), 4FA11: all sequences (SEQ ID NO:690), Framework 1 (SEQ ID NO:705), CDR1 (SEQ ID NO:693), Framework 2 (SEQ ID NO:694), CDR2 (SEQ ID NO:695), Framework 3 (SEQ ID NO:696), CDR3 (SEQ ID NO: 697), Framework 4 (SEQ ID NO:698), F9: all sequences (SEQ ID NO:691), Framework 1 (SEQ ID NO:703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), and Framework 4 (SEQ ID NO: 706).

FIG. 12 demonstrates how the C6D4 epitope overlaps directly with the ligand binding pocket of integrin αVβ8, preventing association of integrin αVβ8 with L-TGFβ and thus activation of L-TGFβ. Representative class averages of integrin complexes observed by negative staining electron microscopy.

FIGS. 13A-B illustrate a model of how the complex is generated from the crystal structure of αVβ3 (PDB ID: 3IJE), with the β8 model based on β3 using CHIMERA and MODELLER (Yang et al., J Struct Biol. 2012 September; 179 (3): 269-78). Refinement of the model to the cryo-electron microscopy map is done in rigid body in COOT (Emsley P, et al., Acta Crystallographica Section D—Biological Crystallography. 2010, 66:486-501), followed by complete refinement in PHENIX (Adams et al., Acta Cryst. 2010; D66: 213-221).

FIG. 14 illustrates interaction of C6D4 Vk CDR1 (SEQ ID NO:616) with integrin αV (SEQ ID NO:622, positions 46-52 and 75-79): Model of the αVβ8/C6D4 Fab complex. Interacting residues are represented as sticks. The dashed lines represent inter-atom distances comprised between 2.5 and 4.0 Å indicating potential interactions.

FIG. 15 illustrates interaction of C6D4 with the SDL region of integrin β8: Model of the αVβ8/C6D4 Fab complex. Interacting residues are represented as sticks. The dashed lines represent inter-atom distances comprised between 2.5 and 4.0 Å indicating potential interactions. C6D4 VH CDR1 (SEQ ID NO:707), C6D4 VH CDR3 (SEQ ID NO:615), β8, SDL (SEQ ID NO: 620), C6D4 Vk CDR1 (SEQ ID NO:616), C6D4 Vk CDR2 (SEQ ID NO:708), and C6D4 Vk CDR3 (SEQ ID NO:618).

FIG. 16 illustrates interaction of C6D4 Vk CDR2 (SEQ ID NO:617) with the al helix of integrin β8 (SEQ ID NO:619): Model of the αVβ8/C6D4 Fab complex. Interacting residues are represented as sticks. The dashed lines represent inter-atom distances comprised between 2.5 and 4.0 Å indicating potential interactions.

FIG. 17 illustrates interaction of C6D4 Vk CDR1 (SEQ ID NO:613) with the α2 helix of integrin β8 (SEQ ID NO:621): Model of the αVβ8/C6D4 Fab complex. Interacting residues are represented as sticks. The dashed lines represent inter-atom distances comprised between 2.5 and 4.0 Å indicating potential interactions.

FIG. 18 illustrates that C6D4 blocks the access of L-TGFβ to the ligand binding pocket of integrin β8 and C6D4 bound to integrin αVβ8 directly clashes with the position of the RGDLGRLKK loop of L-TGFβ (SEQ ID NO:712). The surface of the αVβ8/C6D4 Fab complex is shown. The surface is αVβ8, while the cartoon is C6D4. In sticks are superimposed the residues RGDLGRLKK (SEQ ID NO:712) from the integrin binding loop of L-TGFβ as found when bound to integrin αVβ6 (PDB 4UM9) ((4) Structural determinants of integrin β-subunit specificity for latent TGF-β. Dong X, et al. Nat. Struct. Mol. Biol. 2014 December; 21 (12): 1091-6).

FIG. 19 shows that C6D4 is a potent inhibitor of binding of secreted αvβ8 to L-TGF-b3 peptide.

FIG. 20 shows that C6D4 is a potent inhibitor of cell αvβ8-mediated cell adhesion to L-TGF-b3 peptide.

FIGS. 21A-D show immunodetection of the integrin b8 subunit in formalin fixed paraffin embedded sections from patient infected with H. Pylori (A,B) or showing normal histology (C,D). The sections were stained with clone F9 in rabbit IgG format and detected using TSA signal amplification (Perkin Elmer). The brown precipitate indicates positive staining and the nuclei are counterstained with hematoxylin. The arrows indicate examples of positive crypts with stained crypt epithelial cells. The results show that the b8 integrin subunit is increased in expression in the stomachs of patients with H. Pylori.

FIG. 22 shows quantification of Immunodetection of the integrin b8 subunit in formalin fixed paraffin embedded sections from patient infected with H. Pylori, showing normal histology or mild chronic inflammation. The sections were stained with clone F9 in rabbit IgG format and detected using TSA signal amplification (Perkin Elmer). The following scoring system was devised to capture the crypto-epithelial staining, 0=no stain, 1=just contrast+, 2-scattered, 3=diffuse and stromal staining, 0=no stain, 1-just contrast+, 2=scattered, 3=diffuse. Shown is a combined score and the n is shown. ANOVA with Sidak's multiple comparisons test. ** p<0.01, * p<0.05

FIG. 23 shows binding assay of alkaline phosphatase (AP) αvβ3, αvβ6 and αvβ8 fusion proteins to CagL, the MAP RGD peptide derived from the TGFB3 sequence DDHGRGDLGRLK (SEQ ID NO:713), Fibronectin, Vitronectin or a MAP peptide derived from the TGFB2 sequence that corresponds to the RGD containing sequence of TGFB1 and TGFB3. All proteins are coated on ELISA plates at 5 μg/ml and input of AP receptors are normalized to AP activity. Results shown represent signal above BSA coated wells. The results show that αvβ8 (and αvβ6) binds to CagL as well as to TGFb3 peptide, whereas αvβ3 binds to FN and VN, poorly to TGFB3 and not at all to CagL. αvβ3 and αvβ8 show no binding and αvβ6 shows very weak binding to the control TGFb2 peptide. Shown are S.E.M.

FIG. 24 shows binding assay of alkaline phosphatase (AP) αvβ8 fusion protein to CagL in the presence of C6D4, an allosteric inhibitor, B5, or a non-blocking antibody to the same epitope as B5, clone 68 which serves as a negative control. CagL is coated on ELISA plates at 5 μg/ml. Results shown represent signal above BSA coated wells. The results show that αvβ8 binding to CagL is completely inhibited by C6D4 and are partially inhibited by B5.

FIG. 25 shows adhesion assay of CHO Lec cells stably expressing human ITGAV and ITGB8 to recombinant CagL protein at the indicated concentrations (gift of Eric. Sundberg, University of Maryland, MD). Binding is compared to wells coated with a multiple antigen presenting peptide containing the RGD peptide derived from the TGFB3 sequence DDHGRGDLGRLK (SEQ ID NO:713), which corresponds to aa 257-268 of human TGF-b3 (NP_003230). 50×10\hat{ }3 cells were allowed to attach to the wells for 30 min at RT. Unbound cells were washed off with PBS. Results were presented as stained cells detected after staining with crystal violet (OD590). Results shown represent signal above the nominal binding of mock transfected CHO Lec cells to CagL or TGFB3 peptide coated wells (5 μg·ml). The results show that αvβ8 mediates cell adhesion to CagL as well as to TGFb3 peptide. Shown are S.E.M. Significance was determined by ANOVA and Sidak's multiple comparison test. ****=p<0.00001

FIG. 26 shows adhesion assay of CHO Lec cells stably expressing human ITGAV and ITGB8 to the TGF-b3 RGD MAP peptide (DDHGRGDLGRLK (SEQ ID NO:713)) (coating concentration 5 μg/ml). 50×10\hat{ }3 cells were preincubated with cagL at the indicated concentrations of CagL vs PBS control for 15 min at RT and then the cells allowed to attach to the wells for 30 min at RT. Unbound cells were washed off with PBS. Results were presented as stained cells detected after staining with crystal violet (OD590). Results shown represent signal above the nominal binding of mock transfected CHO Lec cells to TGFB3 peptide coated wells (5 μg/ml). The results show that αvβ8 mediates cell adhesion to CagL is RGD dependent. Shown are S.E.M. N=3

FIG. 27 shows adhesion assay of modified Chinese Hamster Ovary Cells (CHO Lec) cells stably expressing human ITGAV and ITGB8 to recombinant CagL protein at 5 μg/ml coating concentration, 50×10\hat{ }3 cells were mixed with the Mabs at the indicated concentrations and allowed to attach to the wells for 30 min at RT. B5 is a previously described allosteric inhibitor of αvβ8-binidng to TGF-B and L230 is a previously described anti-av blocking antibody. Unbound cells were washed off with PBS. Results are presented as stained cells detected after staining with crystal violet (OD590). Results shown represent % inhibition relative to the control binding defined by binding in presence of an isotype control antibody (anti-SV5) at the same concentration. Shown are S.E.M. Significance was determined by ANOVA and Sidak's multiple comparison test. ****=p<0.00001, *** p<0.001, *<0.05. The results show that C6D4 more efficiently blocks αvβ8 mediates cell adhesion to CagL than B5 or L230.

FIG. 28 shows a mouse model for evaluating lung metastasis using the LLC tumor cell line which does not express integrins αvβ6 or αvβ8. The LLC tumor cell line is syngenic to the host C57B/6 strain. The LLC.1 cell line has been passed though mice one time and regrown from lung metastasis. After two weeks, subcutaneously injected tumor (1×10⁶) LLC.1 cells form large tumor nodules (˜1 cm). The tumors are removed surgically and when animals lose 20% body weight they are euthanized.

FIGS. 29A and 29B show the effect of C6D4 on mouse survival using the LLC tumor cell line model set forth in FIG. 28. Survival curves (FIG. 29A) represent mice euthanized for reasons of local recurrence or weight loss. FIG. 29B shows the survival curve when animals removed for local recurrence are excluded. At autopsy, the animals with 20% weight loss all have metastatic implants in their lungs. Here, C6D4 antibodies have been injected for up to 90 days in surviving animals. This experiment was performed eleven times, each time providing similar results (data not shown). Additionally, post-mortem examination did not reveal any abnormal inflammatory response in the tissues examined.

FIGS. 30A-F show the effect of CD64 on tumor growth and tumor immune response using the LLC tumor cell line model set forth in FIG. 28. Here, resected LLC.1 primary tumors in mice that received two injections of isotype control (B5, which only reacts with human and not mouse b8) or C6D4 (which cross reacts with mouse and human), the primary tumor weights are recorded, dimensions are measured, and tumors are enzymatically disaggregated and immune cells isolated and counted. Flow cytometry was performed on the tumor infiltrating immune cells, and the tumor infiltrating immune cells are separated from tumor cells using Percoll gradient centrifugation. Shown here is one of three experiments each providing similar results. In each group n is equal to or greater than 10.

FIG. 31 shows a mouse model for evaluating metastatic disease using B16-F10 tumor cells. The B16-F10 highly metastatic tumor cell line is syngenic to the host C57B/6 strain. This line does not express integrins αvβ6 or αvβ8. The B16-F10 was transfected with murine itgb8 and after selection and sorting expresses surface αvβ8 at high levels. When injected intravenously via the tail vein, visible lung metastases appear by 14 days.

FIGS. 32A-H are lung adenocarcinoma samples stained with anti-b8 (FIGS. 32E-H) or anti-PD-L1 (E1L3N, Cell signaling) FIG. 32A-D. Here, it was observed that beta 8 expression inversely correlated with PD-L1 expression.

FIG. 33A shows distribution of lung adenocarcinoma samples of FIG. 32 (n=29) with staining for either PD-L1 or beta 8. FIG. 33B shows in all cases that stained at least 30% for beta 8 or PD-L1 were grouped together and the staining proportions were correlated.

FIGS. 34A-C shows the inhibition of B16 lung metastases as compared to an isotype sample. FIG. 34A are photographs of representative lungs in anterior and posterior views and visible lung metastases were counted and the total lung surface area involved with metastases was assessed. FIG. 34B shows the effect of C6D4 on total number of lung metastases. The B16-F10 highly metastatic tumor cell line is syngenic to the host C57B/6 strain and does not express integrins αvβ6 or αvβ8. The B16-F10 tumor cells were transfected with murine itgb8. After selection in G418 and two rounds of sorting a pool of high expressing αvβ8 cells was injected intravenously via the tail vein. After three injections (i.p.) of isotype control (SV5) or C6D4, both at 7 mg/kg at days 0, 7 and 14, the mice were euthanized at day 18. FIG. 34C shows the effect of C6D4 as measured by percentage of total lung surface area involved by metastatic melanoma.

FIGS. 35A-H show that C6D4 effects macrophage polarization to a proinflammatory phenotype. Increases in MHCII expression by tumor associated macrophages and dendritic cells is important in host immune responses to tumor antigens.

FIGS. 36A-F shows that C6D4 increases MHCII expression by tumor associated dendritic cells. Increases in MHCII by antigen presenting cells will increase antigen presentation.

FIGS. 37A-G are scatterplots showing integrin mediated differentiation of mouse Treg cells. Tumor associated CD4+T regulatory cells play an important role in suppressing the host immune response and help tumors escape immune surveillance. The differentiation of Treg requires TGF-beta. It is thought that TGF-beta provided by mechanisms such as integrin αvβ8 mediated activation are important for Treg differentiation and function. Here, we immobilized the ectodomains of various integrins (2 mg/ml) on ELISA plates (co-coated with anti-CD3) and plate naïve murine splenic CD4+ cells with hIL-2 and retinoic acid. After 5 days the cells were fixed, permeabilized and stained with anti-CD4 and FoxP3. As a positive and negative control, cells were plated on wells with only anti-CD3 (no integrin) in the presence (+) or absence (−) of rTGF-b. The percentage of FoxP3 expressing cells are shown in each of the scatterplots (Q2).

FIGS. 38A-D shows structural representations of a C6D4 derivative (termed “RGD3” or “CD64-RGD3”) that is cross-reactive to αvβ6 and αvβ8 but not αvβ1, αvβ3, or αvβ5. FIG. 38A shows a close-up view of C6D4-RGD3 (gold) in complex with αvβ8 derived from cryoEM maps. Green is the αv subunit and blue is the 8 subunit. Shown in red is the LTGF-B1 peptide derived from structures of Latent-TGFB1 in complex with the integrin αvβ6. (αvβ6 (SEQ ID NO: 709), αIIbβ3 (SEQ ID NO:710 (GRGDSP) and SEQ ID NO:711 (AKQRGDV). FIG. 38B shows sequence alignments of hTGFβ1-3 and the position of the RGD domains in TGFβ1 (SEQ ID NO: 714) and TGFβ3 (SEQ ID NO:715). TGFβ2 (SEQ ID NO:716) does not have an RGD sequence. FIG. 38C shows the sequence of three mutant D4 Vk CDR1 loops containing portions of the hTGFB3 RGD sequence (in red) developed herein (C6D4 vk (SEQ ID NO:717); C6D4-RDG1 (SEQ ID NO:718); C6D4-RDG2 (SEQ ID NO:719); and C6D4-RDG3 (SEQ ID NO: 720). FIG. 38D shows a zoomed image of the D4 loop (shown in gold) and the clash with the position of the bound RGD sequence of TGFβ1 in complex with integrin αvβ6.

FIG. 39 shows cell surface staining experiments of C6VH expressed with either RGD1, RGD2 or RGD3 mutants (as set forth in FIG. 38) as rabbit IgG. Binding to human Cho cells expressing αvβ8 was expressed as a percentage of binding of C6D4.

FIG. 40 shows cell surface staining experiments of C6VH expressed with either D4 Vk or RGD1, RGD2 or RGD3 mutants as rabbit IgG. Binding to Cho cells expressing human αvβ8 or SW480 cells expressing αvβ6 are shown. Relative binding is defined as staining compared to staining of non-transfected Cho or SW480 cells.

FIG. 41 shows binding experiments of C6VH expressed with either D4 Vk or RGD1, RGD2 or RGD3 mutants as rabbit IgG, to various av-integrins. All integrins were coated on ELISA plates at 2 mg/ml, blocked with BSA, and antibodies were allowed to bind. Binding of C6D4 and C6D4-RGD3 were detected with anti-rabbit HRP. The results are shown relative to control wells coated with anti-av (clone 8B8) where av-integrins were detected with another av-antibody recognizing an non-overlapping epitope (L230-biotin), followed by SA-HRP.

FIG. 42 shows the effect of cations on the binding of C6D4 and C6D4-RGD3 to various receptors. Binding in EDTA containing buffer defines cation-dependence because EDTA binds to Ca⁺⁺ and Mg⁺⁺. Magnesium cation buffers contains 1 mM Ca⁺⁺ and 1 mM Mg⁺⁺. Here, the results are relative to anti-av, clone 11D12V2. All antibodies were coated on ELISA plates at 5 μg/ml. The αvβ8 or αvβ6 receptors (0.5 μg/ml) were bound for 1 hour and were then detected with biotinylated anti-av clone 8B8. The small amount of αvβ8 binding to C6D4-RGD3 in EDTA buffer (compared with no αvβ6 binding to C6D4-RGD3 in EDTA buffer) suggests that the binding to αvβ6 is more dependent on the RGD binding loop of Vk CDR1 than the binding to αvβ8.

FIGS. 43A and 43B show inhibition of αvβ8 adhesion and TGF-beta activation. Cho 3.2.8.1 cells transfected with b8 were used in adhesion assays to wells coated with branched GRGDLGRLK peptide (SEQ ID NO:721) (10 μg/ml). Cho-b8 cells were allowed to bind in adhesion assays (FIG. 43A) in the presence of C6D4, RGD3 or control Mab at various concentrations. Cho-b8 cells were allowed to attach to wells with TMLC TGF-β reporter cells in the presence of C6D4, RGD3 or control Mab at various concentrations (FIG. 43B). The values are shown as a proportion of control Mab (SV5) control. The results indicate that C6D4-RGD3 and C6D4 block αvβ8 function similarly.

FIGS. 44A-B show adhesion assays and FIGS. 44C-D show TGF-beta activation. Here, Cho3.2.8.1 cells were transfected with GARP and LTGFB1. GARP (LRRC32) is a cell surface scaffolding molecule present on the surface of Treg cells and binds LTGF-b to the cell surface. Upper panels (FIGS. 44A and 44B) show adhesion assays of Cho cells expressing GARP/LTGFB adhering to immobilized αvβ8 (FIG. 44A) or αvβ6 (FIG. 44B) performed in the presence of anti-β8 (C6D4), 3G9 (anti-β6) or the bispecific antibody RGD3 (anti-β6 and anti-β8). In the lower panels (FIGS. 44C and 44D), the TGF-beta reporters cells TMLC, were added to each well to determine the amount of TGF-β activation in response to αvβ8 (FIG. 44C) or αvβ6 (FIG. 44D) performed in the presence of anti-β8 (C6D4), 3G9 (anti-β6) or the bispecific antibody RGD3 (anti-β6 and anti-β8). The results are reported as relative luciferase activity to wells treated with isotype control antibody (SV5). Below each graph is the EC50 of each inhibitory antibody.

FIG. 45 shows binding assay of αvβ6 to TGFβ3 peptide. Mab 3G9 is a potent inhibitor of αvβ6-mediated TGF-b activation. C6D4-RGD3 shows cross-competition with 3G9 binding suggesting that they have overlapping binding footprints or allosterically influence each other's binding. However, these antibodies have different modes of action as 3G9 binding to αvβ6 is not cation-dependent while C6D4-RGD3 binding is cation dependent.

FIG. 46 illustrates heavy and light chain amino acid sequences for clones used in the construction of the composite humanized antibody C6D4. VH sequences-C6D4: all sequences (SEQ ID NO:722), Framework 1 (SEQ ID NO:732), CDR1 (SEQ ID NO:733), Framework 2 (SEQ ID NO:734), CDR2 (SEQ ID NO:735), Framework 3 (SEQ ID NO:736), CDR3 (SEQ ID NO: 737), Framework 4 (SEQ ID NO:738); HuC6D4 V1: all sequences (SEQ ID NO:723), Framework 1 (SEQ ID NO:739), CDR1 (SEQ ID NO:733), Framework 2 (SEQ ID NO:740), CDR2 (SEQ ID NO:735), Framework 3 (SEQ ID NO:741), CDR3 (SEQ ID NO:737), Framework 4 (SEQ ID NO:738); Mutclone A3: all sequences (SEQ ID NO:724), Framework 1 (SEQ ID NO:739), CDR1 (SEQ ID NO:733), Framework 2 (SEQ ID NO:740), CDR2 (SEQ ID NO: 735), Framework 3 (SEQ ID NO:741), CDR3 (SEQ ID NO:737), Framework 4 (SEQ ID NO: 738); Mutclone B7: all sequences (SEQ ID NO:725), Framework 1 (SEQ ID NO:742), CDR1 (SEQ ID NO:733), Framework 2 (SEQ ID NO:740), CDR2 (SEQ ID NO:735), Framework 3 (SEQ ID NO:743), CDR3 (SEQ ID NO:744), Framework 4 (SEQ ID NO:738); Mutclone E5: all sequences (SEQ ID NO:726), Framework 1 (SEQ ID NO:739), CDR1 (SEQ ID NO: 733), Framework 2 (SEQ ID NO:740), CDR2 (SEQ ID NO:735), Framework 3 (SEQ ID NO: 741), CDR3 (SEQ ID NO:744), and Framework 4 (SEQ ID NO:738). VK sequences-C6D4: all sequences (SEQ ID NO:727), Framework 1 (SEQ ID NO:745), CDR1 (SEQ ID NO: 746), Framework 2 (SEQ ID NO:747), CDR2 (SEQ ID NO:748), Framework 3 (SEQ ID NO: 749), CDR3 (SEQ ID NO:750), Framework 4 (SEQ ID NO:751); HuC6D4 V1: all sequences (SEQ ID NO:728), Framework 1 (SEQ ID NO:752), CDR1 (SEQ ID NO:746), Framework 2 (SEQ ID NO:747), CDR2 (SEQ ID NO:748), Framework 3 (SEQ ID NO:753), CDR3 (SEQ ID NO:750), Framework 4 (SEQ ID NO:754); Mutclone A3: all sequences (SEQ ID NO: 729), Framework 1 (SEQ ID NO:755), CDR1 (SEQ ID NO:756), Framework 2 (SEQ ID NO: 747), CDR2 (SEQ ID NO:748), Framework 3 (SEQ ID NO:753), CDR3 (SEQ ID NO: 750), Framework 4 (SEQ ID NO:754); Mutclone B7: all sequences (SEQ ID NO:730), Framework 1 (SEQ ID NO:757), CDR1 (SEQ ID NO:746), Framework 2 (SEQ ID NO:747), CDR2 (SEQ ID NO:748), Framework 3 (SEQ ID NO:758), CDR3 (SEQ ID NO:750), Framework 4 (SEQ ID NO:754); Mutclone E5: all sequences (SEQ ID NO:731), Framework 1 (SEQ ID NO:752), CDR1 (SEQ ID NO:756), Framework 2 (SEQ ID NO:747), CDR2 (SEQ ID NO: 748), Framework 3 (SEQ ID NO:753), CDR3 (SEQ ID NO:750), and Framework 4 (SEQ ID NO: 754).

FIG. 47 shows binding assay of humanized C6D4 or RDG3 to recombinant αvβ8. Humanized C6D4 or RGD3 (Frameworks and CH1 are human; hinge and CH2-3 are mouse) were immobilized on ELISA plates at the indicated concentrations. As a negative control, some wells were coated with anti-SV5 at the same concentrations. Non-specific binding sites were blocked with BSA. Recombinant αvβ8 ectodomain (0.5 g/ml) was added to each well and after binding and washing in binding buffer (1 mM Ca and Mg), the bound αvβ8 was detected with biotinylated anti-αv (8b8) and detected with SA-HRP. Results are shown as specific binding (minus SV5 control).

FIG. 48A-D shows superposition of C6D4/αvβ8 cartoon model with wire map of C6D4/αvβ8 (FIGS. 48A and 48C) compared to a superposition of the same C6D4/αvβ8 cartoon model with wire map of C6D4-RGD3/αvβ8 (FIGS. 48B and D). The comparison of the two maps shows a different orientation of the CDR1 Vk loop of C6D4-RGD3 towards the beta8 subunit ligand binding site.

FIGS. 49A-C shows CryoEM maps of C6D4 and C6D4-RGD αvβ8 complexes having similar positioning. Here, C6D4 Fab-αvβ8 (FIG. 49A) is compared with RGD3-αvβ8 map (FIG. 49B), or in overlay (FIG. 49C), based on cryoEM derived density maps. The anti-av 11D12V2 Fab was used to increase molecular mass of the complex and to assist in particle orientation.

FIG. 50 illustrates heavy chain amino acid sequences for clones used in the construction of the composite humanized antibodies C6D4 and C6D4-RGD3. Consensus sequences for the humanized version of C6D4 and C6D4-RGD3 are provided. VH sequences-HuC6D4V1: all (SEQ ID NO:395), Framework 1 (SEQ ID NO:396), CDR1 (SEQ ID NO:397), Framework 2 (SEQ ID NO:398), CDR2 (SEQ ID NO:399), Framework 3 (SEQ ID NO:400), CDR3 (SEQ ID NO:401), and Framework 4 (SEQ ID NO:402); HuC6D4A3: all (SEQ ID NO: 403), Framework 1 (SEQ ID NO:404), CDR1 (SEQ ID NO:405), Framework 2 (SEQ ID NO: 406), CDR2 (SEQ ID NO:407), Framework 3 (SEQ ID NO:408), CDR3 (SEQ ID NO:409), and Framework 4 (SEQ ID NO:410); HuC6D4B7: all (SEQ ID NO:411), Framework 1 (SEQ ID NO: 412), CDR1 (SEQ ID NO:413), Framework 2 (SEQ ID NO:414), CDR2 (SEQ ID NO:415), Framework 3 (SEQ ID NO:416), CDR3 (SEQ ID NO:417), and Framework 4 (SEQ ID NO:418); HuC6D4E5: all (SEQ ID NO:419), Framework 1 (SEQ ID NO:420), CDR1 (SEQ ID NO:421), Framework 2 (SEQ ID NO:422), CDR2 (SEQ ID NO:423), Framework 3 (SEQ ID NO:424), CDR3 (SEQ ID NO:425), and Framework 4 (SEQ ID NO:426); C6D4: all sequences (SEQ ID NO: 722), Framework 1 (SEQ ID NO:732), CDR1 (SEQ ID NO:733), Framework 2 (SEQ ID NO: 734), CDR2 (SEQ ID NO:735), Framework 3 (SEQ ID NO:736), CDR3 (SEQ ID NO: 737), and Framework 4 (SEQ ID NO:738); HuC6D4: all (SEQ ID NO:427), Framework 1 (SEQ ID NO:428), CDR1 (SEQ ID NO:429), Framework 2 (SEQ ID NO:430), CDR2 (SEQ ID NO: 431), Framework 3 (SEQ ID NO:432), CDR3 (SEQ ID NO:433), and Framework 4 (SEQ ID NO: 434); C6D4-RGD3: all (SEQ ID NO:435), Framework 1 (SEQ ID NO:436), CDR1 (SEQ ID NO: 437), Framework 2 (SEQ ID NO:438), CDR2 (SEQ ID NO:439), Framework 3 (SEQ ID NO: 440), CDR3 (SEQ ID NO:441), and Framework 4 (SEQ ID NO:442); HuC6D4-RGD3: all (SEQ ID NO:443), Framework 1 (SEQ ID NO:444), CDR1 (SEQ ID NO:445), Framework 2 (SEQ ID NO:446), CDR2 (SEQ ID NO:447), Framework 3 (SEQ ID NO:448), CDR3 (SEQ ID NO: 449), and Framework 4 (SEQ ID NO:450); and Consensus VH: Framework 1 (SEQ ID NO: 558), CDR1 (SEQ ID NO:563), Framework 2 (SEQ ID NO:559), CDR2 (SEQ ID NO: 563), Framework 3 (SEQ ID NO:560), CDR3 (SEQ ID NO:564), and Framework 4 (SEQ ID NO: 561).

FIG. 51 illustrates light chain amino acid sequences for clones used in the construction of the composite humanized antibodies C6D4 and C6D4-RGD3. Consensus sequences for the humanized version of C6D4 and C6D4-RGD3 are provided. VL sequences-HuC6D4V1: all (SEQ ID NO:451), Framework 1 (SEQ ID NO:452), CDR1 (SEQ ID NO:453), Framework 2 (SEQ ID NO:454), CDR2 (SEQ ID NO:455), Framework 3 (SEQ ID NO:456), CDR3 (SEQ ID NO: 457), and Framework 4 (SEQ ID NO:458); HuC6D4A3: all (SEQ ID NO:459), Framework 1 (SEQ ID NO:460), CDR1 (SEQ ID NO:461), Framework 2 (SEQ ID NO:462), CDR2 (SEQ ID NO: 463), Framework 3 (SEQ ID NO:464), CDR3 (SEQ ID NO:465), and Framework 4 (SEQ ID NO: 466); HuC6D4B7: all (SEQ ID NO:467), Framework 1 (SEQ ID NO:468), CDR1 (SEQ ID NO: 469), Framework 2 (SEQ ID NO:470), CDR2 (SEQ ID NO:471), Framework 3 (SEQ ID NO: 472), CDR3 (SEQ ID NO:473), and Framework 4 (SEQ ID NO:474); HuC6D4E5: all (SEQ ID NO: 475), Framework 1 (SEQ ID NO:476), CDR1 (SEQ ID NO:478), Framework 2 (SEQ ID NO: 479), CDR2 (SEQ ID NO:480), Framework 3 (SEQ ID NO:481), CDR3 (SEQ ID NO:482), and Framework 4 (SEQ ID NO:483); C6D4: all sequences (SEQ ID NO:727), Framework 1 (SEQ ID NO:745), CDR1 (SEQ ID NO:746), Framework 2 (SEQ ID NO:747), CDR2 (SEQ ID NO: 748), Framework 3 (SEQ ID NO:749), CDR3 (SEQ ID NO:750), and Framework 4 (SEQ ID NO: 751); HuC6D4: all sequences (SEQ ID NO:484), Framework 1 (SEQ ID NO:485), CDR1 (SEQ ID NO:486), Framework 2 (SEQ ID NO:487), CDR2 (SEQ ID NO:488), Framework 3 (SEQ ID NO:489), CDR3 (SEQ ID NO:490), and Framework 4 (SEQ ID NO: 491); C6D4-RGD3: all (SEQ ID NO:492), Framework 1 (SEQ ID NO:493), CDR1 (SEQ ID NO: 494), Framework 2 (SEQ ID NO:495), CDR2 (SEQ ID NO:496), Framework 3 (SEQ ID NO: 497), CDR3 (SEQ ID NO:498), and Framework 4 (SEQ ID NO:499); HuC6D4-RGD3: all (SEQ ID NO:500), Framework 1 (SEQ ID NO:501), CDR1 (SEQ ID NO:502), Framework 2 (SEQ ID NO:503), CDR2 (SEQ ID NO:504), Framework 3 (SEQ ID NO:505), CDR3 (SEQ ID NO: 506), and Framework 4 (SEQ ID NO:507); and Consensus VL: Framework 1 (SEQ ID NO: 565), CDR1 (SEQ ID NO:569), Framework 2 (SEQ ID NO:566), CDR2 (SEQ ID NO: 570), Framework 3 (SEQ ID NO:567), CDR3 (SEQ ID NO:571), and Framework 4 (SEQ ID NO: 568). RDG3 loop (SEQ ID NO:721).

FIG. 52 illustrates heavy chain amino acid sequences for clones used in the construction of the composite antibody F9. Sequences-4F1: all sequences (SEQ ID NO:624), Framework 1 (SEQ ID NO:625), CDR1 (SEQ ID NO:628), Framework 2 (SEQ ID NO:632), CDR2 (SEQ ID NO:634), Framework 3 (SEQ ID NO:637), CDR3 (SEQ ID NO:651), Framework 4 (SEQ ID NO:655), 6B9: all sequences (SEQ ID NO:656), Framework 1 (SEQ ID NO: 625), CDR1 (SEQ ID NO:629), Framework 2 (SEQ ID NO:632), CDR2 (SEQ ID NO:635), Framework 3 (SEQ ID NO:638), CDR3 (SEQ ID NO:652), Framework 4 (SEQ ID NO:655), 6B9.1: all sequences (SEQ ID NO:657), Framework 1 (SEQ ID NO:625), CDR1 (SEQ ID NO: 629), Framework 2 (SEQ ID NO:632), CDR2 (SEQ ID NO:636), Framework 3 (SEQ ID NO: 638), CDR3 (SEQ ID NO:653), Framework 4 (SEQ ID NO:655), A1: all sequences (SEQ ID NO: 658), Framework 1 (SEQ ID NO:626), CDR1 (SEQ ID NO:629), Framework 2 (SEQ ID NO: 633), CDR2 (SEQ ID NO:636), Framework 3 (SEQ ID NO:639), CDR3 (SEQ ID NO:654), Framework 4 (SEQ ID NO:655), A2: all sequences (SEQ ID NO:659), Framework 1 (SEQ ID NO: 625), CDR1 (SEQ ID NO:629), Framework 2 (SEQ ID NO:633), CDR2 (SEQ ID NO:636), Framework 3 (SEQ ID NO:640), CDR3 (SEQ ID NO:654), Framework 4 (SEQ ID NO:655), A8: all sequences (SEQ ID NO:660), Framework 1 (SEQ ID NO:625), CDR1 (SEQ ID NO:629), Framework 2 (SEQ ID NO:633), CDR2 (SEQ ID NO:636), Framework 3 (SEQ ID NO:641), CDR3 (SEQ ID NO:654), Framework 4 (SEQ ID NO:655), A11: all sequences (SEQ ID NO: 661), Framework 1 (SEQ ID NO:625), CDR1 (SEQ ID NO:630), Framework 2 (SEQ ID NO: 633), CDR2 (SEQ ID NO:636), Framework 3 (SEQ ID NO:638), CDR3 (SEQ ID NO:654), Framework 4 (SEQ ID NO:655), B1: all sequences (SEQ ID NO:662), Framework 1 (SEQ ID NO: 625), CDR1 (SEQ ID NO:629), Framework 2 (SEQ ID NO:632), CDR2 (SEQ ID NO:636), Framework 3 (SEQ ID NO:642), CDR3 (SEQ ID NO:654), Framework 4 (SEQ ID NO:655), B3: all sequences (SEQ ID NO:663), Framework 1 (SEQ ID NO:625), CDR1 (SEQ ID NO:629), Framework 2 (SEQ ID NO:633), CDR2 (SEQ ID NO:636), Framework 3 (SEQ ID NO:643), CDR3 (SEQ ID NO:654), Framework 4 (SEQ ID NO:655), C4=F10: all sequences (SEQ ID NO: 664), Framework 1 (SEQ ID NO:625), CDR1 (SEQ ID NO:629), Framework 2 (SEQ ID NO: 633), CDR2 (SEQ ID NO:636), Framework 3 (SEQ ID NO:644), CDR3 (SEQ ID NO:654), Framework 4 (SEQ ID NO:655), C7=D1: all sequences (SEQ ID NO:665), Framework 1 (SEQ ID NO: 625), CDR1 (SEQ ID NO:629), Framework 2 (SEQ ID NO:633), CDR2 (SEQ ID NO: 636), Framework 3 (SEQ ID NO:644), CDR3 (SEQ ID NO:654), Framework 4 (SEQ ID NO: 655), D3=F1: all sequences (SEQ ID NO:666), Framework 1 (SEQ ID NO:625), CDR1 (SEQ ID NO:629), Framework 2 (SEQ ID NO:633), CDR2 (SEQ ID NO:636), Framework 3 (SEQ ID NO:645), CDR3 (SEQ ID NO:654), Framework 4 (SEQ ID NO:655), D10=E5: all sequences (SEQ ID NO:667), Framework 1 (SEQ ID NO:625), CDR1 (SEQ ID NO:629), Framework 2 (SEQ ID NO:633), CDR2 (SEQ ID NO:636), Framework 3 (SEQ ID NO:646), CDR3 (SEQ ID NO:654), Framework 4 (SEQ ID NO:655), E8: all sequences (SEQ ID NO: 667), Framework 1 (SEQ ID NO:625), CDR1 (SEQ ID NO:629), Framework 2 (SEQ ID NO: 633), CDR2 (SEQ ID NO:636), Framework 3 (SEQ ID NO:646), CDR3 (SEQ ID NO:654), Framework 4 (SEQ ID NO:655), F2: all sequences (SEQ ID NO:667), Framework 1 (SEQ ID NO: 625), CDR1 (SEQ ID NO:629), Framework 2 (SEQ ID NO:633), CDR2 (SEQ ID NO:636), Framework 3 (SEQ ID NO:646), CDR3 (SEQ ID NO:654), Framework 4 (SEQ ID NO:655), G4: all sequences (SEQ ID NO:668), Framework 1 (SEQ ID NO:625), CDR1 (SEQ ID NO:629), Framework 2 (SEQ ID NO:633), CDR2 (SEQ ID NO:636), Framework 3 (SEQ ID NO:647), CDR3 (SEQ ID NO:654), Framework 4 (SEQ ID NO:655), C4: all sequences (SEQ ID NO: 669), Framework 1 (SEQ ID NO:625), CDR1 (SEQ ID NO:629), Framework 2 (SEQ ID NO: 633), CDR2 (SEQ ID NO:636), Framework 3 (SEQ ID NO:650), CDR3 (SEQ ID NO:654), Framework 4 (SEQ ID NO:655), D10: all sequences (SEQ ID NO:670), Framework 1 (SEQ ID NO: 625), CDR1 (SEQ ID NO:629), Framework 2 (SEQ ID NO:633), CDR2 (SEQ ID NO:636), Framework 3 (SEQ ID NO:646), CDR3 (SEQ ID NO:654), Framework 4 (SEQ ID NO:655), 4F1A11: all sequences (SEQ ID NO:671), Framework 1 (SEQ ID NO:625), CDR1 (SEQ ID NO: 629), Framework 2 (SEQ ID NO:632), CDR2 (SEQ ID NO:636), Framework 3 (SEQ ID NO: 650), CDR3 (SEQ ID NO:654), Framework 4 (SEQ ID NO:655), 4F1E1: all sequences (SEQ ID NO:672), Framework 1 (SEQ ID NO:625), CDR1 (SEQ ID NO:631), Framework 2 (SEQ ID NO:632), CDR2 (SEQ ID NO:636), Framework 3 (SEQ ID NO:638), CDR3 (SEQ ID NO: 654), Framework 4 (SEQ ID NO:655), 4F1G3: all sequences (SEQ ID NO:673), Framework 1 (SEQ ID NO:625), CDR1 (SEQ ID NO:631), Framework 2 (SEQ ID NO:632), CDR2 (SEQ ID NO:636), Framework 3 (SEQ ID NO:648), CDR3 (SEQ ID NO:654), Framework 4 (SEQ ID NO:655), 4F1E10: all sequences (SEQ ID NO:674), Framework 1 (SEQ ID NO: 627), CDR1 (SEQ ID NO:631), Framework 2 (SEQ ID NO:632), CDR2 (SEQ ID NO: 636), Framework 3 (SEQ ID NO:638), CDR3 (SEQ ID NO:654), Framework 4 (SEQ ID NO: 655), 4F1E9: all sequences (SEQ ID NO:675), Framework 1 (SEQ ID NO:625), CDR1 (SEQ ID NO:629), Framework 2 (SEQ ID NO:632), CDR2 (SEQ ID NO:636), Framework 3 (SEQ ID NO:638), CDR3 (SEQ ID NO:654), Framework 4 (SEQ ID NO:655), 4F1H12: all sequences (SEQ ID NO:676), Framework 1 (SEQ ID NO:625), CDR1 (SEQ ID NO:631), Framework 2 (SEQ ID NO:632), CDR2 (SEQ ID NO:636), Framework 3 (SEQ ID NO:649), CDR3 (SEQ ID NO:654), Framework 4 (SEQ ID NO:655), F9: all sequences (SEQ ID NO: 677), Framework 1 (SEQ ID NO:625), CDR1 (SEQ ID NO:631), Framework 2 (SEQ ID NO: 632), CDR2 (SEQ ID NO:636), Framework 3 (SEQ ID NO:638), CDR3 (SEQ ID NO:654), and Framework 4 (SEQ ID NO:655).

FIG. 53 illustrates light chain amino acid sequences for clones used in the construction of the composite antibody F9. VL Sequences-4F1: all sequences (SEQ ID NO:678), Framework 1 (SEQ ID NO:692), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), 6B9: all sequences (SEQ ID NO:679), Framework 1 (SEQ ID NO: 699), CDR1 (SEQ ID NO: 700), Framework 2 (SEQ ID NO:701), CDR2 (SEQ ID NO: 695), Framework 3 (SEQ ID NO:696), CDR3 (SEQ ID NO:702), Framework 4 (SEQ ID NO: 698), 6B9.1: all sequences (SEQ ID NO:680), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), A1: all sequences (SEQ ID NO:681), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), A2: all sequences (SEQ ID NO: 681), Framework 1 (SEQ ID NO:703), CDR1 (SEQ ID NO:693), Framework 2 (SEQ ID NO: 694), CDR2 (SEQ ID NO:695), Framework 3 (SEQ ID NO:696), CDR3 (SEQ ID NO: 697), Framework 4 (SEQ ID NO:698), A8: all sequences (SEQ ID NO:682), Framework 1 (SEQ ID NO:703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 698), A11: all sequences (SEQ ID NO:683), Framework 1 (SEQ ID NO:704), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), B1: all sequences (SEQ ID NO:684), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), B3: all sequences (SEQ ID NO: 685), Framework 1 (SEQ ID NO:703), CDR1 (SEQ ID NO:693), Framework 2 (SEQ ID NO: 694), CDR2 (SEQ ID NO:695), Framework 3 (SEQ ID NO:696), CDR3 (SEQ ID NO: 697), Framework 4 (SEQ ID NO:698), C4=F10: all sequences (SEQ ID NO:681), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), C7=D1: all sequences (SEQ ID NO:681), Framework 1 (SEQ ID NO: 703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 698), D3=F1: all sequences (SEQ ID NO:681), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), D10=E5: all sequences (SEQ ID NO:686), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), E8: all sequences (SEQ ID NO: 686), Framework 1 (SEQ ID NO:703), CDR1 (SEQ ID NO:693), Framework 2 (SEQ ID NO: 694), CDR2 (SEQ ID NO:695), Framework 3 (SEQ ID NO:755), CDR3 (SEQ ID NO: 697), Framework 4 (SEQ ID NO:698), F2: all sequences (SEQ ID NO:681), Framework 1 (SEQ ID NO:703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 698), G4: all sequences (SEQ ID NO:681), Framework 1 (SEQ ID NO:703), CDR1 (SEQ ID NO: 693), Framework 2 (SEQ ID NO:694), CDR2 (SEQ ID NO:695), Framework 3 (SEQ ID NO: 696), CDR3 (SEQ ID NO:697), Framework 4 (SEQ ID NO:698), C4: all sequences (SEQ ID NO:687), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO: 697), Framework 4 (SEQ ID NO:706), D10: all sequences (SEQ ID NO:688), Framework 1 (SEQ ID NO:699), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 706), 4F1E1=1F1G3=4F1B5=4F1G11=4F1A9=4F1B9=4F1H9=4F1D10=4F1E9=4F1F10=4F1H11=4F1H12: all sequences (SEQ ID NO:689), Framework 1 (SEQ ID NO: 703), CDR1 (SEQ ID NO:693), Framework 2 (SEQ ID NO:694), CDR2 (SEQ ID NO: 695), Framework 3 (SEQ ID NO:696), CDR3 (SEQ ID NO:697), Framework 4 (SEQ ID NO: 698), 4FA11: all sequences (SEQ ID NO:690), Framework 1 (SEQ ID NO:705), CDR1 (SEQ ID NO:693), Framework 2 (SEQ ID NO:694), CDR2 (SEQ ID NO:695), Framework 3 (SEQ ID NO:696), CDR3 (SEQ ID NO:697), Framework 4 (SEQ ID NO:698), F9: all sequences (SEQ ID NO:691), Framework 1 (SEQ ID NO:703), CDR1 (SEQ ID NO:693), Framework 2 (SEQ ID NO:694), CDR2 (SEQ ID NO:695), Framework 3 (SEQ ID NO:696), CDR3 (SEQ ID NO:697), and Framework 4 (SEQ ID NO:706).

FIG. 54A-54D are graphs showing percentage of cells staining positive for various cell surface markers. Mice were injected with Lewis lung carcinoma (LLC) cells and SV5 (isotype control) or C6D4 at a dosage of 7 mg/kg once per week.

### DETAILED DESCRIPTION OF THE INVENTION

### I. Introduction

The inventors have discovered certain antibodies that bind to human integrin αvβ8 and cause at least partial reduction in ligand binding function. Based on that discovery, they have developed detailed structural models to aid in the discovery of antibodies that bind to integrin αvβ8 at particular epitopes that optimally block the ligand binding site of integrin αvβ8. Some of the antibodies identified bind to both the αv-integrin subunit head domain and the β8-integrin subunit head domain to effectively cover the ligand binding site of the integrin αvβ8 without engaging to the ligand binding site itself (i.e. acting as a ligand-mimetic).

Further, the inventors have discovered that blocking ligand binding to integrin αvβ8 is effective in inhibiting cancer (including but not limited to metastatic cancer) and also is effective in treating viral infections. Without intending to limit the scope of the described invention, it is believed that integrin αvβ8 plays a role in blocking regulatory T cells (Tregs) function and/or development and thus that the antibodies described herein stimulate immunity to tumor cells and viruses. Accordingly, antibodies and methods of their use, among other aspects, are provided herein.

The inventors have also identified introduced an “RGDL” sequence (SEQ ID NO:756) into a CDR of the anti-αvβ8 antibody and have shown that such an introduction renders the antibody able to bind αvβ6 while maintaining substantially the same binding activity for αvβ8.

### II. Antibodies

Provided herein are antibodies that bind human (and in some embodiments other mammalian, e.g., such as mouse, guinea pig, pig, and rabbit) integrin αvβ8. In some embodiments, the antibodies are isolated, are chimeric (comprising at least some heterologous amino acid sequence), are labeled or covalently linked to another molecule such a cytotoxic agent or a combination thereof. In some embodiments, the antibodies specifically bind human integrin αvβ8 and block binding of a ligand to human integrin αvβ8. Exemplary ligands can include, for example, TGFβ and LAP. In some embodiments, the antibodies bind in a cation-dependent manner or have enhanced binding in the presence of cations.

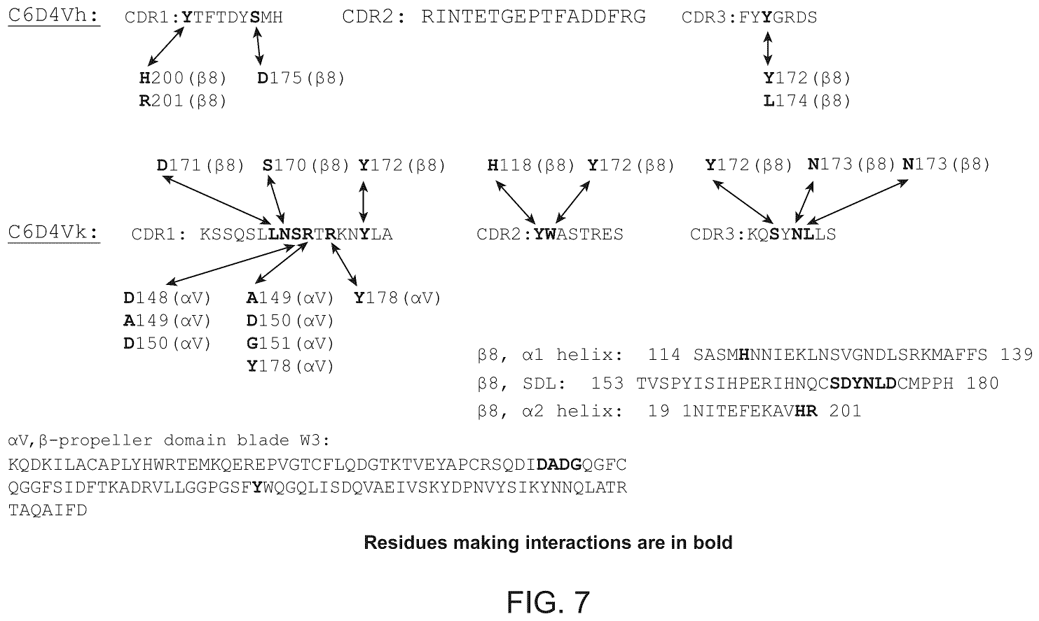

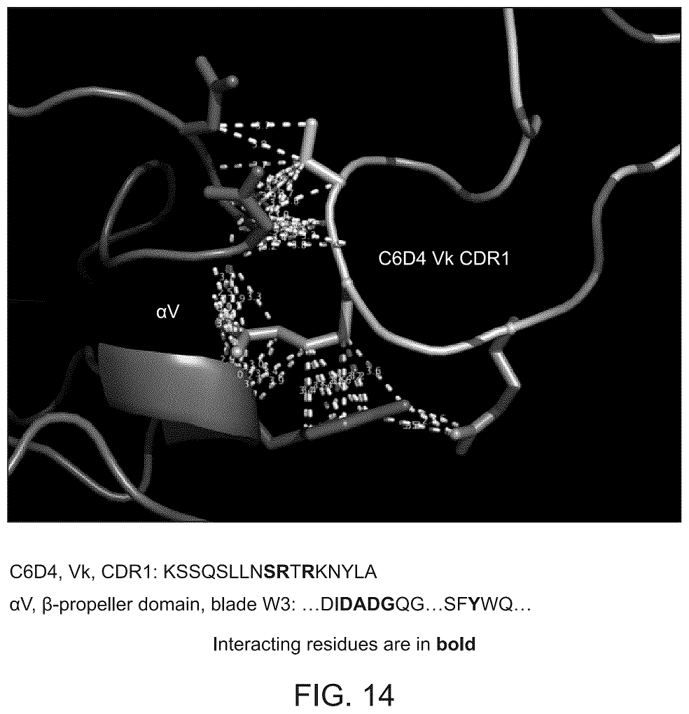

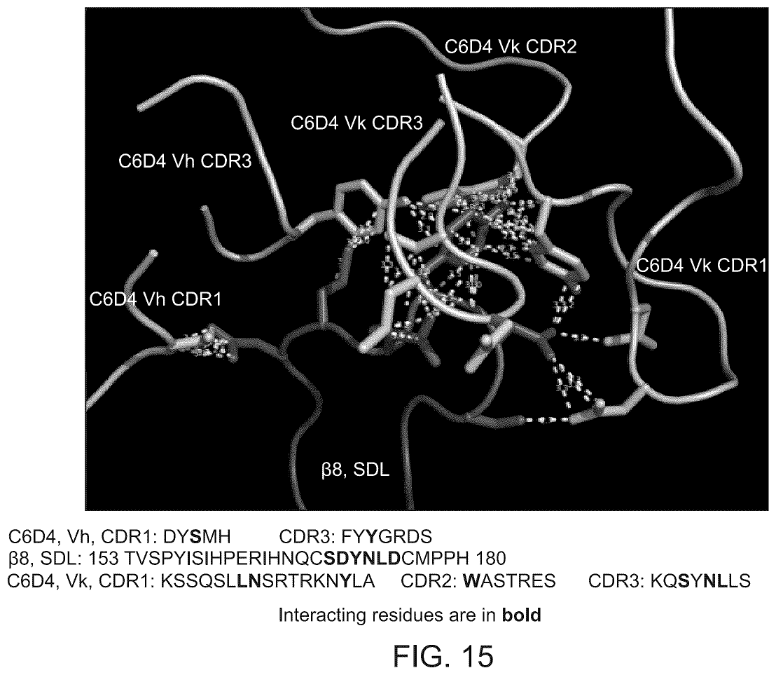

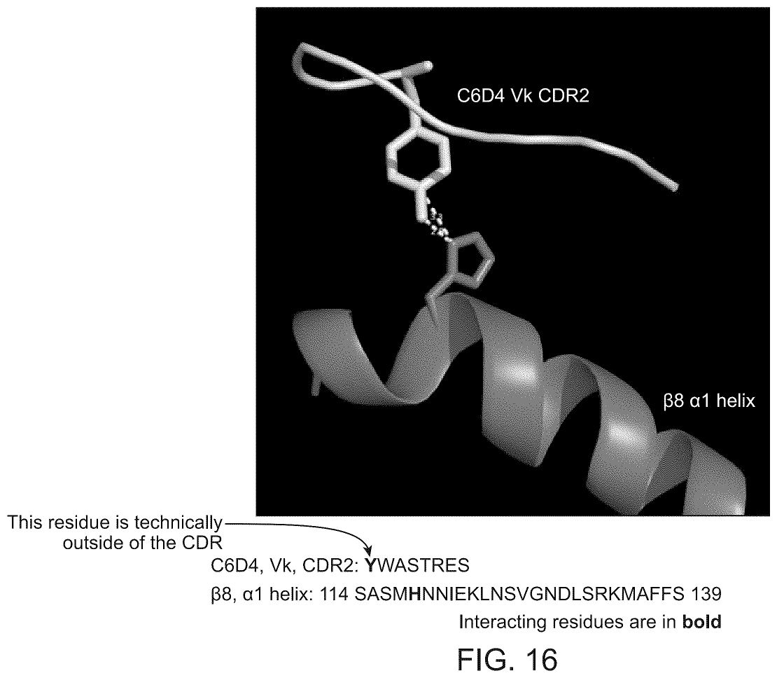

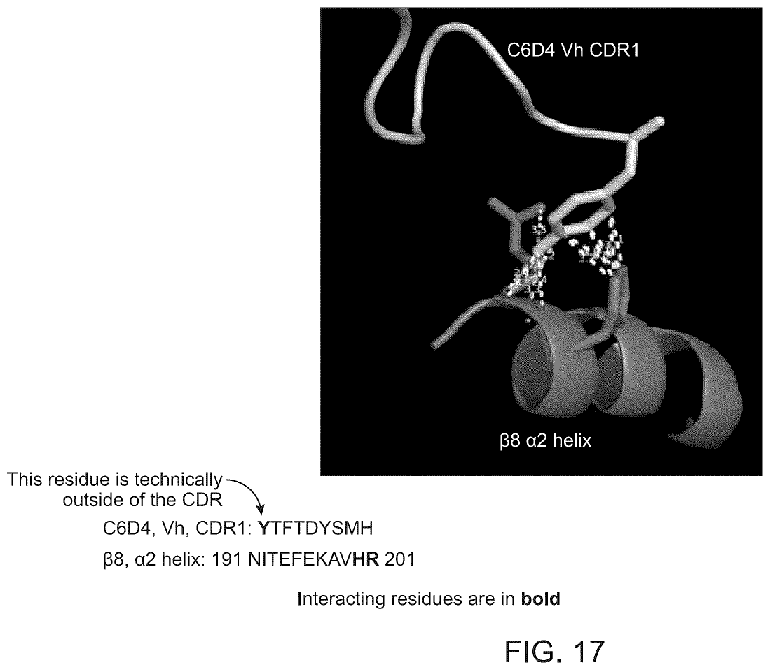

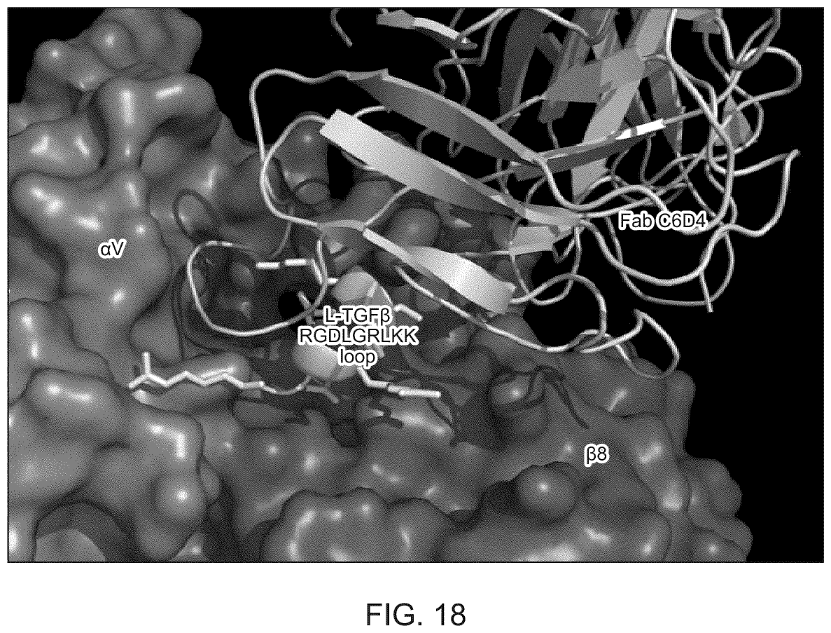

In some embodiments the epitope bound by the antibodies described herein on human integrin αvβ8 comprise amino acids in (1) the specificity determining loop (SDL) of the integrin β8 protein (e.g., TVSPYISIHPERIHNQCSDYNLDCMPPH (SEQ ID NO:620)), (2) in the al (e.g., SASMHNNIEKLNSVGNDLSRKMAFFS (SEQ ID NO:619)) or α2 (e.g., NITEFEKAVHR (SEQ ID NO:621)) helices of the β8 integrin protein, (3) the head of the αv protein (e.g., DADGQ (SEQ ID NO:757); SFYWQ (SEQ ID NO:758); FDDSY (SEQ ID NO: 759)) or other portions of KQDKILACAPLYHWRTEMKQEREPVGTCFLQDGTKTVEYAPCRSQDIDADGQGFCQGG FSIDFTKADRVLLGGPGSFYWQGQLISDQVAEIVSKYDPNVYSIKYNNQLATRTAQAIFD (SEQ ID NO:760) or (4) a combination thereof (e.g., 1 and 2, 2 and 3, 1 and 3, or 1, 2, and 3) as they occur in the native human integrin αvβ8 protein, including for example to all of the listed portions of human integrin αvβ8. In some embodiments, the antibody binds to one or more or all amino acid in the SDL selected from: D175 (e.g., in NLDCM (SEQ ID NO:761)), L174 (e.g., in YNLDC (SEQ ID NO:762)), or S170, D171, or Y172 (e.g., in QCSDYNL (SEQ ID NO: 763)), or combinations thereof, wherein the numbering is based on the human integrin β8 protein (SEQ ID NO:394). See, e.g., FIG. 7. In some embodiments, the antibody binds to the amino acid H118 (e.g., in SMHNN) (SEQ ID NO:764) in the al helix of the β8 integrin protein), wherein the numbering is based on the human integrin β8 protein (SEQ ID NO:394). In some embodiments, the antibody binds to the amino acid H200 or R201 (e.g., in AVHRQ) in the α2 helix of the β8 integrin protein, or combinations thereof, wherein the numbering is based on the human integrin β8 protein (SEQ ID NO:394). In some embodiments, the antibody binds to one or more or all amino acid (underlined) in the head of the αv protein selected from: D148, A149, D150, G151, or Y178 (e.g., in SFYWQ (SEQ ID NO:758)) or combinations thereof, wherein the numbering is based on the human integrin αv protein (SEQ ID NO:393). In some embodiments, the antibody binds to each of the above indicated (underlined) amino acids described in this paragraph. As can be seen from FIGS. 12-18, interaction with the above-described domains of integrin αvβ8 is beneficial.

As noted above, in some embodiments, the antibodies specifically bind human integrin αvβ8 and block binding of a ligand to human integrin αvβ8. The ability of an antibody to block αvβ8 integrin binding of a ligand can be determined by inhibition of binding of a soluble form of αvβ8 or a full-length form of αvβ8 expressed on the surface of cells to immobilized latent-TGF-beta or a portion thereof containing the sequence RGDL See, e.g., Ozawa, A, et al. J Biol Chem. 291 (22): 11551-65 (2016).

In some embodiments, the antibodies comprise one or more CDR (or all of the heavy chain CDRs of a clone, or all of the light chain CDRs of a clone) as follows:

| Heavy | Clone | | | |
|---|---|---|---|---|
| Chains | name | CDR1 VH (SEQ ID:) | CDR2 VH (SEQ ID:) | CDR3 VH (SEQ ID:) |
| Heavy | B2B2 | TFTDYSMH (313) | RINTETGEPTFADDFGG | YYYGRDS (315) |
| | | | (314) | |
| Heavy | B13C4 | TFTDYSMH (316) | WIKTETGEPTYADDFKG | YYYGRDS (318) |
| | | | (317) | |
| Heavy | B13H3 | TFTDYSMH (319) | WIKTETDEPTYADDFKE | YYYGRDS (321) |
| | | | (320) | |
| Heavy | B15B11 | TFTDYSMH (322) | RINTETGEPTFADDFRG | YYYGRDS (324) |
| | | | (323) | |
| Heavy | B13C12 | TFTDYSIH (325) | WIKTETGEPTYADDFNG | YYYGRDS (327) |
| | | | (326) | |
| Heavy | A1 | TFTDYSMH (328) | RINTETGEPTFADDFRG | YYYGRDT (330) |
| | | | (329) | |
| Heavy | C6 | TFTDYSMH (331) | RINTETGEPTFADDFRG | FYYGRDS (333) |
| | | | (332) | |

| Light | Clone | | | |
|---|---|---|---|---|
| Chains | name | CDR1 Vk | CDR2 Vk | CDR3 Vk |
| Light | B2B2 | KASQDINSYLS (334) | RANRLVD (335) | LQYDEFPPLT (336) |
| Light | B13C4 | KSSQSLLNSRTRKNYLA | WASTRES (338) | KQSYNLLT (339) |
| | | (337) | | |
| Light | B13H3 | KSSQSLLNSRIRKNYLA | WASTRES (341) | KQSYNLLT (342) |
| | | (340) | | |
| Light | B15B11.1 | SASSSVSYMH (343) | DTSNLAS (344) | QQWSSNPLT (345) |
| Light | B15B11.2 | SASSSVSYMH (346) | DTSNLAS (347) | QQWSSNPPT (348) |
| Light | B15B11.3 | KSSQSLLNSRTRKNYLA | WASTRES (350) | KQSYNLLT (351) |
| | | (349) | | |
| Light | B13C12.1 | SASSSVSYMH (352) | DTSKLAS (353) | QQWSSNPFT (354) |
| Light | B13C12.2 | SASSSVSYMH (355) | GTSNLAS (356) | QQWSSNPPT (357) |
| Light | B13C12.3 | KSSQSLLHSRTRKNYLA | WASTRES (359) | KQSYNLLT (360) |
| | | (358) | | |
| Light | D4 | KSSQSLLNSRTRKNYLA | WASTRES (362) | KQSYNLLS (363) |
| | | (361) | | |

In some embodiments, the antibodies comprise one or more CDR (or all of the heavy chain CDRs of a clone, or all of the light chain CDRs of a clone) as follows:

| Heavy | Clone | | | |
|---|---|---|---|---|
| Chains | name | CDR1 VH (SEQ ID:) | CDR2 VH (SEQ ID:) | CDR3 VH (SEQ ID:) |
| Heavy | HuC6D4V1 | DYSMH (397) | RINTETGEPTFADDFRG | FYYGRDS (401) |
| | | | (399) | |
| Heavy | HuC6D4A3 | DYSMH (405) | RINTETGEPTFADDFRG | FYYGRDS (409) |
| | | | (407) | |
| Heavy | HuC6D4B7 | DYSMH (413) | RINTETGEPTFADDFRG | FYYGRDT (417) |
| | | | (415) | |
| Heavy | HuC6D4E5 | DYSMH (421) | RINTETGEPTFADDFRG | FYYGRDT (425) |
| | | | (423) | |
| Heavy | HuC6D4 | DYSMH (429) | RINTETGEPTFADDFRG | FYYGRDT (433) |
| | | | (431) | |
| Heavy | C6D4-RGD3 | DYSMH (437) | RINTETGEPTFADDFRG | FYYGRDS (441) |
| | | | (439) | |
| Heavy | HuC6D4-RGD3 | DYSMH (445) | RINTETGEPTFADDFRG | FYYGRDT (449) |
| | | | (447) | |

| Light | Clone | | | |
|---|---|---|---|---|
| Chains | name | CDR1 Vk (SEQ ID:) | CDR2 Vk (SEQ ID:) | CDR3 Vk (SEQ ID: |
| Light | HuC6D4V1 | KSSQSLLNSRTRKNYLA | WASTRES (530) | KQSYNLLS (531) |
| | | (529) | | |
| Light | HuC6D4A3 | KSSQSLLNSRSRKNYLA | WASTRES (533) | KQSYNLIS (534) |
| | | (532) | | |
| Light | HuC6D4B7 | KSSQSLLNSRTRKNYLA | WASTRES (536) | KQSSNLIS (537) |
| | | (535) | | |
| Light | HuC6D4E5 | KSSQSLLNSRSRKNYLA | WASTRES (539) | KQSYNLLS (540) |
| | | (538) | | |
| Light | HuC6D4 | KSSQSLLNSRSRKNYLA | WASTRES (542) | KQSYNLLS (543) |
| | | (541) | | |
| Light | C6D4-RGD3 | KSSQSLLGRGDLGRLKKNALA | WASTRES (545) | KQSYNLLS (546) |
| | | (544) | | |
| Light | HuC6D4-RGD3 | KSSQSLLGRGDLGRLKKNALA | WASTRES (548) | KQSYNLLS (549) |
| | | (547) | | |

In some embodiments, an antibody described herein comprises heavy and light chain CDRs as paired in the following table:

| Combinations | Clone | | | |
|---|---|---|---|---|
| (H + L | name | CDR1 (SEQ ID:) | CDR2 (SEQ ID:) | CDR3 (SEQ ID:) |
| H | B2B2 | TFTDYSMH (313) | RINTETGEPTFADDFGG | YYYGRDS (315) |
| | | | (314) | |
| L | B2B2 | KASQDINSYLS | RANRLVD (335) | LQYDEFPPLT (336) |
| | | (334) | | |
| H | B13H3 | TFTDYSMH (319) | WIKTETDEPTYADDEKE | YYYGRDS (321) |
| | | | (320) | |
| L | B13H3 | KSSQSLLNSRIRKNYLA | WASTRES (341) | KQSYNLLT (342) |
| | | (340) | | |
| H | B13C4 | TFTDYSMH (316) | WIKTETGEPTYADDEKG | YYYGRDS (318 |
| | | | (317( | |
| L | B13C4 | KSSQSLLNSRTRKNYLA | WASTRES (338) | KQSYNLLT (339) |
| | | (337) | | |
| H | B15B11 | TFTDYSMH (322) | RINTETGEPTFADDFRG | YYYGRDS (324) |
| | | | (323) | |
| H | B15B11.1 | SASSSVSYMH (343) | DTSNLAS (344) | QQWSSNPLT (345) |
| H | B15B11 | TFTDYSMH (322) | RINTETGEPTFADDFRG | YYYGRDS (324) |
| | | | (323) | |
| L | B15B11.2 | SASSSVSYMH (346) | DTSNLAS (347) | QQWSSNPPT (348) |
| H | B15B11 | TFTDYSMH (322) | RINTETGEPTFADDFRG | YYYGRDS (324) |
| | | | (323) | |
| L | B15B11.3 | KSSQSLLNSRTRKNYLA | WASTRES (359) | KQSYNLLT (360) |
| | | (358) | | |
| H | B13C12 | TFTDYSIH (325) | WIKTETGEPTYADDFNG | YYYGRDS (327) |
| | | | (326) | |
| L | B13C12.1 | SASSSVSYMH (352) | DTSKLAS (353) | QQWSSNPFT (354) |
| H | B13C12 | TFTDYSIH (325) | WIKTETGEPTYADDFNG | YYYGRDS (327) |
| | | | (326) | |
| L | B13C12.2 | SASSSVSYMH (355) | GTSNLAS (356) | QQWSSNPPT (357) |
| H | B13C12 | TFTDYSIH (325) | WIKTETGEPTYADDFNG | YYYGRDS (327) |
| | | | (326) | |
| L | B13C12.3 | KSSQSLLHSRTRKNYLA | WASTRES (359) | KQSYNLLT (360) |
| | | (358) | | |
| H | RSDLVH-3 | TFTDYSIH (367) | WIKTETGEPTYADDFNG | YYYGRDS (369) |
| | | | (368) | |
| L | RSDLVK-10 | KSSQSLLNSRTRKNYLA | WASTRES (374) | KQSYNLLT (375) |
| | | (373) | | |
| H | RSDLVH-1 | TFTDYSIH (364) | WIKTETGEPTYADDFKG | YYYGRDS (366) |
| | | | (365) | |
| L | RSDLVK-10 | KSSQSLLNSRTRKNYLA | WASTRES (374) | KQSYNLLT (375) |
| | | (373) | | |
| H | RSDLVH-3 | TFTDYSIH (367) | WIKTETGEPTYADDFNG | YYYGRDS (369) |
| | | | (368) | |
| L | RSDLVK-13 | KSSQSLLHSRTRKNYLA | WASTRES (377) | KQSYNLLT (378) |
| | | (376) | | |
| H | RSDLVH-16 | TFTDYSMH (370) | RINTETGEPTFADDFRG | YYYGRDS (372) |
| | | | (371) | |
| L | RSDLVK-10 | KSSQSLLNSRTRKNYLA | WASTRES (374) | KQSYNLLT (375) |
| | | (373) | | |
| H | C6H | TFTDYSMH (766) | RINTETGEPTFADDFRG | FYYGRDS (768) |
| | | | (767) | |
| L | C6K | KSSQSLLNSRTRKNYLA | WASTRES (383) | KQSYNLLT (384) |
| | | (382) | | |
| H | D4H | TFTDYSMH (379) | RINTETGEPTFADDFRG | YYYGRDS (381) |
| | | | (380) | |
| L | D4K | KSSQSLLNSRTRKNYLA | WASTRES (362) | KQSYNLLS (363) |
| | | (361) | | |
| H | C6H | TFTDYSMH (766) | RINTETGEPTFADDFRG | FYYGRDS (768) |
| | | | (767) | |
| L | D4K | KSSQSLLNSRTRKNYLA | WASTRES (362) | KQSYNLLS (363) |
| | | (361) | | |

In some embodiments, an antibody described herein comprises heavy and light chain CDRs as paired in the following table:

| Combinations | Clone | | | |
|---|---|---|---|---|
| (H + L) | name | CDR1 (SEQ ID:) | CDR2 (SEQ ID:) | CDR3 (SEQ ID: |
| H | HuC6D4V1 | DYSMH (397) | RINTETGEPTFADDFRG | FYYGRDS (399) |
| | | | (398) | |
| L | HuC6D4V1 | KSSQSLLNSRTRKNYLA (529) | WASTRES (530) | KQSYNLLS |
| | | | | (531) |
| H | HuC6D4A3 | DYSMH (405) | RINTETGEPTFADDFRG | FYYGRDS |
| | | | (407) | (409) |
| L | HuC6D4A3 | KSSQSLLNSRSRKNYLA (532) | WASTRES (533) | KQSYNLIS |
| | | | | (534) |
| H | HuC6D4B7 | DYSMH (413) | RINTETGEPTFADDFRG | FYYGRDT |
| | | | (415) | (417) |
| L | HuC6D4B7 | KSSQSLLNSRTRKNYLA (535) | WASTRES (536) | KQSSNLIS |
| | | | | (537) |
| H | HuC6D4E5 | DYSMH (421) | RINTETGEPTFADDFRG | FYYGRDT |
| | | | (423) | (425) |
| L | HuC6D4E5 | KSSQSLLNSRSRKNYLA | WASTRES (539) | KQSYNLLS |
| | | (538) | | (540) |
| H | HuC6D4 | DYSMH (429) | RINTETGEPTFADDFRG | FYYGRDT |
| | | | (431) | (433) |
| L | HuC6D4 | KSSQSLLNSRSRKNYLA (541) | WASTRES (542) | KQSYNLLS |
| | | | | (543) |
| H | C6D4-RGD3 | DYSMH (437) | RINTETGEPTFADDFRG | FYYGRDS |
| | | | (439) | (441) |
| L | C6D4-RGD3 | KSSQSLLGRGDLGRLKKNALA | WASTRES | KQSYNLLS |
| | | (544) | (545) | (546) |
| H | HuC6D4-RGD3 | DYSMH (445) | RINTETGEPTFADDFRG | FYYGRDT |
| | | | (447) | (449) |
| L | HuC6D4-RGD3 | KSSQSLLGRGDLGRLKKNALA | WASTRES (548) | KQSYNLLS |
| | | (547) | | (549) |
| H | C6D4 | DYSMH (123) | RINTETGEPTFADDFRG | FYYGRDS |
| | | | (125) | (127) |
| L | C6D4 | KSSQSLLNSRSRKNYLA (291) | WASTRES (293) | KQSYNLLS |
| | | | | (295) |
| H | C6RGD2 | DYSMH (769) | RINTETGEPTFADDFRG | FYYGRDS |
| | | | (770) | (771) |
| L | C6RGD2 | KSSQSLLNSGRGDLGNALA | WASTRES (773) | KQSYNLIS |
| | | (772) | | (774) |
| H | C6RGD3-1 | DYSMH (775) | RINTETGEPTFADDFRG | FYYGRDT |
| | | | (776) | (777) |
| L | C6RGD3-1 | KSSQSLLGRGDLGRLKKQKDHNALA | WASTRES | KQSSNLIS |
| | | (778) | (779) | (780) |
| H | C6RGD3-2 | DYSMH (781) | RINTETGEPTFADDFRG | FYYGRDY |
| | | | (782) | (783) |
| L | C6RGD3-2 | KSSQSLLGRGDLGRLKKQKDNALA | WASTRES (785) | KQSYNLLS |
| | | (784) | | (786) |
| H | C6RGD3-3 | DYSMH (787) | RINTETGEPTFADDFRG | FYYGRDT |
| | | | (788) | (789) |
| L | C6RGD3-3 | KSSQSLLGRGDLGRLKKQKNALA | WASTRES | KQSYNLLS |
| | | (790) | (791) | (792) |
| H | C6RGD3-4 | DYSMH (793) | RINTETGEPTFADDFRG | FYYGRDS |
| | | | (794) | (795) |
| L | C6RGD3-4 | KSSQSLLGRGDLGRLKKONALA | WASTRES | KQSYNLLS |
| | | (796) | (797) | (798) |
| H | C6RGD3 | DYSMH (799) | RINTETGEPTFADDFRG | FYYGRDT (801) |
| | | | (800) | |
| L | C6RGD3 | KSSQSLLGRGDLGRLKKNALA | WASTRES (803) | KQSYNLLS |
| | | (802) | | (804) |
| H | C6RGD3-6 | DYSMH (805) | RINTETGEPTFADDFRG | FYYGRDS (807) |
| | | | (806) | |
| L | C6RGD3-6 | KSSQSLLGRGDLGRLKNALA | WASTRES (809) | KQSYNLLS |
| | | (808) | | (810) |
| H | C6RDG3-7 | DYSMH (811) | RINTETGEPTFADDFRG | FYYGRDS |
| | | | (812) | (813) |
| L | C6RGD3-7 | KSSQSLLGRGDLGRLNALA | WASTRES (815) | KQSYNLIS |
| | | (814) | | (816) |
| H | C6RGD3-8 | DYSMH (817) | RINTETGEPTFADDFRG | FYYGRDT |
| | | | (818) | (819) |
| L | C6RGD3-8 | KSSQSLLGRGDLGRNALA | WASTRES (821) | KQSSNLIS |
| | | (820) | | (822) |
| H | C6RGD1 | DYSMH (823) | RINTETGEPTFADDFRG | FYYGRDY |
| | | | (824) | (825) |
| L | C6RGD1 | KSSQSLLGRGDLGNALA (826) | WASTRES (827) | KQSYNLLS |
| | | | | (828) |
| H | C6RGD3-9 | DYSMH (829) | RINTETGEPTFADDFRG | FYYGRDT |
| | | | (830) | (831) |
| L | C6RGD3-9 | KSSQSLLGRGDLGRLKKQKDHH | WASTRES (833) | KQSYNLLS |
| | | (832) | | (834) |
| H | C6RGD3-10 | DYSMH (835) | RINTETGEPTFADDFRG | FYYGRDS |
| | | | (836) | (837) |
| L | C6RGD3-10 | KSSQSLLGRGDLGRLKKQKDH | WASTRES (839) | KQSYNLLS |
| | | (838) | | (840) |
| H | C6RGD3-11 | DYSMH (841) | RINTETGEPTFADDFRG | FYYGRDT |
| | | | (842) | (843) |
| L | C6RGD3-11 | KSSQSLLGRGDLGRLKKQKD | WASTRES (845) | KQSYNLLS |
| | | (844 | | (846) |
| H | C6RGD3-12 | DYSMH (847) | RINTETGEPTFADDFRG | FYYGRDT |
| | | | (848) | (849) |
| L | C6RGD3-12 | KSSQSLLGRGDLGRLKKQK | WASTRES (851) | KQSSNLIS |
| | | (850) | | (852) |
| H | C6RGD3-13 | DYSMH (853) | RINTETGEPTFADDFRG | FYYGRDY |
| | | | (854) | (855) |
| L | C6RGD3-13 | KSSQSLLGRGDLGRLKKQ (856) | WASTRES (857) | KQSYNLLS |
| | | | | (858) |
| H | C6RGD3-14 | DYSMH (859) | RINTETGEPTFADDFRG | FYYGRDT |
| | | | (860) | (861) |
| L | C6RGD3-14 | KSSQSLLGRGDLGRLKK (862) | WASTRES (863) | KQSYNLLS |
| | | | | (864) |
| H | C6RGD3-15 | DYSMH (865) | RINTETGEPTFADDFRG | FYYGRDS |
| | | | (866) | (867) |
| | C6RGD3-15 | KSSQSLLGRGDLGRLK (868) | WASTRES (869) | KQSYNLLS |
| | | | | (870) |
| H | C6RGD3-16 | DYSMH (871) | RINTETGEPTFADDFRG | FYYGRDT |
| | | | (872) | (873) |
| L | C6RGD3-16 | KSSQSLLGRGDLGRL (874) | WASTRES (875) | KQSYNLLS |
| | | | | (876) |

In some embodiments, an antibody as described herein comprises one, two, three or all four of the framework sequences as provided here:

| Frameworks | Fr1 (SEQ ID NO:) | Fr2 (SEQ ID NO:) |
|---|---|---|
| H | (Q)IQL(L)(Q)SGPELKKPGETVKISCKASGY | WVKQAPGKGLKW(V)A (386) |
| | (385) | M |
| | E M E | |
| | Where (X) can be specified AA | |
| L | (D)IVM(T)QSPSSLAV(S)AGE(K)VT(M)SC | WYQQKPGOSP(R)LLIY (390) |
| | (389) | |
| | E S P N V | K |
| | Where (X) can be specified AA all | |
| | alternatives listed under | |
| Frameworks | Fr3 (SEQ ID NO:) | Fr4 (SEQ ID NO:) |
| H | RFA(V)SLETSASTAYLQINNLKNEDTATYFCAI (387( | WGQGTT(L)TVSS (388 |
| | F | V |
| L | GVPDRFTGSGSGTDFTLTISSVQAEDLAVY(Y)C (391) | FGAGT(K)LE(L)K (392) |
| | E | R I |

In some embodiments, an antibody as described herein comprises one, two, three or all four of the framework sequences as provided here:

| Frameworks | Fr1 (SEQ ID NO:) | Fr2 (SEQ ID NO:) |
|---|---|---|
| H | QIQLVQSG(P)(E)(L)KKPG(E)(T)VKISCKASGYTFT (550) | WV(K)QAPG(K)GL(K)WVA (551) |
| | A K V A S | R Q E |
| | Where (X) can be specified AA | |
| L | (D)IVMTQ(S)P(S)(S)L(A)VS(A)GE(K)VTMSC (554) | WYQQKPGQSPRLLIY (555) |
| | E T A T S P R | A |
| | V I | |

| Frameworks | Fr3 (SEQ ID NO:) |
|---|---|
| H | RF(A)V(S)L(E)TS(A)STAYL(Q)I(N)(N)L(K)(N)(E)DTA(T)YFCAI (552) |
| | T T D T E R S R S D V |
| | S T |
| | Where (X) can be specified AA all |
| | alternatives listed under |
| L | (G)VP(D)RF(T)GSGSGT(D)FTLTISSVQ(A)ED(L)AVYYC (556) |
| | D A S E S E |
| Frameworks | Fr4 (SEQ ID NO:) |
| H | WGQGT(T)LTVSS (553) |
| | A |
| L | FG(A)GT(K)LE(L)KR (557) |
| | Q V I |

In some embodiments, an antibody as described herein comprises one, two, three or all four of the framework sequences as provided here:

| Frameworks | Fr1 (SEQ ID NO:) | Fr2 (SEQ ID NO:) |
|---|---|---|
| H | QIQL(V)QSG(P)(E)(L)KKPG(E)(T)VKISCKASGYTFT (550) | WV(K)QAPG(K)GL(K)W(V)(A) (877) |
| | L A K V A S | R Q E M G |
| | Where (X) can be specified AA | |
| L | (D)IVM(T)Q(S)P(S)(S)L(A)VS(A)GE(K)VTMSC (880) | WYQQKPGQ(S)PRLLIY (881) |
| | E S T A T S P R | A |
| | V I | |

| Frameworks | Fr3 (SEQ ID NO:) |
|---|---|
| H | RF(A)(V)(S)L(E)TS(A)(S)TA(Y)L(Q)I(N)(N)L(K)(N)(E)DTA(T)YFCAI (878) |
| | T F T D T T N E R S R S I V |
| | S I K |
| | T |
| | Where (X) can be specified AA all |
| | alternatives listed under |
| L | (G)VP(D)RF(T)GSGSGT(D)FTLTISSVQ(A)ED(L)AVYYC (882) |
| | D A S E S F |
| | D |
| Frameworks | Fr4 (SEQ ID NO:) |
| H | WGQGT(T)LTVSS (879) |
| | A |
| L | FG(A)GT(K)LE(I)KR (883) |
| | Q V L |

In some embodiments, the antibodies comprise the CDR1, CDR2, and CDR3 heavy chain sequences as provided herein, including but not limited to, e.g.,
SEQ ID NO:3, SEQ ID NO:5, and SEQ ID NO:7;
SEQ ID NO:11, SEQ ID NO:13, and SEQ ID NO:15;
SEQ ID NO:19, SEQ ID NO:21, and SEQ ID NO:23;
SEQ ID NO:27, SEQ ID NO:29, and SEQ ID NO:31;
SEQ ID NO:35, SEQ ID NO:37, and SEQ ID NO:39;
SEQ ID NO:43, SEQ ID NO:45, and SEQ ID NO:47;
SEQ ID NO:51, SEQ ID NO:53, and SEQ ID NO:55;
SEQ ID NO:59, SEQ ID NO:61, and SEQ ID NO:63;
SEQ ID NO:67, SEQ ID NO:69, and SEQ ID NO:71;
SEQ ID NO:75, SEQ ID NO:77, and SEQ ID NO:79;
SEQ ID NO:83, SEQ ID NO:85, and SEQ ID NO:87;
SEQ ID NO:91, SEQ ID NO:93, and SEQ ID NO:95;
SEQ ID NO:99, SEQ ID NO:101, and SEQ ID NO:103;
SEQ ID NO:107, SEQ ID NO:109, and SEQ ID NO:111;
SEQ ID NO:115, SEQ ID NO:117, and SEQ ID NO:119;
SEQ ID NO: 123, SEQ ID NO: 125, and SEQ ID NO: 127;
SEQ ID NO:291, SEQ ID NO:293, and SEQ ID NO:295;
SEQ ID NO:313, SEQ ID NO:314, and SEQ ID NO:315;
SEQ ID NO:316, SEQ ID NO:317, and SEQ ID NO:318;
SEQ ID NO:319, SEQ ID NO:320, and SEQ ID NO:321;
SEQ ID NO:322, SEQ ID NO:323, and SEQ ID NO:324;
SEQ ID NO:325, SEQ ID NO:326, and SEQ ID NO:327;
SEQ ID NO:328, SEQ ID NO:329, and SEQ ID NO:330;
SEQ ID NO:331, SEQ ID NO:332, and SEQ ID NO:333;
SEQ ID NO:367, SEQ ID NO:368, and SEQ ID NO:369;
SEQ ID NO:364, SEQ ID NO:365, and SEQ ID NO:366;
SEQ ID NO:370, SEQ ID NO:371, and SEQ ID NO:372;
SEQ ID NO:379, SEQ ID NO:380, and SEQ ID NO:381;
SEQ ID NO:397, SEQ ID NO:399, and SEQ ID NO:401;
SEQ ID NO:405, SEQ ID NO:407, and SEQ ID NO:409;
SEQ ID NO:413, SEQ ID NO:415, and SEQ ID NO:417;
SEQ ID NO:421, SEQ ID NO:423, and SEQ ID NO:425; or
SEQ ID NO:429, SEQ ID NO:431, and SEQ ID NO:433.

In some embodiments, the antibodies comprise the heavy chain CDR1, CDR2, and CDR3 sequences described above but contain 1, 2, or 3 conservative amino acid substitutions in one, two or more CDR sequences compared to those listed above.

In some embodiments, the antibodies comprise the light chain CDR1, CDR2, and CDR3 sequences as provided herein, including but not limited to, e.g.,
SEQ ID NO:131, SEQ ID NO:133, and SEQ ID NO:135;
SEQ ID NO:139, SEQ ID NO: 141, and SEQ ID NO: 143;
SEQ ID NO:147, SEQ ID NO:149, and SEQ ID NO: 151;
SEQ ID NO: 155, SEQ ID NO:157, and SEQ ID NO:159;
SEQ ID NO:163, SEQ ID NO: 165, and SEQ ID NO:167;
SEQ ID NO:171, SEQ ID NO: 173, and SEQ ID NO: 175;
SEQ ID NO: 179, SEQ ID NO: 181, and SEQ ID NO: 183;
SEQ ID NO:187, SEQ ID NO:189, and SEQ ID NO: 191;
SEQ ID NO: 195, SEQ ID NO: 197, and SEQ ID NO: 199;
SEQ ID NO:203, SEQ ID NO:205, and SEQ ID NO:207;
SEQ ID NO:211, SEQ ID NO:213, and SEQ ID NO:215;
SEQ ID NO:219, SEQ ID NO:221, and SEQ ID NO:223;
SEQ ID NO:227, SEQ ID NO:229, and SEQ ID NO:231;
SEQ ID NO:243, SEQ ID NO:245, and SEQ ID NO:247;
SEQ ID NO:251, SEQ ID NO:253, and SEQ ID NO:255;
SEQ ID NO:259, SEQ ID NO:261, and SEQ ID NO:263;
SEQ ID NO:267, SEQ ID NO:269, and SEQ ID NO:271;
SEQ ID NO:275, SEQ ID NO:277, and SEQ ID NO:279;
SEQ ID NO:283, SEQ ID NO:285, and SEQ ID NO:287;
SEQ ID NO:291, SEQ ID NO:293, and SEQ ID NO:295;
SEQ ID NO:307, SEQ ID NO:309, and SEQ ID NO:311;
SEQ ID NO:334, SEQ ID NO:335, and SEQ ID NO:336;
SEQ ID NO:337, SEQ ID NO:338, and SEQ ID NO:339;
SEQ ID NO:340, SEQ ID NO:341, and SEQ ID NO:342;
SEQ ID NO:343, SEQ ID NO:344, and SEQ ID NO:345;
SEQ ID NO:346, SEQ ID NO:347, and SEQ ID NO:348;
SEQ ID NO:349, SEQ ID NO:350, and SEQ ID NO:351;
SEQ ID NO:352, SEQ ID NO:353, and SEQ ID NO:354;
SEQ ID NO:355, SEQ ID NO:356, and SEQ ID NO:357;
SEQ ID NO:358, SEQ ID NO:359, and SEQ ID NO:360;
SEQ ID NO:361, SEQ ID NO:362, and SEQ ID NO:363;
SEQ ID NO:373, SEQ ID NO:374, and SEQ ID NO:375;
SEQ ID NO:376, SEQ ID NO:377, and SEQ ID NO:378;
SEQ ID NO:382, SEQ ID NO:383, and SEQ ID NO:384;
SEQ ID NO:453, SEQ ID NO:455, and SEQ ID NO:457;
SEQ ID NO:461, SEQ ID NO:463, and SEQ ID NO:465;
SEQ ID NO:469, SEQ ID NO:471, and SEQ ID NO:473;
SEQ ID NO:478, SEQ ID NO:480, and SEQ ID NO:482; or
SEQ ID NO:486, SEQ ID NO:488, and SEQ ID NO:490.

In some embodiments, the antibodies comprise the light chain CDR1, CDR2, and CDR3 sequences described above but contain 1, 2, or 3 conservative amino acid substitutions in one, two or more CDR sequences compared to those listed above. In some embodiments, the light chain CDR1 sequence is 12-18 amino acids long, e.g., 14-17, e.g., 12, 13, 14, 15, 16, 17, or 18 amino acids long.

In some embodiments, the antibodies comprise the heavy and light chain CDR1, CDR2, and CDR3 sequences as provided herein, including but not limited to, e.g.,
heavy chain CDRs SEQ ID NO:313, SEQ ID NO:314, and SEQ ID NO:315; and light chain CDRs SEQ ID NO:334, SEQ ID NO:335, and SEQ ID NO:336; or
heavy chain CDRs SEQ ID NO:319, SEQ ID NO:320, and SEQ ID NO:321; and light chain CDRs SEQ ID NO:340, SEQ ID NO:341, and SEQ ID NO:342; or
heavy chain CDRs SEQ ID NO:316, SEQ ID NO:317, and SEQ ID NO:318; and light chain CDRs SEQ ID NO:337, SEQ ID NO:338, and SEQ ID NO:339; or
heavy chain CDRs SEQ ID NO:322, SEQ ID NO:323, and SEQ ID NO:324; and light chain CDRs SEQ ID NO:343, SEQ ID NO:344, and SEQ ID NO:345; or
heavy chain CDRs SEQ ID NO:322, SEQ ID NO:323, and SEQ ID NO:324; and light chain CDRs SEQ ID NO:346, SEQ ID NO:347, and SEQ ID NO:348; or
heavy chain CDRs SEQ ID NO:322, SEQ ID NO:323, and SEQ ID NO:324; and light chain CDRs SEQ ID NO:349, SEQ ID NO:350, and SEQ ID NO:351; or
heavy chain CDRs SEQ ID NO:325, SEQ ID NO:326, and SEQ ID NO:327; and light chain CDRs SEQ ID NO:352, SEQ ID NO:353, and SEQ ID NO:354; or
heavy chain CDRs SEQ ID NO:325, SEQ ID NO:326, and SEQ ID NO:327; and light chain CDRs SEQ ID NO:355, SEQ ID NO:356, and SEQ ID NO:357; or
heavy chain CDRs SEQ ID NO:325, SEQ ID NO:326, and SEQ ID NO:327; and light chain CDRs SEQ ID NO:358, SEQ ID NO:359, and SEQ ID NO:360; or
heavy chain CDRs SEQ ID NO:367, SEQ ID NO:368, and SEQ ID NO:369; and light chain CDRs SEQ ID NO:373, SEQ ID NO:374, and SEQ ID NO:375; or
heavy chain CDRs SEQ ID NO:364, SEQ ID NO:365, and SEQ ID NO:366; and light chain CDRs SEQ ID NO:373, SEQ ID NO:374, and SEQ ID NO:375; or
heavy chain CDRs SEQ ID NO:367, SEQ ID NO:368, and SEQ ID NO:369; and light chain CDRs SEQ ID NO:376, SEQ ID NO:377, and SEQ ID NO:378; or
heavy chain CDRs SEQ ID NO:370, SEQ ID NO:371, and SEQ ID NO:372; and light chain CDRs SEQ ID NO:373, SEQ ID NO:374, and SEQ ID NO:375; or
heavy chain CDRs SEQ ID NO:331, SEQ ID NO:332, and SEQ ID NO:333; and light chain CDRs SEQ ID NO:382, SEQ ID NO:383, and SEQ ID NO:384; or
heavy chain CDRs SEQ ID NO:379, SEQ ID NO:380, and SEQ ID NO:381; and light chain CDRs SEQ ID NO:361, SEQ ID NO:362, and SEQ ID NO:363; or
heavy chain CDRs SEQ ID NO:331, SEQ ID NO:332, and SEQ ID NO:333; and light chain CDRs SEQ ID NO:361, SEQ ID NO:362, and SEQ ID NO:363; or
heavy chain CDRs SEQ ID NO:508, SEQ ID NO:509, and SEQ ID NO:510; and light chain CDRs SEQ ID NO:529, SEQ ID NO:530, and SEQ ID NO:531; or
heavy chain CDRs SEQ ID NO:511, SEQ ID NO:512, and SEQ ID NO:513; and light chain CDRs SEQ ID NO:532, SEQ ID NO:533, and SEQ ID NO:534; or
heavy chain CDRs SEQ ID NO:514, SEQ ID NO:515, and SEQ ID NO:516; and light chain CDRs SEQ ID NO:535, SEQ ID NO:536, and SEQ ID NO:537; or
heavy chain CDRs SEQ ID NO:517, SEQ ID NO:518, and SEQ ID NO:519; and light chain CDRs SEQ ID NO:538, SEQ ID NO:539, and SEQ ID NO:540; or
heavy chain CDRs SEQ ID NO:520, SEQ ID NO:521, and SEQ ID NO:522; and light chain CDRs SEQ ID NO:541, SEQ ID NO:542, and SEQ ID NO:543; or
heavy chain CDRs SEQ ID NO:523, SEQ ID NO:524, and SEQ ID NO:525; and light chain CDRs SEQ ID NO:544, SEQ ID NO:545, and SEQ ID NO:546; or
heavy chain CDRs SEQ ID NO:526, SEQ ID NO:527, and SEQ ID NO:528; and light chain CDRs SEQ ID NO:547, SEQ ID NO:548, and SEQ ID NO:549.

In some embodiments, the antibodies comprise the heavy and light chain CDR1, CDR2, and CDR3 sequences described above but contain 1, 2, or 3 conservative amino acid substitutions in one, two or more CDR sequences compared to those listed above.

In some embodiments, any antibody described herein can comprise a light chain CDR1 comprising a RGD sequence, e.g., as provided in the following table:

| CDR_{L1} | Vk |
|---|---|
| KSSQSLLNSRSRKNYLA (SEQ ID NO:5 72) | D4 |
| KSSQSLLNSGRGDLGNALA (SEQ ID NO: 574) | RGD2 |
| KSSQSLLGRGDLGRLKKQKDHNALA (SEQ ID NO: 576) | RGD3-1 |
| KSSQSLLGRGDLGRLKKQKDNALA (SEQ ID NO: 577) | RGD3-2 |
| KSSQSLLGRGDLGRLKKQKNALA (SEQ ID NO: 578) | RGD3-3 |
| KSSQSLLGRGDLGRLKKQNALA (SEQ ID NO: 579) | RGD3-4 |
| KSSQSLLGRGDLGRLKKNALA (SEQ ID NO: 575) | RGD3 |
| KSSQSLLGRGDLGRLKNALA (SEQ ID NO: 580) | RGD3-6 |
| KSSQSLLGRGDLGRLNALA (SEQ ID NO: 581) | RGD3-7 |
| KSSQSLLGRGDLGRNALA (SEQ ID NO: 582) | RGD3-8 |
| KSSQSLLGRGDLGNALA (SEQ ID NO: 573) | RGD1 |
| KSSQSLLGRGDLGRLKKQKDHH (SEQ ID NO: 583) | RGD3-9 |
| KSSQSLLGRGDLGRLKKQKDH (SEQ ID NO: 584) | RGD3-10 |
| KSSQSLLGRGDLGRLKKQKD (SEQ ID NO: 585) | RGD3-11 |
| KSSQSLLGRGDLGRLKKQK (SEQ ID NO: 586) | RGD3-12 |
| KSSQSLLGRGDLGRLKKQ (SEQ ID NO: 587) | RGD3-13 |
| KSSQSLLGRGDLGRLKK (SEQ ID NO: 588) | RGD3-14 |
| KSSQSLLGRGDLGRLK (SEQ ID NO: 589) | RGD3-15 |
| KSSQSLLGRGDLGRL (SEQ ID NO: 590) | RGD3-16 |

In some embodiments, any of the antibodies described herein can comprise as CDR1 one of the CDRs selected from SEQ ID NO:572, SEQ ID NO:573, SEQ ID NO:574, SEQ ID NO: 575, SEQ ID NO:576, SEQ ID NO:577, SEQ ID NO:578, SEQ ID NO:579, SEQ ID NO: 580, SEQ ID NO:581, SEQ ID NO:582, SEQ ID NO:583, SEQ ID NO:584, SEQ ID NO: 585, SEQ ID NO:586, SEQ ID NO:587, SEQ ID NO:588, SEQ ID NO:589, and SEQ ID NO: 590.

In some embodiments, the antibody can comprise heavy and light chain CDR1, CDR2, and CDR3 sequences as provided below, including but not limited to, e.g.,
heavy chain CDRs SEQ ID NO:523, SEQ ID NO:524, and SEQ ID NO:525; and light chain CDRs SEQ ID NO:572, SEQ ID NO:545, and SEQ ID NO:546; or
heavy chain CDRs SEQ ID NO:523, SEQ ID NO:524, and SEQ ID NO:525; and light chain CDRs SEQ ID NO:573, SEQ ID NO:545, and SEQ ID NO:546; or
heavy chain CDRs SEQ ID NO:523, SEQ ID NO:524, and SEQ ID NO:525; and light chain CDRs SEQ ID NO:574, SEQ ID NO:545, and SEQ ID NO:546; or
heavy chain CDRs SEQ ID NO:523, SEQ ID NO:524, and SEQ ID NO:525; and light chain CDRs SEQ ID NO:575, SEQ ID NO:545, and SEQ ID NO:546; or
heavy chain CDRs SEQ ID NO:523, SEQ ID NO:524, and SEQ ID NO:525; and light chain CDRs SEQ ID NO:576, SEQ ID NO:545, and SEQ ID NO:546; or
heavy chain CDRs SEQ ID NO:523, SEQ ID NO:524, and SEQ ID NO:525; and light chain CDRs SEQ ID NO:577, SEQ ID NO:545, and SEQ ID NO:546; or
heavy chain CDRs SEQ ID NO:523, SEQ ID NO:524, and SEQ ID NO:525; and light chain CDRs SEQ ID NO:578, SEQ ID NO:545, and SEQ ID NO:546; or
heavy chain CDRs SEQ ID NO:523, SEQ ID NO:524, and SEQ ID NO:525; and light chain CDRs SEQ ID NO:579, SEQ ID NO:545, and SEQ ID NO:546; or
heavy chain CDRs SEQ ID NO:523, SEQ ID NO:524, and SEQ ID NO:525; and light chain CDRs SEQ ID NO:580, SEQ ID NO:545, and SEQ ID NO:546; or
heavy chain CDRs SEQ ID NO:523, SEQ ID NO:524, and SEQ ID NO:525; and light chain CDRs SEQ ID NO:581, SEQ ID NO:545, and SEQ ID NO:546; or
heavy chain CDRs SEQ ID NO:523, SEQ ID NO:524, and SEQ ID NO:525; and light chain CDRs SEQ ID NO:582, SEQ ID NO:545, and SEQ ID NO:546; or
heavy chain CDRs SEQ ID NO:523, SEQ ID NO:524, and SEQ ID NO:525; and light chain CDRs SEQ ID NO:583, SEQ ID NO:545, and SEQ ID NO:546; or
heavy chain CDRs SEQ ID NO:523, SEQ ID NO:524, and SEQ ID NO:525; and light chain CDRs SEQ ID NO:584, SEQ ID NO:545, and SEQ ID NO:546; or
heavy chain CDRs SEQ ID NO:523, SEQ ID NO:524, and SEQ ID NO:525; and light chain CDRs SEQ ID NO:585, SEQ ID NO:545, and SEQ ID NO:546; or
heavy chain CDRs SEQ ID NO:523, SEQ ID NO:524, and SEQ ID NO:525; and light chain CDRs SEQ ID NO:586, SEQ ID NO:545, and SEQ ID NO:546; or
heavy chain CDRs SEQ ID NO:523, SEQ ID NO:524, and SEQ ID NO:525; and light chain CDRs SEQ ID NO:587, SEQ ID NO:545, and SEQ ID NO:546; or
heavy chain CDRs SEQ ID NO:523, SEQ ID NO:524, and SEQ ID NO:525; and light chain CDRs SEQ ID NO:589, SEQ ID NO:545, and SEQ ID NO:546; or
heavy chain CDRs SEQ ID NO:523, SEQ ID NO:524, and SEQ ID NO:525; and light chain CDRs SEQ ID NO:590, SEQ ID NO:545, and SEQ ID NO:546; or
heavy chain CDRs SEQ ID NO:523, SEQ ID NO:524, and SEQ ID NO:525; and light chain CDRs SEQ ID NO:572, SEQ ID NO:545, and SEQ ID NO:534; or
heavy chain CDRs SEQ ID NO:523, SEQ ID NO:524, and SEQ ID NO:525; and light chain CDRs SEQ ID NO:573, SEQ ID NO:545, and SEQ ID NO:534; or
heavy chain CDRs SEQ ID NO:523, SEQ ID NO:524, and SEQ ID NO:525; and light chain CDRs SEQ ID NO:574, SEQ ID NO:545, and SEQ ID NO:534; or
heavy chain CDRs SEQ ID NO:523, SEQ ID NO:524, and SEQ ID NO:525; and light chain CDRs SEQ ID NO:575, SEQ ID NO:545, and SEQ ID NO:534; or
heavy chain CDRs SEQ ID NO:523, SEQ ID NO:524, and SEQ ID NO:525; and light chain CDRs SEQ ID NO:576, SEQ ID NO:545, and SEQ ID NO:534; or
heavy chain CDRs SEQ ID NO:523, SEQ ID NO:524, and SEQ ID NO:525; and light chain CDRs SEQ ID NO:577, SEQ ID NO:545, and SEQ ID NO:534; or
heavy chain CDRs SEQ ID NO:523, SEQ ID NO:524, and SEQ ID NO:525; and light chain CDRs SEQ ID NO:578, SEQ ID NO:545, and SEQ ID NO:534; or
heavy chain CDRs SEQ ID NO:523, SEQ ID NO:524, and SEQ ID NO:525; and light chain CDRs SEQ ID NO:579, SEQ ID NO:545, and SEQ ID NO:534; or
heavy chain CDRs SEQ ID NO:523, SEQ ID NO:524, and SEQ ID NO:525; and light chain CDRs SEQ ID NO:580, SEQ ID NO:545, and SEQ ID NO:534; or
heavy chain CDRs SEQ ID NO:523, SEQ ID NO:524, and SEQ ID NO:525; and light chain CDRs SEQ ID NO:581, SEQ ID NO:545, and SEQ ID NO:534; or
heavy chain CDRs SEQ ID NO:523, SEQ ID NO:524, and SEQ ID NO:525; and light chain CDRs SEQ ID NO:582, SEQ ID NO:545, and SEQ ID NO:534; or
heavy chain CDRs SEQ ID NO:523, SEQ ID NO:524, and SEQ ID NO:525; and light chain CDRs SEQ ID NO:583, SEQ ID NO:545, and SEQ ID NO:534; or
heavy chain CDRs SEQ ID NO:523, SEQ ID NO:524, and SEQ ID NO:525; and light chain CDRs SEQ ID NO:584, SEQ ID NO:545, and SEQ ID NO:534; or
heavy chain CDRs SEQ ID NO:523, SEQ ID NO:524, and SEQ ID NO:525; and light chain CDRs SEQ ID NO:585, SEQ ID NO:545, and SEQ ID NO:534; or
heavy chain CDRs SEQ ID NO:523, SEQ ID NO:524, and SEQ ID NO:525; and light chain CDRs SEQ ID NO:586, SEQ ID NO:545, and SEQ ID NO:534; or
heavy chain CDRs SEQ ID NO:523, SEQ ID NO:524, and SEQ ID NO:525; and light chain CDRs SEQ ID NO:587, SEQ ID NO:545, and SEQ ID NO:534; or
heavy chain CDRs SEQ ID NO:523, SEQ ID NO:524, and SEQ ID NO:525; and light chain CDRs SEQ ID NO:588, SEQ ID NO:545, and SEQ ID NO:534; or
heavy chain CDRs SEQ ID NO:523, SEQ ID NO:524, and SEQ ID NO:525; and light chain CDRs SEQ ID NO:589, SEQ ID NO:545, and SEQ ID NO:534; or
heavy chain CDRs SEQ ID NO:523, SEQ ID NO:524, and SEQ ID NO:525; and light chain CDRs SEQ ID NO:590, SEQ ID NO:545, and SEQ ID NO:534; or
heavy chain CDRs SEQ ID NO:523, SEQ ID NO:524, and SEQ ID NO:525; and light chain CDRs SEQ ID NO:572, SEQ ID NO:545, and SEQ ID NO:537; or
heavy chain CDRs SEQ ID NO:523, SEQ ID NO:524, and SEQ ID NO:525; and light chain CDRs SEQ ID NO:573, SEQ ID NO:545, and SEQ ID NO:537; or
heavy chain CDRs SEQ ID NO:523, SEQ ID NO:524, and SEQ ID NO:525; and light chain CDRs SEQ ID NO:574, SEQ ID NO:545, and SEQ ID NO:537; or
heavy chain CDRs SEQ ID NO:523, SEQ ID NO:524, and SEQ ID NO:525; and light chain CDRs SEQ ID NO:575, SEQ ID NO:545, and SEQ ID NO:537; or
heavy chain CDRs SEQ ID NO:523, SEQ ID NO:524, and SEQ ID NO:525; and light chain CDRs SEQ ID NO:576, SEQ ID NO:545, and SEQ ID NO:537; or
heavy chain CDRs SEQ ID NO:523, SEQ ID NO:524, and SEQ ID NO:525; and light chain CDRs SEQ ID NO:577, SEQ ID NO:545, and SEQ ID NO:537; or
heavy chain CDRs SEQ ID NO:523, SEQ ID NO:524, and SEQ ID NO:525; and light chain CDRs SEQ ID NO:578, SEQ ID NO:545, and SEQ ID NO:537; or
heavy chain CDRs SEQ ID NO:523, SEQ ID NO:524, and SEQ ID NO:525; and light chain CDRs SEQ ID NO:579, SEQ ID NO:545, and SEQ ID NO:537; or
heavy chain CDRs SEQ ID NO:523, SEQ ID NO:524, and SEQ ID NO:525; and light chain CDRs SEQ ID NO:580, SEQ ID NO:545, and SEQ ID NO:537; or
heavy chain CDRs SEQ ID NO:523, SEQ ID NO:524, and SEQ ID NO:525; and light chain CDRs SEQ ID NO:581, SEQ ID NO:545, and SEQ ID NO:537; or
heavy chain CDRs SEQ ID NO:523, SEQ ID NO:524, and SEQ ID NO:525; and light chain CDRs SEQ ID NO:582, SEQ ID NO:545, and SEQ ID NO:537; or
heavy chain CDRs SEQ ID NO:523, SEQ ID NO:524, and SEQ ID NO:525; and light chain CDRs SEQ ID NO:583, SEQ ID NO:545, and SEQ ID NO:537; or
heavy chain CDRs SEQ ID NO:523, SEQ ID NO:524, and SEQ ID NO:525; and light chain CDRs SEQ ID NO:584, SEQ ID NO:545, and SEQ ID NO:537; or
heavy chain CDRs SEQ ID NO:523, SEQ ID NO:524, and SEQ ID NO:525; and light chain CDRs SEQ ID NO:585, SEQ ID NO:545, and SEQ ID NO:537; or
heavy chain CDRs SEQ ID NO:523, SEQ ID NO:524, and SEQ ID NO:525; and light chain CDRs SEQ ID NO:586, SEQ ID NO:545, and SEQ ID NO:537; or
heavy chain CDRs SEQ ID NO:523, SEQ ID NO:524, and SEQ ID NO:525; and light chain CDRs SEQ ID NO:587, SEQ ID NO:545, and SEQ ID NO:537; or
heavy chain CDRs SEQ ID NO:523, SEQ ID NO:524, and SEQ ID NO:525; and light chain CDRs SEQ ID NO:588, SEQ ID NO:545, and SEQ ID NO:537; or
heavy chain CDRs SEQ ID NO:523, SEQ ID NO:524, and SEQ ID NO:525; and light chain CDRs SEQ ID NO:589, SEQ ID NO:545, and SEQ ID NO:537; or
heavy chain CDRs SEQ ID NO:523, SEQ ID NO:524, and SEQ ID NO:525; and light chain CDRs SEQ ID NO:590, SEQ ID NO:545, and SEQ ID NO:537.

In some embodiments, the antibodies comprise the heavy and light chain CDR1, CDR2, and CDR3 sequences described above but contain 1, 2, or 3 conservative amino acid substitutions in one, two or more CDR sequences compared to those listed above.

In some embodiments, any of the antibodies disclosed herein can comprise one of the heavy chain variable regions selected from SEQ ID NO:1, SEQ ID NO:9, SEQ ID NO:17, SEQ ID NO: 25, SEQ ID NO:33, SEQ ID NO:41, SEQ ID NO:49, SEQ ID NO:57, SEQ ID NO:65, SEQ ID NO:73, SEQ ID NO:81, SEQ ID NO:89, SEQ ID NO:97, SEQ ID NO: 105, SEQ ID NO: 113, SEQ ID NO: 121, or SEQ ID NO:297, or SEQ ID NO:395, SEQ ID NO:403, SEQ ID NO: 411, SEQ ID NO:419, SEQ ID NO:427, SEQ ID NO:435, or SEQ ID NO:443.

In some embodiments, any of the antibodies disclosed herein can comprise one of the light chain variable regions selected from SEQ ID NO: 129, SEQ ID NO:137, SEQ ID NO:145, SEQ ID NO:153, SEQ ID NO:161, SEQ ID NO:169, SEQ ID NO:177, SEQ ID NO:185, SEQ ID NO: 193, SEQ ID NO:201, SEQ ID NO:209, SEQ ID NO:217, SEQ ID NO:225, SEQ ID NO: 233, SEQ ID NO:241, SEQ ID NO:249, SEQ ID NO:257, SEQ ID NO:265, SEQ ID NO: 273, SEQ ID NO:281, SEQ ID NO:289, SEQ ID NO:305, or SEQ ID NO:451, SEQ ID NO: 459, SEQ ID NO:467, SEQ ID NO:475, SEQ ID NO:484, SEQ ID NO:492, or SEQ ID NO: 500.

In some embodiments, the antibodies disclosed here can comprise one or more or all of the light chain variable regions (CDRs or framework regions) selected from SEQ ID NO:565, SEQ ID NO:566, SEQ ID NO:567, SEQ ID NO:568, SEQ ID NO:569, SEQ ID NO:570, or SEQ ID NO: 571.

In some embodiments, any of the antibodies disclosed herein can comprise one or more or all of the heavy chain variable regions (CDRs or framework regions) selected from SEQ ID NO: 558, SEQ ID NO:559, SEQ ID NO:560, SEQ ID NO:561, SEQ ID NO:562, SEQ ID NO: 563, or SEQ ID NO:564.

Heavy chain variable regions can be paired with light chain regions as desired, including or not limited to for variable regions comprising the paired CDRs as set forth above.

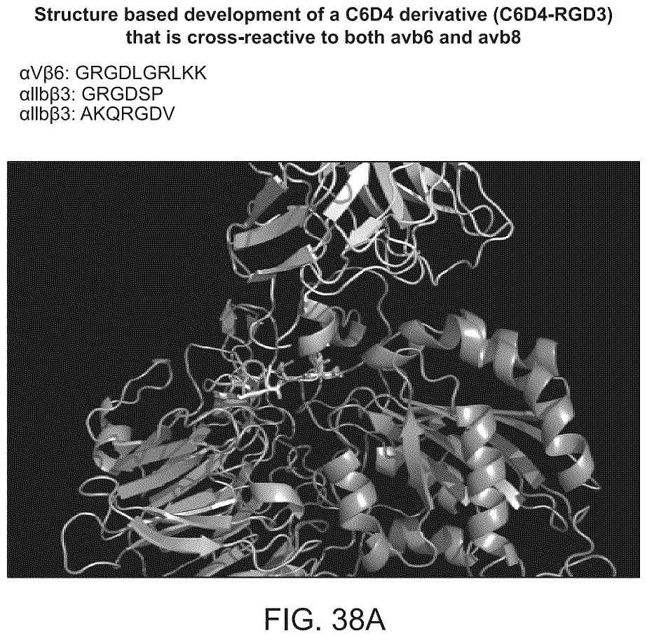

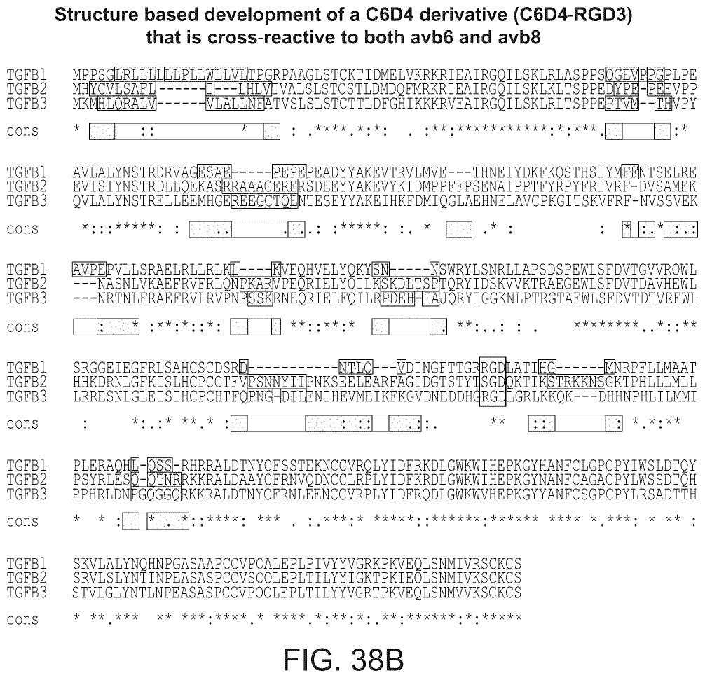

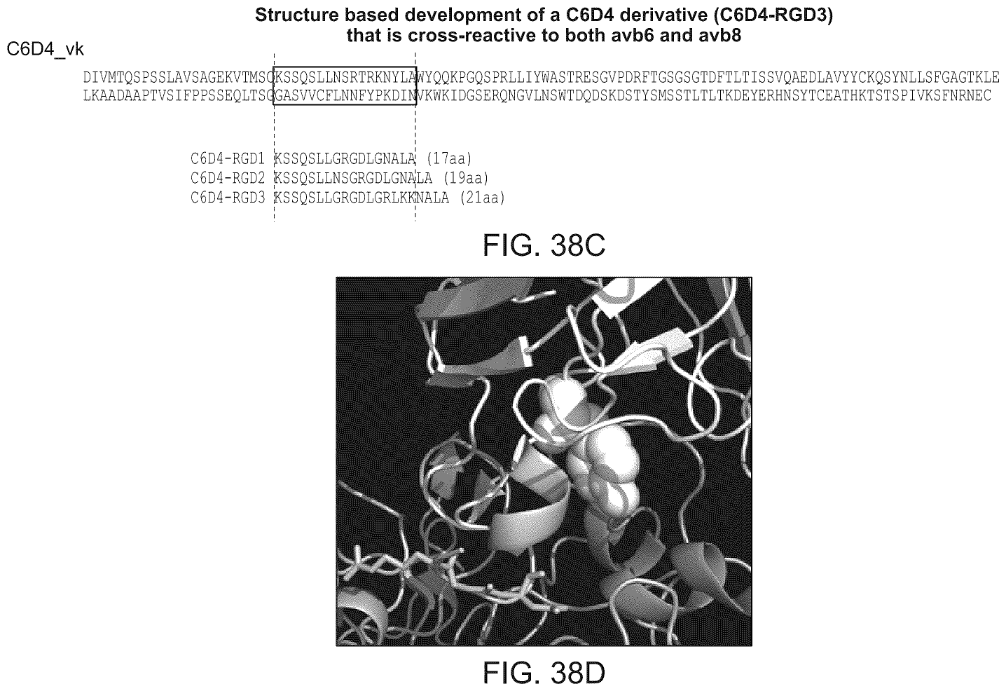

In addition, as noted above, the inventors have discovered that an RGDL sequence (SEQ ID NO:756) can be inserted into a light chain CDR1 sequence in an αvβ8-binding antibody to obtain an antibody that has six CDRs in total and that binds both αvβ8 and αvβ6. The antibodies at least partially block ligand binding function. See, e.g., FIGS. 38A-D. Thus in some embodiments, antibodies are provided that bind to αvβ8 and αvβ6 and comprise an RGDL sequence (SEQ ID NO:756) in the light chain CDR1 sequence. For instance, in some embodiments the light chain CDR1 is between 20-22 amino acids (e.g., 21 amino acids) an optionally comprises KSSQSLLGRGDLGRLKK (SEQ ID NO:765) or a sequence containing 1, 2, or 3 conservative amino acid substitutions.

Additionally, the inventors have discovered that an RGDL sequence (SEQ ID NO:756) can be inserted into a light chain CDR1 sequence in an αvβ8-binding antibody to obtain an antibody that has six CDRs and that binds αvβ8, αvβ6 and αvβ3 (i.e., is tri-specific). See, Example 12.

In some embodiments, any antibody described herein can comprise a light chain CDR1 sequence selected from, but not limited to, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO: 580, SEQ ID NO:581, SEQ ID NO:582, SEQ ID NO:583, SEQ ID NO:584, SEQ ID NO: 585, SEQ ID NO:586, SEQ ID NO:587, SEQ ID NO:588, SEQ ID NO:589, and SEQ ID NO: 590. In some embodiments, any of the light chain CDR1 sequences set forth in this paragraph can be combined with any light chain CDR2, light chain CDR3, heavy chain CDR1, heavy chain CDR2 and heavy chain CDR3, set forth herein.

In some embodiments, antibodies comprising the light chain CDR1 sequences described in the preceding paragraph can contain 1, 2, or 3 conservative amino acid substitutions in the CDR1 sequence compared to those listed above (i.e., SEQ ID NO:572-590).

In some embodiments, the antibodies can comprise the heavy chain CDR1, CDR2, and CDR3 sequences as provided herein, including but not limited to, e.g.,
SEQ ID NO:437, SEQ ID NO:439, and SEQ ID NO:441; or
SEQ ID NO: 445, SEQ ID NO:447, and SEQ ID NO:449.

In some embodiments, the antibodies can comprise the heavy chain CDR1, CDR2, and CDR3 sequences described above but contain 1, 2, or 3 conservative amino acid substitutions in one, two or more CDR sequences compared to those listed above.

In some embodiments, the antibodies can comprise the light chain CDR1, CDR2, and CDR3 sequences as provided herein, including but not limited to, e.g.,
SEQ ID NO:494, SEQ ID NO:496, and SEQ ID NO:498; or
SEQ ID NO:502, SEQ ID NO:504, and SEQ ID NO:506.

In some embodiments, the antibodies can comprise the light chain CDR1, CDR2, and CDR3 sequences described above but contain 1, 2, or 3 conservative amino acid substitutions in one, two or more CDR sequences compared to those listed above.

Heavy chain variable regions can be paired with light chain regions as desired, including or not limited to for variable regions comprising the paired CDRs as set forth above.

For preparation and use of suitable antibodies as described herein, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

An antibody as described herein can also be produced in various formats, including as a Fab, a Fab′, a F(ab′)₂, a scFv, or a dAB. The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In some embodiments, an anti-β8 antibody comprises F(ab′)₂ fragments that specifically bind β8. An antibody of the invention can also include a human constant region. See, e.g., Fundamental Immunology (Paul ed., 4d ed. 1999); Bird, et al., Science 242:423 (1988); and Huston, et al., Proc. Natl. Acad. Sci. USA 85:5879 (1988).

Methods for humanizing or primatizing non-human antibodies are also known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20:227, 2007). The antibody or antibody fragment can also be labeled, or conjugated to a therapeutic agent as described below.

The specificity of antibody binding can be defined in terms of the comparative dissociation constants (Kd) of the antibody for the target (e.g., β8) as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

The desired affinity for an antibody, e.g., high (pM to low nM), medium (low nM to 100 nM), or low (about 100 nM or higher), may differ depending upon whether it is being used as a diagnostic or therapeutic. For example, an antibody with medium affinity may be more successful in localizing to desired tissue as compared to one with a high affinity. Thus, antibodies having different affinities can be used for diagnostic and therapeutic applications.

A targeting moiety will typically bind with a Kd of less than about 1000 nM, e.g., less than 250, 100, 50, 20 or lower nM. In some embodiments, the Kd of the affinity agent is less than 15, 10, 5, or 1 nM. In some embodiments, the Kd is 1-100 nM, 0.1-50 nM, 0.1-10 nM, or 1-20 nM. The value of the dissociation constant (Kd) can be determined by well-known methods, and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The Kd, Kon, and Koff can also be determined using surface plasmon resonance (SPR), e.g., as measured by using a Biacore T100 system or using kinetic exclusion assays (e.g., KinExAR). SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding. Kinetic exclusion assays is the preferred method to determine affinity unless indicated otherwise. This technique is described in, e.g. Darling et al., Assay and Drug Development Technologies Vol. 2, number 6 647-657 (2004).

Binding affinity can also be determined by anchoring a biotinylated interactant to a streptavidin (SA) sensor chip. The other interactant is then contacted with the chip and detected, e.g., as described in Abdessamad et al. (2002) Nuc. Acids Res. 30: e45.

Also provided are polynucleotides encoding the antibodies described herein, or binding fragments thereof comprising at least heavy chain or light chain CDRs or both, e.g., polynucleotides, expression cassettes (e.g., a promoter linked to a coding sequence), or expression vectors encoding heavy or light chain variable regions or segments comprising the complementary determining regions as described herein. In some embodiments, the polynucleotide sequence is optimized for expression, e.g., optimized for mammalian expression or optimized for expression in a particular cell type.

### III. Methods of Treatment

The anti-αvβ8 antibodies described herein (including αvβ8 binding fragments thereof, labeled antibodies, immunoconjugates, pharmaceutical compositions, etc.) as well as antibodies that bind both αvβ8 and αvβ6 as described herein or binding fragments thereof can be used to detect, treat, ameliorate, or prevent chronic obstructive pulmonary disease (COPD) and asthma, inflammatory bowel disease, inflammatory brain autoimmune disease, multiple sclerosis, a demylinating disease (e.g., transverse myelitis, Devic's disease, Guillain-Barré syndrome), neuroinflammation, kidney disease, or glioma, arthritis, fibrotic disorders, such as airway fibrosis, idiopathic pulmonary fibrosis, non-specific interstitial pneumonia, post-infectious lung fibrosis, diffuse alveolar damage, collagen-vascular disease associated lung fibrosis, drug-induced lung fibrosis, silicosis, asbestos-related lung fibrosis, respiratory bronchiolitis, respiratory bronchiolitis interstitial lung disease, desquamative interstitial fibrosis, cryptogenic organizing pneumonia, chronic hypersensitivity pneumonia, drug-related lung or hepatic fibrosis, renal fibrosis, and liver fibrosis (e.g., induced by alcohol, drug use, steatohepatitis, viral infection (e.g., hepatitis B or C), choleostasis, etc., and cancer, including but not limited to adenocarcinoma, squamous carcinoma, breast carcinoma, and cancer growth and metastasis. Accordingly, the antibodies and pharmaceutical compositions described herein can be administered to a human having or suspected of having one of the above-listed diseases in an appropriate dosage to ameliorate or treat one of the disease or at least one symptom thereof.

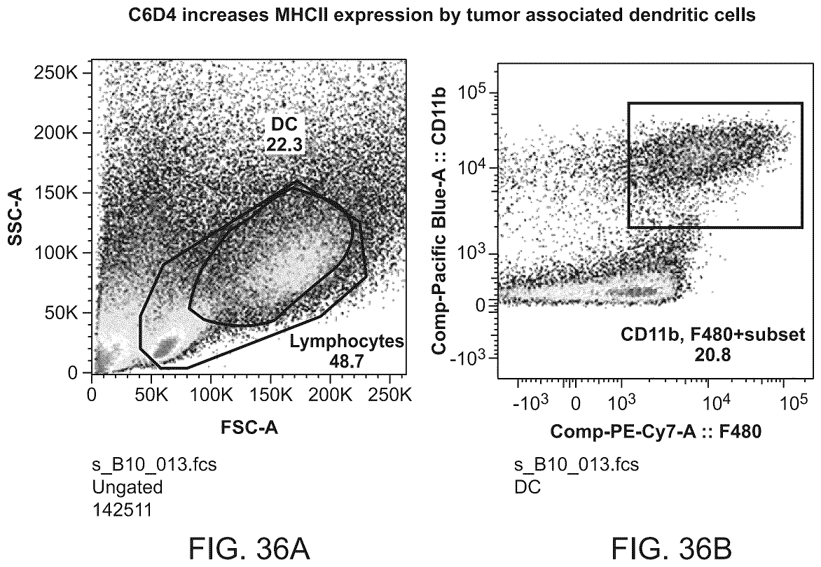

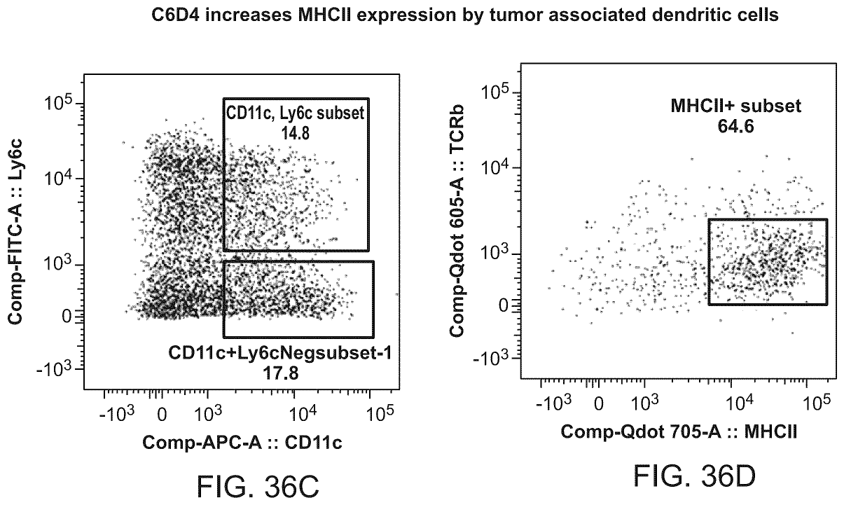

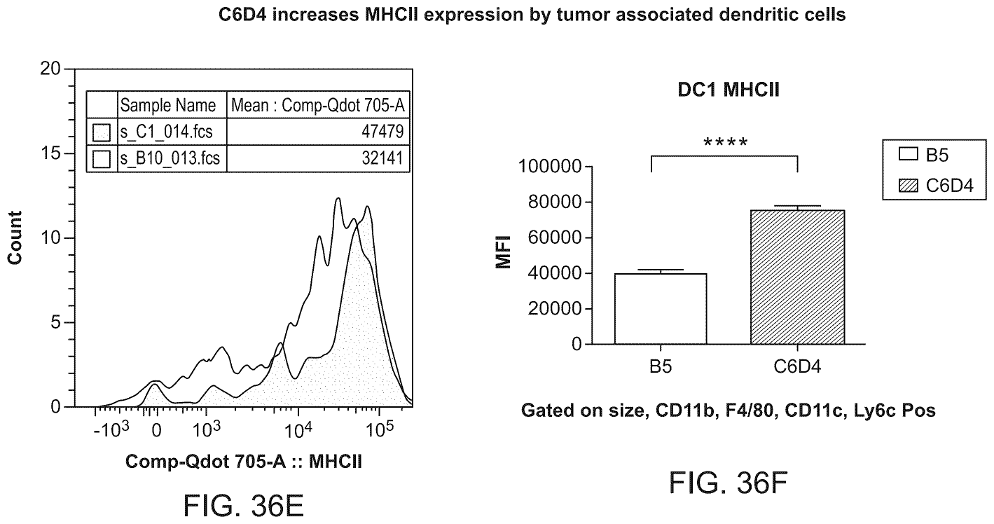

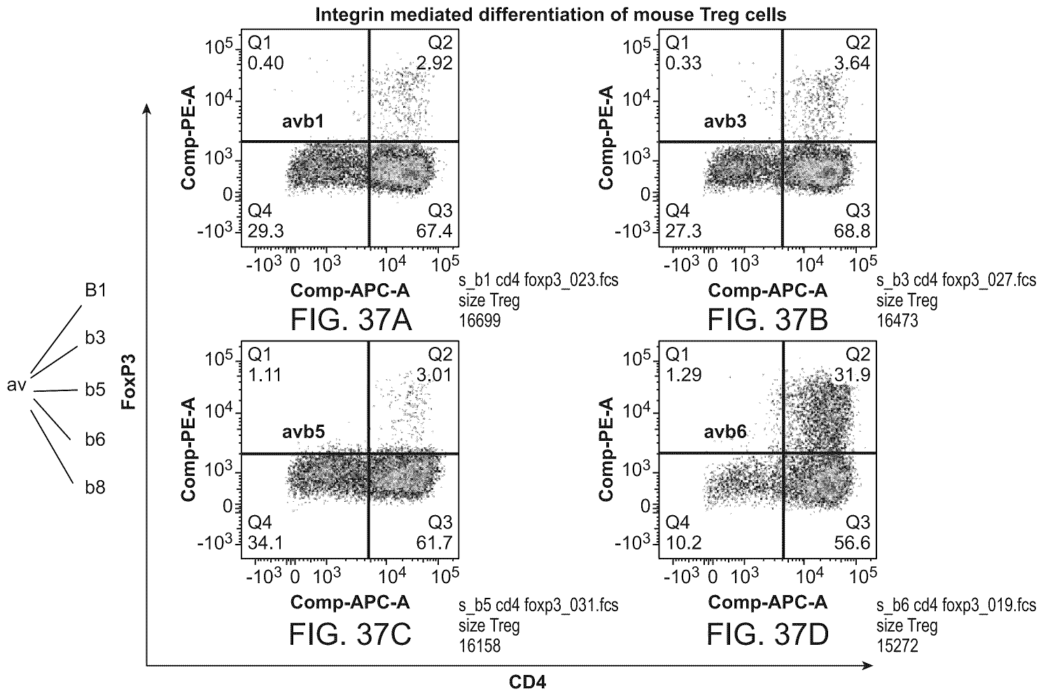

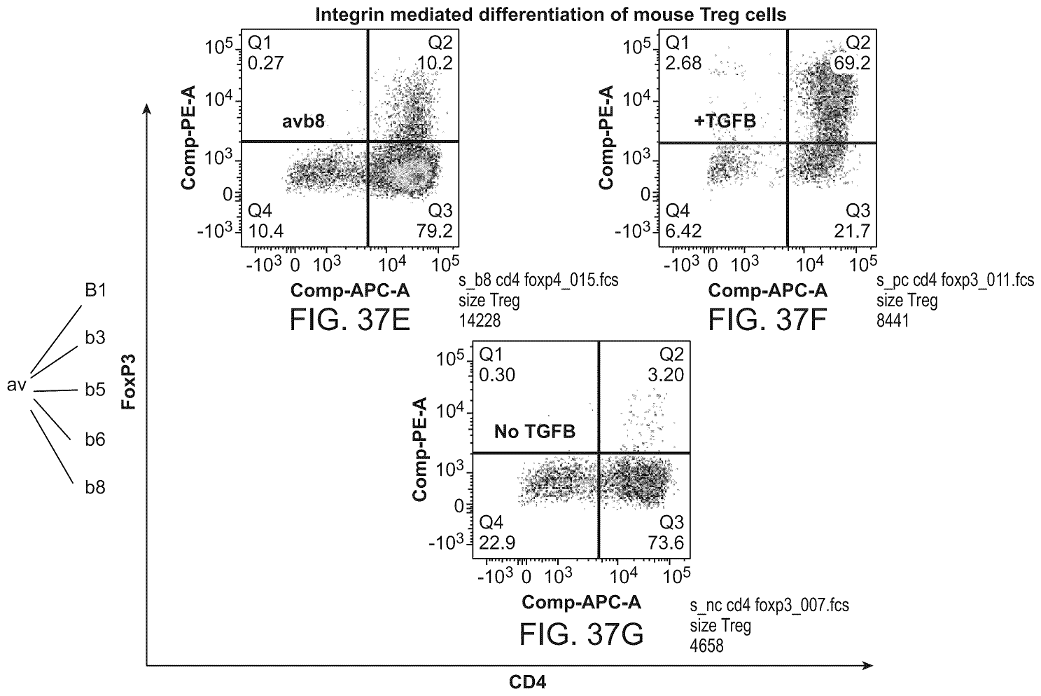

Without intending to limit the scope of the invention, in some embodiments it is believed that antibodies described herein function in part by triggering an increase in MHCII expression in antigen presenting cells. See, e.g., FIG. 36A-F.

Moreover, the anti-αvβ8 antibodies described herein (including αvβ8 binding fragments thereof, labeled antibodies, immunoconjugates, pharmaceutical compositions, etc.) can be used to treat, ameliorate, or prevent viral infections (e.g., by stimulating an immune response). Other antibodies that specifically bind to αvβ8 and that block binding of one or more αvβ8 ligand, for example such as described in WO2011/103490 or WO2015/026004 can also be used to treat, ameliorate, or prevent viral infections. Exemplary viral infections include but are not limited to hepatitis A, B (HBV), and C (HCV), herpes simplex virus (e.g., HSVI, HSVII), HIV, and influenza infections, all of which are enhanced by Treg-mediated immune suppression (Keynan, Y, et al., Clin Infect Dis. 2008 Apr. 1; 46 (7): 1046-52.

Also provided are pharmaceutical compositions comprising the present anti-αvβ8 antibodies or antigen-binding molecules as well as antibodies that bind both αvβ8 and αvβ6 as described herein or binding fragments thereof, either of which can be formulated together with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, intranasal, inhalational, spinal or epidermal administration (e.g., by injection or infusion). Depending on the route of administration, the active compound, i.e., antibody, may be coated in a material to protect the compound from the action of acids and other natural conditions that may inactivate the compound.

The antibodies, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions of the present invention. Applicable methods for formulating the antibodies and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the anti-αvβ8 antibody is employed in the pharmaceutical compositions of the invention. The anti-αvβ8 antibodies are formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

In some embodiments, the pharmacological compositions comprise a mixture of the anti-αvβ8 antibody or antigen binding molecule (e.g. that blocks ligand binding or blocks activation by ligand binding) and a second pharmacological agent. Without intending to limit the invention, it is noted that the inventors have found that thymic stromal lymphopoietin (TSLP) is an inducer of viral clearance in a mouse model of acute and chronic HBV and thus is useful to combine TSLP with an antibody as described herein for anti-viral treatments. Moreover, the inventors have found that OX40 agonists are effective in stimulating an immune response to HBV in combination with an antibody as described herein.

As an alternative to mixing the anti-αvβ8 antibody and second pharmacological agent in a pharmacological composition, the anti-αvβ8 antibody and second pharmacological agent can be separately administered to the human in need thereof within a time frame (e.g., within 3, 2, o 1 day or within 24, 13, 6, or 3 hours of each other).

### IV. Diagnostic Compositions and Applications

Integrin αvβ8 is expressed on fibroblasts, stellate cells, chondrocytes, activated macrophages and subsets of T and B-cells. Integrin αvβ8 is increased in expression in fibroblasts in COPD and pulmonary fibrosis, and can be used as a surrogate marker for increased fibroblast cell mass. Thus the presently disclosed antibodies can be broadly applicable to bioimaging strategies to detect fibroinflammatory processes. The presently described therapeutic and diagnostic antibodies can be applied to: inflammatory bowel disease (IBD), chronic obstructive pulmonary disease (COPD), asthma, arthritis, a hepatic fibroinflammatory disorder, alcohol induced liver injury, non-alcoholic steatohepatitis (NASH), viral hepatitis, and primary biliary cirrhosis (PBC), graft rejection after liver transplantation, autoimmune hepatitis, an autoimmune disorder, lupus erythematosus, scleroderma, dermatomyositis, bullous pemphigoid, pemphigus vulgaris, a pulmonary fibrotic disorder, an inflammatory brain autoimmune disease, multiple sclerosis, a demyelinating disease, neuroinflammation, kidney disease, glomerulonephritis, hepatocellular carcinoma (HCC), adenocarcinoma, squamous carcinoma, glioma, melanoma, prostate, ovarian, uterine and breast carcinoma.

The inventors have found that β8 and PD-L1 expression inversely correlate. Thus, anti-αvβ8 antibodies described herein can be used as a marker for PD-L1 expression and optionally for selecting individuals most likely to benefit from anti-αvβ8 treatment.

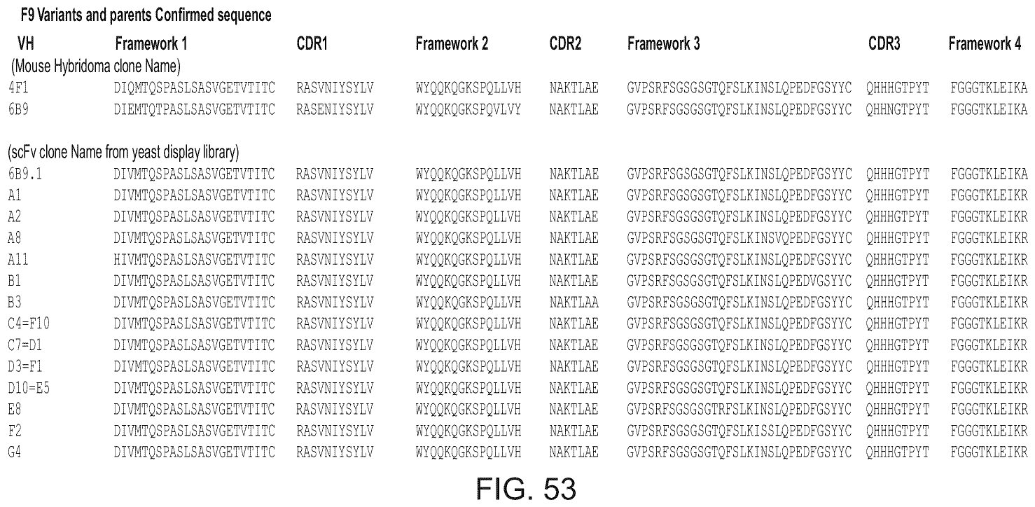

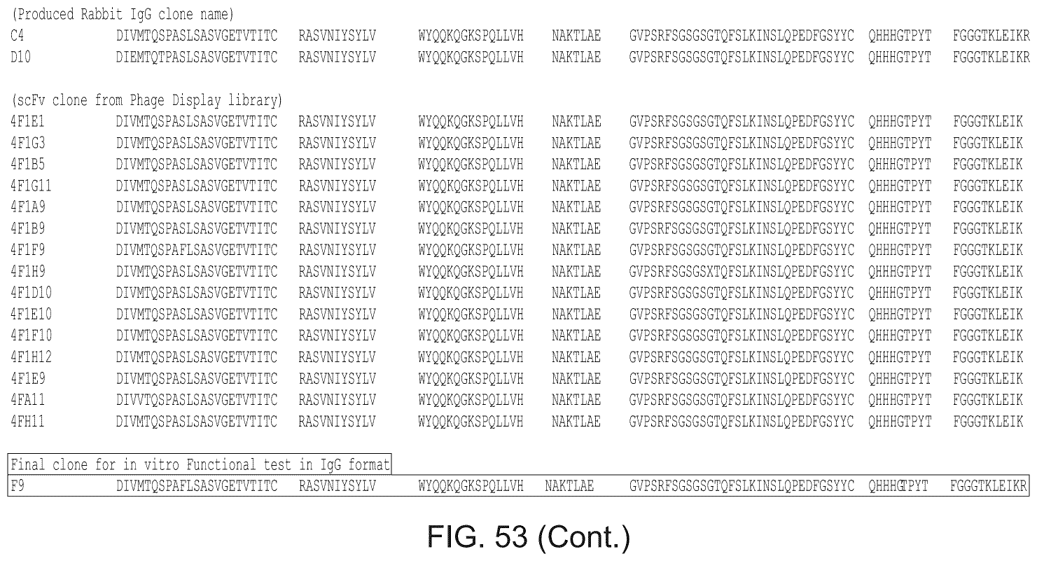

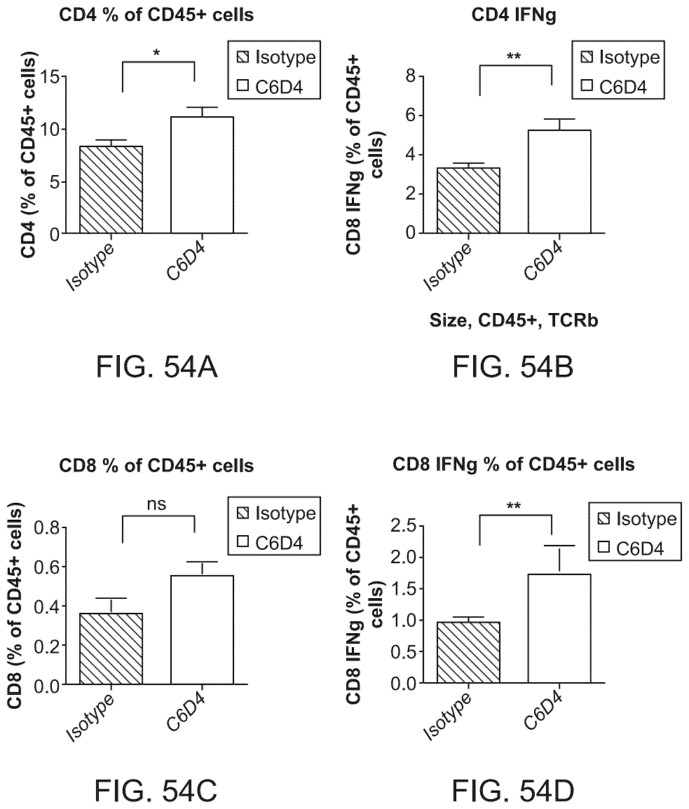

Anti-αvβ8 antibodies described herein (including αvβ8 binding fragments thereof, affinity matured variants, or scFvs) can be used for diagnosis, either in vivo or in vitro (e.g., using a biological sample obtained from an individual). In addition to the above-described antibodies, antibodies having the following CDRs can be used for diagnosis and prognosis: heavy chain CDRs SEQ ID NO:299, SEQ ID NO:301, and SEQ ID NO:303; and light chain CDRs SEQ ID NO:307, SEQ ID NO:309, and SEQ ID NO:311. In some embodiments, the antibodies have a heavy chain variable region comprising SEQ ID NO:297 and a light chain variable region of SEQ ID NO:305. Alternatively, any antibodies having heavy chain CDRs or a heavy chain variable region as set forth in FIG. 53 and light chain CDRs or a light chain variable region from a corresponding sequence as set forth in FIG. 54 can be used. The antibodies are particularly useful in detecting αvβ8 in samples that have been fixed, for example in formalin-fixed samples, including for example formalin-fixed paraffin-embedded (FFPE) biological (e.g., tissue or cell) samples.

When used for detection or diagnosis, the antibody is typically conjugated or otherwise associated with a detectable label. The association can be direct e.g., a covalent bond, or indirect, e.g., using a secondary binding agent, chelator, or linker.

A labeled antibody can be provided to an individual to determine the applicability of an intended therapy. For example, a labeled antibody may be used to detect the integrin β8 density within a diseased area. For therapies intended to target TGFβ or αvβ8 activity (to reduce TGFβ or αvβ8 activity), the density of β8 is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.). Such in vivo methods can be carried out using any of the presently disclosed antibodies.

Any of the presently disclosed antibodies can also be used for in vitro diagnostic or monitoring methods, e.g., using cells or tissue from a patient sample. In some embodiments, labeled F9 (or a β8 binding fragment or affinity-matured variant) is used, as it can bind fixed cells as well as non-fixed cells.

In some embodiments, the diagnostic antibody is a single-chain variable fragment (scFv). Intact antibodies (e.g., IgG) can be used for radioimmunotherapy or targeted delivery of therapeutic agents because they exhibit high uptake and retention. In some cases, the persistence in circulation of intact mAbs can result in high background (Olafsen et al. (2012) Tumour Biol. 33:669-77; Cai et al. (2007) J Nucl Med. 48:304-10). ScFvs, typically with a molecular mass of ˜ 25 kD, are rapidly excreted by the kidneys, but are monovalent and can have lower affinity. The issues of monovalency can be overcome with advanced antibody engineering (as shown herein), where affinities can be improved to the low nM to pM range. Such antibodies have short enough half-lives to be useful as imaging agents and have suitable binding characteristics for tissue targeting (Cortez-Retamozo et al. (2004) Cancer Res. 64:2853-7). As shown herein, we have created a very high affinity scFV antibody derivatives of 4F1, 6B9, called F9, that can be converted to humanized scFV platforms. These improved antibodies are not function blocking, and thus can be used in combination with a therapeutic agent that targets β8.

A diagnostic agent comprising an antibody described herein can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). The terms “detectable agent,” “detectable moiety,” “label,” “imaging agent,” and like terms are used synonymously herein. A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. PET is particularly sensitive and quantitative, and thus valuable for characterizing fibrotic processes in vivo (Olafsen et al. (2012) Tumour Biol. 33:669-77; Cai et al. (2007) J Nucl Med. 48:304-10). This is useful beyond a companion diagnostic and would be generally useful to diagnose, clinically stage and follow fibrotic patients during any treatment regimen.

A radioisotope can be incorporated into the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹ In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹ In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1 (1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl) methyl] benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8, 11-tetraazacyclotetradecane-1,4,8,11-tetraacetic acid (TETA), 1,4,7,10-tetraazacyclododecane-1,4,7,10-tetraacetic acid (DOTA), N1,N1-bis(pyridin-2-ylmethyl) ethane-1,2-diamine (ENPy2) and derivatives thereof.

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

### EXAMPLES

The following examples are offered to illustrate, but not to limit the claimed invention.

### Example 1. Construction of Composite Antibody C6D4

ITGB-8 knockout mice were immunized with recombinant Human Integrin alpha V beta 8 (αvβ8) protein. Approximately 5000 hybridomas were generated and screened for their ability to bind to αvβ8 in an enzyme-linked immunosorbent assay (ELISA). Results were confirmed by cell staining, and function blocking was determined with the use of a transforming growth factor-beta (TGF-β) bioassay. Blocking antibodies were screened against a recombinant form of αvβ8 engineered to lack the specificity determining loop (SDL) of the β8 head domain. Antibodies not binding this engineered αvβ8 were then selected.

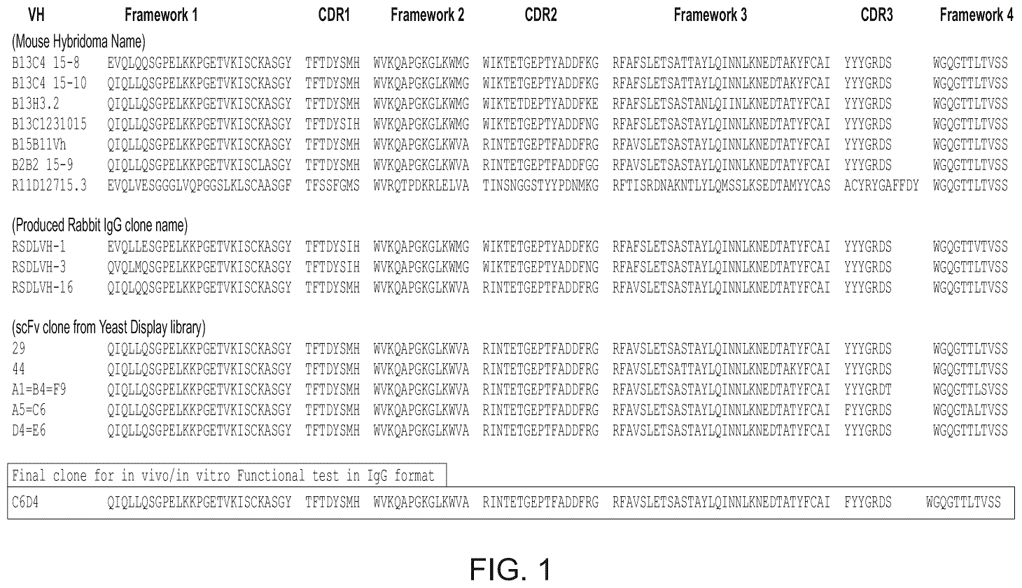

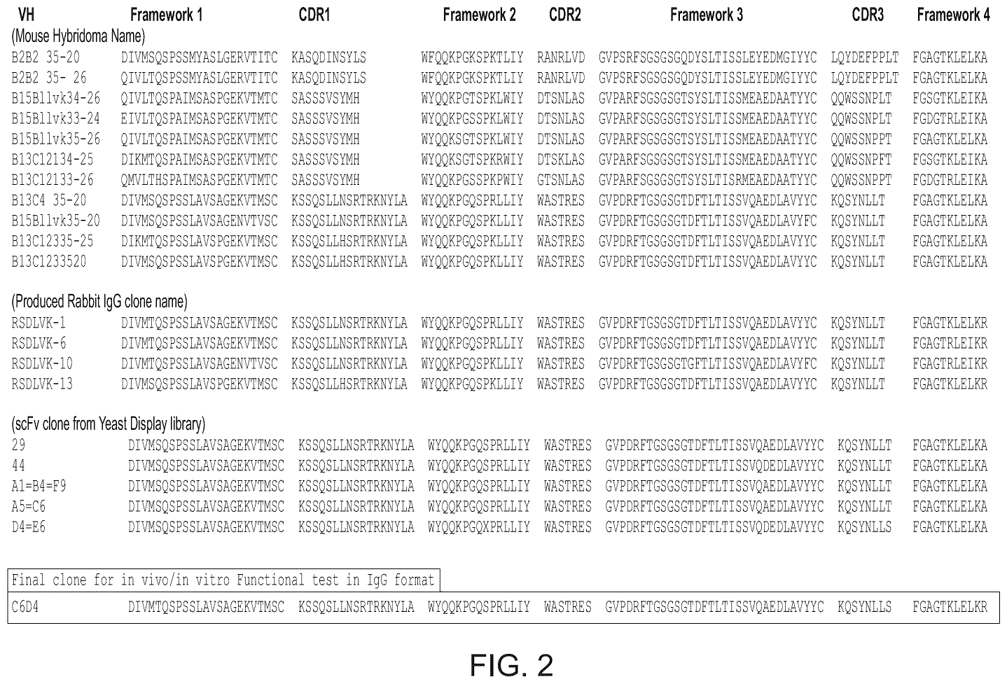

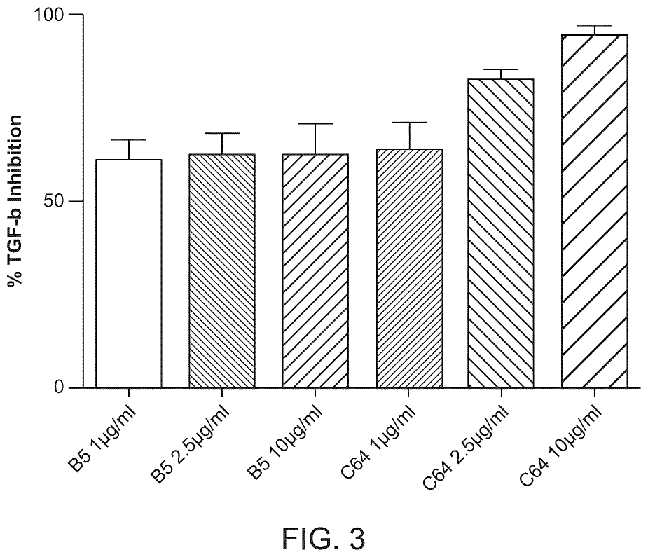

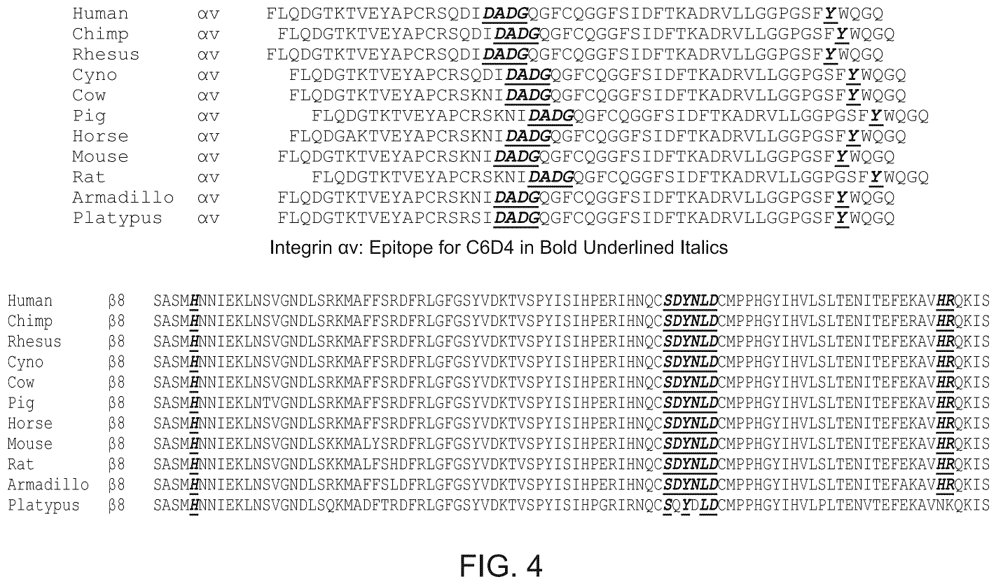

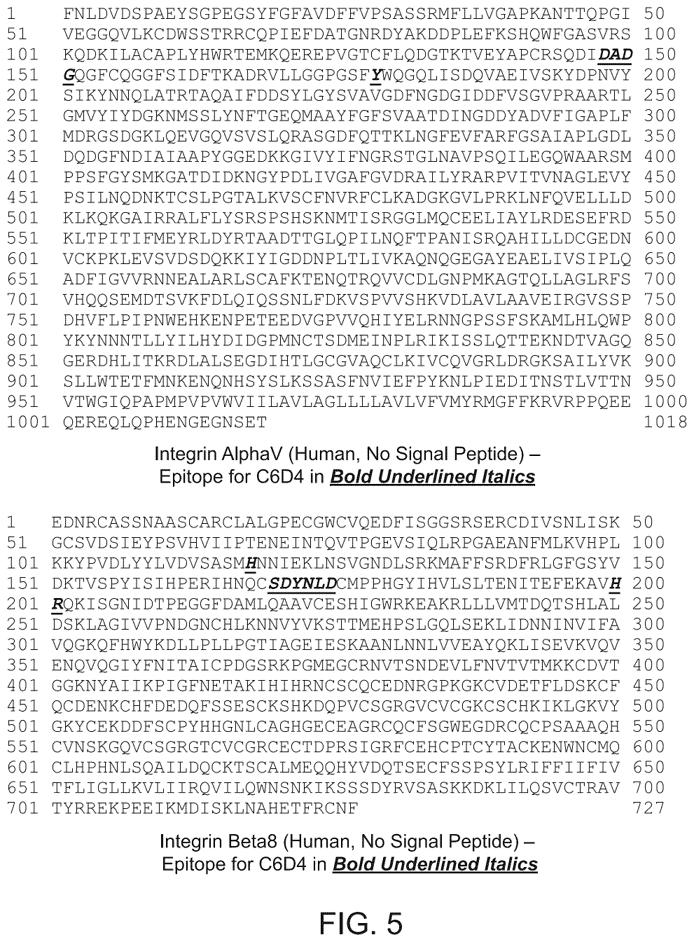

Variable (V) genes from eight hybridomas were next isolated, sequenced, and found to comprise seven V_{H} and eleven V_{K} genes that were unique but related. FIG. 1 and FIG. 2 provide sequence information for the products of these V_{H} and V_{K} genes. Sequence information is using the Kabat numbering scheme. Each V gene was amplified under mutagenic conditions, and a single-chain variable fragment (scFV) library was constructed by mixing the amplified cDNA and using splice overlap. The library served as an amplification template using primers designed to complement rabbit IgG expressing dual V_{H} and V_{L} vectors. Eleven distinct V_{H} genes and sixteen distinct V_{K} genes were identified after sequencing >100 random clones and transfected in 165 different combinations into 293 cells. The eight pairs that produced the best binders were determined by cell staining and FACS analysis, and by measuring binding affinity for CHO cells expressing αvβ8. The eight pairs each comprised a Vu domain selected from RSDLVH-1, RSDLVH-3, and RSDLVH-16; and a V_{K} domain selected from RSDLVK-1, RSDLVK-6, RSDLVK-10, and RSDLVK-13; the sequences of which are shown in FIG. 1 and FIG. 2.

These eight rabbit IgG V_{H}/V_{K} pairs were then used to create a new mutagenic scFV yeast display library that was inserted into a yeast expression library vector. Two high-affinity binders from this selection and affinity maturation step were identified and designated clone 29 and clone 44. Random mutation mutagenic libraries were next made from genes of clones 29 and 44, and from these libraries the higher-affinity binding clones C6 and D4 were selected and determined (FIG. 1 and FIG. 2). Mutations in the complementarity-determining regions (CDRs) of C6 V_{H} and D4 V_{K} were identified, and the two chains were combined to create the composite antibody C6D4 (FIG. 1 and FIG. 2).

### Example 2. Characterization of C6D4 Binding Affinity

A Kinetic Exclusion Assay (KINEXA®) was used to measure the binding affinity of C6D4. The affinity as a murine IgG2a was measured as 832 pM. As a recombinant IgG, C6D4 was found to result in substantially complete blockage of αvβ8-mediated TGF-β activation. This result implies blockage that is better than with B5, an allosteric inhibitor of αvβ8-mediated TGF-β activation. (Minagawa, et al, Sci Trans Med. 2014 Jun. 18; 6 (241): 241ra79)

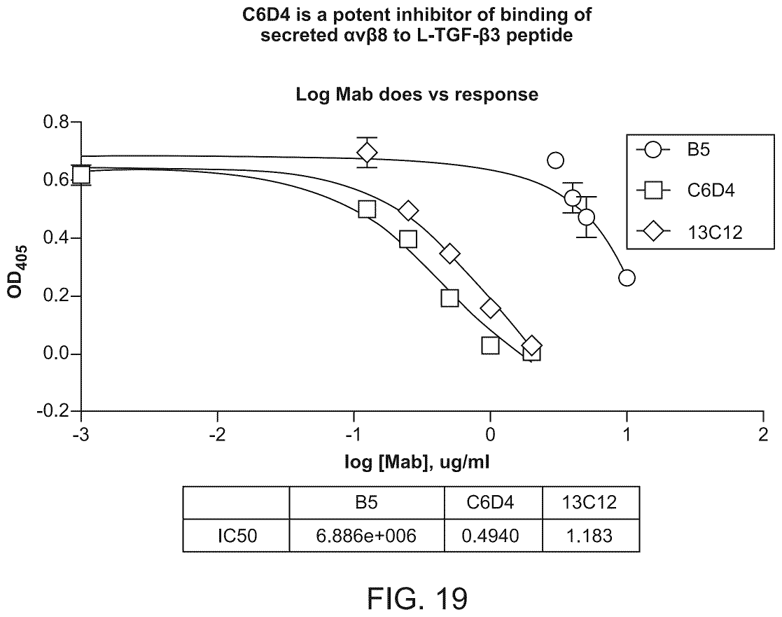

C6D4 was also shown to block adhesion of cells to immobilized latent TGF-β. A peptide with the sequence DDHGRGDLGRLK (SEQ ID NO:713), which corresponds to aa 257-268 of human TGF-β3 (NP_003230) was synthesized on an 8 lysine core (Multiple antigen presenting peptide, BioSyn) and used at 1 μg/ml to coat a 96 well ELISA plate. A truncated secreted form of αvβ8 which was fused in frame to alkaline phosphatase (Gline S E, et al. J Biol Chem. 2004 Dec. 24; 279 (52): 54567-72) was added with Mab at the indicated concentrations. The results (FIG. 19) show the superiority of C6D4 over B5 and the improvement of C6D4 compared to Clone 13C12. The table gives the IC50 values in μg/ml.

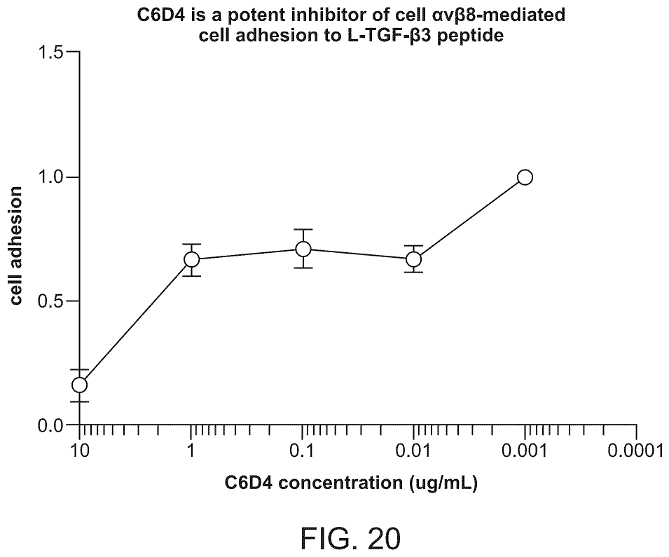

Further, a peptide with the sequence DDHGRGDLGRLK (SEQ ID NO:713), which corresponds to aa 257-268 of human TGF-β3 (NP_003230) was synthesized on an 8 lysine core (Multiple antigen presenting peptide, BioSyn) and used at 0.51 μg/ml to coat a 96 well ELISA plate. CHO lec cells stably transfected with αvβ8 were allowed to bind to the peptide coated wells for 30 min at RT. Unbound cells were washed off with PBS. The Mab C6D4 was added at the indicated concentrations. Results were presented as stained cells detected after staining with crystal violet (OD590). The results (FIG. 20) show that C6D4 almost completely blocks cell adhesion to the peptide.

### Example 3. Characterization of C6D4 Binding Structure

The current understanding of integrin structure is faced with the hurdle of having to reconcile two polar opposite views of integrin conformation. One camp proposes that integrins are always bent. The other believes that integrins must undergo a significant conformational “switchblade” change from a bent conformation to an extended conformation upon activation, opening the “headpieces” of the integrins to be fully functional. This model of integrin extension proposes one of the largest tertiary and quaternary structural rearrangements in biology.

Proof of such conformational extremes has been hampered by compromises and shortcomings associated with techniques routinely used in structural biology. Traditional crystallography produces crystal structures with atomic resolution but is reliant on the conformations and conditions under which crystals can be formed. In the case of integrins, only compact, closed conformations have been seen by crystallography. Alternatively, size exclusion chromatography (SEC) of integrins under activating conditions have demonstrated large shifts in size consistent with integrin extension. Such changes in conformation have been directly visualized using negative stain electron microscopy (EM) studies but at low resolution. Thus, the atomic details of the integrin ligand binding and the integrin activation mechanism remains unresolved.

Single-particle cryo-electron microscopy (cryoEM) can be used to determine the structure of biological macromolecules without crystals, thus offering an alternative that circumvents the obstacles of crystalizing integrins in the extended form. Recent hardware and software developments demonstrate that single-particle cryoEM has the power to provide atomic-level structural understanding of molecules that are traditionally challenging to study. Because single-particle cryoEM does not require the formation of crystals, and allows examination in the native functional conformations unaffected by crystal packing forces or high-salt crystallization buffers, this method is uniquely suited to understanding structures of proteins or integrin-ligand or integrin-Fab complexes that are difficult to crystallize. Here, we have used single particle cryoEM to address some of the biggest mysteries in structural biology, the structural mechanisms of integrin activation and conversely the mechanism of action of integrin inhibitors.

Previously published crystal structures of the latent TGF-β arginine-glycyine-aspartic acid (RGD) peptide of αvβ6 show the positioning of the TGF-β RGD in the αvβ6 binding pocket, as well as the positioning of the R of the TGF-β RGD proximate to the αv head. Cryo-electron microscopy of the new composite antibody C6D4 structure have now produced a ˜4-5-angstrom-resolution structure of the C6D4 Fab binding to αvβ8. To generate the structures of αvβ8 in complex with C6D4, purified recombinant αvβ8 and C6D4 Fab complexes were isolated by size exclusion chromatography and then plunge frozen on grids in liquid nitrogen. Images of ˜61,000 individual particle images captured by electron microscopy were selected to produce a 3D electron density map which was used to build model of αvβ8 in complex with C6D4 Fab using existing Protein Data Bank (PDB) entries for the integrin αvβ3, αIIbβ3, and Fabs with similar CDRs.

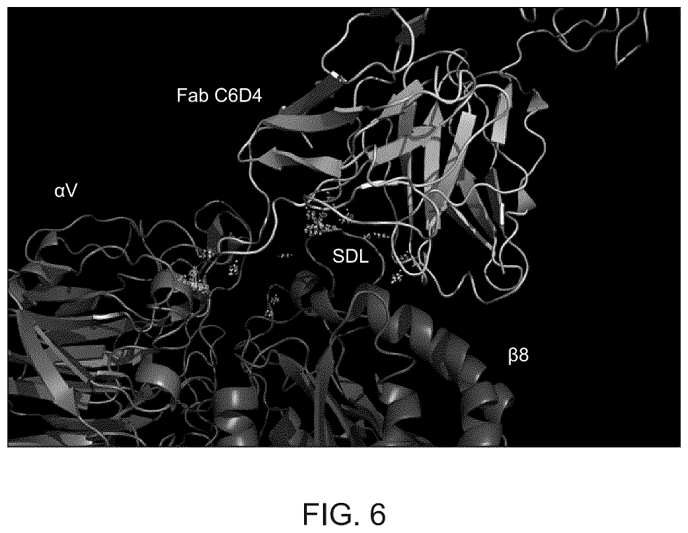

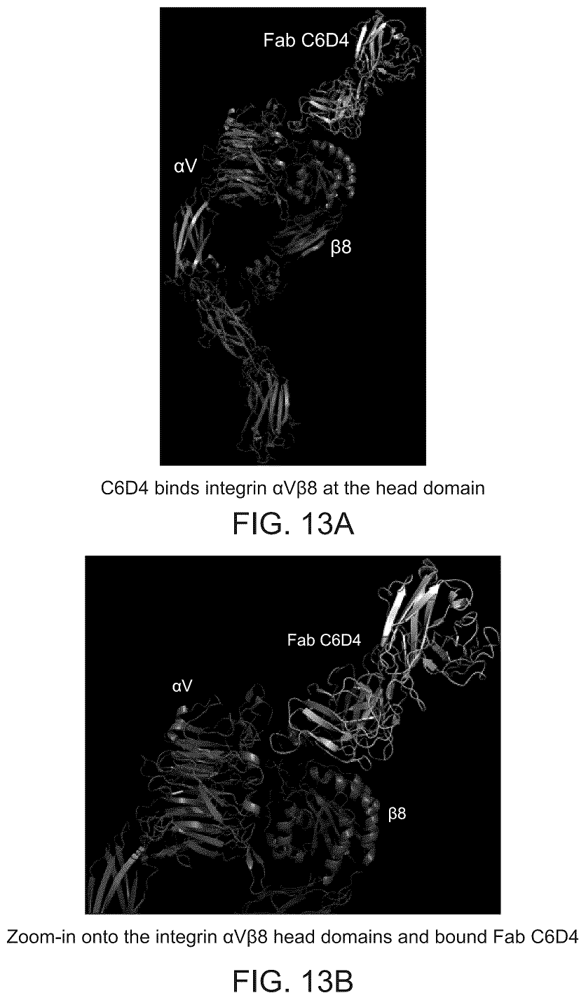

FIGS. 13A and 13B presents cryoEM results showing binding of the C6D4 Fab to the integrin αvβ8 at the head domain. FIGS. 13A and 13B illustrate this binding between C6D4 and αvβ8 in closer detail. From the C6D4 antibody footprint of FIG. 6, it can be seen that C6D4 binds primarily to the SDL loop of β8, making additional contacts with other secondary structures on the 8 α1 and α2 helices and on the head of αv. Together, these components of the binding configuration result in the almost complete occlusion of the ligand binding pocket. The residues of the β8 α1 and α2 helices and αv head that directly interact with C6D4 are further detailed in FIG. 7.

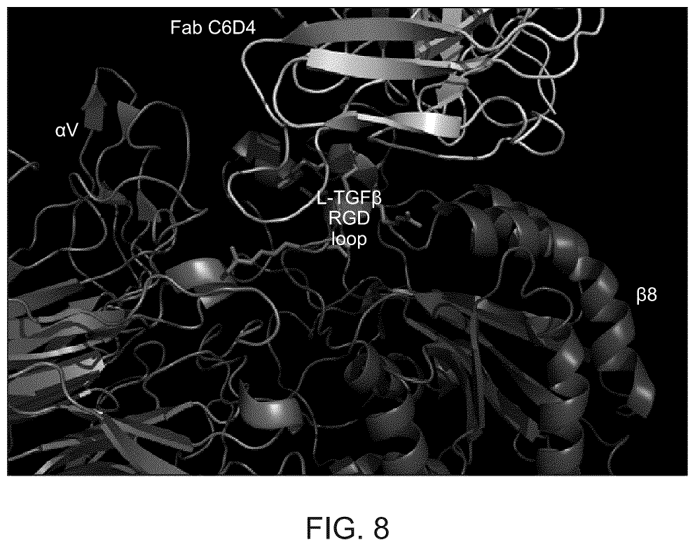

The elucidated structure shows that the CDR1 domain of the D4 V_{L} binds close to the contact site for the R of RGD in the previously published αvβ6-RGD crystal structure. Because the αv subunit is shared by both αvβ6 and αvβ8, this finding suggests that the CDR1 loop of D4 V_{L} is optimally positioned to sterically inhibit the binding of the R of RGD of latent TGF-β to αvβ8. On the other side of the SDL is a hydrophobic binding pocket having an L that immediately follows the RGD, forming an RGDL peptide. This hydrophobic pocket has been shown to be essential as a secondary binding site for the binding of the latent TGF-β RGD peptide to αvβ6. See, e.g., Shi M, et al., Nature 474 (7351): 343-9 (2011). The L or RGDL has also been shown to be essential for the binding of the latent TGF-β RGD peptide to αvβ8. (See, e.g., Ozawa, A, et al. J Biol Chem. 291 (22): 11551-65 (2016). The CDR3 loop of C6 Vu has now been shown to bind in such a way as to substantially cover the hydrophobic binding pocket located on the β8 subunit head domain. Additionally, C6D4 was found to interact extensively with the SDL of β8. FIG. 8 illustrates the overlapping of the C6D4 epitope with the ligand binding pocket of integrin αvβ8, showing how it can prevent the association of the integrin with latent TGF-β, and thus the activation of latent TGF-β. Importantly, all contact residues with C6D4 are believed to be conserved in αvβ8 across all mammalian species. This is in contrast to the allosteric inhibitor B5, which only reacts significantly against human αvβ8.

### Example 4. Modeling of C6D4 Effects on Lung Cancer Survival

Syngeneic models for the study of lung cancer are very limited. The Lewis lung carcinoma (LLC) model is the only reproducible syngeneic lung cancer model currently widely in use. LLC is a cell line established from the lung of a C57BL mouse bearing a primary Lewis lung carcinoma. This line is highly tumorigenic and is used to model pulmonary metastasis that results after resection of the primary tumor. In this way the model mimics the clinical scenario closely. It is a useful model for evaluating the efficacy of chemotherapeutic agents in vivo. An advantage of the LLC model is that tumor cells are immunologically compatible, unlike the immunodeficient strains used in most other xenograft models. The LLC model was used as a preclinical model to evaluate vinorelbine prior to its use in clinical trials. The LLC cell line is injected subcutaneously into the subcutis of C57B6 mice, and within two weeks primary tumors reproducibly reach sizes of 10 mm. After resection of the primary tumor, lung metastasis appears in 2-4 weeks. The primary endpoints in this model are weight loss and lung metastasis number.

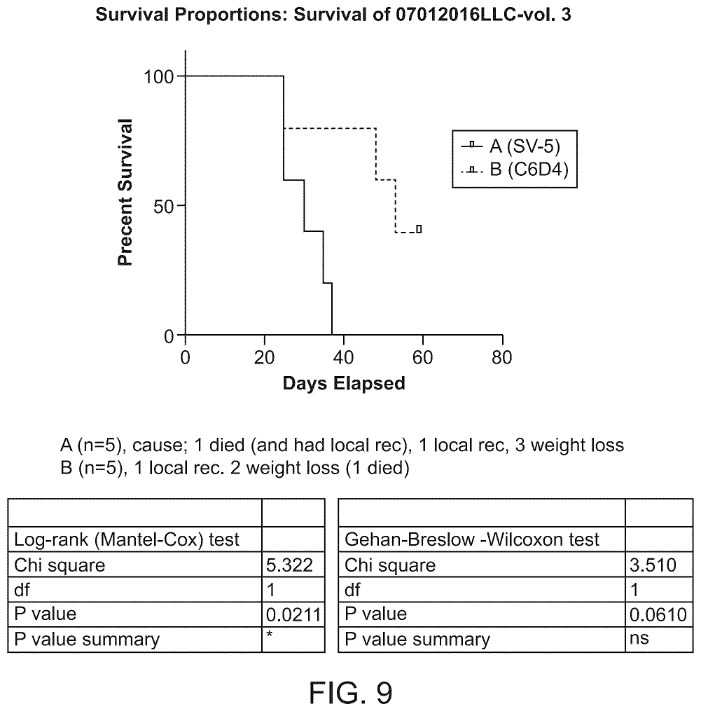

FIG. 9 presents result indicating that C6D4 increases survival in the LLC model. Mice received intraperitoneal injections of either C6D4 murine IgG2a or SV5 isotype control (7 mg/kg) at the time of primary tumor removal (day 0), and then once every week until weight loss exceeded 20%. The positive results indicate the first demonstration of an anti-β8 antibody inhibiting lung cancer metastasis. The fact that C6D4 inhibits lung cancer metastasis in this model indicates its potential as a treatment to prevent lung cancer metastasis. Because the mechanism of this antibody in cancer likely involves inhibiting the function or development of immunosuppressive Treg cells, C6D4 can have broad applications to any number of cancers where Treg cells play an immunosuppressive role.

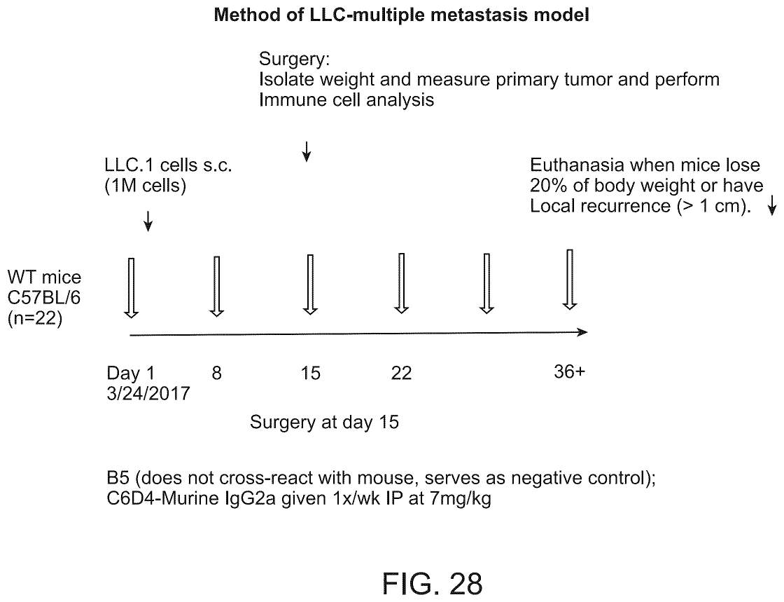

FIG. 28 provides a schematic of the LLC model used herein to evaluate lung metastasis. The LLC tumor cell line is syngeneic to the host C57B/6 strain. This cell line does not express the integrins αvβ6 or αvβ8. The LLC. 1 cell line has been passed though mice one time and regrown from lung metastasis. After two weeks, subcutaneously injected tumor (1×10⁶) LLC.1 cells form large tumor nodules (˜1 cm). The tumors are removed surgically and when animals lose 20% of their body weight they are euthanized.

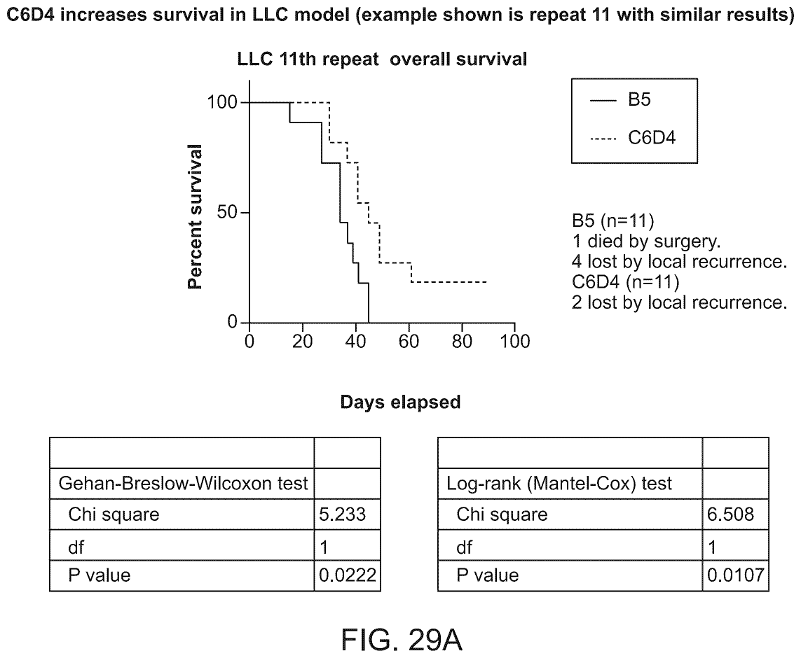

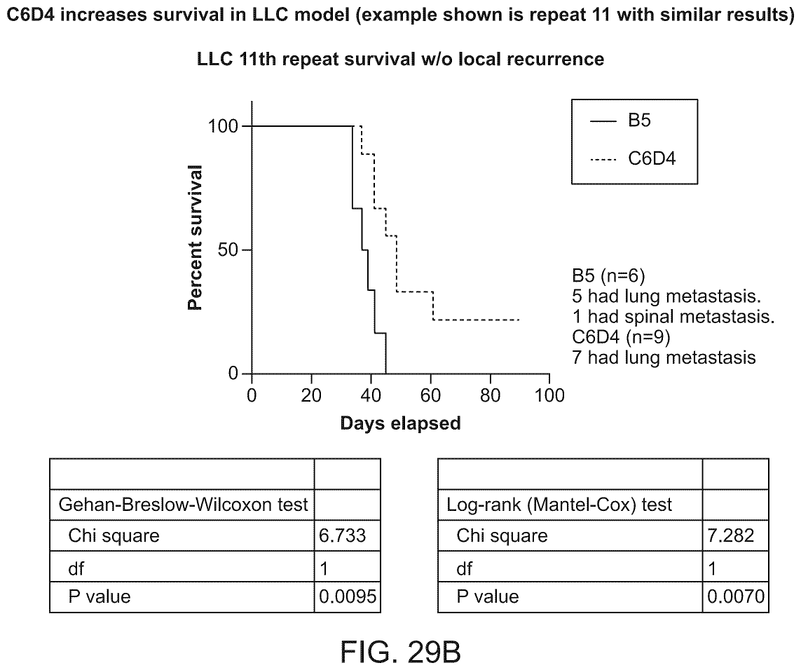

The LLC model lung metastasis experiment described in the preceding paragraph was repeated eleven (11) times and the results in each of the eleven experiments were found to be similar (data not shown). FIGS. 29A and 29B present data from the eleventh experiment indicating that C6D4 increases survival in the LLC model. In each instance, mice received intraperitoneal injections of either C6D4 murine IgG2a or SV5 isotype control (7 mg/kg) at the time of primary tumor removal (day 0), and then once every week until weight loss exceeded 20%. The results indicate the anti-β8 antibody (C6D4) inhibits lung cancer metastasis. Survival curves in FIG. 29A represent mice euthanized for reasons of local recurrence or weight loss. In FIG. 29B, the animals removed for local recurrence are excluded. At autopsy, the animals with 20% weight loss all have metastatic implants in their lungs. The C6D4 antibodies were injected for up to 90 days in surviving animals. Interestingly, post-mortem examination did not reveal any abnormal inflammatory response in the tissues examined. The fact that C6D4 inhibits lung cancer metastasis in this model indicates its potential as a treatment to prevent lung cancer metastasis. Because the mechanism of this antibody in cancer likely involves inhibiting the function or development of immunosuppressive Treg cells, C6D4 can have broad applications to any number of cancers where Treg cells play an immunosuppressive role.

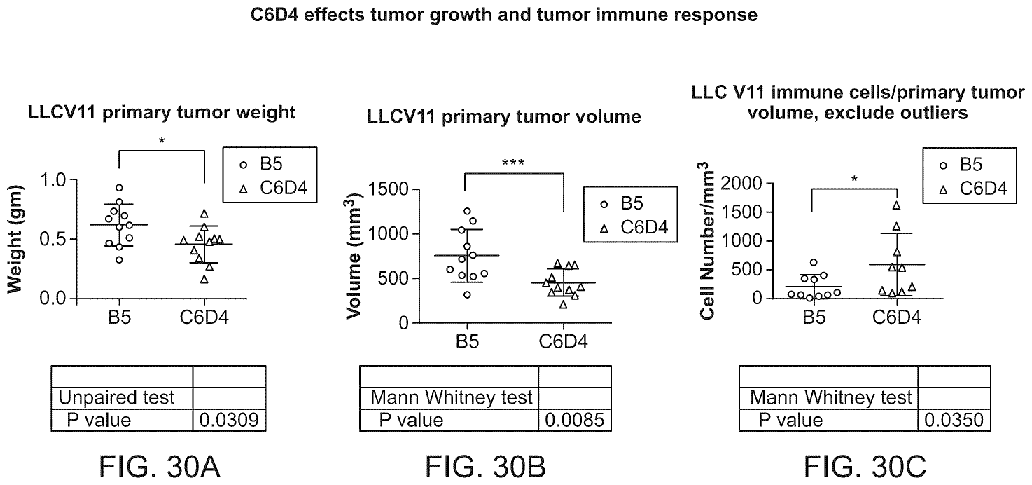

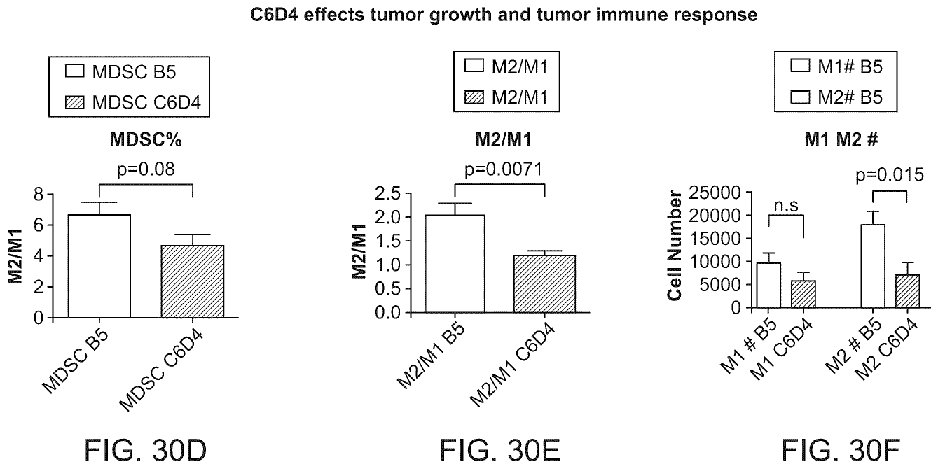

The effect of C6D4 was also evaluated with respect to tumor growth and tumor immune response. From the resected LLC.1 primary tumors in mice that received two injections of isotype control (B5, which only cross reacts with human and not mouse b8) or C6D4 (which cross-reacts with mouse and human), the primary tumor weights were recorded and dimensions measured. The tumors were enzymatically disaggregated and immune cells isolated and counted. Flow cytometry was performed and tumor infiltrating immune cells separated from tumor cells using Percoll gradient centrifugation. FIGS. 30A-F is one of three experiments with similar results (remaining data not shown). In each experiment, n was greater than, or equal to, 10 in each test group.

### Example 5. C6D4 Effects on Metastatic Disease Using a Melanoma Disease Model

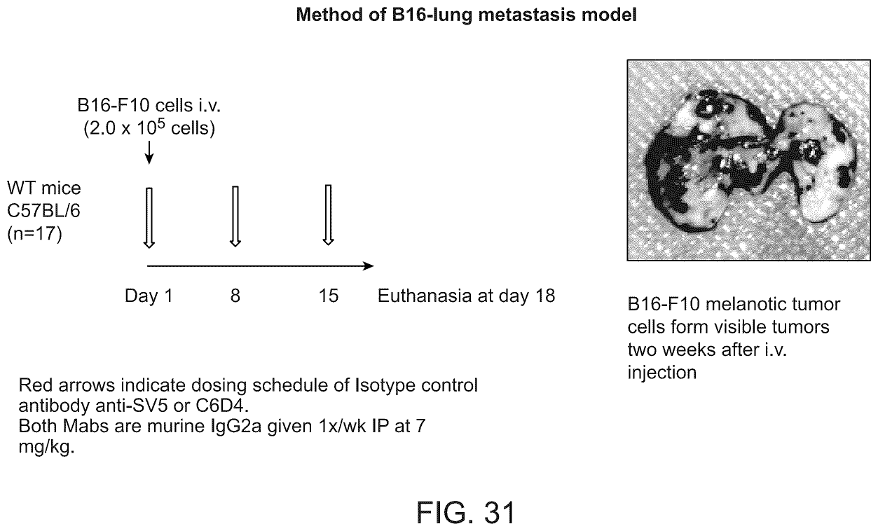

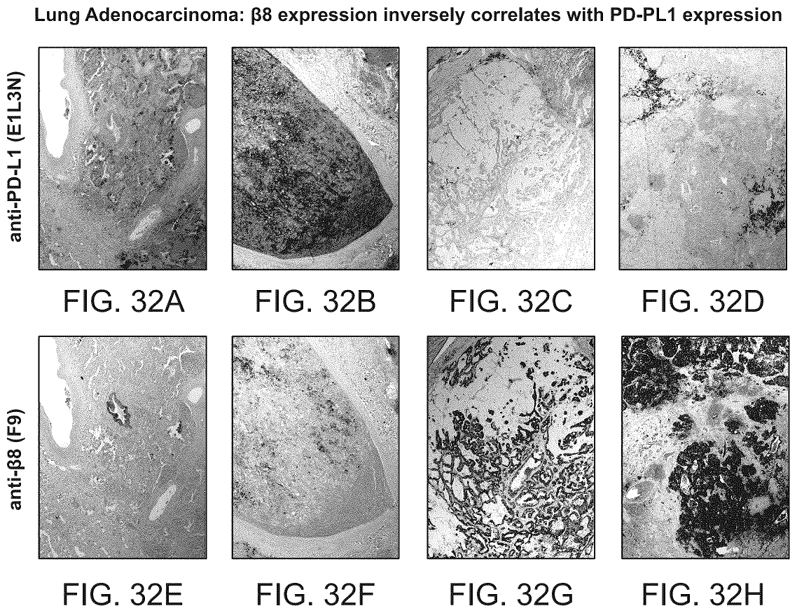

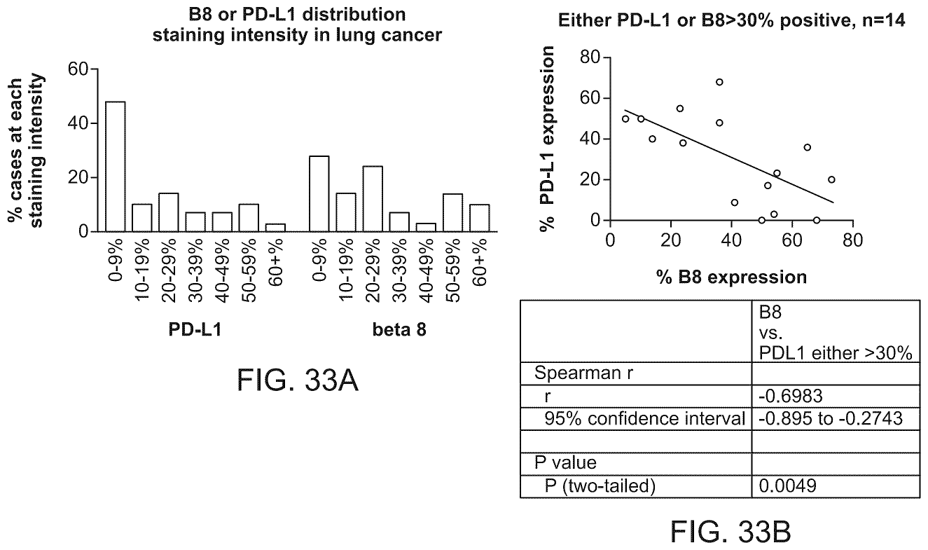

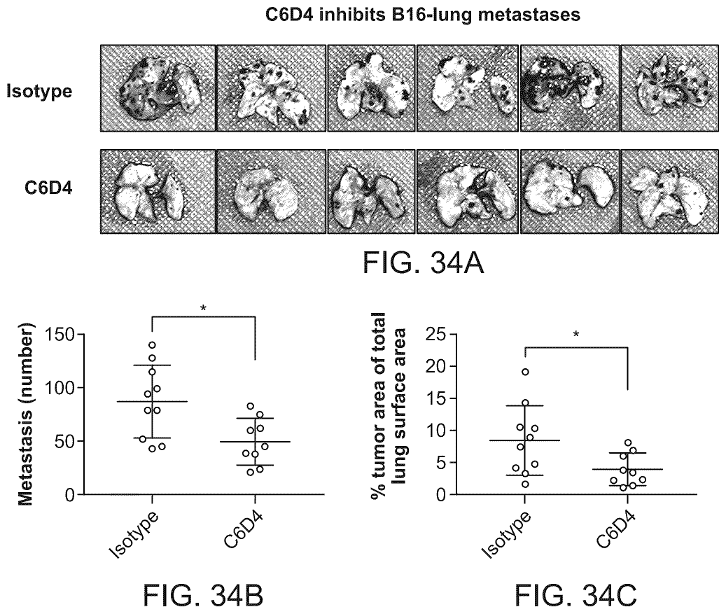

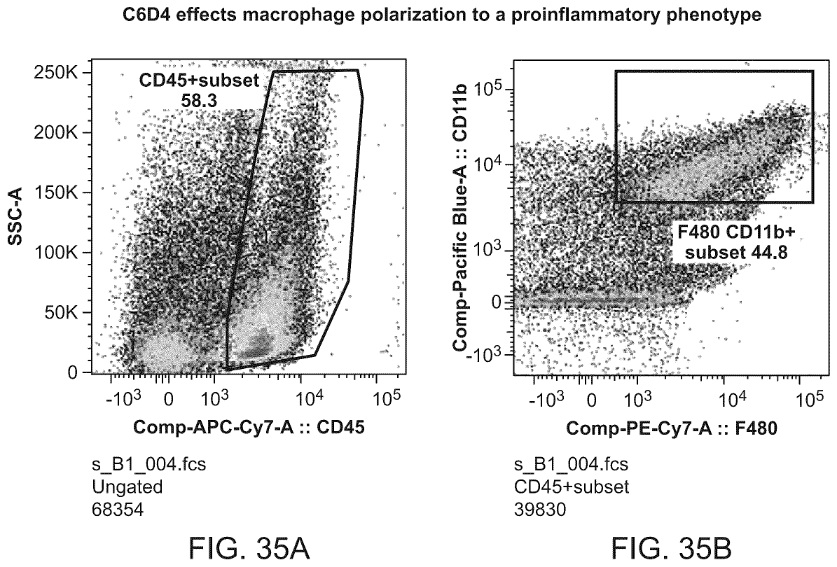

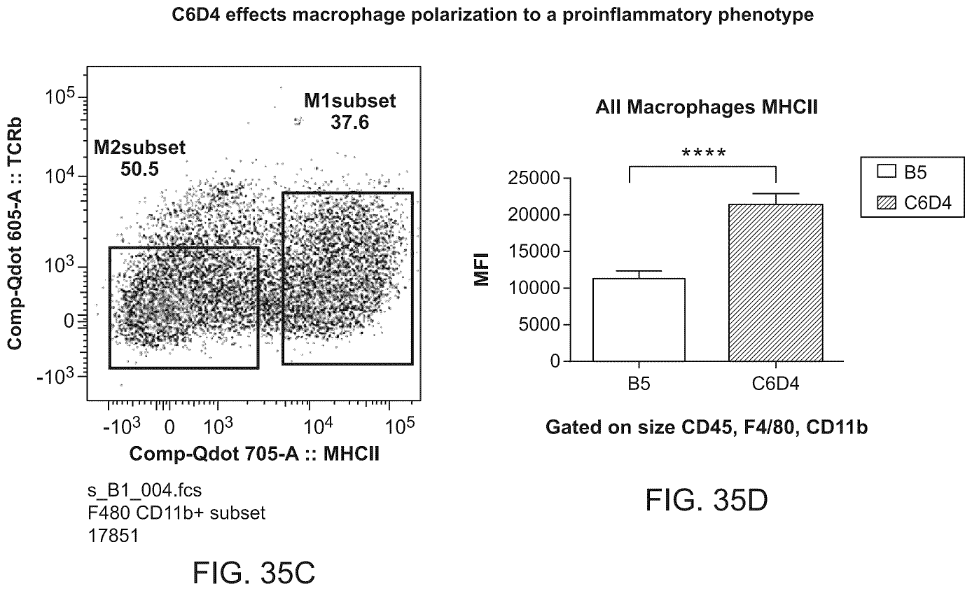

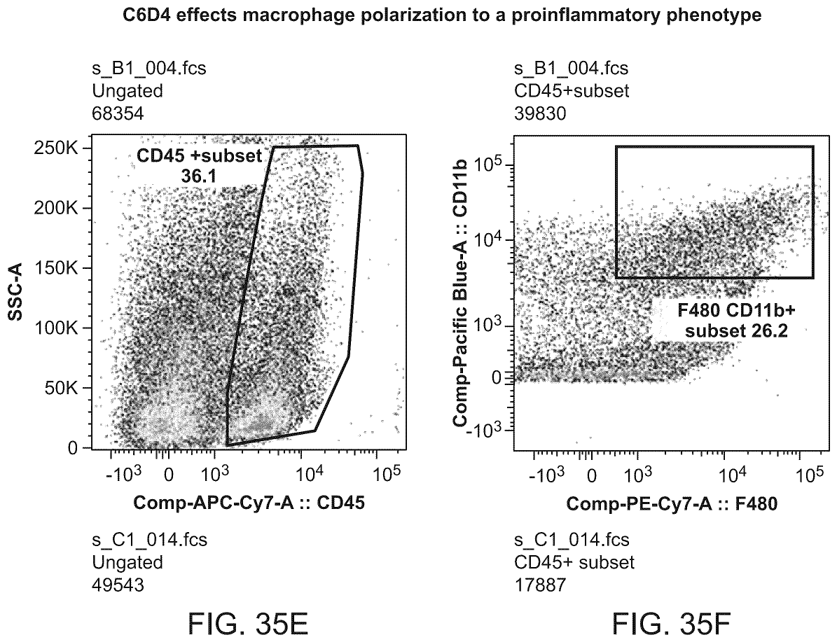

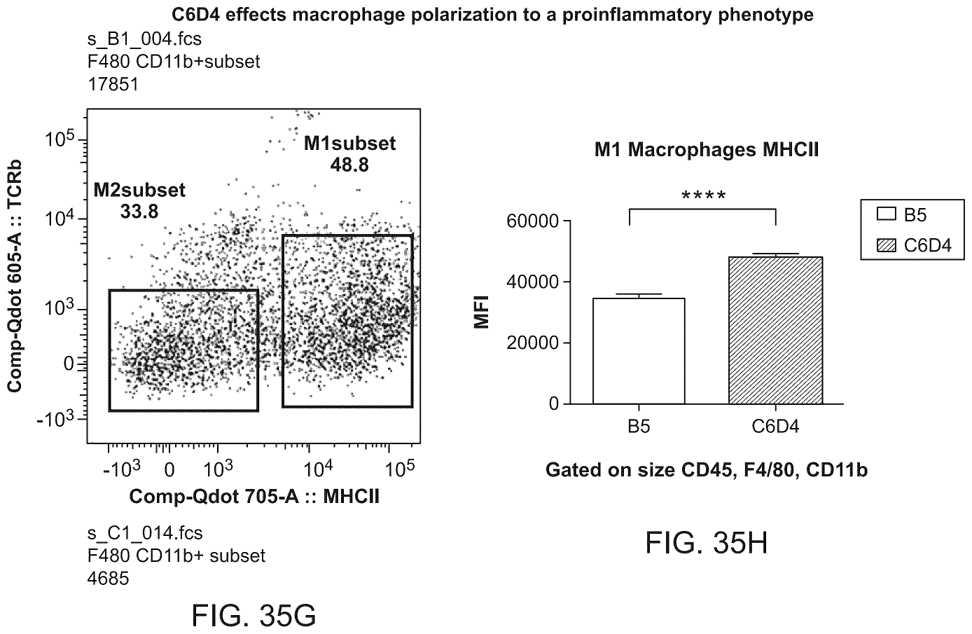

A model for the study of metastasis was tested herein that utilized the B16-F10 tumor cell line. The B16-F10 highly metastatic tumor cell line is syngenic to the host C57B/6 strain. This line does not express the integrins αvβ6 or αvβ8. The B16-F10 cell line was transfected with murine ITGb8 and after selection in G418 and two rounds of sorting, a pool of high expressing αvβ8 cells were identified. When injected intravenously via the tail vein, visible lung metastases appeared within 14 days. A schematic of the metastatic disease melanoma model described in this paragraph is provided in FIG. 31. After three injections (i.p.) of isotype control (SV5) or C6D4, both at 7 mg/kg, at days 0, 7 and 14, the mice were euthanized at day 18. FIG. 34A shows photographs of representative lungs in anterior and posterior views; visible lung metastases were counted and the total lung surface area involved with metastases was assessed.

FIG. 34B shows the total number of metastases and FIG. 34C shows the percentage of total lung surface area involved in metastatic melanoma.

### Example 6. Modeling of C6D4 Effects on Hepatitis B Infection and Disease Outcome

Because the hepatitis B virus (HBV) does not infect mice, research has typically focused on using transgenic and knockout mouse models to study HBV immunity. In this model, viral antigens in the liver are exposed to an immune system that is not immunologically tolerant, and that has not been previously exposed to HBV. The goal is to mimic the immunologic events that would normally occur during primary HBV infection. In addition, this model permits manipulation of the immune system that is exposed to the virus, to be able to identify and dissect the cells, cytokines, and chemokines contributing to chronic hepatitis or disease resolution.

To generate the model, the resident (tolerized) immune system of the HBV-transgenic mice is ablated by backcrossing to immune-deficient strains (Mombaerts et al. (1992) Nature 360:225 and Mombaerts et al. (1992) Cell 68:869). This breeding strategy generates animals expressing high levels of viral antigen (HBV-Env) or virus (HBV-replication) in the liver, in the absence of a tolerant immune system (Baron et al. (2002) Immunity 16:583). Into these mice, HBV-naive syngeneic splenocytes (the equivalent of a whole spleen) are transferred from wild-type mice to reconstitute the immune system, mimic the point of primary infection, and test the importance of cellular and soluble mediators in HBV pathogenesis. Careful monitoring of immune responses and pathologic outcomes has revealed the utility of this model in mimicking or modifying acute and chronic HBV infection (Publicover et al. (2011) J. Clin. Investigation 2011:1154 and Publicover et al. (2013) J. Clin. Investigation 123:3728). In this way, the mouse model provides an experimental system to examine the reversibility of the altered immune priming that facilitates HBV persistence, and to test immune-modulatory therapeutics.

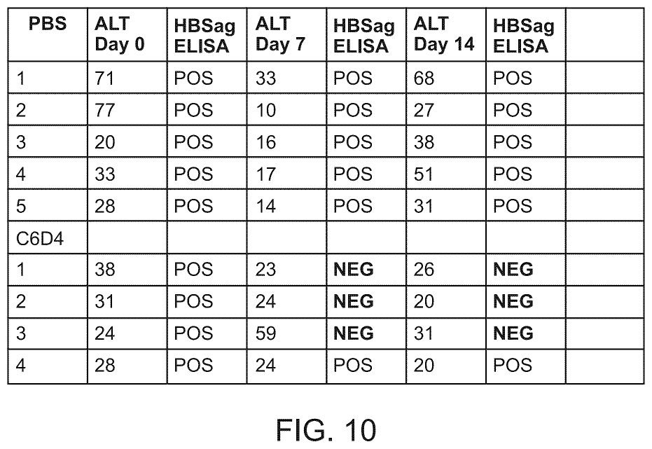

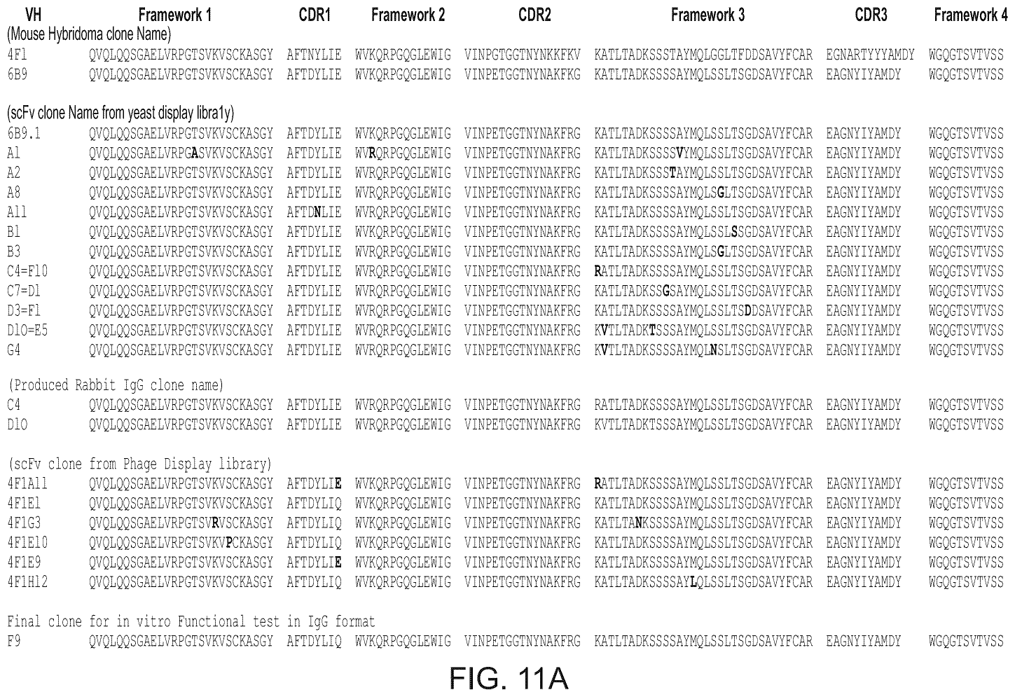

Results shown in FIG. 10 indicate that C6D4 induces HBV viral clearance in the chronic infection mouse model without causing hepatitis. In the figure, HepB surface antigen (HBSag) is a surrogate for intact HBV. Clearance of HBSag is a marker of HBV clearance. ALT is the liver enzyme monitored to measure liver inflammation and damage. The normal range of ALT in mice is 15-40. It can be seen from the data that the C6D4 antibody promoted HBsAg clearance in three of four chronic HBV model mice.

### Example 7. Construction and Characterization of Composite Antibody 4F1F9

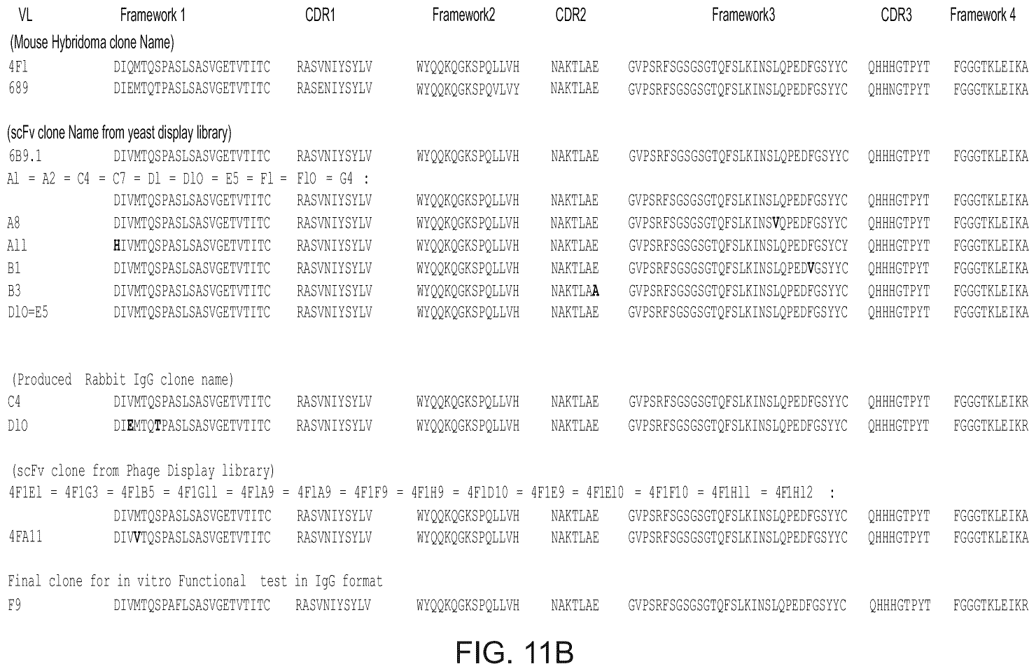

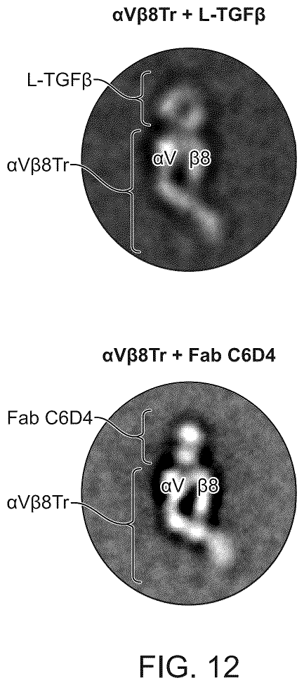

A yeast display scFV library was created using V-genes from hybridoma clones 6B9 and 4F1, a new clone 6B9.1 was selected from this library, then another yeast display scFV library was created using the V-gene of 6B9.1 and random mutagenesis, sixteen affinity-matured variant from this second library were characterized in terms of binding affinity and two clones C4 and D10 were transformed in to rabbit IgG format, both reacts weakly with human β8 in formalin-fixed paraffin-embedded tissue. A third mutagenic scFV library was then created from the variable regions of these two antibodies and inserted in a phage display vector and displayed as scFv on the phage surface (FIG. 11A-B). The induced phage library was screened against immobilized paraffin-embedded human αvβ8. Multiple rounds of selection were carried out, and fifteen phage clones were characterized in detail before the final clone F9 (FIG. 11A-B) was picked and transformed into IgG format for in vitro characterization.

Clone F9 in the IgG format was found to work efficiently in formalin-fixed paraffin-embedded tissues. The clone can be suitable for use as a companion diagnostic, for example to determine tumors expressing αvβ8 or infiltrated by immune cells expressing αvβ8 (i.e. dendritic cells, Treg cells), as a bioimaging reagent for measuring β8-specific tumor uptake and for informing C6D4 treatment decisions. The F9 antibody can also be used to detect αvβ8 in fluid or tissue lysate samples using ELISA.

### Example 8. Methods to Inhibit and/or Treat H. Pylori Pathogenicity

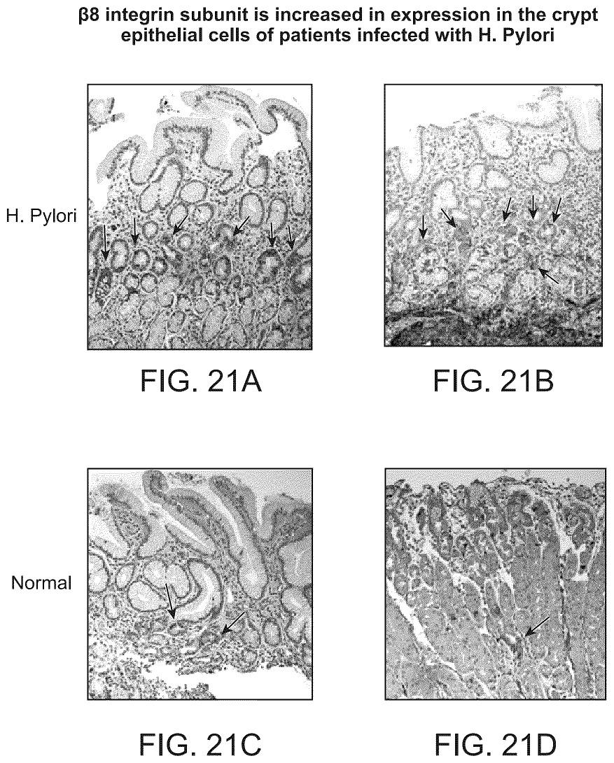

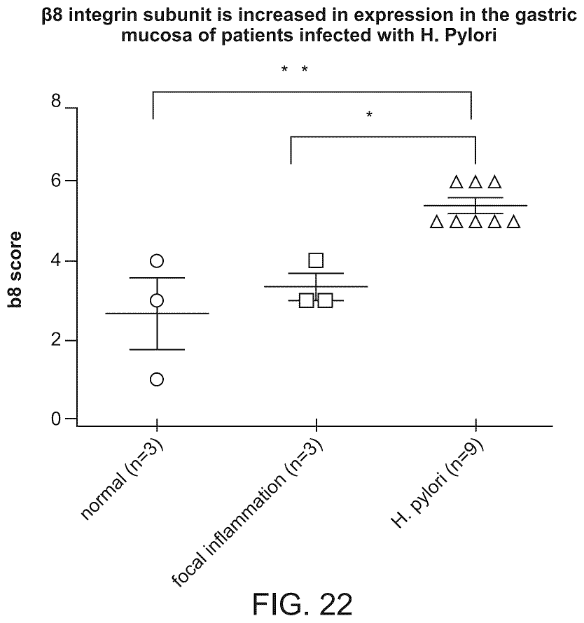

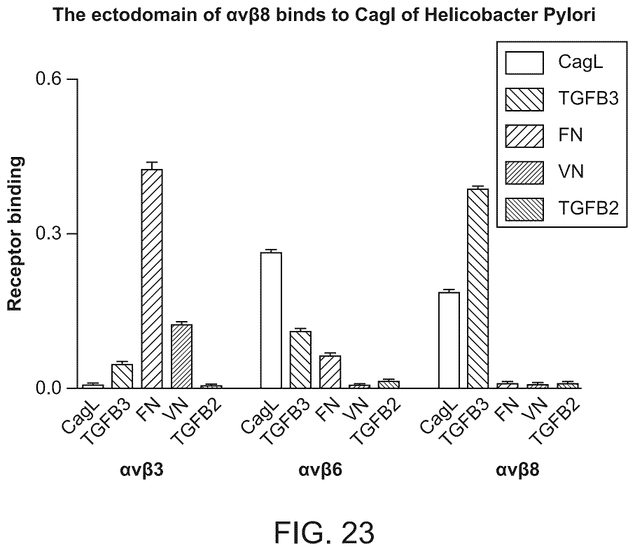

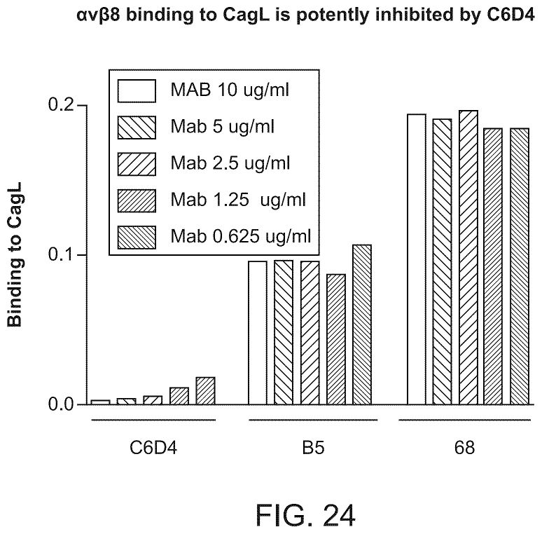

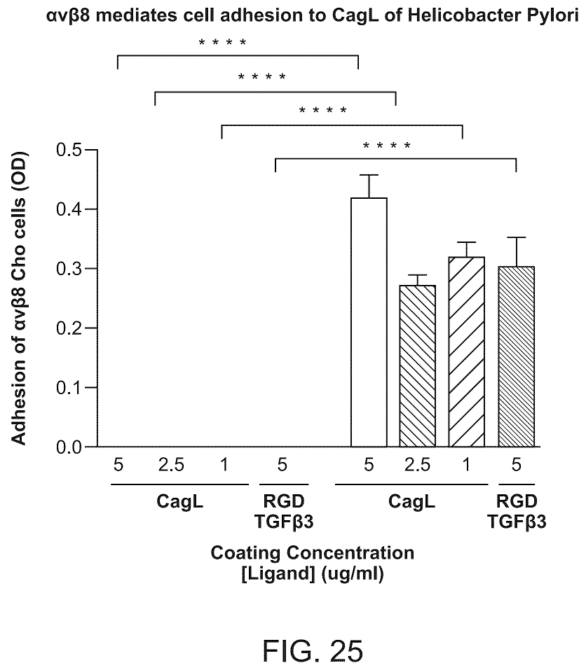

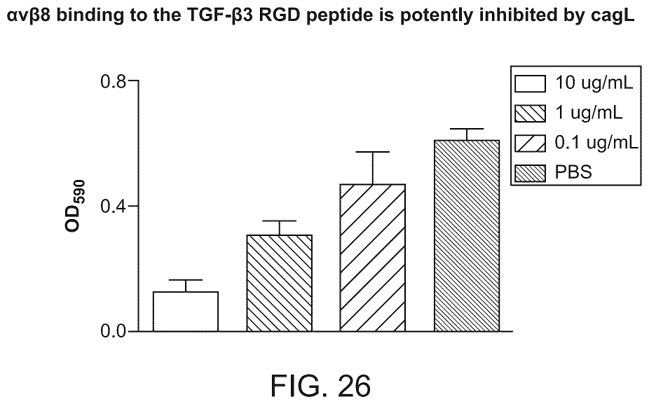

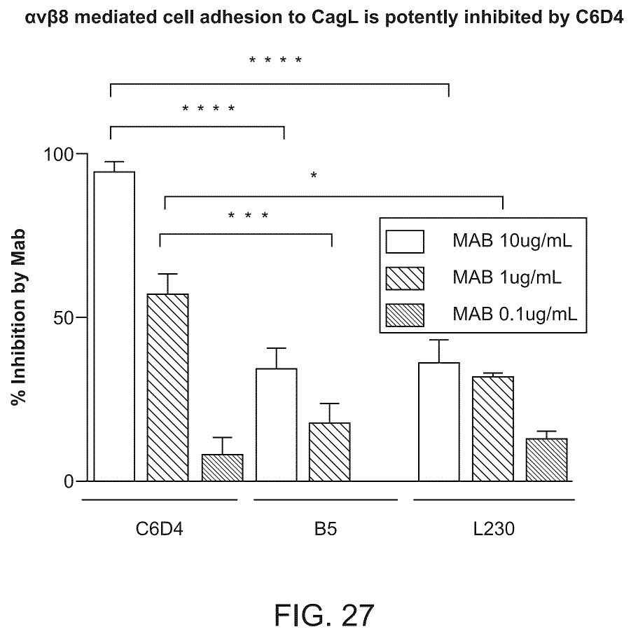

The bacterium Helicobacter pylori (H. pylori) infects the stomachs of approximately half of the world's population and is associated with peptic ulcer disease, gastric carcinoma and gastric lymphoma (MALToma). The pathogenicity of Helicobacter pylori is linked to a type IV secretion system and the cytotoxicity-associated gene pathogenicity island cagPAI. The cagPAI proteins are transcribed from a 40 kb stretch of H. pylori DNA encoding ˜31 genes of which one, cagL, contains an RGDL integrin binding motif. This RGDL motif is thought to act as a receptor for integrins so that the H. pylori pilus can interact with gastric epithelial cells and then penetrate the cell membrane and the oncogenic toxin cagA can be injected into the cell (see Kwok, et al, Nature, 2007449, 862-866, and Barden, et al, Journal of Molecular Biology, 2015, 427 (6) Part B, 1304-1315). We have used the anti-β8 clone F9 to stain human stomach biopsies and have found that the integrin αvβ8 is expressed by gastric crypt epithelial cells and this expression is increased in patients with chronic active gastritis due to H. pylori infection (see FIGS. 21 and 22). The ectodomain of integrins αvβ6 and αvβ8, but not other RGD-binding integrins (αvβ1, αvβ3, αvβ5 and α5β1) have been shown to preferentially bind to CagL via an RGDL dependent mechanism (see Barden, et al, Gastroenterology, 2010, 138 (3). Previously, it was thought that the α5β1 integrin was the main CagL receptor on gastric epithelial cells (see Kwok, et al, Nature, 2007, 449 (7164): 862-6. We have found that the integrins αvβ6 and αvβ8 bind with similar efficiency to CagL while the αvβ3 integrin does not bind to CagL (See FIG. 23). The αvβ8-mediated binding to CagL can be efficiently blocked by C6D4 (See FIG. 24). The αvβ8 integrin also mediates strong cell adhesion to CagL (see FIG. 25) and CagL can compete for αvβ8-mediated cell adhesion to the TGF-β3 RGD peptide, indicating that αvβ8 binds to the RGD site of CagL (See FIG. 26). C6D4 can efficiently block cell adhesion to CagL (See FIG. 27).

Blocking αvβ8-mediated binding of CagL with C6D4 or its derivatives (i.e. IgA, monomeric or dimeric) can be used as a method to inhibit H. Pylori pathogenicity (i.e. peptic ulcer disease, gastric carcinoma or MALToma) by blocking entry of the oncogenic toxin CagA. In addition, C6D4 could provide protection against H. Pylori itself or from its indirect oncogenic and toxic effects by inhibiting Treg function and increasing more effective immunity against H. Pylori, gastric carcinoma, and MALToma. Such effects can be predicted by findings in murine models where H. Pylori immune escape has been shown to be mediated by dendritic cell-induced Treg skewing and Th17 suppression (see Kao, et al, Gastroenterology, 2010 138 (3): 1046-54). Because the integrin αvβ8-mediated TGF-activation has been shown to be required for Treg development and function (see Worthington, et al, Immunity, 2015, Volume 42, Issue 5, pp. 903-915), inhibiting αvβ8-mediated TGF-β activation using C6D4 or its derivatives will protect against the oncogenic effects of H. Pylori infection by enhancing immunity to H. Pylori itself while simultaneously increasing anti-tumoral immunity. Another possible mechanism by which blocking αvβ8-mediated TGF-β activation with C6D4 or its derivatives could block Treg function is by inhibiting migration of Tregs to the H. Pylori infected gastric mucosa. The chemokine CCL20 is a potent chemokine for Tregs and dendritic cells, which are required for Treg differentiation, and αvβ8-mediated TGF-β activation provides a major contribution to CCL20 production and function (see Cook, et al, Gut (2014), 63 (10): 1550-9; Brand, et al, J Biol Chem, 2015, 290 (23): 14717-28, Hashimoto, et al, J Immunol 195 (3): 1182-90). Therefore, treating patients with C6D4 or another anti-αvβ8 antibody alone, in combination with antibodies to other CagL binding integrins (α5β1, Act-1, or αvβ6, 3G9) or in combination with standard H. Pylori therapy (i.e. bismuth salts, proton pump inhibitors, macrolides, amoxicillin, metronidazole) would treat not only the pathogenic mechanism of H. Pylori but would enhance immunity to more efficiently eliminate H. Pylori, while at the same time protecting and/or treating the malignant complications of chronic H. pylori infection.

### Example 9. Construction of Composite Humanized Antibody C6D4

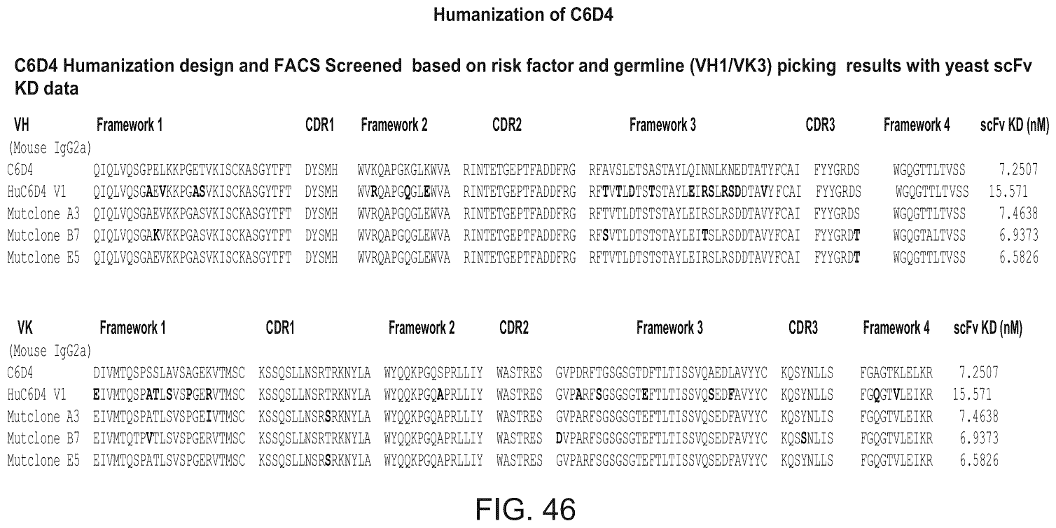

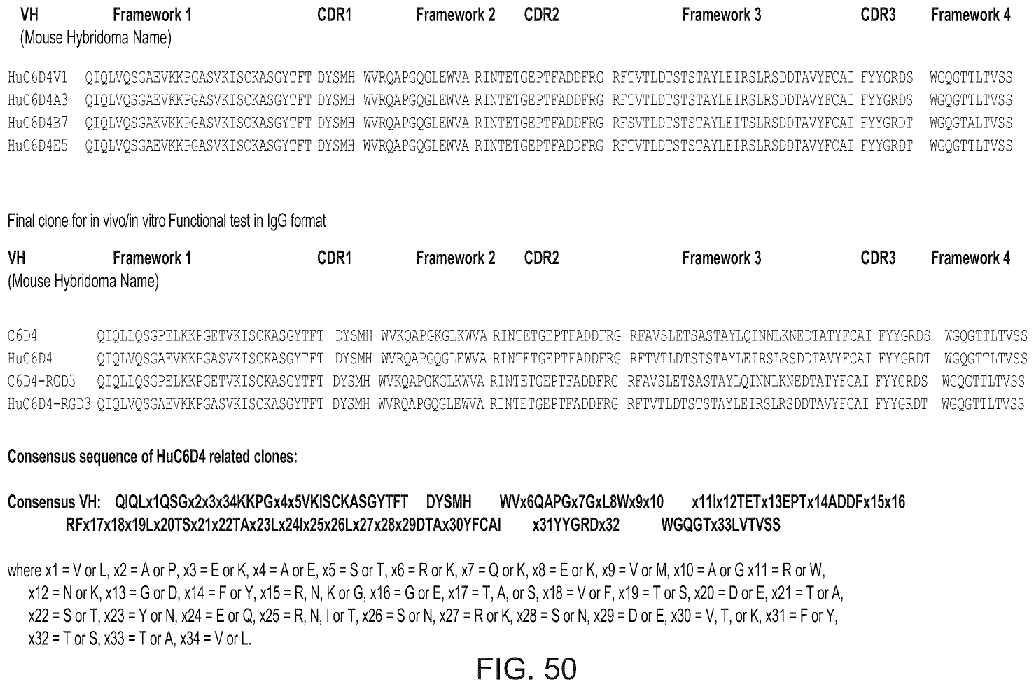

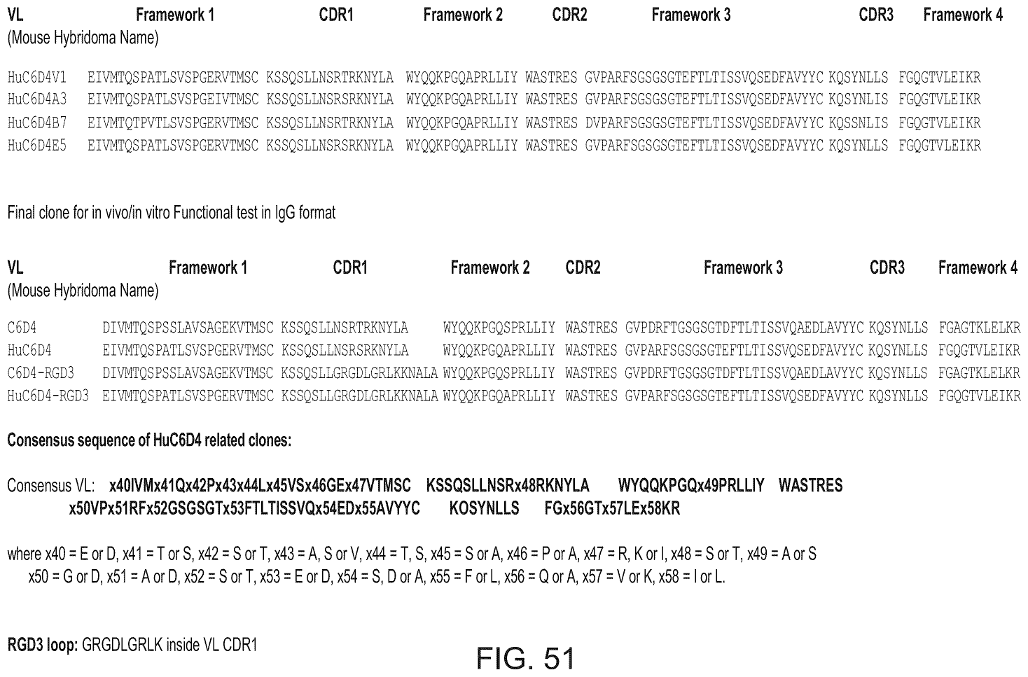

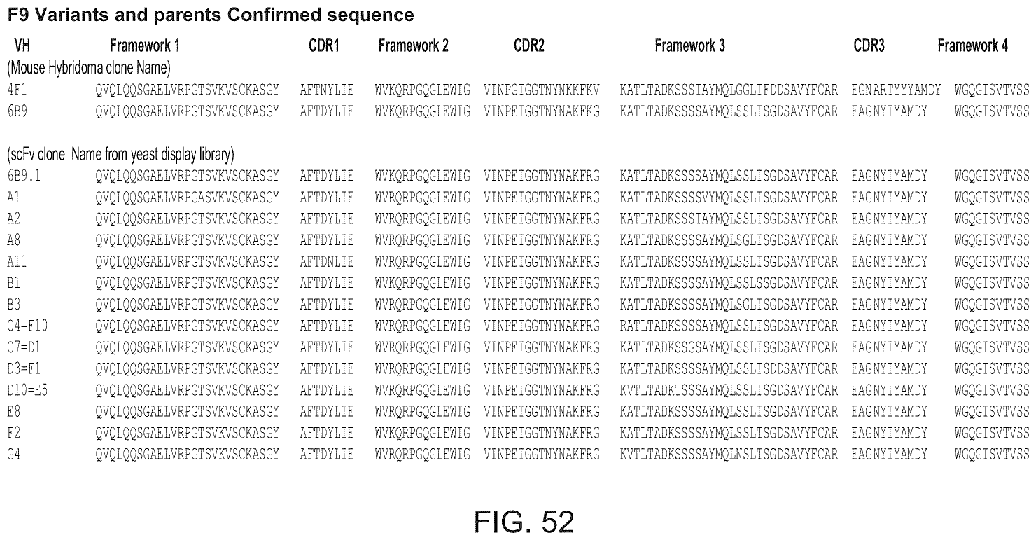

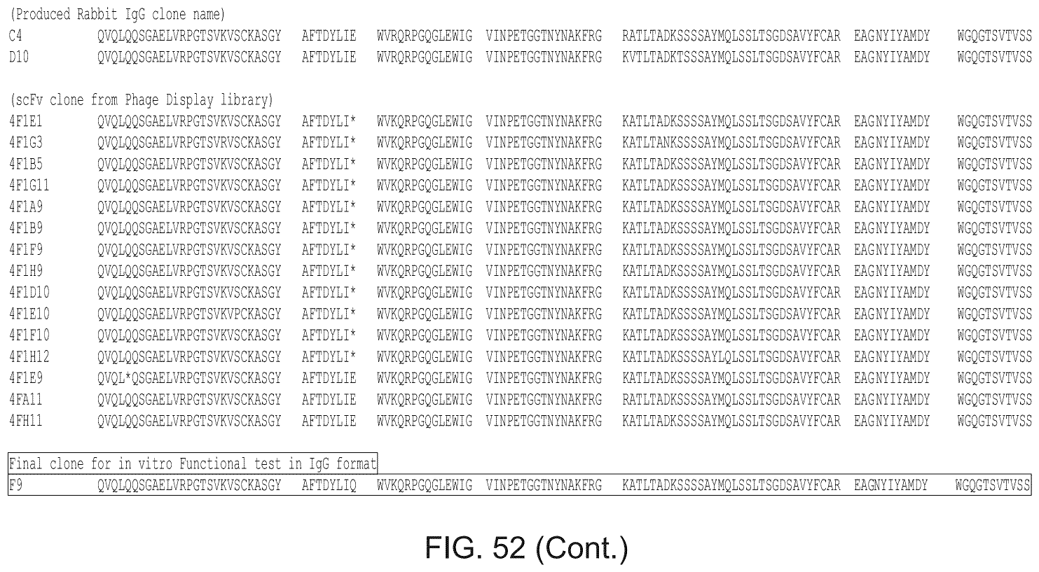

FIG. 46, FIG. 50, and FIG. 51, show sequence alignment of various C6D4 humanized clones. FIGS. 50 and 51 also provide heavy chain and light chain amino acid consensus sequences for the humanized C6D4 related clones. The C6D4 antibody humanization focused on the V domain framework region of both the heavy and light chain. The humanization process was performed to include three criteria:
(1) The humanized version of antibody (HuC6D4) should have similar or improved affinity and specificity for αvβ8 as the murine version C6D4;
(2) The final amino acid in the HuC6D4 antibody framework region should be as close as possible to the translated antibody framework region of the human germline version that was selected as the target gene family (VH1/VK3);
(3) Production levels of the final humanized version (HuC6D4) in IgG or other format should be scalable for industry application.

We designed a potential humanized lead version of the murine C6D4 based on the chosen germline of human antibody (VH1/VK3), and the humanization algorithm developed at UCSF, and other published information for antibody human drug development, with main consideration on IgG general structure, VH-VL interface, IgG folding packing, surface accessibility, vernier zone impact, humanization hotspots and other risk factors.

These designed lead versions were synthesized and expressed as scFV using yeast display. The measured Kd showed an approximate 2-fold decrease from the parent murine C6D4 scFv.

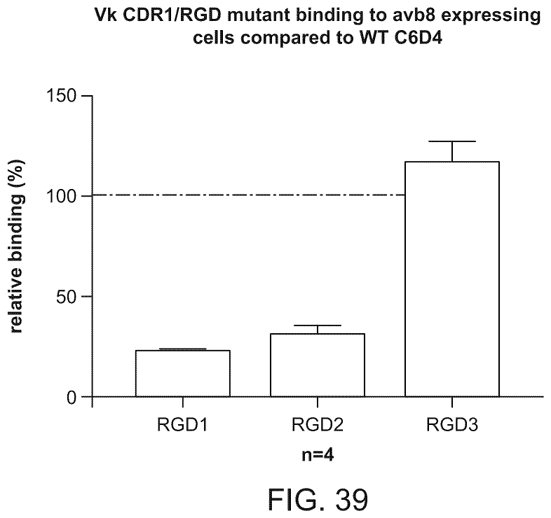

Next, a random mutation based yeast scFv display library was created using the humanized lead version as the starting point, and FACS sorting performed to pick the best binders to αvβ from the displayed yeast library. Three mutant candidates (C6D4-RGD1, C6D4-RGD2 and C6D4-RGD3) were chosen for further testing in IgG format (See, for example FIG. 38C and FIG. 39).

### Example 10. Characterization of Humanized C6D4 and CD64-RGD3 Binding Affinity

Shown in FIG. 39 is cell surface staining experiments of C6VH expressed with either RGD1, RGD2, or RGD3 mutants (as disclosed in Example 8) as rabbit IgG. Binding to human Cho cells expressing αvβ8 was expressed as a percentage of binding of C6D4. The results show that RGD3 mutant has substantially higher relative binding to αvβ8 as compared to wildtype C6D4, RGD1 mutant or RDG2 mutant.

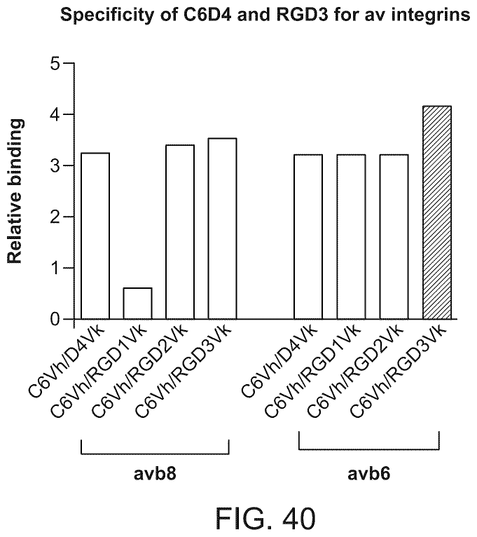

FIG. 40 shows cell surface staining experiments of C6VH expressed with either D4 Vk or RGD1, RGD2 or RGD3 mutants (as disclosed in Example 8) as rabbit IgG. Binding to Cho cells expressing human αvβ8 or SW480 cells expressing αvβ6 are shown. Relative binding is defined as staining compared to staining of non-transfected Cho or SW480 cells. The results show that the C6D4-RGD3 mutant has substantially higher relative binding to αvβ6 as compared to wildtype C6D4, RGD1 mutant, or RDG2 mutant.

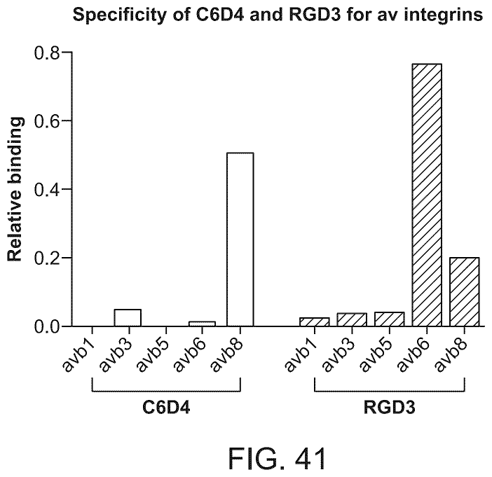

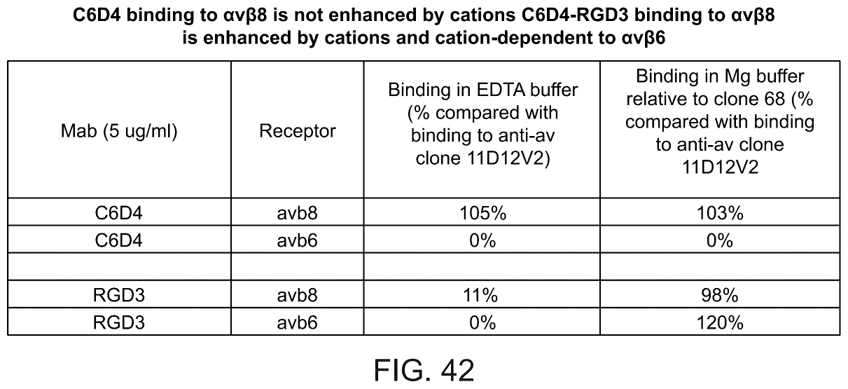

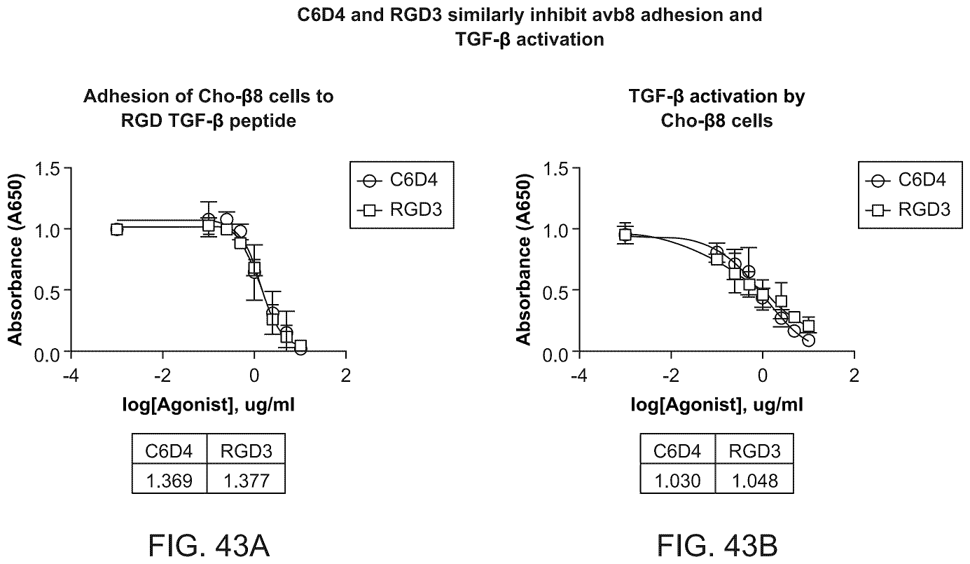

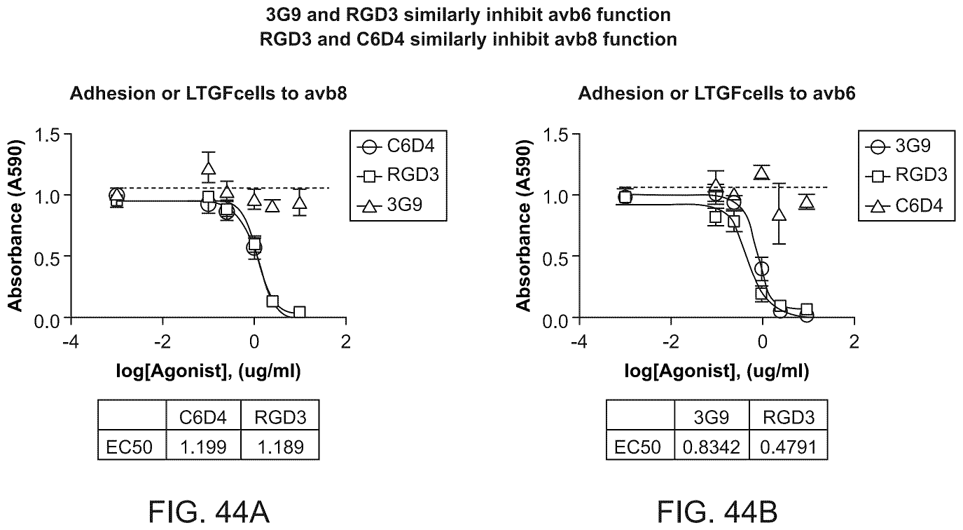

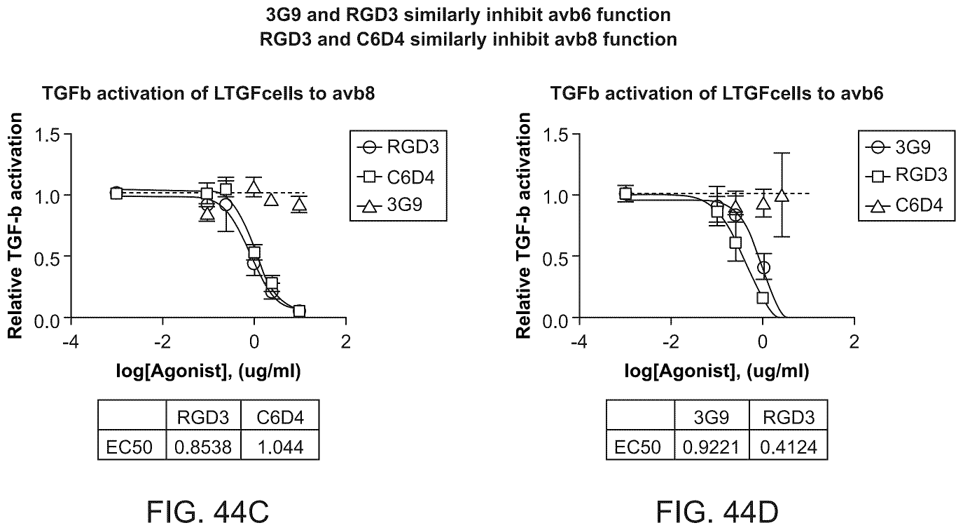

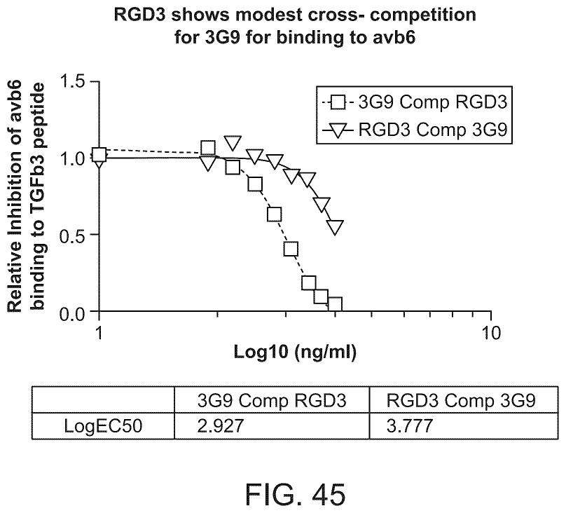

Shown in FIG. 41 is a binding experiment of C6VH expressed with either D4 Vk or RGD1, RGD2 or RGD3 mutants (as disclosed in Example 8) as rabbit IgG to various av-integrins. The integrins αvβ1, αvβ3, αvβ5, αvβ6 and αvβ8 were purchased from R&D systems. All integrins were coated on ELISA plates at 2 mg/ml, blocked with BSA, and antibodies were allowed to bind. Binding of C6D4 and RGD3 was detected with anti-rabbit HRP. The results shown are relative to control wells coated with anti-av (clone 8B8) where av-integrins were detected with another av-antibody recognizing an non-overlapping epitope (L230-biotin), followed by SA-HRP. The results show that RGD3 mutant has substantially higher binding to αvβ6, while C6D4 has higher relative binding to αvβ8.

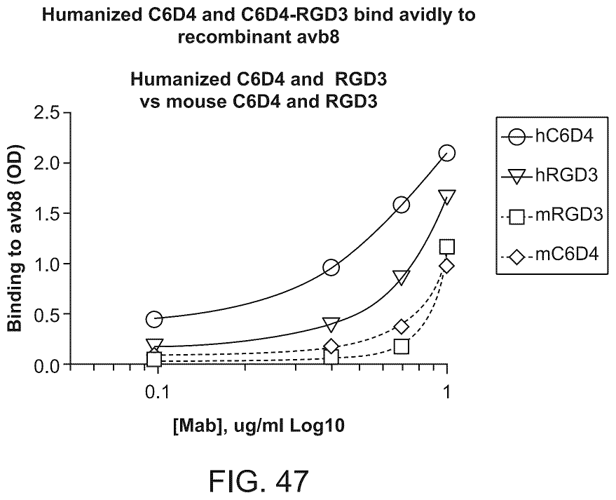

C6D4 and C6D4-RGD3 were also shown to bind avidly to αvβ8. Humanized C6D4 or C6D4-RGD3 (Frameworks and CH1 are human; hinge and CH2-3 are mouse) were immobilized on ELISA plates at the indicated concentrations. As a negative control, some wells were coated with anti-SV5 at the same concentrations. Non-specific binding sites were blocked with BSA. Recombinant αvβ8 ectodomain (0.5 g/ml) was added to each well and after binding and washing in binding buffer (1 mM Ca⁺⁺ and Mg⁺⁺), the bound αvβ8 was detected with biotinylated anti-αv (8b8) and detected with SA-HRP. The results of this experiment are shown as specific binding (minus SV5 control) (FIG. 47). The results show that C6D4 and C6D4-RGD3 outperform murine C6D4 and C6D4-RGD3 antibodies by avidly binding αvβ8.

### Example 11. Characterization of Humanized C6D4-RGD3 Binding Structure

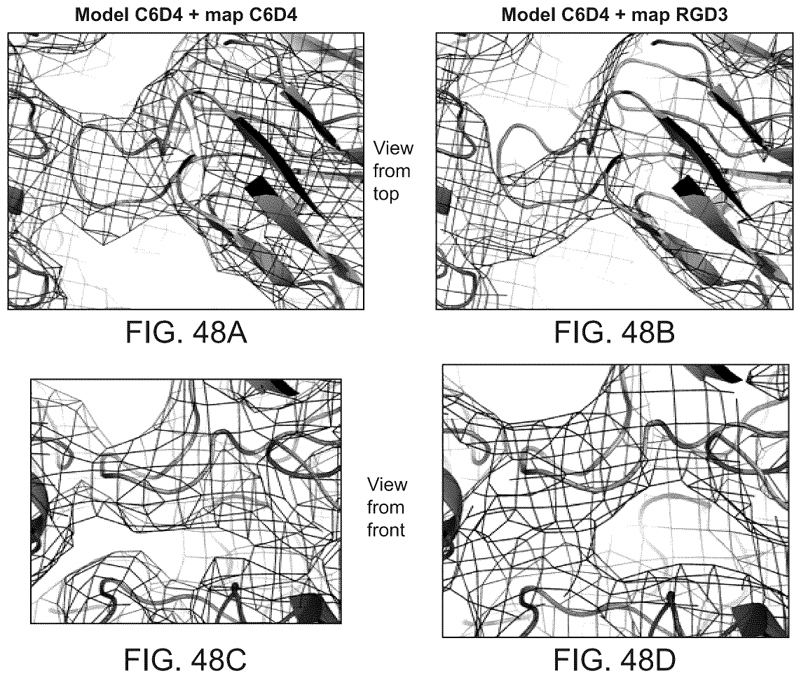

As set forth in Example 3, modeling and CryoEM maps can be used to provide structural information with respect to antibody binding. FIG. 48A-D presents a map of RGD3 binding to the ligand pocket of αvβ8. The map is derived from C6D4 in complex with αvβ8 and is compared to C6D4-RGD3 in complex with αvβ8. The density map when compared with the headpiece of αvβ6 in complex with LTGFβ1 shows the similarity of the position of the RGD residues of LTGFB1 with the RGD residues of C6D4-RGD3. Magenta wire represent s RGD3+αvβ8 density map, Black represents C6D4+αvβ8 density map; Gold represents C6D4 Fab; Green represents the αv subunit; Blue represents the β8 subunit.

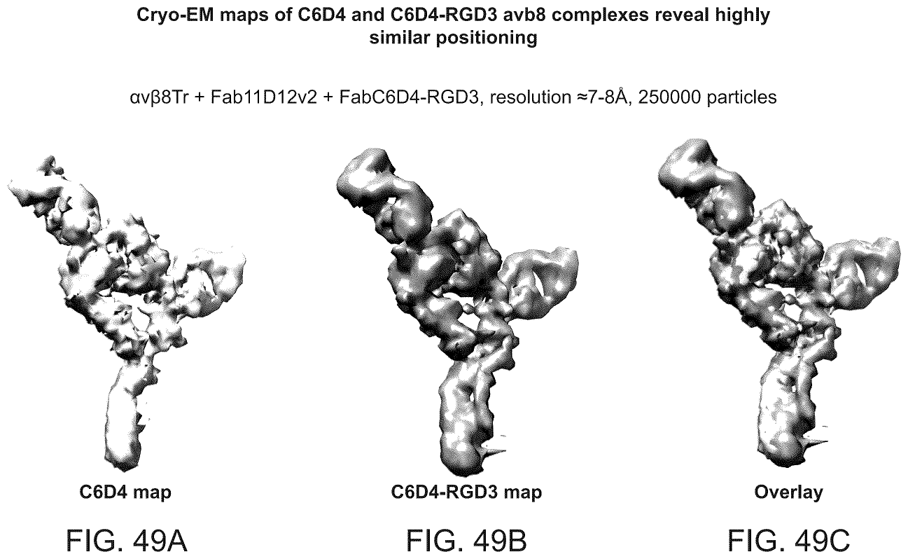

FIG. 49 is a cryoEM map showing the CDR Vk1 loop of C6D4-RGD3 occupies the ligand binding pocket of αvβ8. Here, models of C6D4 Fab-αvβ8 (FIG. 49A) are compared with RGD3-αvβ8 map (FIG. 49B) or in overlay (FIG. 49C) based on cryoEM derived density maps. The anti-αv 11D12V2 Fab was used to increase molecular mass of the complex and to assist in particle orientation. The results show that the C6D4 and C6D4-RGD3 complexes possess highly similar positioning.

### Example 12. Characterization of D4-RGD3 Mutants Having Various Loop Length of the RGD and Flanking Sequence of Pro-TGF-Beta 3

There is an amphipathic alpha-helix following the R-G-D sequence of Latent-TGF-beta1 and Latent-TGF-beta3. Of the 3 engineered versions (RGD1, RGD2, RGD3) of D4 only RGD3 contained the amphipathic helix. Therefore, we engineered various loops containing portions of the RGD and flanking sequences of Pro-TGF-beta 3 to determine if loop length altered affinity, specificity or production of each clone. Because the VH was not altered, we cloned all new constructs into the CDRL1 region of the C6D4 murine IgG expression vector and transfected the various new D4-RGD3-mutants into 293 cells. After 10 days, protein expression was compared using an murine IgG ELISA (shown as relative expression levels in the Table provided below). Integrins ανβ1, ανβ3, ανβ5, ανβ6 or ανβ8 (R&D systems) were coated on Immulon 4HBX ELISA plates (Thermo Scientific) for 1 hour at room temperature followed by blocking with a 5% bovine serum albumin solution (Sigma-Aldrich) overnight at 4° C. Supernatants with various RGD3 mutant antibodies were applied at 1/10 dilutions onto the wells for 1 hour at room temperature. Antibodies bound to the integrins were detected with an anti-5 mouse IgG-HRP antibody (GE Healthcare) and revealed with TMB substrate (Pierce). Binding was quantified by intensity as 0-4 (0 representing no apparent binding; 4 representing robust binding) and results normalized to expression. As can be seen from the data provided in the table below, different CDR_{L1} swaps into Vk D4 show distinct binding specificities. As a result, we identified several mutants having bi-specific (e.g., RGD3-2 and RGD3-3) or tri-specific (e.g., RGD3-7 and RGD3-8) binding specificities.

| | Murine | | | | | | | |
|---|---|---|---|---|---|---|---|---|
| Inserted Vk CDR_{L1} domain | IgG H + L | | IgG ELISA | Binding to recombinant human | | | | |
| swap into D4 | Vector | | Expression | integrins | | | | |
| CDR_{L1} | VH | Vk | Level | αvβ1 | αvβ3 | αvβ5 | αvβ6 | αvβ8 |
| KSSQSLLNSRSRKNYLA | C6 | D4 | 4 | 0 | 0 | 0 | 0 | 4 |
| (SEQ ID NO: 572) | | | | | | | | |
| KSSQSLLNSGRGDLGNALA | C6 | RGD2 | 4 | 0 | 0 | 0 | 0 | 2 |
| (SEQ ID NO: 574) | | | | | | | | |
| KSSQSLLGRGDLGRLKKQKDHNALA | C6 | RGD3- | 3 | 0 | 0 | 0 | 4 | 1 |
| (SEQ ID NO: 576) | | 1 | | | | | | |
| KSSQSLLGRGDLGRLKKQKDNALA | C6 | RGD3- | 3 | 0 | 0 | 0 | 4 | 4 |
| (SEQ ID NO: 577) | | 2 | | | | | | |
| KSSQSLLGRGDLGRLKKQKNALA | C6 | RGD3- | 3 | 0 | 0 | 0 | 4 | 4 |
| (SEQ ID NO: 578) | | 3 | | | | | | |
| KSSQSLLGRGDLGRLKKQNALA | C6 | RGD3- | 3 | 0 | 0 | 0 | 4 | 4 |
| (SEQ ID NO: 579) | | 4 | | | | | | |
| KSSQSLLGRGDLGRLKKNALA | C6 | RGD3 | 3 | 0 | 0 | 0 | 4 | 4 |
| (SEQ ID NO: 575) | | | | | | | | |
| KSSQSLLGRGDLGRLKNALA | C6 | RGD3- | 3 | 0 | 0 | 0 | 4 | 4 |
| (SEQ ID NO: 580) | | 6 | | | | | | |
| KSSQSLLGRGDLGRLNALA | C6 | RGD3- | 3 | 0 | 4 | 0 | 2 | 2 |
| (SEQ ID NO: 581) | | 7 | | | | | | |
| KSSQSLLGRGDLGRNALA | C6 | RGD3- | 3 | 0 | 3 | 0 | 3 | 3 |
| (SEQ ID NO: 582) | | 8 | | | | | | |
| KSSQSLLGRGDLGNALA | C6 | RGD1 | 2 | 0 | 0 | 0 | 0 | 1 |
| (SEQ ID NO: 573) | | | | | | | | |
| KSSQSLLGRGDLGRLKKQKDHH | C6 | RGD3- | 1 | 0 | 0 | 0 | 3 | 0 |
| (SEQ ID NO: 583) | | 9 | | | | | | |
| KSSQSLLGRGDLGRLKKQKDH | C6 | RGD3- | 2 | 0 | 0 | 0 | 1 | 0 |
| (SEQ ID NO: 584) | | 10 | | | | | | |
| KSSQSLLGRGDLGRLKKQKD | C6 | RGD3- | 2 | 0 | 0 | 0 | 2 | 1 |
| (SEQ ID NO: 585) | | 11 | | | | | | |
| KSSQSLLGRGDLGRLKKQK | C6 | RGD3- | 2 | 0 | 1 | 0 | 2 | 0 |
| (SEQ ID NO: 586) | | 12 | | | | | | |
| KSSQSLLGRGDLGRLKKQ | C6 | RGD3- | 2 | 0 | 0 | 0 | 2 | 0 |
| (SEQ ID NO: 587) | | 13 | | | | | | |
| KSSQSLLGRGDLGRLKK | C6 | RGD3- | 3 | 0 | 0 | 0 | 1 | 1 |
| (SEQ ID NO: 588) | | 14 | | | | | | |
| KSSQSLLGRGDLGRLK | C6 | RGD3- | 3 | 0 | 0 | 0 | 0 | 0 |
| (SEQ ID NO: 589) | | 15 | | | | | | |
| KSSQSLLGRGDLGRL | C6 | RGD3- | 3 | 0 | 0 | 0 | 0 | 0 |
| (SEQ ID NO: 590) | | 16 | | | | | | |

### Example 13. C6D4 Induces Th1 Bias and Increases CD8 IFN-γ Producing Cells

Seventeen C57B/7 mice were injected with 106 Lewis lung carcinoma (LLC) tumor cells and 8 were injected IP with anti-SV5 (isotype control) or 9 mice with C6D4 (both groups at 7 mg/kg). Mab injections were repeated at day 7 and tumors were harvested at day 11. Tumor infiltrating lymphoid cells were isolated from tumors by enzyme digestion and Percoll gradient centrifugation and stained for CD45, TCRb, CD4, CD8 and surface capture assay for IFNg. Live CD45+ cells were gated and B220, Ly6g, CD11c, CD11b negative, TCRb positive cells were segregated in CD4, CD8, IFN-g positive subsets. The results from this experiment are shown in FIG. 54A-54D. Shown are percentages. * p<0.05, ** p<0.01.

All documents (for example, patents, patent applications, books, journal articles, or other publications) cited herein are incorporated by reference in their entirety and for all purposes, to the same extent as if each individual document was specifically and individually indicated to be incorporated by reference in its entirety for all purposes. To the extent such documents incorporated by reference contradict the disclosure contained in the specification, the specification is intended to supersede and/or take precedence over any contradictory material.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

1-32. (canceled)

33. A method of enhancing an immune response to cancer in a human individual, the method comprising administering a sufficient amount of an antibody to the individual, thereby enhancing an immune response to the cancer, wherein:
(a) the antibody specifically binds human αvβ8 and blocks binding of TGFβ peptide to αvβ8, wherein the antibody comprises:
(i) a heavy chain variable region comprising heavy chain complementarity determining regions (CDR) 1, 2, and 3 comprising SEQ ID NOS: 520, 521 and 522, respectively; and
(ii) a light chain variable region comprising light chain CDRs 1, 2 and 3 comprising SEQ ID NOS: 541, 542 and 543, respectively; or
(b) the antibody binds to αvβ8 and αvβ6, wherein the antibody comprises:
(i) a heavy chain variable region comprising heavy chain CDRs 1, 2, and 3 comprising SEQ ID NOS: 526, 527, and 528, respectively; and
(ii) a light chain variable region comprising light chain CDRs 1, 2 and 3 comprising SEQ ID NOS: 547, 548, and 549, respectively.

34. The method of claim 33, wherein the heavy chain variable region comprises SEQ ID NO: 427.

35. The method of claim 33, wherein the light chain variable region comprises SEQ ID NO: 484.

36. The method of claim 33, wherein the heavy chain variable region comprises SEQ ID NO: 427 and the light chain variable region comprises SEQ ID NO:484.

37. The method of claim 33, wherein the heavy chain variable region comprises SEQ ID NO: 443.

38. The method of claim 33, wherein the light chain variable region comprises SEQ ID NO: 500.

39. The method of claim 33, wherein the heavy chain variable region comprises SEQ ID NO: 443 and the light chain variable region comprises SEQ ID NO:500.

40. The method of claim 33, wherein the antibody is an IgG.

41. The method of claim 33, wherein the antibody is humanized.

42. The method of claim 33, wherein the antibody is linked to a detectable label.

43. The method of claim 33, wherein the cancer is lung cancer.

44. The method of claim 33, wherein the cancer is a metastatic cancer.

45. The method of claim 33, wherein the cancer is a primary cancer.
